(19) United States
(12) Patent Application Publication
(10) Pub. No.: US 2024/0293557 A1
(43) Pub. Date: Sep. 5, 2024

(54) PROTEOLYSIS TARGETING CHIMERAS AND METHODS OF USE THEREOF

(71) Applicant: University of Maryland, Baltimore, Baltimore, MD (US)

(72) Inventors: Steven Fletcher, Baltimore, MD (US); Alexandria Marie Carlson, Baltimore, MD (US)

(21) Appl. No.: 18/568,126

(22) PCT Filed: Jun. 8, 2022
(86) PCT No.: PCT/US2022/032690

§ 371 Date: Dec. 7, 2023

Related U.S. Application Data
(60) Provisional application No. 63/208,345, filed on Jun. 8, 2021.

(51) Int. Cl. A61K 47/55; A61K 47/54; A61K 47/64; A61P 35/02

(52) U.S. Cl. CPC A61K 47/55; A61K 47/545; A61K 47/64; A61P 35/02

(57) ABSTRACT

Proteolysis-targeting chimeras (PROTACs) that indirectly inhibit Myeloid Cell Leukemia-1 (Mcl-1) oncoprotein, and methods of using the same, are provided for treating disease.

### CROSS-REFERENCE TO RELATED APPLICATIONS

The present application is an International Application which claims priority to U.S. Provisional Application No. 63/208,345 filed Jun. 8, 2021, the contents of which are incorporated herein by reference in their entirety.

### FIELD

The disclosure relates generally to Proteolysis-targeting chimeras (PROTACs) and methods of using the same for treating conditions characterized by the overexpression or unregulated activity of the protein Myeloid Cell Leukemia-1 (Mcl-1).

### BACKGROUND

The B-Cell Lymphoma-2 (Bcl-2) family of proteins regulates the intrinsic apoptosis pathway that is responsible for programmed cell death. The pathway involves protein-protein interactions (PPIs) between pro-apoptotic members of the Bcl-2 family, such as Bim, Bak and Bad, and anti-apoptotic members, such as Bcl-xL and myeloid cell leukemia-1 (Mcl-1). Through conserved hydrophobic crevices, the anti-apoptotic Bcl-2 proteins capture the BH3 α-helical domains of their pro-apoptotic counterparts, effectively “neutralizing” their cell killing functions. Evasion of apoptosis is a hallmark of cancer, and is also one culprit for the development of resistance to current chemo- and radiotherapies.

Mcl-1 overexpression and/or amplification of the Mcl-1 gene immortalizes cells, and has been observed in many human solid tumors, including pancreatic, prostate, cervical, lung and breast cancers, as well as B-cell lymphomas and hematological cancers, including acute myeloid leukemia (AML). While certain Bcl-_{XL}/Bcl-2 inhibitors perform well in clinical trials, their low affinity for Mcl-1 is a contributing factor to the observed resistance of several tumor cell lines. Moreover, the upregulation of Mcl-1 has been directly linked to the reduced efficacy of several FDA-approved anti-cancer chemotherapies. Accordingly, the pharmacologic inhibition of Mcl-1 is an attractive, complementary, and/or adjuvant strategy towards the execution of cancer cells by re-activating apoptosis.

In a similar vein to the inhibition of Bcl-_{xL}, the development of synthetic agents capable of disrupting the interaction between Mcl-1 and the BH3 α-helical “death” domains of pro-apoptotic Bcl-2 proteins could “neutralize” Mcl-1's cell survival role.

### SUMMARY

In an embodiment, the disclosure includes a compound of formula (I), or comprising a substructure of formula (I), or a pharmaceutically acceptable salt, solvate, hydrate, cocrystal, or prodrug thereof.

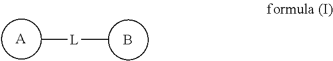

wherein in formula (I):
A comprises an Mcl-1 protein indirect inhibitor moiety;
L is a linking group; and
B comprises an E3 ubiquitin ligase ligand moiety, wherein the Mcl-1 protein inhibitor moiety thereof, and the E3 ubiquitin ligase ligand moiety thereof are each connected to L at any chemically feasible site.

In some embodiments, the Mcl-1 protein indirect inhibitor moiety comprises a CDK9 inhibitor, a dual PI3K/mTOR inhibitor, a MEK1/2 inhibitor, a FLT3 inhibitor, a JAK1/2 inhibitor, a STAT3 inhibitor, an ERK1/2 inhibitor, or any substructure thereof. In some embodiments, the CDK9 inhibitor is selected from AT7519, TG02, PHA 767491, PHA-793887, PHA-848125, BAY 1143572, BAY 1112054, Cdk9 inhibitor II (CAS 140651-18-9 from Calbiochem), DRB, AZD-5438, SNS-032, dinaciclib, LY2857785, flavopiridol, purvalanol B, CDKI-71, CDKI-73, CAN508, FIT-039, CYC065, P276-00, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, shRNAs against CDK9, anti-sense mRNA against CDK9 and anti-CDK9 antibodies, and any substructure thereof. In some embodiments, the dual PI3K/mTOR inhibitor is selected from gedatolisib (PF-05212384; PKI-587), XL765, GDC-0980, BEZ235 (NVP-BEZ235), BGT226, GSK2126458, PF-04691502, and any substructure thereof. In some embodiments, the MEK1/2 inhibitor is selected from PD334581, CI-1040, AZD6244, PD318088, PD98059, RDEA119, 6-Methoxy-7-(3-morpholin-4-yl-propoxy)-4-(4-phenoxy-phenylamino)-quinoline-3-carbonitrile, 4-[3-Chloro-4-(1-methyl-1H-imidazol-2-ylsulfanyl)-phenylamino]-6-methoxy-7-(3-morpholin-4-yl-propoxy)-quinoline-3-carbonitrile, and any substructure thereof. In some embodiments, the FLT3 inhibitor is selected from midostaurin, quizartinib, crenolanib, gilteritinib, FLX-925 (AMG-925), G-749, and any substructure thereof. In some embodiments, the JAK1/2 inhibitor is selected from Ruxolitinib, Baricitinib, Tofacitinib, and any substructure thereof. In some embodiments, the STAT3 inhibitor is selected from WP1066, S3I-201, C1-C10, and any substructure thereof. In some embodiments, the ERK1/2 inhibitor is selected from WP1066AEZS-131, AEZS-136, BVD-523, SCH-722984, SCH-772984, SCH-900353 (MK-8353), and any substructure thereof. In some embodiments, the Mcl-1 protein indirect inhibitor moiety is selected from AT7519, gedatolisib (PF-05212384; PKI-587), PD334581, TG02, and any substructure thereof.

In some embodiments, the Mcl-1 protein indirect inhibitor moiety is selected from

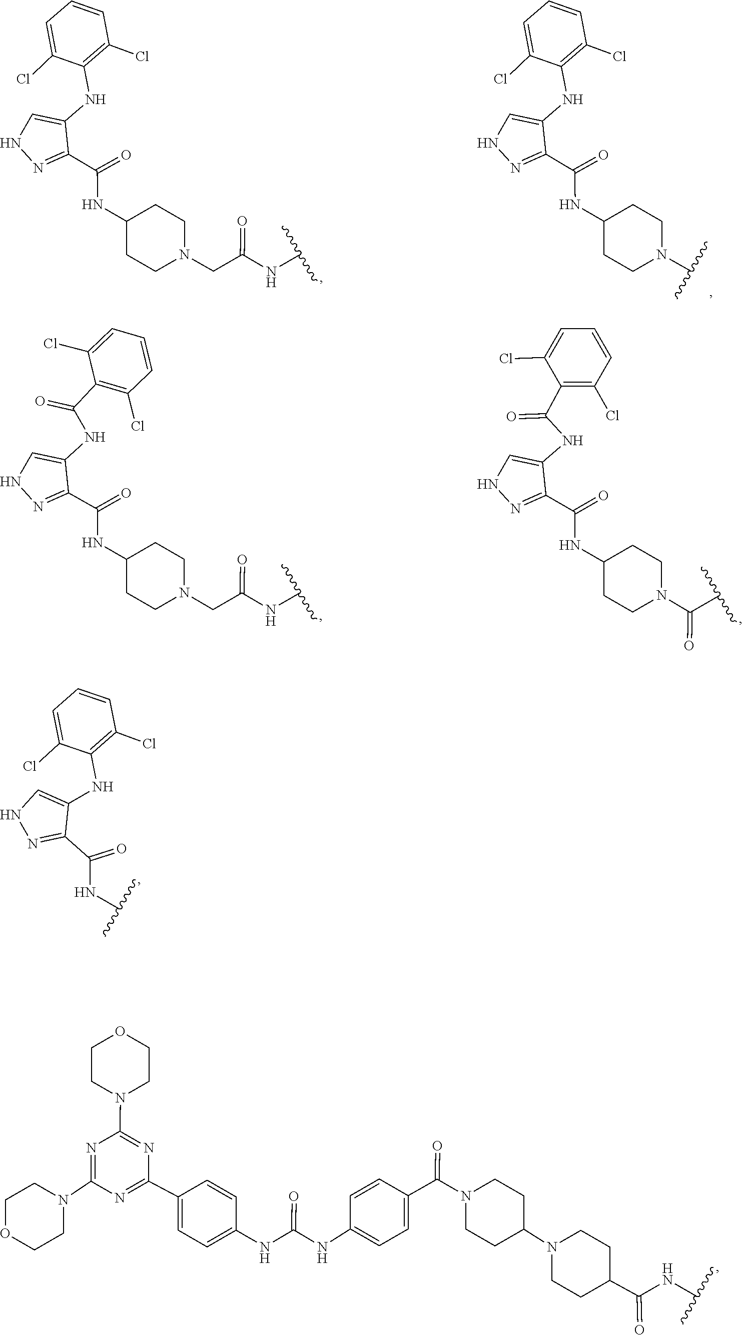 [SMILES: *NC(=O)CN1CCC(NC(=O)c2n[nH]cc2Nc2c(Cl)cccc2Cl)CC1 ; *N1CCC(NC(=O)c2n[nH]cc2Nc2c(Cl)cccc2Cl)CC1 ; *NC(=O)CN1CCC(NC(=O)c2n[nH]cc2NC(=O)c2c(Cl)cccc2Cl)CC1 ; *C(=O)N1CCC(NC(=O)c2n[nH]cc2NC(=O)c2c(Cl)cccc2Cl)CC1 ; *NC(=O)c1n[nH]cc1Nc1c(Cl)cccc1Cl ; *NC(=O)C1CCN(C2CCN(C(=O)c3ccc(NC(=O)Nc4ccc(-c5nc(N6CCOCC6)nc(N6CCOCC6)n5)cc4)cc3)CC2)CC1]

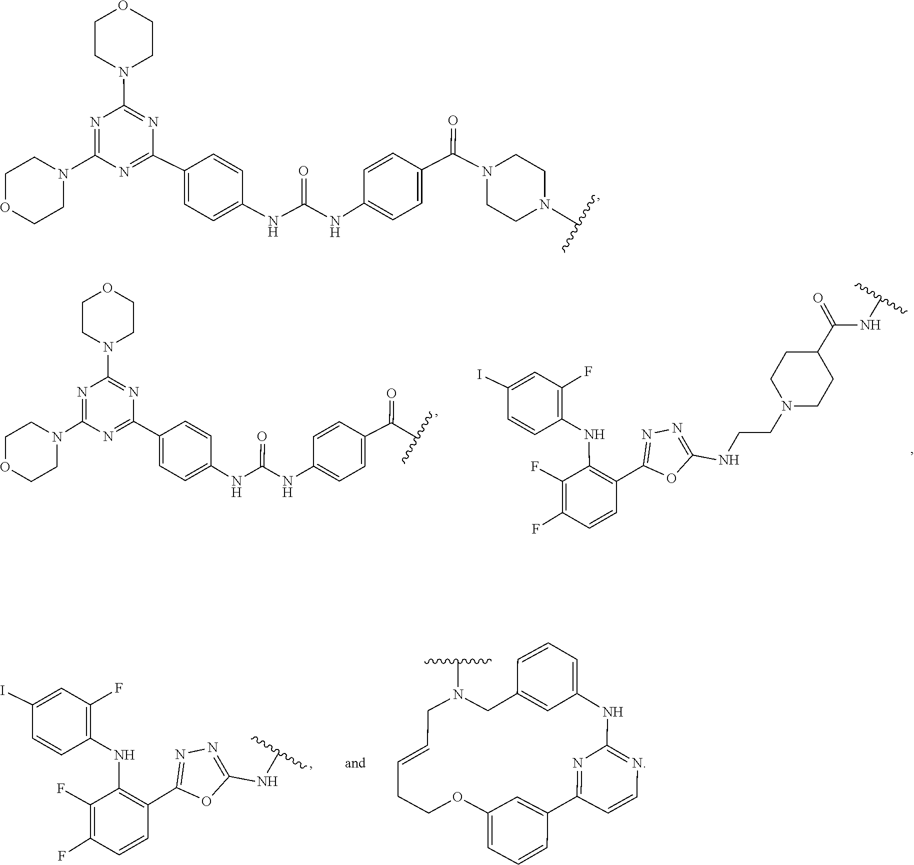 [SMILES: *N1CCN(C(=O)c2ccc(NC(=O)Nc3ccc(-c4nc(N5CCOCC5)nc(N5CCOCC5)n4)cc3)cc2)CC1 ; *C(=O)c1ccc(NC(=O)Nc2ccc(-c3nc(N4CCOCC4)nc(N4CCOCC4)n3)cc2)cc1 ; *NC(=O)C1CCN(CCNc2nnc(-c3ccc(F)c(F)c3Nc3ccc(I)cc3F)o2)CC1 ; *Nc1nnc(-c2ccc(F)c(F)c2Nc2ccc(I)cc2F)o1 ; *N1C/C=C/CCOc2cccc(c2)-c2ccnc(n2)Nc2cccc(c2)C1]

In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(N(R^{b})N(R^{b})—, —N(R^{b})S(O)_{w}—S(O)_{w}N(R^{b})—, —S(O)O—, —OS(O)_{w}—, —OS(O)O—, —O(O)P(OR^{b})O—, (O)P(O—)₃, —O(S)P(OR)O—, and (S)P(O—)₃, wherein w is 1 or 2, and R^{b} is independently hydrogen, optionally substituted alkyl, or optionally substituted aryl. In some embodiments, L comprises one or more linking groups selected from —C₁₋₁₀ alkyl-, —O—C₁₋₁₀ alkyl-, —O—, -cycloalkyl-, -heterocyclyl-, —C(O)—, —C(O)N(R^{b})— wherein R^{b} is hydrogen or optionally substituted alkyl, and —N(R^{b})— wherein R^{b} is optionally substituted alkyl. In some embodiments, the heterocyclyl is piperidyl or piperazinyl.

In some embodiments, L comprises one or more linking groups selected from

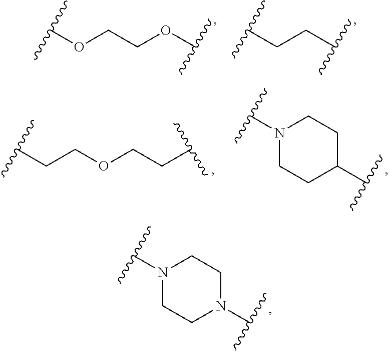

—CH₂C(O)NH—, —C(O)NH—, —C(O)CH₂—, and —OCH₂C(O)—. In some embodiments, L comprises one or more linking groups selected from

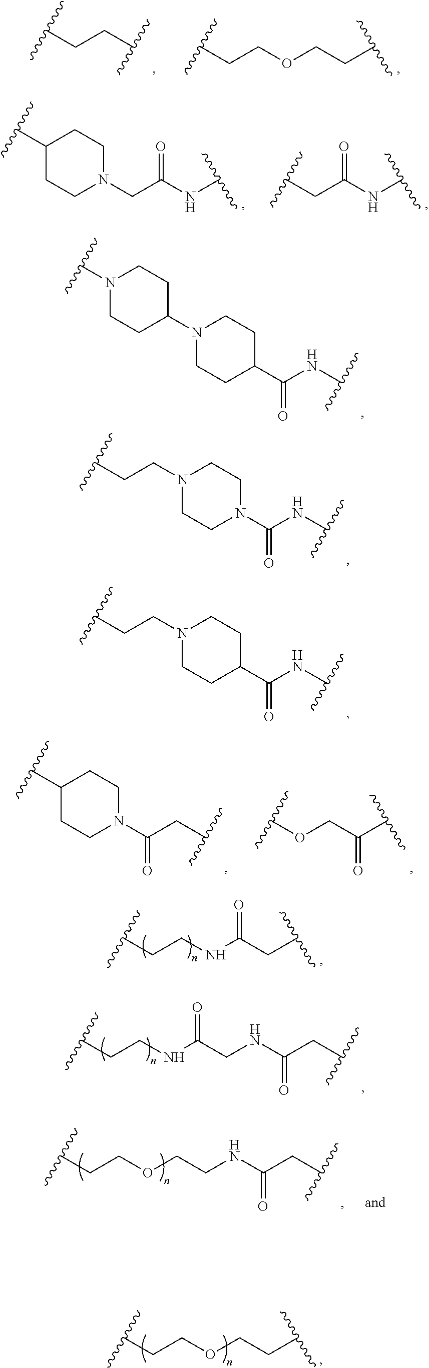

wherein each n is independently n=1-5.

In some embodiments, the E3 ubiquitin ligase ligand moiety comprises cereblon (CRBN) ligand, a mouse double minute 2 (MDM2) ligand, a Von Hippel-Lindau (VHL) ligand, or any substructure thereof. In some embodiments, the CRBN ligand is selected from thalidomide, lenalidomide, pomalidomide, and any substructure thereof. In some embodiments, the MDM2 ligand is selected from idasanutlin, RG7112, RG7388, MI 773/SAR 405838, AMG 232, DS-3032b, RO6839921, RO5045337, RO5503781, CGM-097, MK-8242, and any substructure thereof. In some embodiments, the VHL ligand is selected from VHL ligand 1 (VHL-1), VHL ligand 2 (VHL-2), VH032, and any substructure thereof. In some embodiments, the E3 ubiquitin ligase ligand moiety is selected from thalidomide, idasanutlin, VHL ligand 1 (VHL-1), and any substructure thereof. In some embodiments, the E3 ubiquitin ligase ligand moiety is selected from

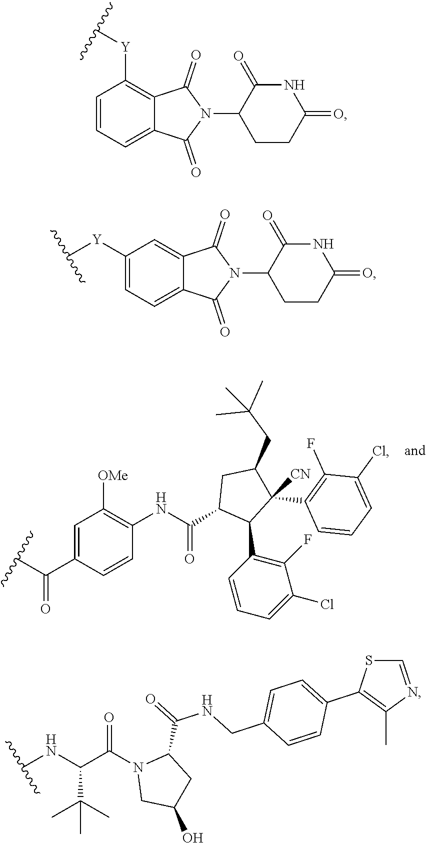

wherein Y is selected from —N(R)—, —N(H)—, and —O—, wherein R is optionally substituted alkyl.

In some embodiments, the E3 ubiquitin ligase ligand moiety is selected from

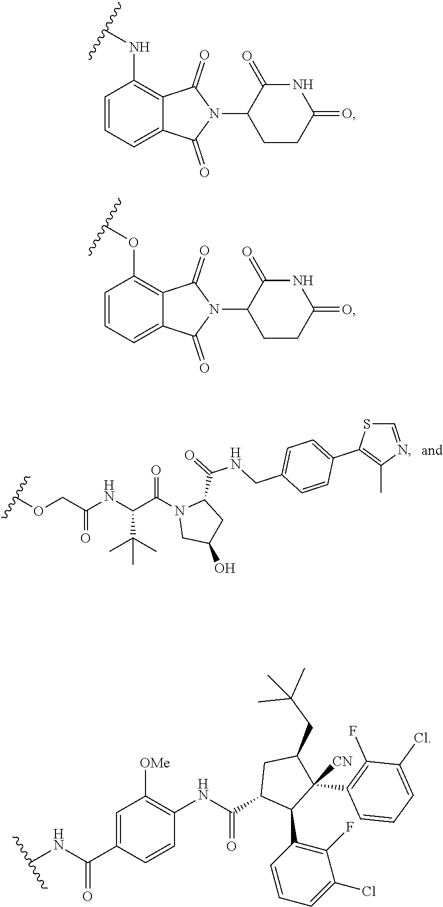 [SMILES: *Nc1cccc2c1C(=O)N(C1CCC(=O)NC1=O)C2=O ; *Oc1cccc2c1C(=O)N(C1CCC(=O)NC1=O)C2=O ; *OCC(=O)N[C@H](C(=O)N1C[C@H](O)C[C@H]1C(=O)NCc1ccc(-c2scnc2C)cc1)C(C)(C)C ; *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1]

In some embodiments, the compound of formula (I) or formula 1001-1460 has a molecular weight not greater than about 2000 g/mol, or about 1900 g/mol, or about 1800 g/mol, or about 1700 g/mol, or about 1600 g/mol, or about 1500 g/mol, or about 1400 g/mol, or about 1300 g/mol, or about 1200 g/mol or about 1100 g/mol, or about 1000 g/mol.

In some embodiments, the compound of formula (I) is a compound of any one of formula 1001-1460, or a pharmaceutically acceptable salt, solvate, hydrate, cocrystal, or prodrug thereof:

In one aspect, the disclosure provides a pharmaceutical composition comprising one or more of compounds of any one of formula (I), formula 1001-1460, or a pharmaceutically acceptable salt thereof, and a physiologically compatible carrier medium.

In one aspect, the disclosure provides a pharmaceutical composition for treating or preventing a disease or disorder alleviated by inhibiting and/or indirectly inhibiting Mcl-1 protein activity, the pharmaceutical composition comprising one or more compounds according to any one of formula (I), formula 1001-1460, or a pharmaceutically acceptable salt thereof, and a physiologically compatible carrier medium. In some embodiments, the disease or disorder is cancer. In some embodiments, the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is a blood cancer. In some embodiments, the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma. In some embodiments, the cancer is acute myeloid leukemia (AML).

In one aspect, the disclosure provides a pharmaceutical composition for treating or preventing from acute myeloid leukemia (AML), the pharmaceutical composition comprising one or more compounds according any one of formula (I), formula 1001-1480, or a pharmaceutically acceptable salt thereof, and a physiologically compatible carrier medium.

In one aspect, the disclosure provides a method of treating or preventing a disease or disorder alleviated by inhibiting and/or indirectly inhibiting Mcl-1 protein activity in a patient in need of said treatment or prevention, the method comprising administering a therapeutically effective amount of one or more compounds of formula (I), formula 1001-1460, or a pharmaceutically acceptable salt thereof. In some embodiments, the disease or disorder is cancer. In some embodiments, the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is a blood cancer. In some embodiments, the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma. In some embodiments, the cancer is acute myeloid leukemia (AML).

In one aspect, the disclosure provides a method of treating or preventing acute myeloid leukemia (AML) in a patient in need of said treatment or prevention, the method comprising administering a therapeutically effective amount of one or more compounds of any one of formula (I), formula 1001-1460, or a pharmaceutically acceptable salt thereof.

### BRIEF DESCRIPTION OF THE DRAWINGS

The foregoing summary, as well as the following detailed description of embodiments of the disclosure, will be better understood when read in conjunction with the appended drawings and figures.

In the drawings:

FIG. 1 illustrates a non-limiting general strategy to prepare indirect MCL-1 PROTACs of the disclosure. X═NHCO or OCH₂CONH depending on whether the E3 ligase ligand presents an acid or an amine for conjugation.

FIG. 2 illustrates the structure of parent AML drugs with solvent-exposed groups in blue; yellow arrows indicate non-limiting examples of grafting points which can be modified to include a carboxylic acid for conjugation purpose while retaining the solubilizing, protonatable amines. Protein targets indicated in bold.

FIG. 3 illustrates the structure of carboxylic acid-modified AML drug-candidates.

FIG. 4 illustrates a non-limiting synthetic scheme of the preparation of E3 ligase ligands and linkers. L is defined as in FIG. 1.

FIG. 5 illustrates the structure of a non-limiting example of an indirect MCL-1 PROTAC based on the CDK9 inhibitor AT7519 that is designed to be degraded by the CRBN E3 ligase. MW=827.

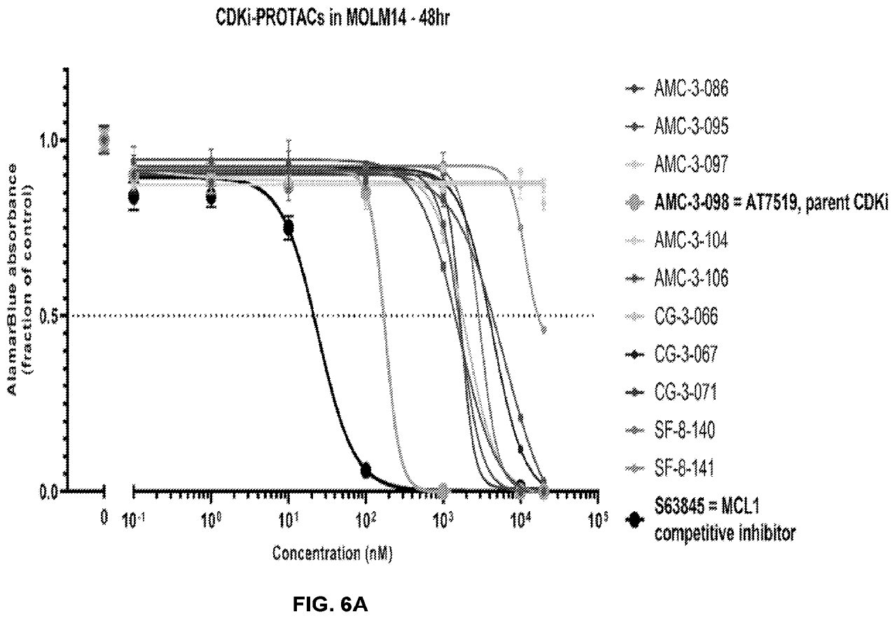

FIGS. 6A-6B illustrate the viability of MOLM14 cells measured in presence of several PROTAC compounds. FIG. 6A illustrates the viability results after 48 hours. FIG. 6B illustrates the viability results after 72 hours.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include the compounds described herein and, more specifically, the one or more compounds of formula (I) or formula 1001-1460.

The terms “active pharmaceutical ingredient” and “drug” may also include those compounds described herein that indirectly inhibit Mcl-1 protein and thereby modulate Mcl-1 protein activity.

The term “isostere” refers to a group or molecule whose chemical and/or physical properties are similar to those of another group or molecule. A “bioisostere” is a type of isostere and refers to a group or molecule whose biological properties are similar to those of another group or molecule. For example, for the compounds described herein, a carboxylic acid may be replaced by one of the following bioisosteres for carboxylic acids, including, without limitation, alkyl esters (COOR), acylsulfonamides (CONR—SO₂R), hydroxamic acids (CONR—OH), hydroxamates (CONR—OR), tetrazoles, hydroxyisoxazoles, isoxazol-3-ones, and sulfonamides (SO₂NR), where each R may independently represent hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “treat,” “treatment,” and/or “treating” may refer to the management of a disease, disorder, or pathological condition (e.g., pain, a neurological disorder, diarrhea, coughing, muscular tension, and glaucoma), or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. In some embodiments, the biological molecules modulated by the methods and compounds of the disclosure to effect treatment may include one or both of the δ-opioid receptor and the μ-opioid receptor.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” or “physilogically compatible” carrier or carrier medium is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

A “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of formulas I, II, IIIa, IIIb, and IV. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of formula I, IL, IIIa, IIIb, and IV) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Acylsulfonamide” refers to the group —C(═O)NR^{a}—S(═O)R^{a}, where each R^{a} is independently hydrogen, alkyl, fluoroalkyl, carbocyclyl, carbocyclylalkyl, aryl, aralkyl, heterocycloalkyl, heterocycloalkylalkyl, heteroaryl, or heteroarylalkyl.

“Carboxaldehyde” refers to a —(C═O)H radical.

“Carbonyl” refers to the group —C(═O)—. Carbonyl groups may be substituted with the following exemplary substituents: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —NR^{a}—OR^{a}—, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, acylsulfonamido, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR, —SRI, —S(O)ₜR^{a}— (where t is 1 or 2), —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, acylsulfonamido, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxyxC═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR, —SRI, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR, —SRI, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR, —S(O)R^{a}— (where t is 1 or 2), —OC(O)—R^{a}, —N(R)₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, amino, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R)₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(R^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo.

The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, acylsulfonamido, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, isoxazol-3-one, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” or “heterocyclyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Hydroxamate” refers to the —C(O)NR^{a}OR^{a} moiety, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(f)” is used to designate a racemic mixture where appropriate.

“Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight.

The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization.

“Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, 3rd Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxamate, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### DETAILED DESCRIPTION

Acute myeloid leukemia (AML) urgently needs novel drugs. AML, one of the deadliest and most rapidly fatal human cancers, most frequently occurs in senior adults, where outcomes are poor, with <2.5% long-term survival in patients >60 years old. Moreover, frail seniors can tolerate only lower-intensity chemotherapies, such as venetoclax (VEN) combined with a hypomethylating agent (HMA), azacytidine or decitabine; however, the median survival with VEN/HMA is only 17.5 months. Not only are multiple potent, low-toxicity, new multi-drug combinations needed to cure AML, but AML provides an excellent setting for translational research that may also extend to other cancers, since 1) AML xenograft models and patients are measurably responsive to many treatments (often not as demonstrable in solid cancers), and 2) leukemia cells, normal cells of origin and microenvironment are accessible for mechanistic, and PK/PD studies.

Mcl-1 is an important target in AML and other cancers. The anti-apoptotic members of the BCL-2 family, commonly BCL-2 itself and/or myeloid cell leukemia 1 (Mcl-1) and/or B-cell lymphoma-extra large (BCL-xL), promote survival of AML cells (and other cancer cell types) by disarming cell killing through seizing pro-apoptotic sister proteins via their BCL-2 homology domain-3 (BH3) death domains. Decades of research culminated in the discovery of Venetoclax (VEN), a BH3-mimetic that specifically disrupts binding of BCL-2 (but not other anti-apoptotic proteins) to pro-apoptotic proteins. With BCL-2 incapacitated, multidomain pro-apoptotic proteins BCL-2 antagonist/killer 1 (BAK) and BCL-2 associated X (BAX) trigger mitochondrial outer membrane permeabilization and cell death through the intrinsic apoptosis pathway. However, although the FDA recently approved VEN in combination with an HMA or low-dose cytarabine for seniors with AML, resistance to these treatments soon emerges, commonly due to Mcl-1 (or BCL-xL) upregulation.

Direct (competitive) inhibition of Mcl-1 demands exquisite affinities—rendering mutational resistance more probable—and may be cardiotoxic. Overexpression of Mcl-1 contributes to AML/cancer development and progression, and to resistance to many antineoplastic drugs.

Accordingly, the development of Mcl-1 inhibitors is a major goal in oncology. Again, through BH3 mimicry, extensive research has culminated in several competitive Mcl-1 inhibitors spanning a range of chemotypes. Perhaps due to underestimation of the strength of interaction between Mcl-1 and its (patho)physiologic binding partners in a cellular setting (single-digit nanomolar binding affinities determined in cell-free systems involve only the BH3 domain of the pro-apoptotic protein, rather than the full protein), picomolar affinities are required for competitive Mcl-1 inhibitors to (re)activate apoptosis. The requirement for exquisite affinities is an early warning sign that even the subtlest of mutations may confer drug resistance, with the native pro-apoptotic ligands winning the game of “cat-and-mouse”. Furthermore, several cell types depend on Mcl-1 function for survival, including hematopoietic stem cells, cardiomyocytes and hepatocytes; Mcl-1 knockout in mice is embryonic lethal. Indeed, Amgen's clinical trial of Mcl-1 inhibitor AMG176 was halted due to cardiotoxicity (with one fatality), and another was voluntarily suspended, although the clinical trial of AstraZeneca's Mcl-1 inhibitor AZD5991 remains ongoing.

PROTACs require only sub-stoichiometries and may be more refractory upregulation and point mutations. Traditional non-covalent drugs are vulnerable to 1) mutational resistance and 2) upregulation of target protein. The emerging class of PROteolysis-TArgeting Chimeras (PROTACs) may address both of these limitations. PROTACs are a type of bivalent inhibitor in which the ligand for the protein of interest (POI) is linked to an E3 ubiquitin ligase ligand, such as thalidomide, which results in polyubiquitination of the POI followed by its recruitment to the proteasome for degradation. Of particular significance is the catalytic nature of a PROTAC, since it is released upon degradation of the POI, permitting additional POI molecules to be degraded. This may markedly reduce the dosing required for therapeutic effect, and is also predicted to counter resistance associated with POI upregulation. Since a PROTAC does not need to be built from a ligand with exquisite affinity for the POI, PROTACs are predicted to be more impervious to binding site mutations than their parent drugs, as observed with PROTAC-driven rescue of BTK mutational resistance to ibrutinib.

Transcriptional level—CDK9: Inhibition of cyclin-dependent kinase 9 (CDK9) downregulates the transcription of several survival genes, including Mcl-1 and MYC, and inhibition of CDK9 is an emerging strategy to downregulate Mcl-1. The CDK9 inhibitors alvocidib and A-1592668 recently demonstrated synergies with VEN in AML. Inhibition of CDK5 also proved synergistic with navitoclax. AT7519 is a CDK9 inhibitor that binds CDK5 as well (albeit less tightly), and is undergoing clinical evaluation in solid and hematological malignancies. While PROTACs of CDK9 inhibitors have been reported, there are no reports of an AT7519 PROTAC nor of a dual CDK5/CDK9 PROTAC.

Translational level—PI3K/Akt/mTOR: The PI3K/Akt/mTOR pathway is hyperactivated in many AML patients, and is involved in Mcl-1 translational control. However, mTOR inhibitors that are selective for only the mTOR complex 1, i.e. mTORC1 inhibitors, have resulted in poor clinical outcomes due to resistance (somewhat owing to the lack of mTORC2 inhibition, which results in a feedback activation of PI3K/Akt, first with a phosphorylating activation of Akt, which then results in activation of PI3K, and so on). This motivated research into the discovery of dual PI3K/mTOR inhibitors that can potently bind both mTORC1 and mTORC2; BEZ235 is such an inhibitor and has demonstrated synergy with VEN through Mcl-1 downregulation at the translational level. Another dual PI3K/mTOR inhibitor, gedatolisib, is presently in combination therapy clinical trials for AML. There are currently no reports of a PROTAC based on gedatolisib, nor of a dual PI3K/mTOR PROTAC.

Post-translational level—MAPK: The RAS/RAF/MEK/ERK, or MAPK, pathway has been targeted at each stage for cancer. The MEK inhibitor cobimetidine has recently demonstrated synergy with VEN through downregulation of Mcl-1, since blockade of the MAPK pathway prevents an ERK-catalyzed stabilizing phosphorylation of T163 of Mcl-1. While an MEK PROTAC was reported in 2020, PROTACs of the disclosure include novel chemotypes, such as in MEK1 inhibitor PD334581.

In one aspect, the present disclosure provides novel PROTACS that function as indirect inhibitors of Mcl-1 and are useful in treating diseases or disorders associated with dysregulation of Mcl-1. In some embodiments, the PROTACs of the disclosure bind to pproteins involved I the regulation of the oncoprotein, and recruit them to the proteasome for destruction. In some embodiments, the PROTACs of the disclosure are useful as treatments for a range of cancers, and indirectly downregulate Mcl-1, is known to promote tumorigenesis and is responsible for chemoresistance.

### Mcl-1 Indirect Inhibitors

In one aspect, the disclosure provides compounds that are Mcl-1 indirect inhibitors and/or indirect modulators of Mcl-1 protein activity.

In some embodiments, the compounds described herein bind other targets associated with a disease or disorder, such as AML, resulting in and as a consequence lead to downregulation of MCL-1. Any target that results in the downregulation and/or inhibition of MCL-1 is contemplated by the disclosure. In some embodiments, the compounds bind to one or more of CDK9, PI3K/mTOR, MEK1/2, FLT3, JAK1/2, STAT3, and ERK1/2. In some embodiments, the compounds inhibit one or more of CDK9, PI3K/mTOR, MEK1/2, FLT3, JAK1/2, STAT3, and ERK1/2.

In some embodiments, the compounds described above may be delivered as listed or as a pharmaceutically acceptable salt, solvate, hydrate, cocrystal, tautomer, or prodrug thereof. In one aspect, the disclosure provides a compound of formula (I), or comprising a substructure of formula (I), or a pharmaceutically acceptable salt, solvate, hydrate, cocrystal, or prodrug thereof:

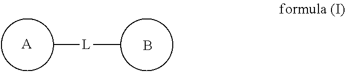

wherein in formula (I):
A comprises an Mcl-1 protein indirect inhibitor moiety;
L is a linking group; and
B comprises an E3 ubiquitin ligase ligand moiety, wherein the Mcl-1 protein inhibitor moiety thereof, and the E3 ubiquitin ligase ligand moiety thereof are each connected to L at any chemically feasible site.

Any chemical moiety or substructure that provides indirect inhibition of the Mcl-1 protein is contemplated by the present disclosure. In some embodiments, the Mcl-1 protein indirect inhibitor moiety comprises a CDK9 inhibitor, a dual PI3K/mTOR inhibitor, a MEK1/2 inhibitor, a FLT3 inhibitor, a JAK1/2 inhibitor, a STAT3 inhibitor, and an ERK1/2 inhibitor, or any substructure thereof.

Any CDK9 inhibitor is contemplated by the present disclosure. Non-limiting examples of CDK9 inhibitors include AT7519, TG02, PHA 767491, PHA-793887, PHA-848125, BAY 1143572, BAY 1112054, Cdk9 inhibitor II (CAS 140651-18-9 from Calbiochem), DRB, AZD-5438, SNS-032, dinaciclib, LY2857785, flavopiridol, purvalanol B, CDKI-71, CDKI-73, CAN508, FIT-039, CYC065, P276-00, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, shRNAs against CDK9, anti-sense mRNA against CDK9 and anti-CDK9 antibodies, and any substructure thereof.

Any dual PI3K/mTOR inhibitor is contemplated by the present disclosure. Non-limiting examples of dual PI3K/mTOR inhibitors include gedatolisib (PF-05212384; PKI-587), XL765, GDC-0980, BEZ235 (NVP-BEZ235), BGT226, GSK2126458, and PF-04691502, and any substructure thereof.

Any MEK1/2 inhibitor is contemplated by the present disclosure. Non-limiting examples of MEK1/2 inhibitors include PD334581, CI-1040, AZD6244, PD318088, PD98059, RDEA119, 6-Methoxy-7-(3-morpholin-4-yl-propoxy)-4-(4-phenoxy-phenylamino)-quinoline-3-carbonitrile, and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, and any substructure thereof.

Any FLT3 inhibitor is contemplated by the present disclosure. Non-limiting examples of FLT3 inhibitors include midostaurin, quizartinib, crenolanib, gilteritinib, FLX-925 also known as AMG-925, and G-749, and any substructure thereof.

Any JAK1/2 inhibitor is contemplated by the present disclosure. Non-limiting examples of JAK1/2 inhibitors include Ruxolitinib, Baricitinib, and Tofacitinib, and any substructure thereof.

Any STAT3 inhibitor is contemplated by the present disclosure. Non-limiting examples of STAT3 inhibitors include WP1066, S3I-201 and C1-C10, and any substructure thereof.

Any ERK1/2 inhibitor is contemplated by the present disclosure. Non-limiting examples of ERK1/2 inhibitors include WP1066AEZS-131, AEZS-136, BVD-523, SCH-722984, SCH-772984, and SCH-900353 (MK-8353), and any substructure thereof.

In some embodiments, the Mcl-1 protein indirect inhibitor moiety is selected from:

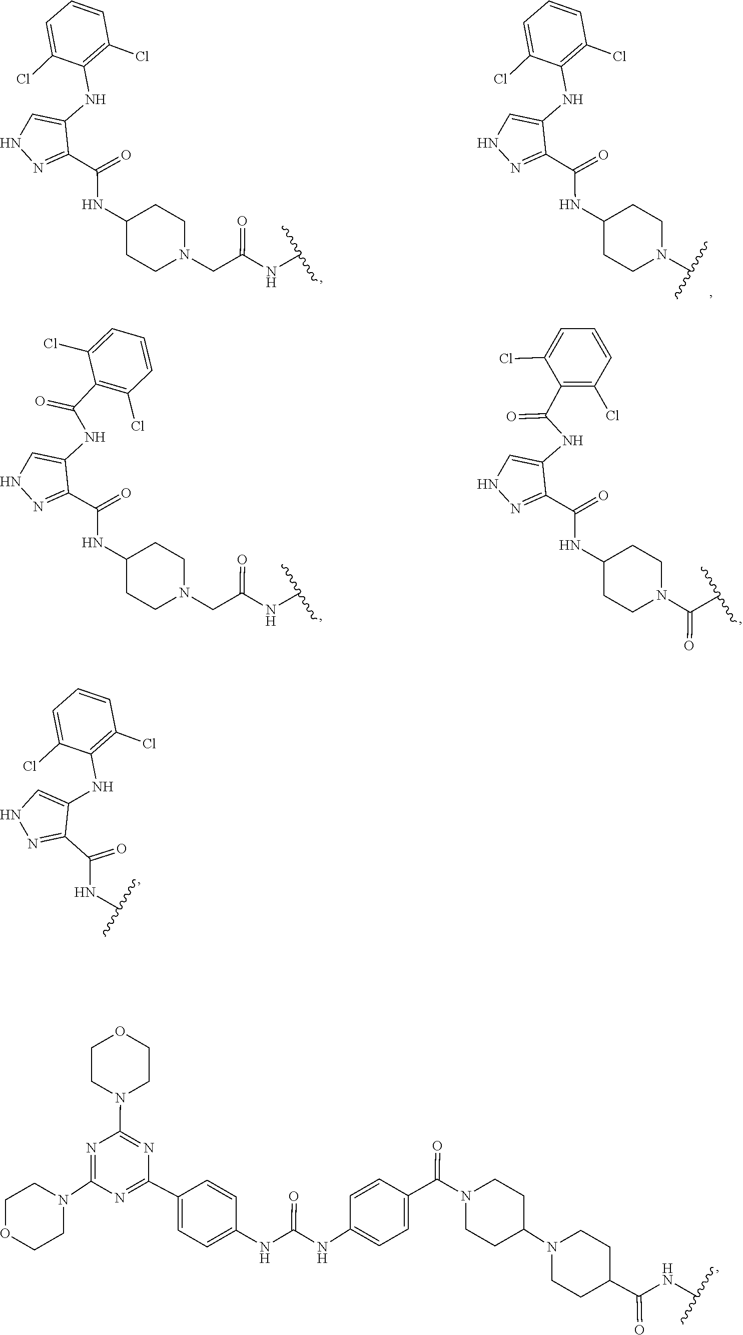 [SMILES: *NC(=O)CN1CCC(NC(=O)c2n[nH]cc2Nc2c(Cl)cccc2Cl)CC1 ; *N1CCC(NC(=O)c2n[nH]cc2Nc2c(Cl)cccc2Cl)CC1 ; *NC(=O)CN1CCC(NC(=O)c2n[nH]cc2NC(=O)c2c(Cl)cccc2Cl)CC1 ; *C(=O)N1CCC(NC(=O)c2n[nH]cc2NC(=O)c2c(Cl)cccc2Cl)CC1 ; *NC(=O)c1n[nH]cc1Nc1c(Cl)cccc1Cl ; *NC(=O)C1CCN(C2CCN(C(=O)c3ccc(NC(=O)Nc4ccc(-c5nc(N6CCOCC6)nc(N6CCOCC6)n5)cc4)cc3)CC2)CC1]

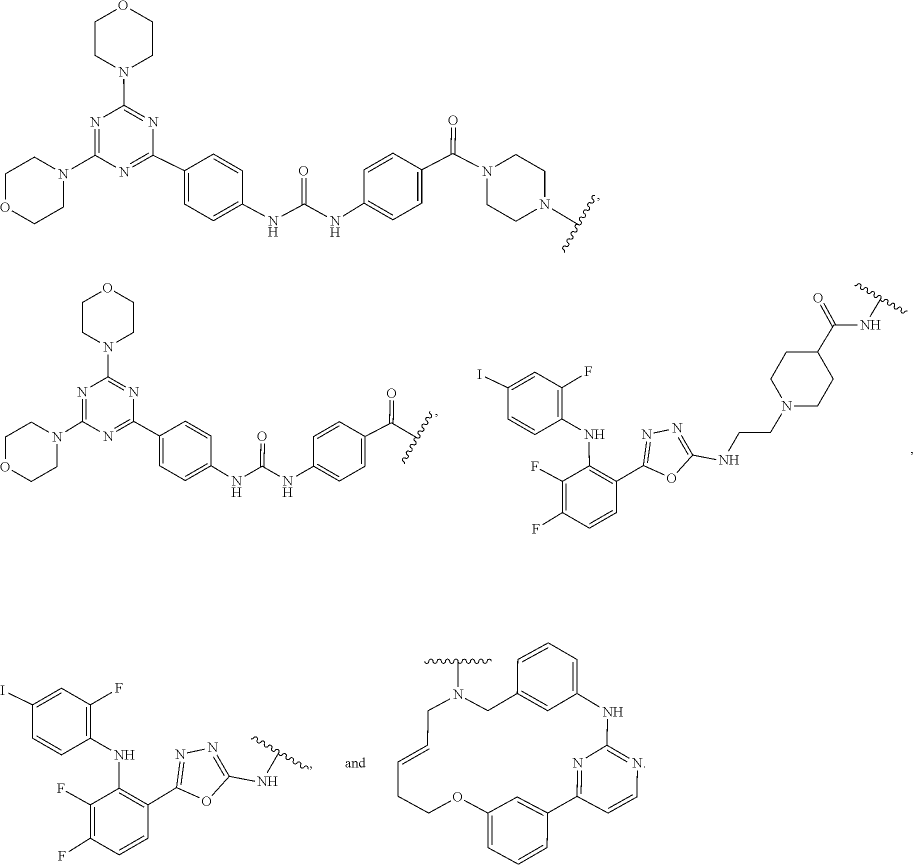 [SMILES: *N1CCN(C(=O)c2ccc(NC(=O)Nc3ccc(-c4nc(N5CCOCC5)nc(N5CCOCC5)n4)cc3)cc2)CC1 ; *C(=O)c1ccc(NC(=O)Nc2ccc(-c3nc(N4CCOCC4)nc(N4CCOCC4)n3)cc2)cc1 ; *NC(=O)C1CCN(CCNc2nnc(-c3ccc(F)c(F)c3Nc3ccc(I)cc3F)o2)CC1 ; *Nc1nnc(-c2ccc(F)c(F)c2Nc2ccc(I)cc2F)o1 ; *N1C/C=C/CCOc2cccc(c2)-c2ccnc(n2)Nc2cccc(c2)C1]

Any organic linker L is contemplated by the present disclosure. In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b}), —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R)C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl. In some embodiments, L comprises one or more linking groups selected from —C₁₋₁₀ alkyl-, —O—C₁₋₁₀ alkyl-, —O—, -cycloalkyl-, -heterocyclyl-, —C(O)—, —C(O)N(R^{b})— wherein R^{b} is hydrogen or optionally substituted alkyl, and —N(R^{b})— wherein R^{b} is optionally substituted alkyl. In some embodiments, the heterocyclyl is piperidyl or piperazinyl.

In some embodiments, L comprises one or more linking groups selected from

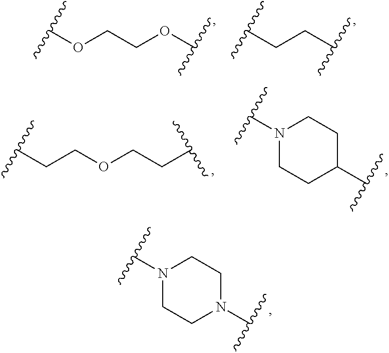

—CH₂C(O)NH—, —C(O)NH—, —C(O)CH₂—, and —OCH₂C(O)—.

In some embodiments, L comprises one or more linking groups selected from

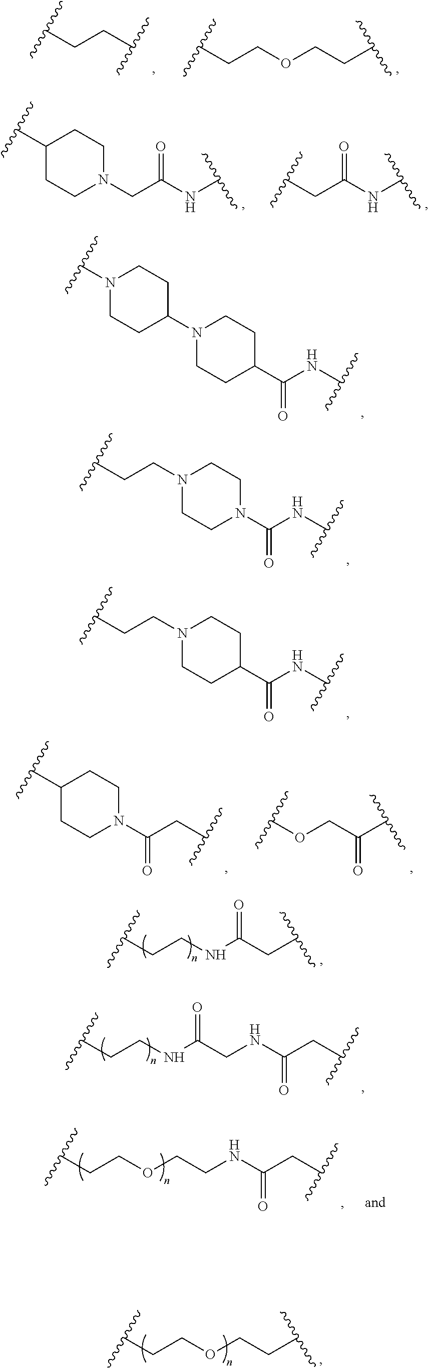

wherein n=1-5.

Any E3 ubiquitin ligase ligand moiety is contemplated by the present disclosure. In some embodiments, the E3 ubiquitin ligase ligand moiety comprises cereblon (CRBN) ligand, a mouse double minute 2 (MDM2) ligand, a Von Hippel-Lindau (VHL) ligand, or any substructure thereof.

Any CRBN ligand is contemplated by the present disclosure. Non-limiting examples CRBN ligands include thalidomide, lenalidomide, pomalidomide, and any substructure thereof.

Any MDM2 ligand is contemplated by the present disclosure. Non-limiting examples MDM2 ligands include idasanutlin, RG7112, RG7388, MI 773/SAR 405838, AMG 232, DS-3032b, RO6839921, RO5045337, RO5503781, CGM-097, MK-8242, and any substructure thereof.

Any VHL ligand is contemplated by the present disclosure. Non-limiting examples MDM2 ligands include VHL ligand 1 (VHL-1), VHL ligand 2 (VHL-2), VH032, and any substructure thereof.

In some embodiments, the E3 ubiquitin ligase ligand moiety is selected from thalidomide, idasanutlin, VHL ligand 1 (VHL-1), and any substructure thereof.

In some embodiments, the E3 ubiquitin ligase ligand moiety is selected from:

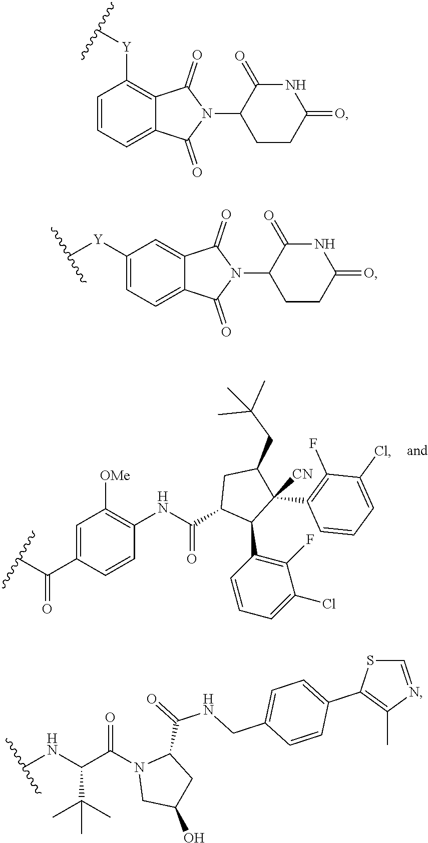

wherein Y is selected from —N(R)—, —N(H)—, and —O—, wherein R is optionally substituted alkyl.

In some embodiments, the E3 ubiquitin ligase ligand moiety is selected from:

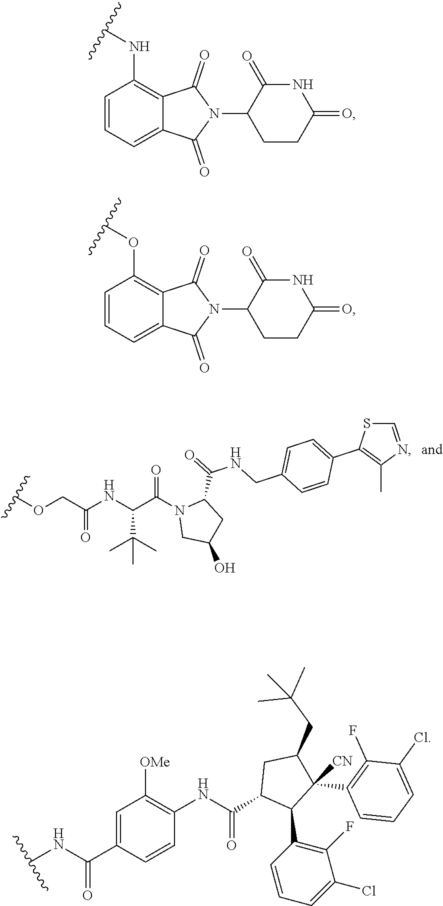 [SMILES: *Nc1cccc2c1C(=O)N(C1CCC(=O)NC1=O)C2=O ; *Oc1cccc2c1C(=O)N(C1CCC(=O)NC1=O)C2=O ; *OCC(=O)N[C@H](C(=O)N1C[C@H](O)C[C@H]1C(=O)NCc1ccc(-c2scnc2C)cc1)C(C)(C)C ; *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1]

In some embodiments, the compound of formula (I) is a compound of any one of formula 1001-1460, or a pharmaceutically acceptable salt, solvate, hydrate, cocrystal, or prodrug thereof:

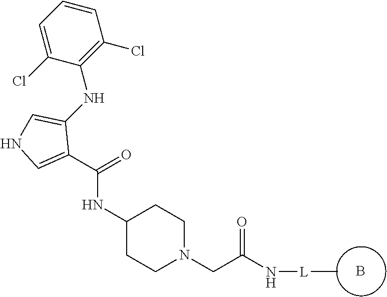

| For- | | | |
|---|---|---|---|
| mu- | | | |
| la | | | |
| No. | L | n | B |
| 1001 | 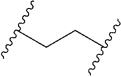 [SMILES: *CC*] | — | 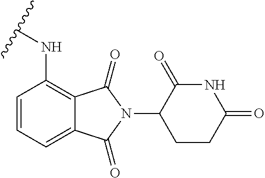 [SMILES: *Nc1cccc2c1C(=O)N(C1CCC(=O)NC1=O)C2=O] |
| 1002 | 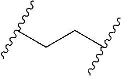 [SMILES: *CC*] | — | 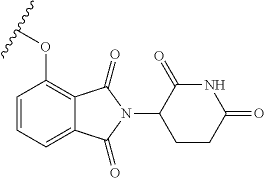 [SMILES: *Oc1cccc2c1C(=O)N(C1CCC(=O)NC1=O)C2=O] |
| 1003 | 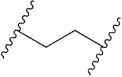 [SMILES: *CC*] | — | 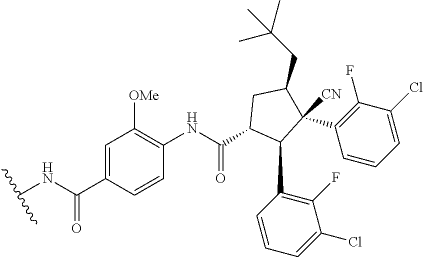 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1004 | 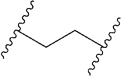 [SMILES: *CC*] | — | 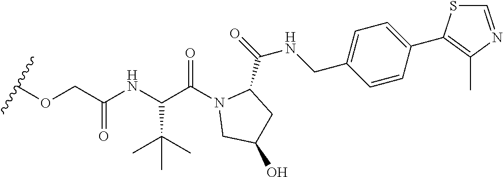 |
| 1005 | 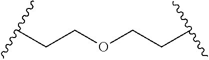 [SMILES: *CCOCC*] | — | 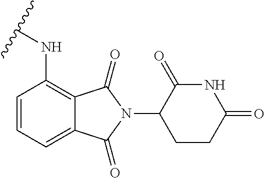 [SMILES: *Nc1cccc2c1C(=O)N(C1CCC(=O)NC1=O)C2=O] |
| 1006 | 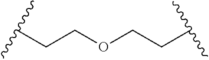 [SMILES: *CCOCC*] | — | 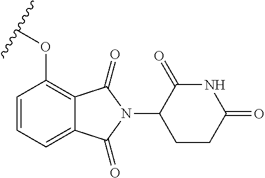 [SMILES: *Oc1cccc2c1C(=O)N(C1CCC(=O)NC1=O)C2=O] |
| 1007 | 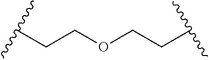 [SMILES: *CCOCC*] | — | 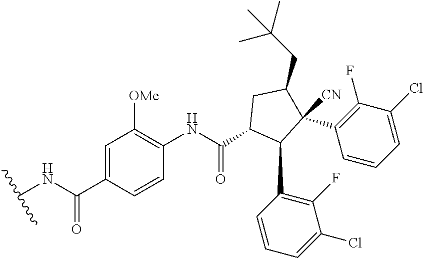 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1008 | 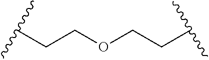 [SMILES: *CCOCC*] | — | 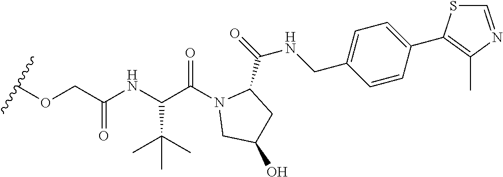 |
| 1009 | 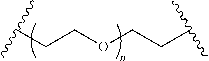 | 1 | 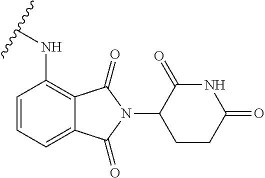 [SMILES: *Nc1cccc2c1C(=O)N(C1CCC(=O)NC1=O)C2=O] |
| 1010 | 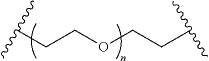 | 2 | 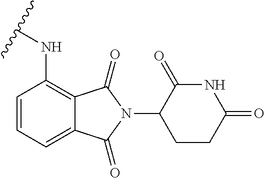 [SMILES: *Nc1cccc2c1C(=O)N(C1CCC(=O)NC1=O)C2=O] |
| 1011 | 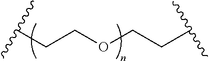 | 3 | 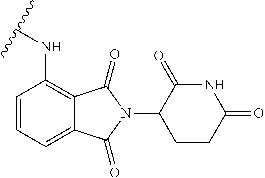 [SMILES: *Nc1cccc2c1C(=O)N(C1CCC(=O)NC1=O)C2=O] |
| 1012 | 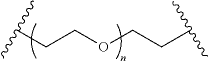 | 4 | 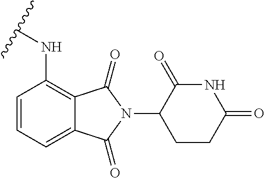 [SMILES: *Nc1cccc2c1C(=O)N(C1CCC(=O)NC1=O)C2=O] |
| 1013 | 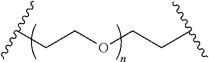 | 5 | 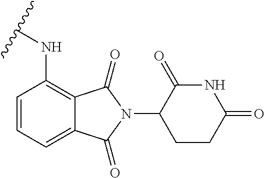 [SMILES: *Nc1cccc2c1C(=O)N(C1CCC(=O)NC1=O)C2=O] |
| 1014 | 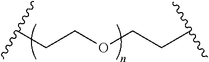 | 1 | 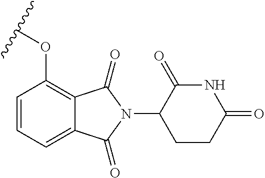 [SMILES: *Oc1cccc2c1C(=O)N(C1CCC(=O)NC1=O)C2=O] |
| 1015 | 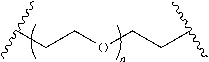 | 2 | 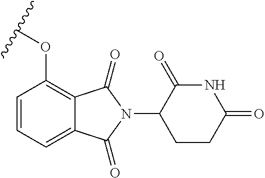 [SMILES: *Oc1cccc2c1C(=O)N(C1CCC(=O)NC1=O)C2=O] |
| 1016 | 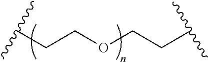 | 3 | 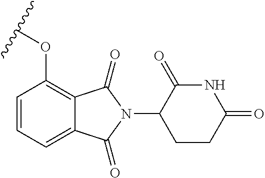 [SMILES: *Oc1cccc2c1C(=O)N(C1CCC(=O)NC1=O)C2=O] |
| 1017 | 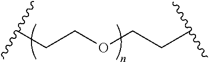 | 4 | 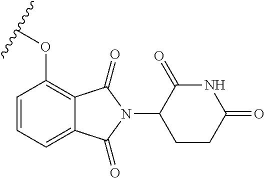 [SMILES: *Oc1cccc2c1C(=O)N(C1CCC(=O)NC1=O)C2=O] |
| 1018 | 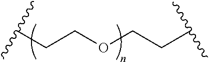 | 5 | 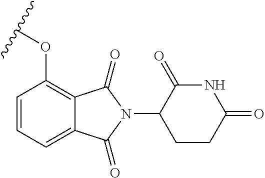 [SMILES: *Oc1cccc2c1C(=O)N(C1CCC(=O)NC1=O)C2=O] |
| 1019 | 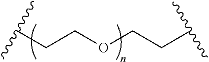 | 1 | 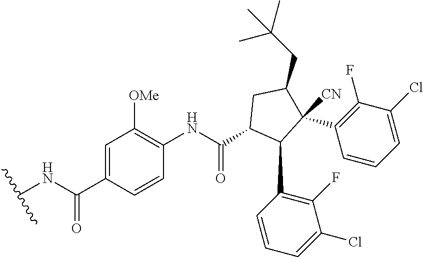 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1020 | 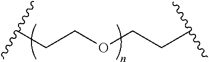 | 2 | 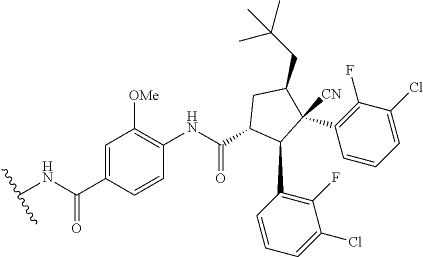 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1021 | 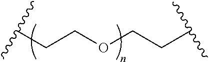 | 3 | 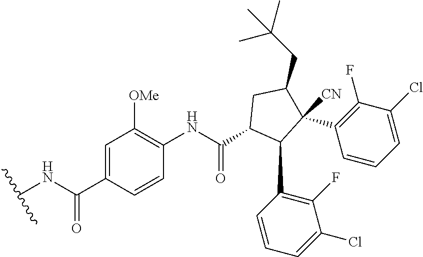 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1022 | 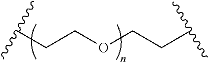 | 4 | 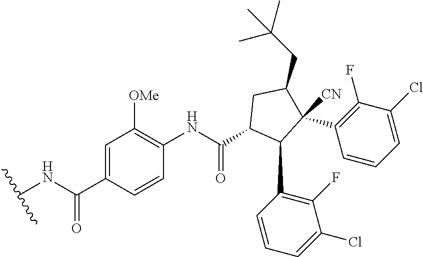 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1023 | 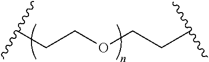 | 5 | 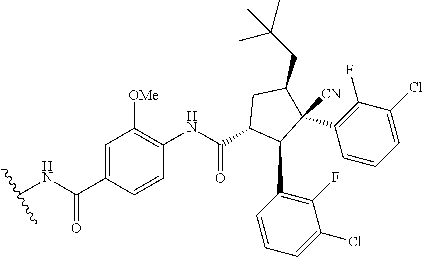 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1024 | 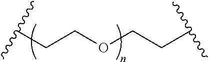 | 1 | 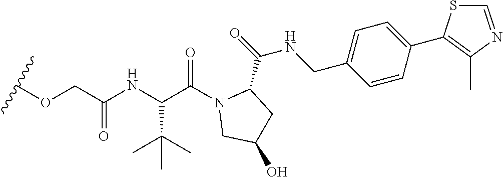 |
| 1025 | 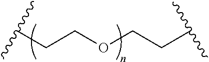 | 2 | 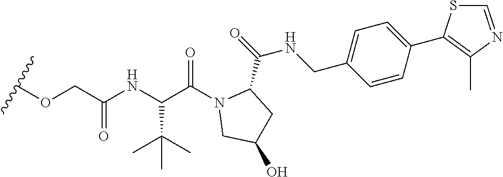 |
| 1026 | 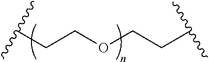 | 3 | 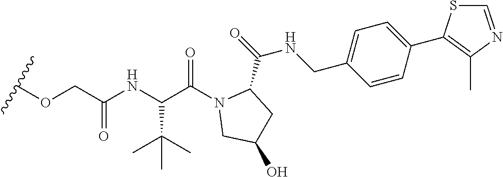 |
| 1027 | 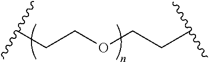 | 4 | 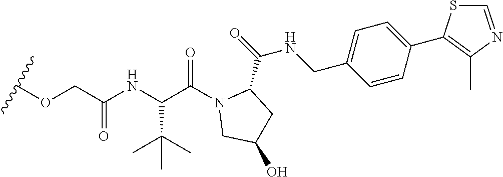 |
| 1028 | 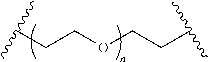 | 5 | 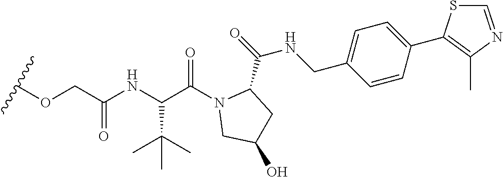 |
| 1029 | 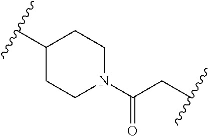 [SMILES: *CC(=O)N1CCC(*)CC1] | — | 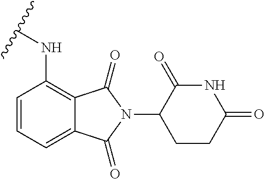 [SMILES: *Nc1cccc2c1C(=O)N(C1CCC(=O)NC1=O)C2=O] |
| 1030 | 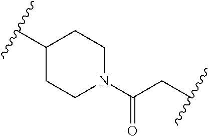 [SMILES: *CC(=O)N1CCC(*)CC1] | — | 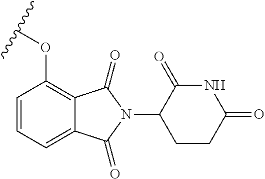 [SMILES: *Oc1cccc2c1C(=O)N(C1CCC(=O)NC1=O)C2=O] |
| 1031 | 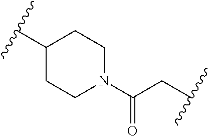 [SMILES: *CC(=O)N1CCC(*)CC1] | — | 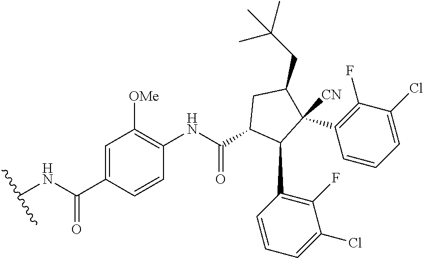 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1032 | 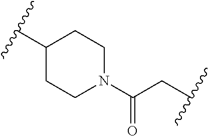 [SMILES: *CC(=O)N1CCC(*)CC1] | — | 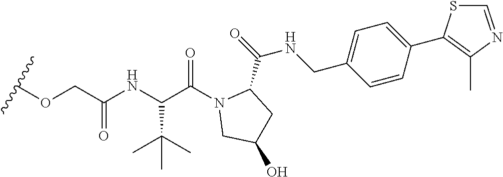 |
| 1033 | 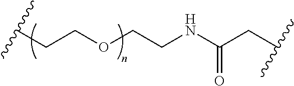 | 1 | 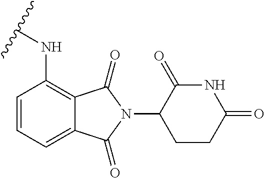 [SMILES: *Nc1cccc2c1C(=O)N(C1CCC(=O)NC1=O)C2=O] |
| 1034 | 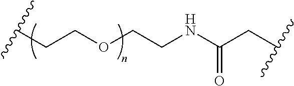 | 2 | 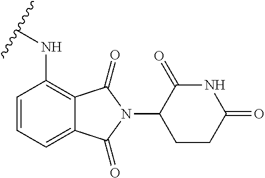 [SMILES: *Nc1cccc2c1C(=O)N(C1CCC(=O)NC1=O)C2=O] |
| 1035 | 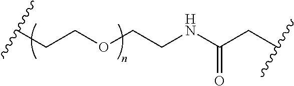 | 3 | 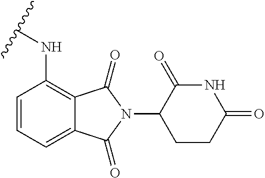 [SMILES: *Nc1cccc2c1C(=O)N(C1CCC(=O)NC1=O)C2=O] |
| 1036 | 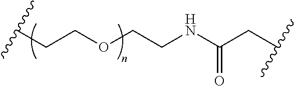 | 4 | 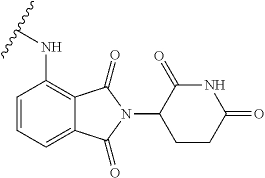 [SMILES: *Nc1cccc2c1C(=O)N(C1CCC(=O)NC1=O)C2=O] |
| 1037 | 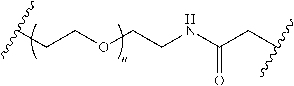 | 5 | 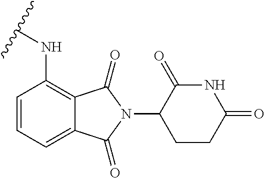 [SMILES: *Nc1cccc2c1C(=O)N(C1CCC(=O)NC1=O)C2=O] |
| 1038 | 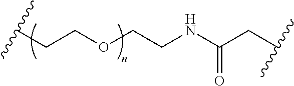 | 1 | 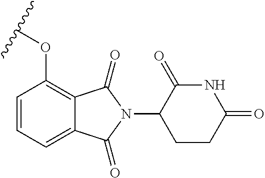 [SMILES: *Oc1cccc2c1C(=O)N(C1CCC(=O)NC1=O)C2=O] |
| 1039 | 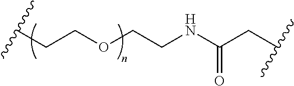 | 2 | 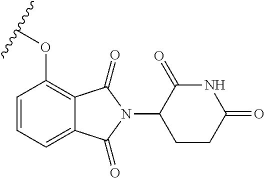 [SMILES: *Oc1cccc2c1C(=O)N(C1CCC(=O)NC1=O)C2=O] |
| 1040 | 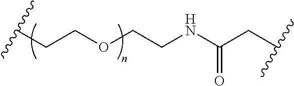 | 3 | 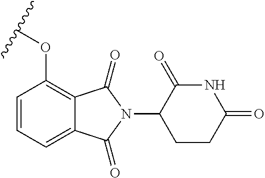 [SMILES: *Oc1cccc2c1C(=O)N(C1CCC(=O)NC1=O)C2=O] |
| 1041 | 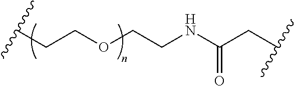 | 4 | 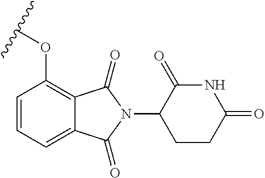 [SMILES: *Oc1cccc2c1C(=O)N(C1CCC(=O)NC1=O)C2=O] |
| 1042 | 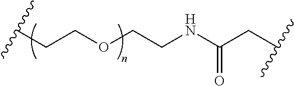 | 5 | 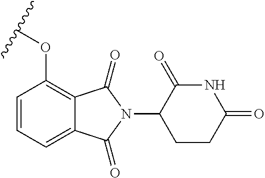 [SMILES: *Oc1cccc2c1C(=O)N(C1CCC(=O)NC1=O)C2=O] |
| 1043 | 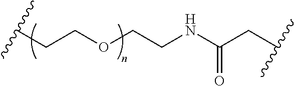 | 1 | 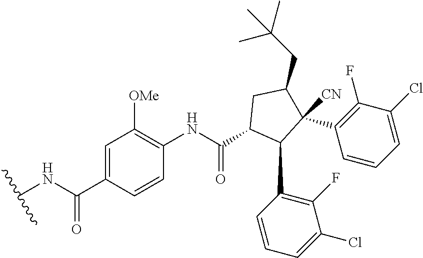 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1044 | 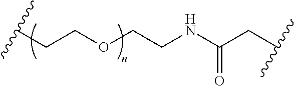 | 2 | 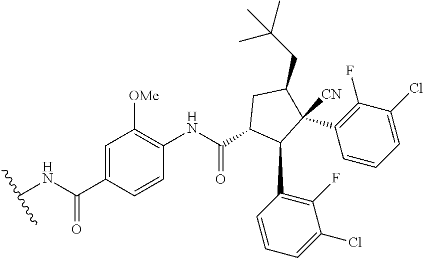 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1045 | 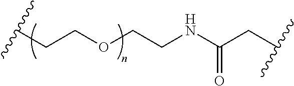 | 3 | 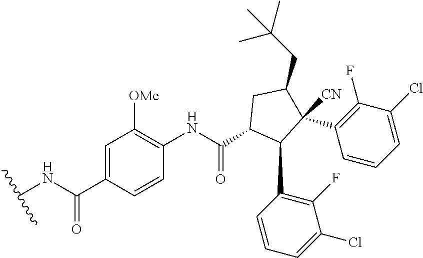 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1046 | 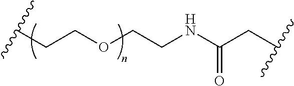 | 4 | 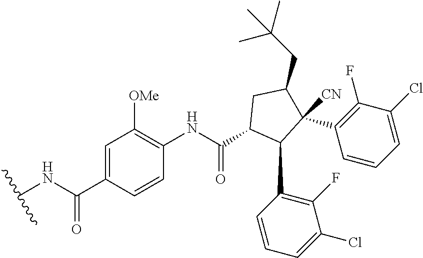 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1047 | 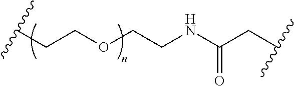 | 5 | 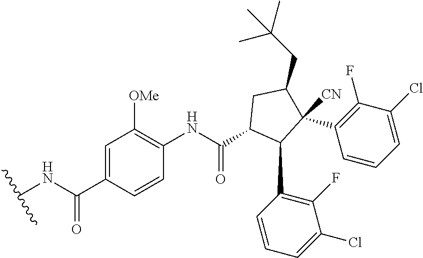 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1048 | 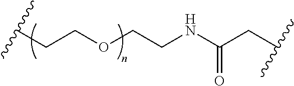 | 1 | 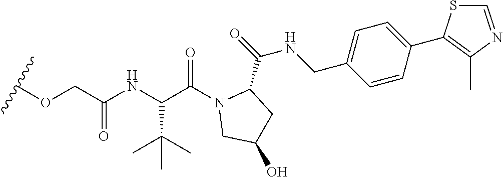 |
| 1049 | 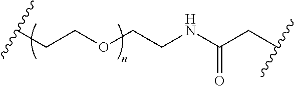 | 2 | 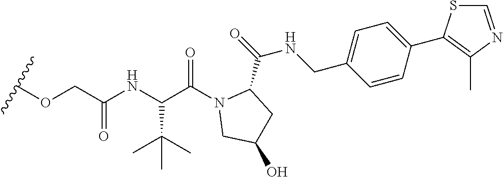 |
| 1050 | 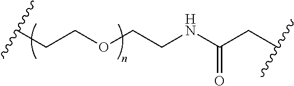 | 3 | 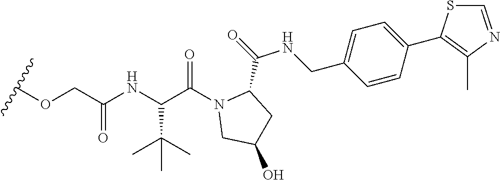 |
| 1051 | 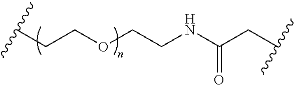 | 4 | 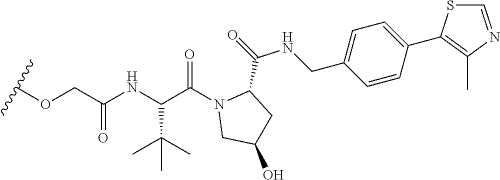 |
| 1052 | 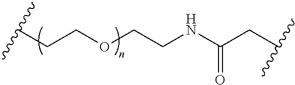 | 5 | 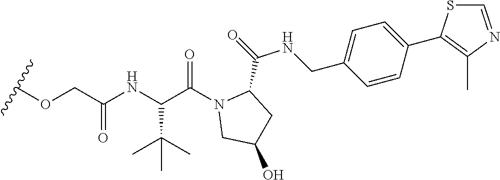 |
| 1053 | 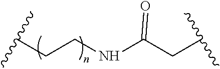 | 1 | 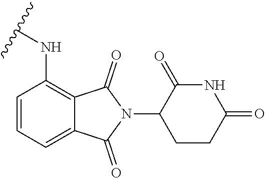 [SMILES: *Nc1cccc2c1C(=O)N(C1CCC(=O)NC1=O)C2=O] |
| 1054 | 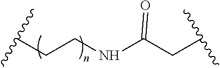 | 2 | 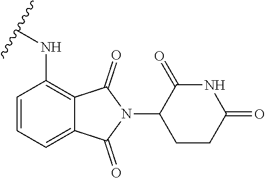 [SMILES: *Nc1cccc2c1C(=O)N(C1CCC(=O)NC1=O)C2=O] |
| 1055 | 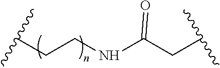 | 3 | 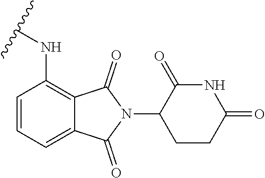 [SMILES: *Nc1cccc2c1C(=O)N(C1CCC(=O)NC1=O)C2=O] |
| 1056 | 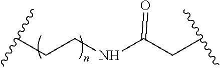 | 4 | 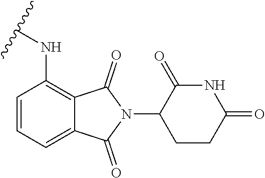 [SMILES: *Nc1cccc2c1C(=O)N(C1CCC(=O)NC1=O)C2=O] |
| 1057 | 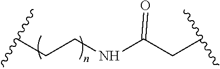 | 5 | 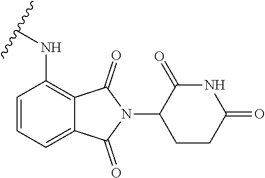 [SMILES: *Nc1cccc2c1C(=O)N(C1CCC(=O)NC1=O)C2=O] |
| 1058 | 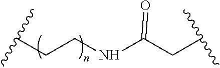 | 1 | 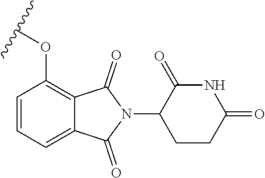 [SMILES: *Oc1cccc2c1C(=O)N(C1CCC(=O)NC1=O)C2=O] |
| 1059 | 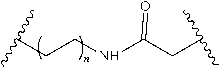 | 2 | 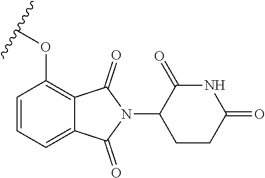 [SMILES: *Oc1cccc2c1C(=O)N(C1CCC(=O)NC1=O)C2=O] |
| 1060 | 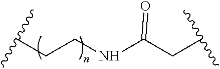 | 3 | 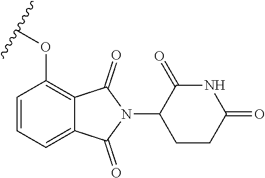 [SMILES: *Oc1cccc2c1C(=O)N(C1CCC(=O)NC1=O)C2=O] |
| 1061 | 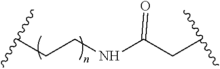 | 4 | 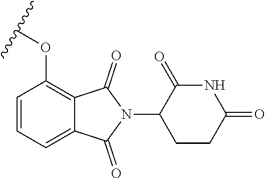 [SMILES: *Oc1cccc2c1C(=O)N(C1CCC(=O)NC1=O)C2=O] |
| 1062 | 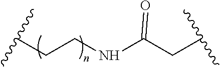 | 5 | 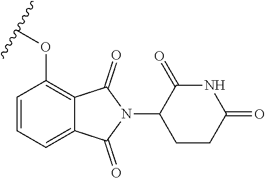 [SMILES: *Oc1cccc2c1C(=O)N(C1CCC(=O)NC1=O)C2=O] |
| 1063 | 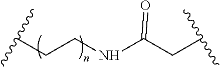 | 1 | 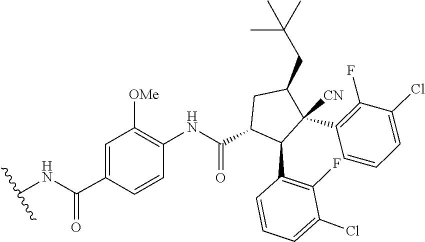 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1064 | 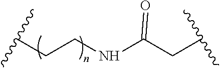 | 2 | 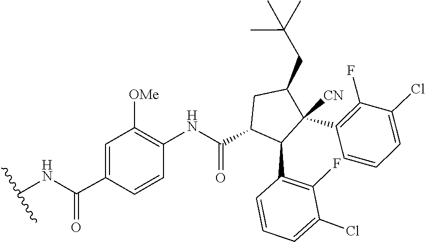 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1065 | 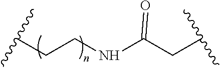 | 3 | 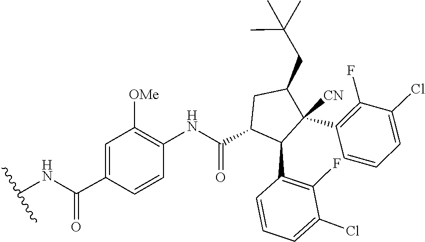 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1066 | 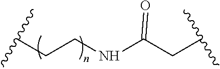 | 4 | 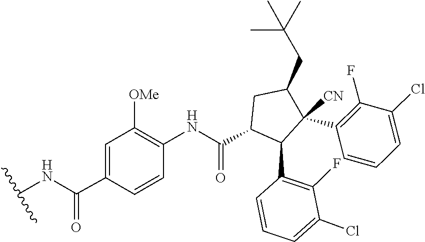 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1067 | 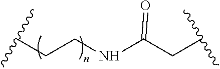 | 5 | 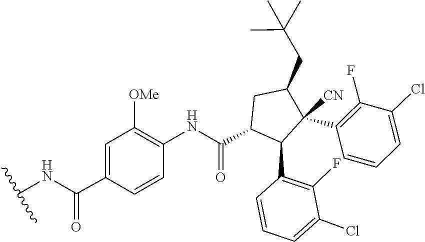 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1068 | 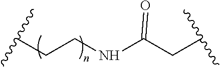 | 1 | 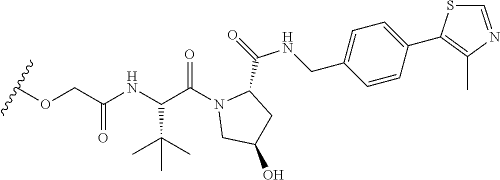 |
| 1069 | 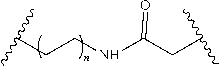 | 2 | 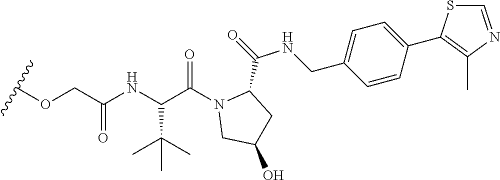 |
| 1070 | 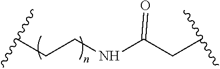 | 3 | 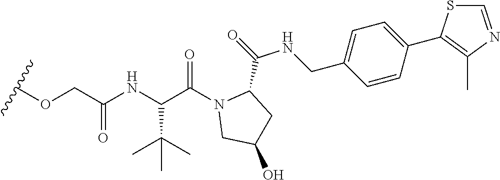 |
| 1071 | 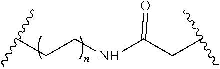 | 4 | 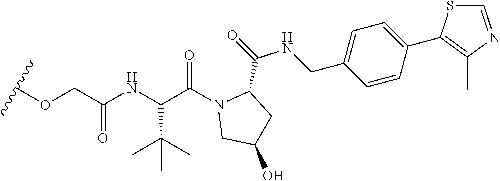 |
| 1072 | 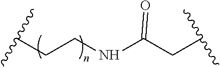 | 5 | 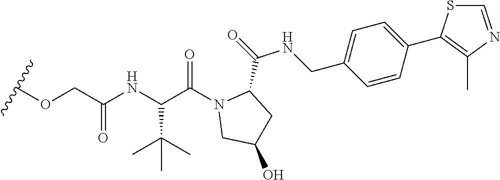 |
| 1073 | 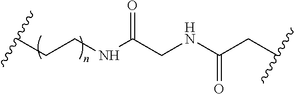 | 1 | 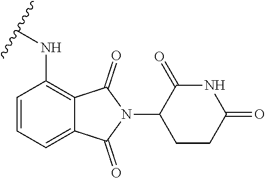 [SMILES: *Nc1cccc2c1C(=O)N(C1CCC(=O)NC1=O)C2=O] |
| 1074 | 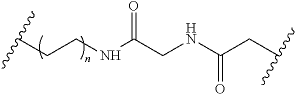 | 2 | 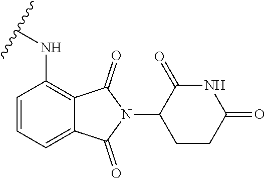 [SMILES: *Nc1cccc2c1C(=O)N(C1CCC(=O)NC1=O)C2=O] |
| 1075 | 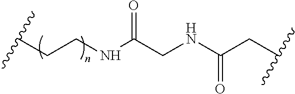 | 3 | 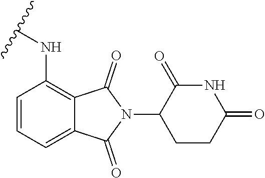 [SMILES: *Nc1cccc2c1C(=O)N(C1CCC(=O)NC1=O)C2=O] |
| 1076 | 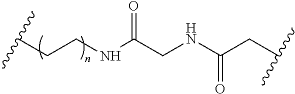 | 4 | 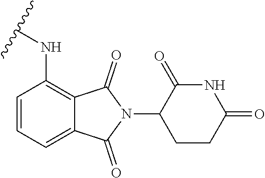 [SMILES: *Nc1cccc2c1C(=O)N(C1CCC(=O)NC1=O)C2=O] |
| 1077 | 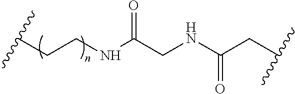 | 5 | 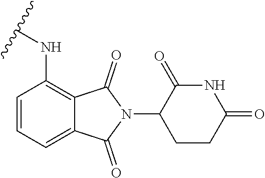 [SMILES: *Nc1cccc2c1C(=O)N(C1CCC(=O)NC1=O)C2=O] |
| 1078 | 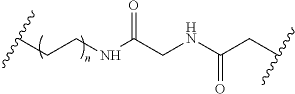 | 1 | 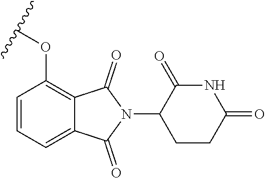 [SMILES: *Oc1cccc2c1C(=O)N(C1CCC(=O)NC1=O)C2=O] |
| 1079 | 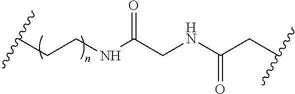 | 2 | 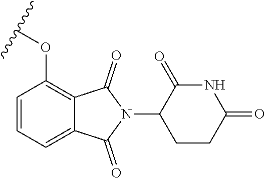 [SMILES: *Oc1cccc2c1C(=O)N(C1CCC(=O)NC1=O)C2=O] |
| 1080 | 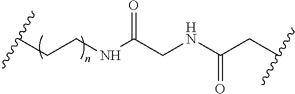 | 3 | 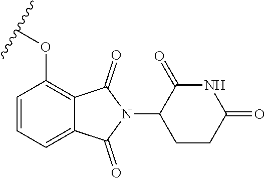 [SMILES: *Oc1cccc2c1C(=O)N(C1CCC(=O)NC1=O)C2=O] |
| 1081 | 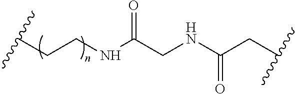 | 4 | 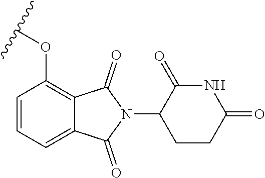 [SMILES: *Oc1cccc2c1C(=O)N(C1CCC(=O)NC1=O)C2=O] |
| 1082 | 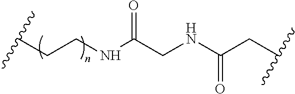 | 5 | 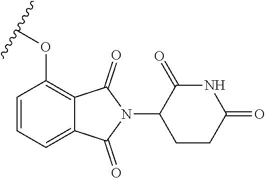 [SMILES: *Oc1cccc2c1C(=O)N(C1CCC(=O)NC1=O)C2=O] |
| 1083 | 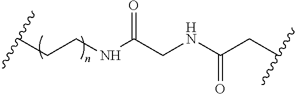 | 1 | 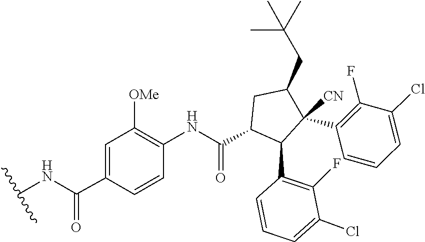 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1084 | 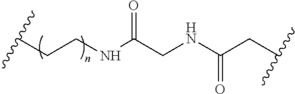 | 2 | 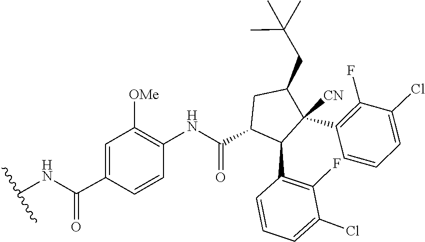 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1085 | 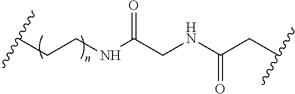 | 3 | 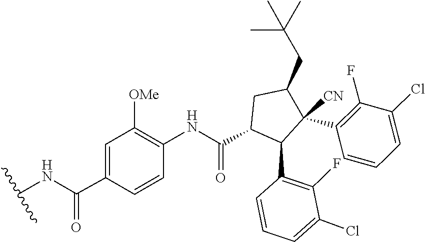 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1086 | 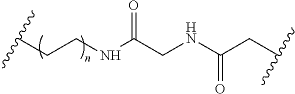 | 4 | 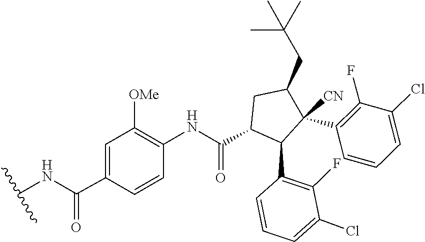 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1087 | 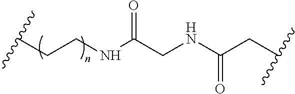 | 5 | 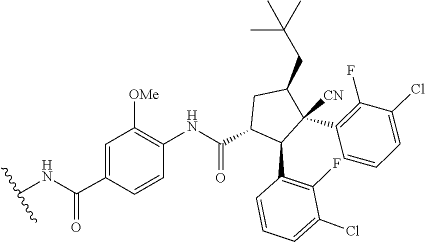 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1088 | 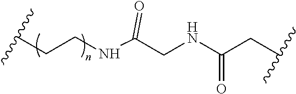 | 1 | 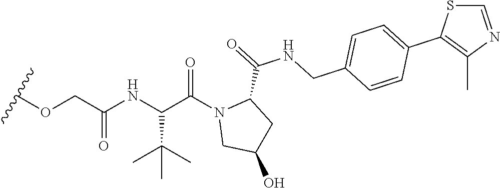 |
| 1089 | 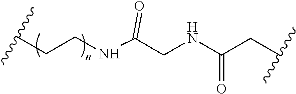 | 2 | 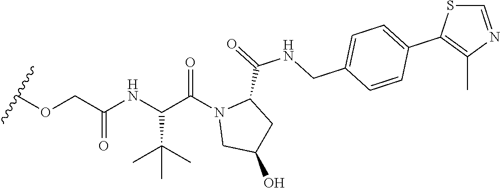 |
| 1090 | 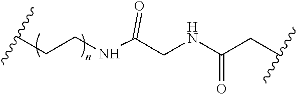 | 3 | 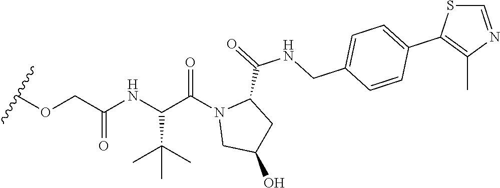 |
| 1091 | 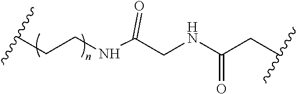 | 4 | 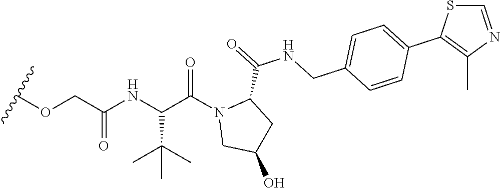 |
| 1092 | 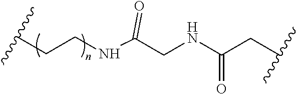 | 5 | 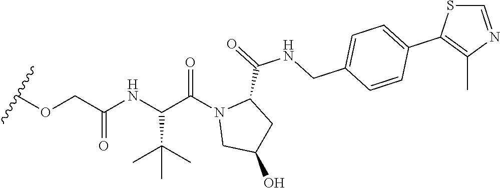 |

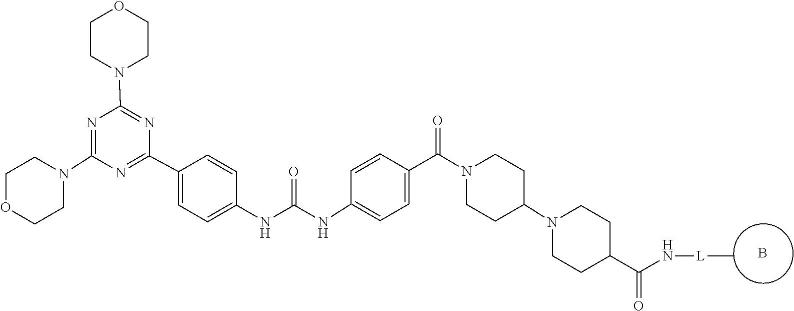

| For- | | | |
|---|---|---|---|
| mu- | | | |
| la | | | |
| No. | L | n | B |
| 1093 | 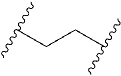 [SMILES: *CC*] | — | 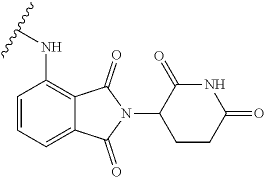 [SMILES: *Nc1cccc2c1C(=O)N(C1CCC(=O)NC1=O)C2=O] |
| 1094 | 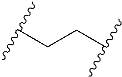 [SMILES: *CC*] | — | 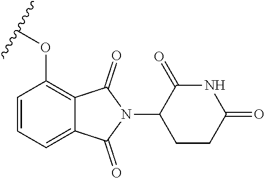 [SMILES: *Oc1cccc2c1C(=O)N(C1CCC(=O)NC1=O)C2=O] |
| 1095 | 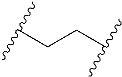 [SMILES: *CC*] | — | 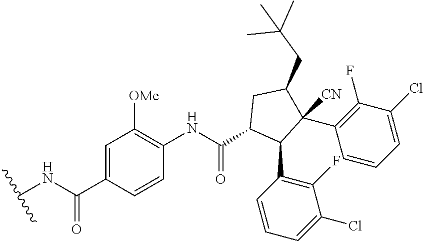 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1096 | 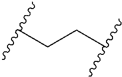 [SMILES: *CC*] | — | 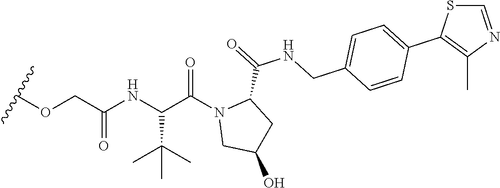 |
| 1097 | 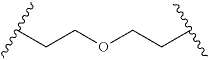 [SMILES: *CCOCC*] | — | 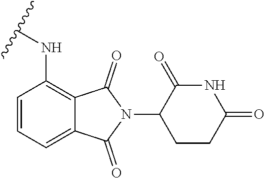 [SMILES: *Nc1cccc2c1C(=O)N(C1CCC(=O)NC1=O)C2=O] |
| 1098 | 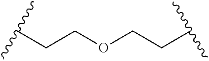 [SMILES: *CCOCC*] | — | 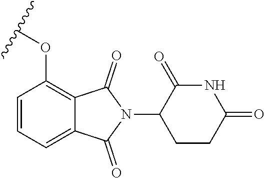 [SMILES: *Oc1cccc2c1C(=O)N(C1CCC(=O)NC1=O)C2=O] |
| 1099 | 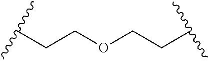 [SMILES: *CCOCC*] | — | 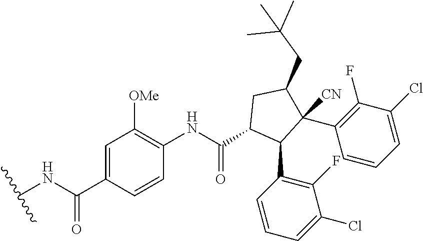 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1100 | 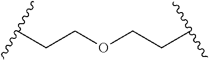 [SMILES: *CCOCC*] | — | 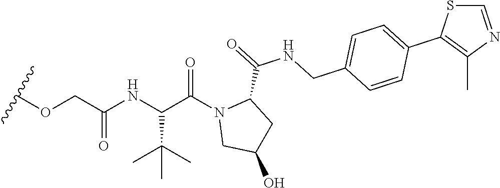 |
| 1101 | 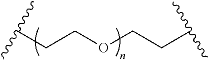 | 1 | 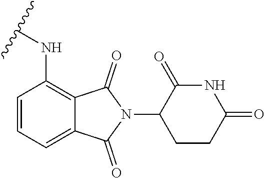 [SMILES: *Nc1cccc2c1C(=O)N(C1CCC(=O)NC1=O)C2=O] |
| 1102 | 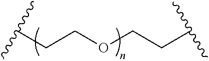 | 2 | 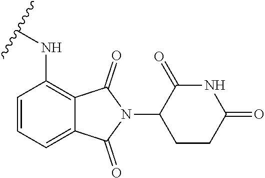 [SMILES: *Nc1cccc2c1C(=O)N(C1CCC(=O)NC1=O)C2=O] |
| 1103 | 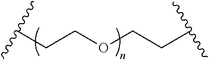 | 3 | 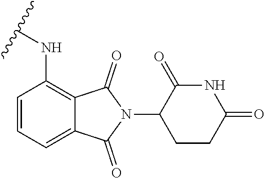 [SMILES: *Nc1cccc2c1C(=O)N(C1CCC(=O)NC1=O)C2=O] |
| 1104 | 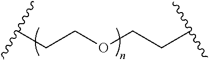 | 4 | 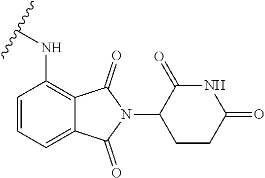 [SMILES: *Nc1cccc2c1C(=O)N(C1CCC(=O)NC1=O)C2=O] |
| 1105 | 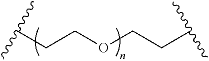 | 5 | 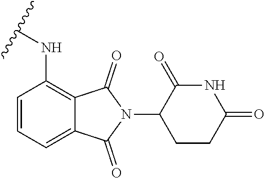 [SMILES: *Nc1cccc2c1C(=O)N(C1CCC(=O)NC1=O)C2=O] |
| 1106 | 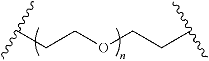 | 1 | 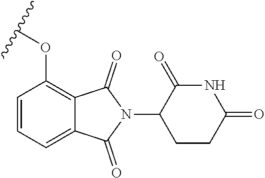 [SMILES: *Oc1cccc2c1C(=O)N(C1CCC(=O)NC1=O)C2=O] |
| 1107 | 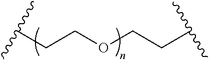 | 2 | 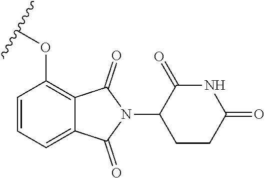 [SMILES: *Oc1cccc2c1C(=O)N(C1CCC(=O)NC1=O)C2=O] |
| 1108 | 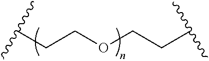 | 3 | 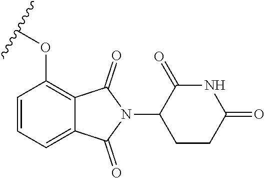 [SMILES: *Oc1cccc2c1C(=O)N(C1CCC(=O)NC1=O)C2=O] |
| 1109 | 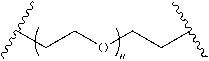 | 4 | 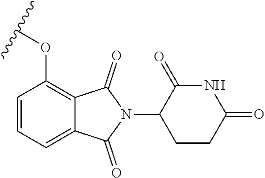 [SMILES: *Oc1cccc2c1C(=O)N(C1CCC(=O)NC1=O)C2=O] |
| 1110 | 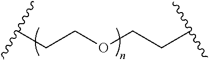 | 5 | 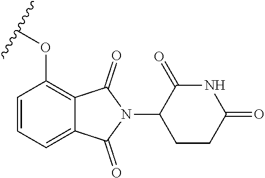 [SMILES: *Oc1cccc2c1C(=O)N(C1CCC(=O)NC1=O)C2=O] |
| 1111 | 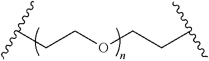 | 1 | 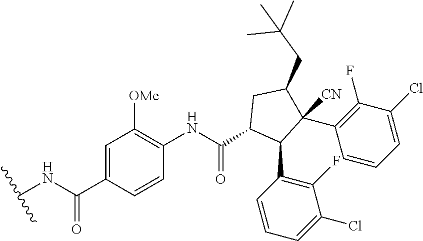 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1112 | 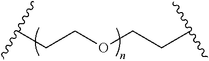 | 2 | 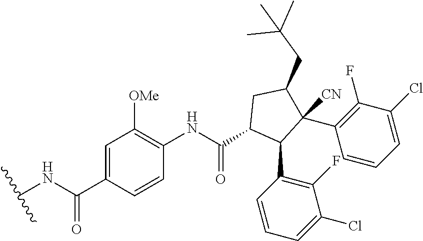 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1113 | 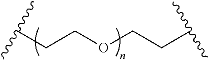 | 3 | 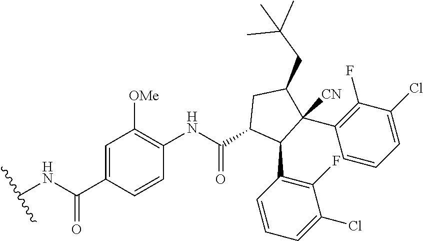 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1114 | 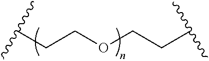 | 4 | 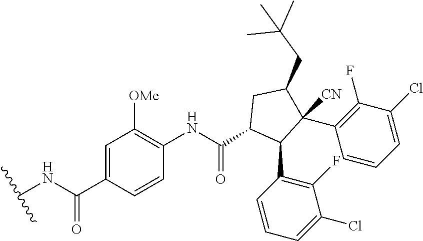 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1115 | 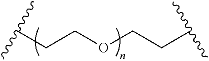 | 5 | 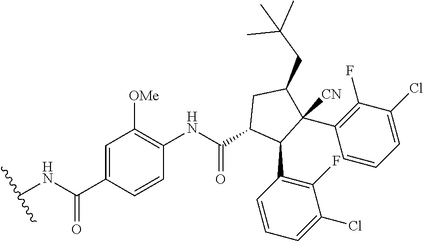 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1116 | 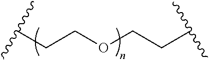 | 1 | 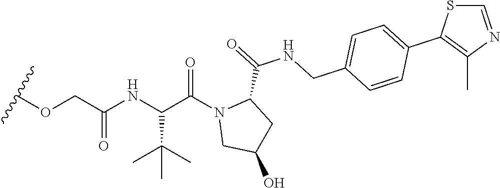 |
| 1117 | 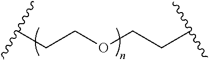 | 2 | 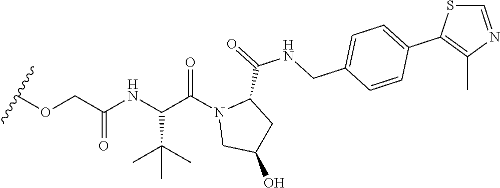 |
| 1118 | 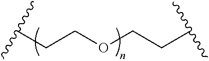 | 3 | 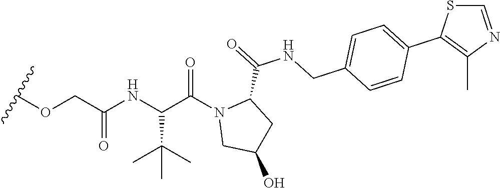 |
| 1119 | 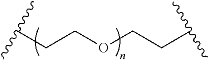 | 4 | 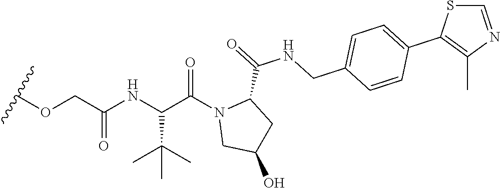 |
| 1120 | 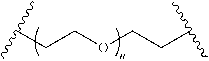 | 5 | 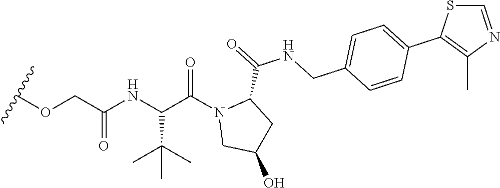 |
| 1121 | 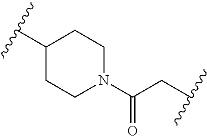 [SMILES: *CC(=O)N1CCC(*)CC1] | — | 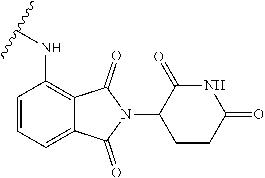 [SMILES: *Nc1cccc2c1C(=O)N(C1CCC(=O)NC1=O)C2=O] |
| 1122 | 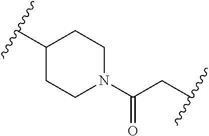 [SMILES: *CC(=O)N1CCC(*)CC1] | — | 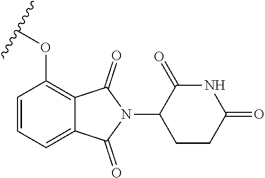 [SMILES: *Oc1cccc2c1C(=O)N(C1CCC(=O)NC1=O)C2=O] |
| 1123 | 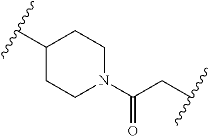 [SMILES: *CC(=O)N1CCC(*)CC1] | — | 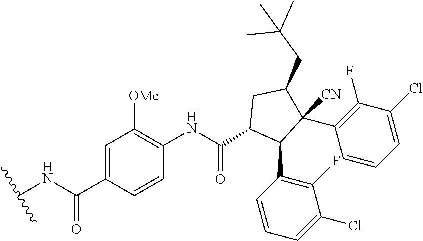 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1124 | 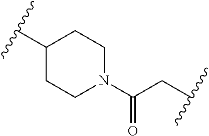 [SMILES: *CC(=O)N1CCC(*)CC1] | — | 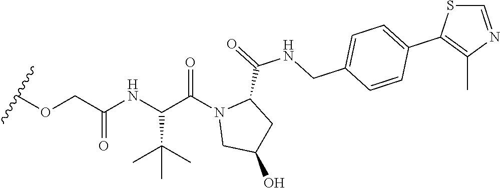 |
| 1125 | 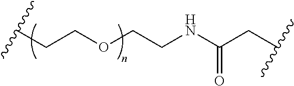 | 1 | 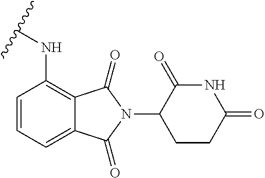 [SMILES: *Nc1cccc2c1C(=O)N(C1CCC(=O)NC1=O)C2=O] |
| 1126 | 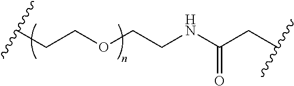 | 2 | 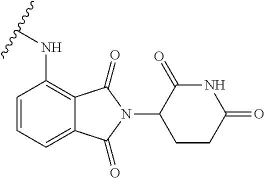 [SMILES: *Nc1cccc2c1C(=O)N(C1CCC(=O)NC1=O)C2=O] |
| 1127 | 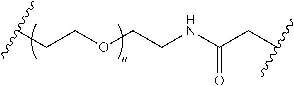 | 3 | 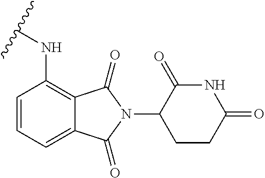 [SMILES: *Nc1cccc2c1C(=O)N(C1CCC(=O)NC1=O)C2=O] |
| 1128 | 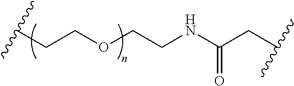 | 4 | 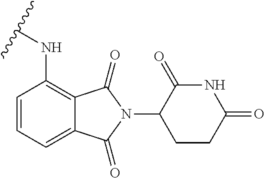 [SMILES: *Nc1cccc2c1C(=O)N(C1CCC(=O)NC1=O)C2=O] |
| 1129 | 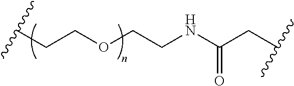 | 5 | 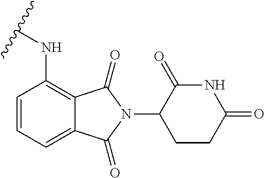 [SMILES: *Nc1cccc2c1C(=O)N(C1CCC(=O)NC1=O)C2=O] |
| 1130 | 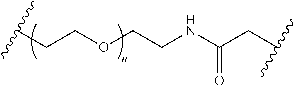 | 1 | 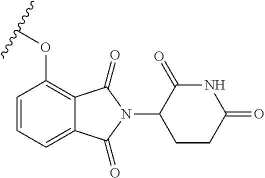 [SMILES: *Oc1cccc2c1C(=O)N(C1CCC(=O)NC1=O)C2=O] |
| 1131 | 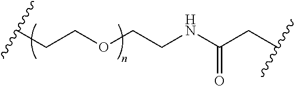 | 2 | 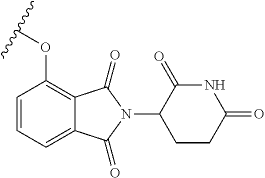 [SMILES: *Oc1cccc2c1C(=O)N(C1CCC(=O)NC1=O)C2=O] |
| 1132 | 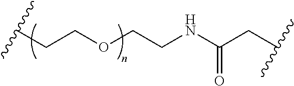 | 3 | 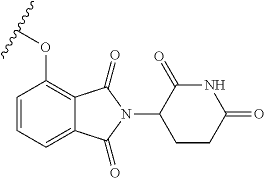 [SMILES: *Oc1cccc2c1C(=O)N(C1CCC(=O)NC1=O)C2=O] |
| 1133 | 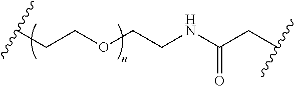 | 4 | 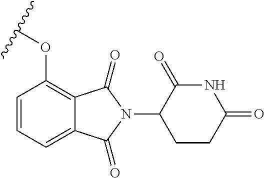 [SMILES: *Oc1cccc2c1C(=O)N(C1CCC(=O)NC1=O)C2=O] |
| 1134 | 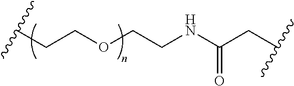 | 5 | 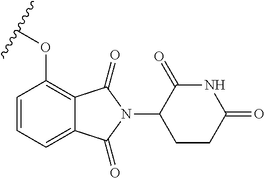 [SMILES: *Oc1cccc2c1C(=O)N(C1CCC(=O)NC1=O)C2=O] |
| 1135 | 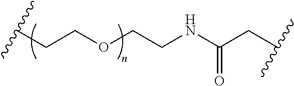 | 1 | 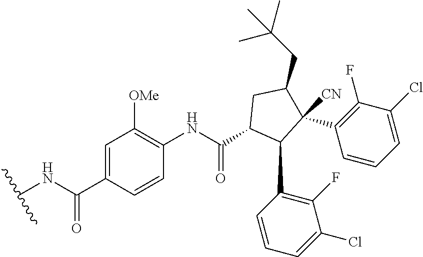 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1136 | 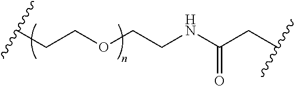 | 2 | 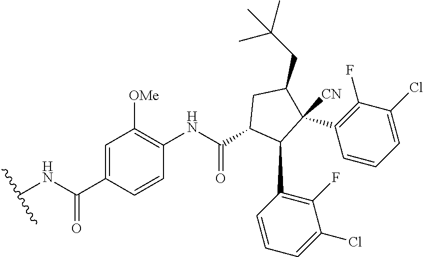 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1137 | 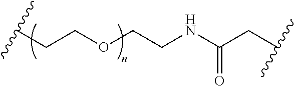 | 3 | 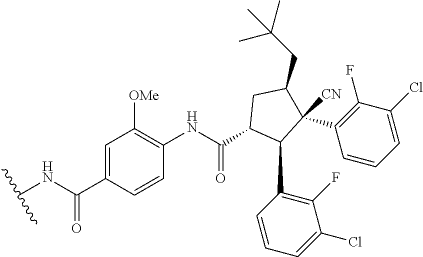 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1138 | 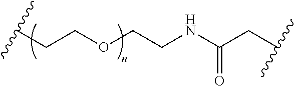 | 4 | 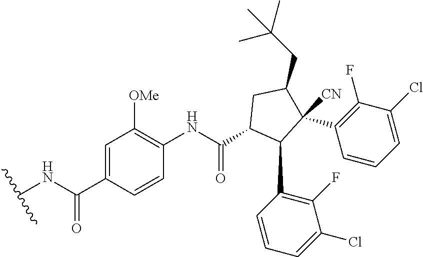 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1139 | 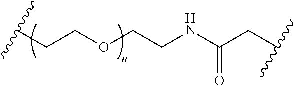 | 5 | 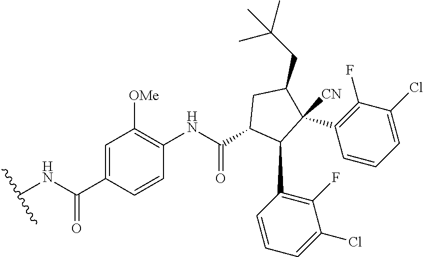 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1140 | 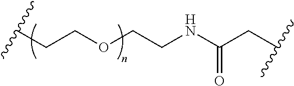 | 1 | 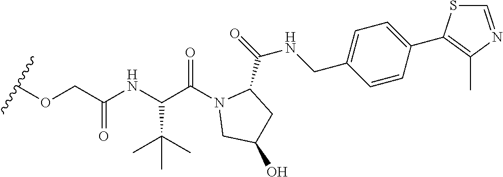 |
| 1141 | 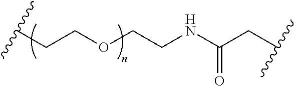 | 2 | 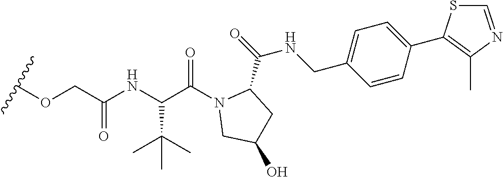 |
| 1142 | 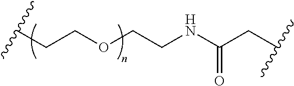 | 3 | 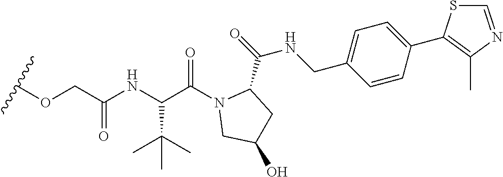 |
| 1143 | 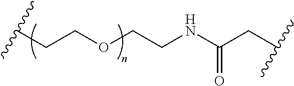 | 4 | 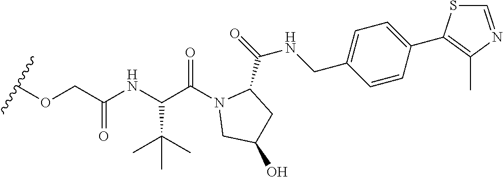 |
| 1144 | 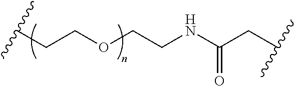 | 5 | 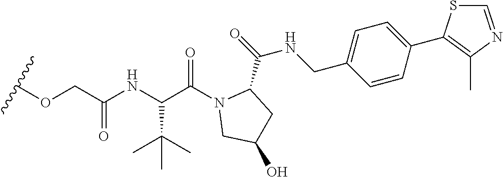 |
| 1145 | 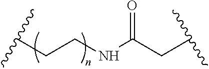 | 1 | 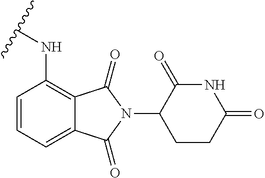 [SMILES: *Nc1cccc2c1C(=O)N(C1CCC(=O)NC1=O)C2=O] |
| 1146 | 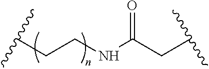 | 2 | 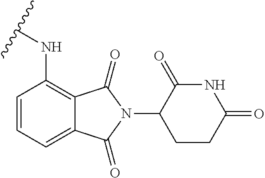 [SMILES: *Nc1cccc2c1C(=O)N(C1CCC(=O)NC1=O)C2=O] |
| 1147 | 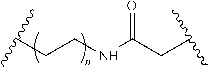 | 3 | 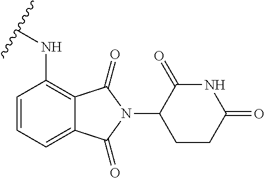 [SMILES: *Nc1cccc2c1C(=O)N(C1CCC(=O)NC1=O)C2=O] |
| 1148 | 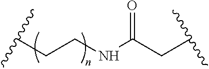 | 4 | 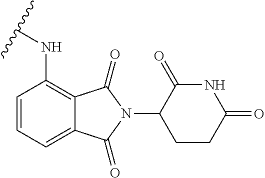 [SMILES: *Nc1cccc2c1C(=O)N(C1CCC(=O)NC1=O)C2=O] |
| 1149 | 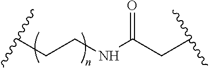 | 5 | 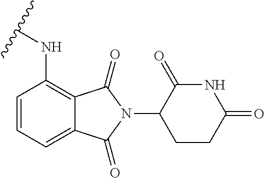 [SMILES: *Nc1cccc2c1C(=O)N(C1CCC(=O)NC1=O)C2=O] |
| 1150 | 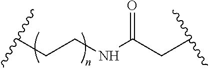 | 1 | 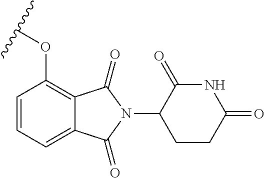 [SMILES: *Oc1cccc2c1C(=O)N(C1CCC(=O)NC1=O)C2=O] |
| 1151 | 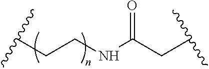 | 2 | 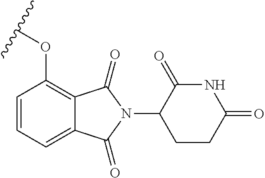 [SMILES: *Oc1cccc2c1C(=O)N(C1CCC(=O)NC1=O)C2=O] |
| 1152 | 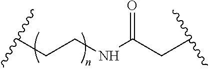 | 3 | 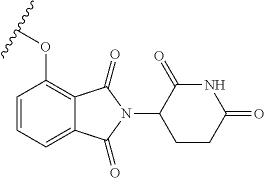 [SMILES: *Oc1cccc2c1C(=O)N(C1CCC(=O)NC1=O)C2=O] |
| 1153 | 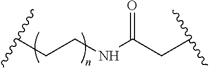 | 4 | 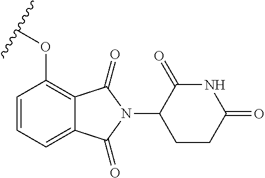 [SMILES: *Oc1cccc2c1C(=O)N(C1CCC(=O)NC1=O)C2=O] |
| 1154 | 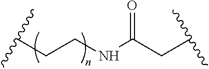 | 5 | 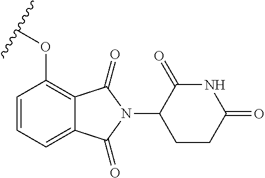 [SMILES: *Oc1cccc2c1C(=O)N(C1CCC(=O)NC1=O)C2=O] |
| 1155 | 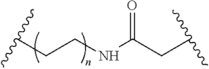 | 1 | 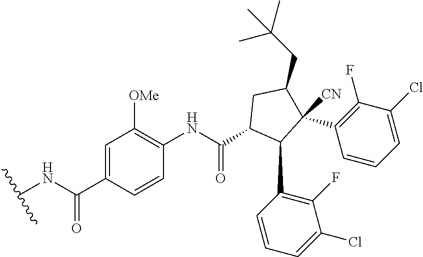 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1156 | 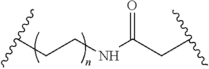 | 2 | 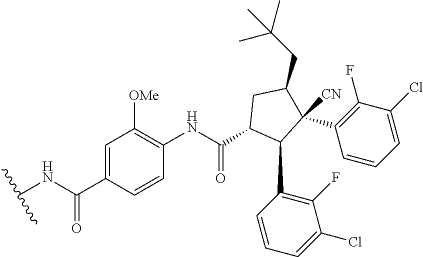 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1157 | 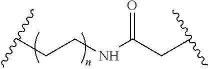 | 3 | 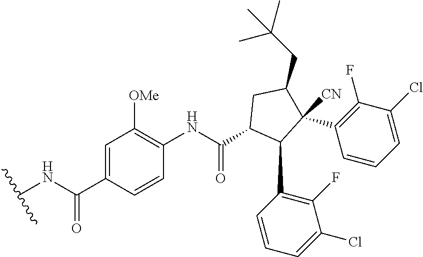 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1158 | 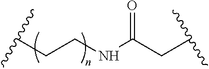 | 4 | 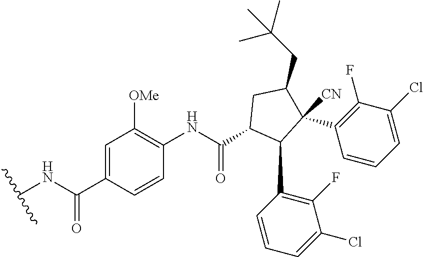 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1159 | 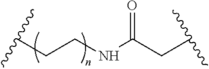 | 5 | 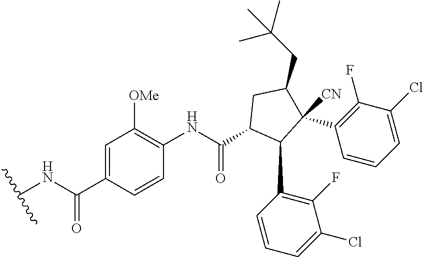 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1160 | 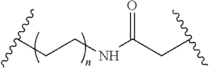 | 1 | 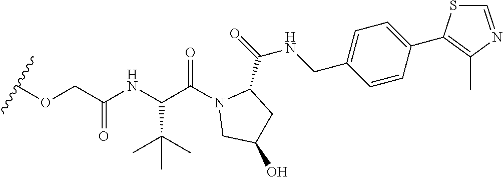 |
| 1161 | 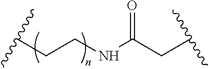 | 2 | 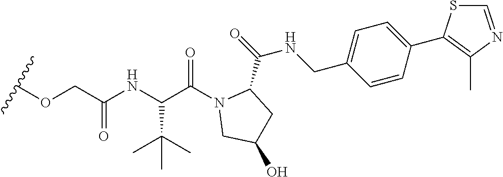 |
| 1162 | 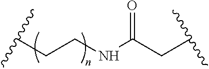 | 3 | 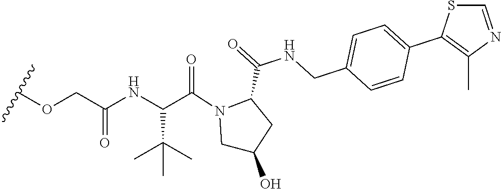 |
| 1163 | 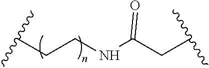 | 4 | 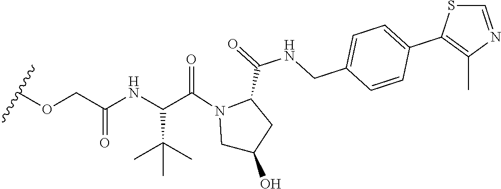 |
| 1164 | 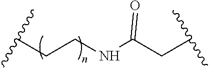 | 5 | 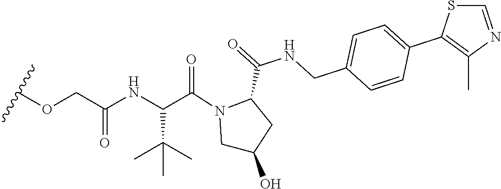 |
| 1165 | 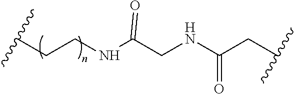 | 1 | 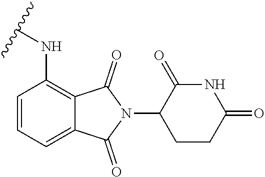 [SMILES: *Nc1cccc2c1C(=O)N(C1CCC(=O)NC1=O)C2=O] |
| 1166 | 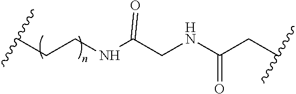 | 2 | 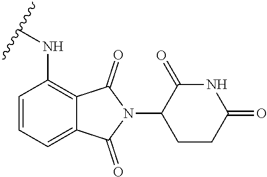 [SMILES: *Nc1cccc2c1C(=O)N(C1CCC(=O)NC1=O)C2=O] |
| 1167 | 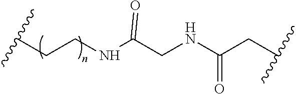 | 3 | 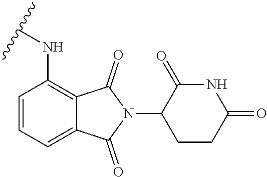 [SMILES: *Nc1cccc2c1C(=O)N(C1CCC(=O)NC1=O)C2=O] |
| 1168 | 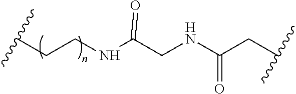 | 4 | 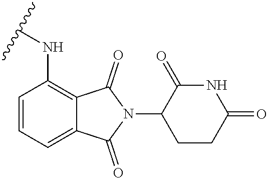 [SMILES: *Nc1cccc2c1C(=O)N(C1CCC(=O)NC1=O)C2=O] |
| 1169 | 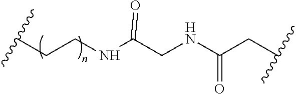 | 5 | 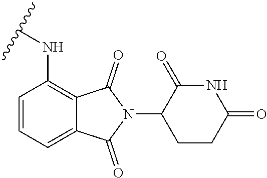 [SMILES: *Nc1cccc2c1C(=O)N(C1CCC(=O)NC1=O)C2=O] |
| 1170 | 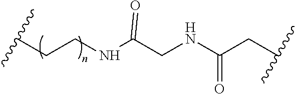 | 1 | 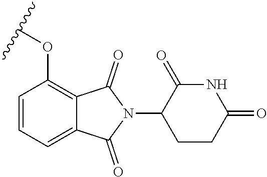 [SMILES: *Oc1cccc2c1C(=O)N(C1CCC(=O)NC1=O)C2=O] |
| 1171 | 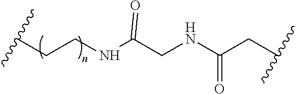 | 2 | 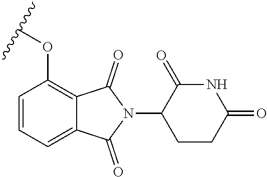 [SMILES: *Oc1cccc2c1C(=O)N(C1CCC(=O)NC1=O)C2=O] |
| 1172 | 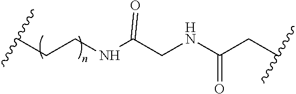 | 3 | 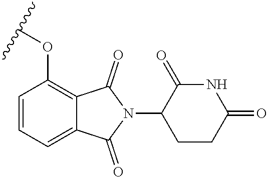 [SMILES: *Oc1cccc2c1C(=O)N(C1CCC(=O)NC1=O)C2=O] |
| 1173 | 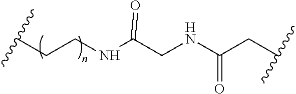 | 4 | 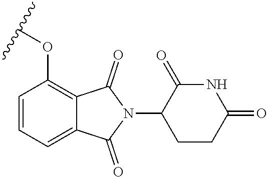 [SMILES: *Oc1cccc2c1C(=O)N(C1CCC(=O)NC1=O)C2=O] |
| 1174 | 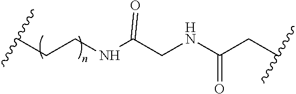 | 5 | 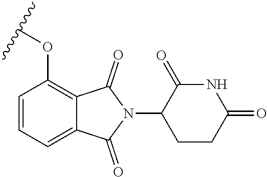 [SMILES: *Oc1cccc2c1C(=O)N(C1CCC(=O)NC1=O)C2=O] |
| 1175 | 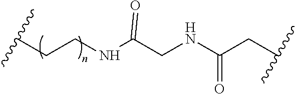 | 1 | 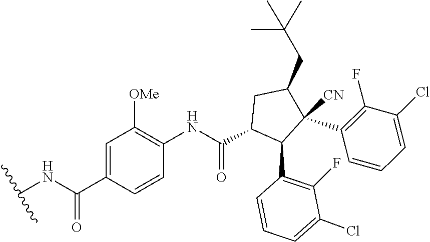 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1176 | 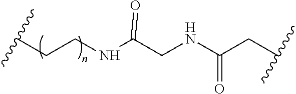 | 2 | 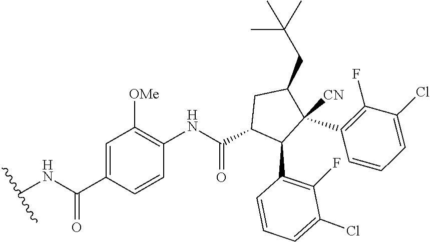 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1177 | 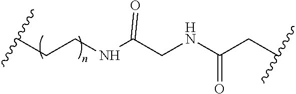 | 3 | 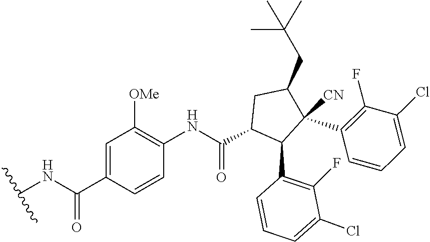 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1178 | 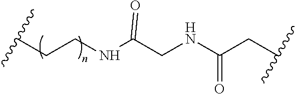 | 4 | 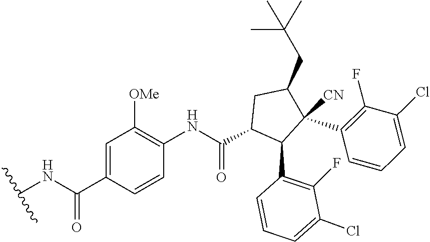 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1179 | 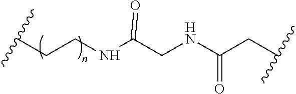 | 5 | 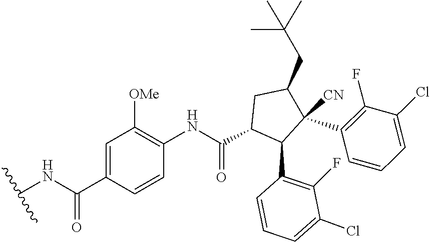 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1180 | 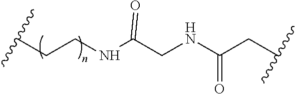 | 1 | 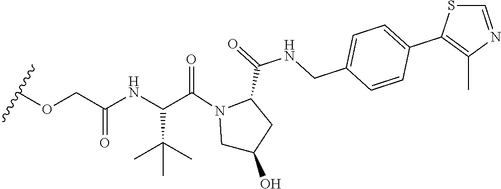 |
| 1181 | 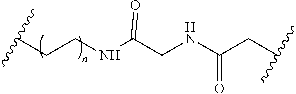 | 2 | 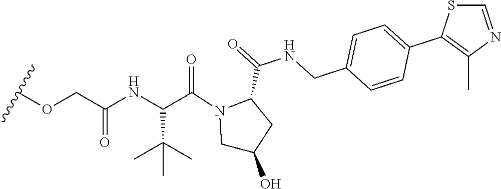 |
| 1182 | 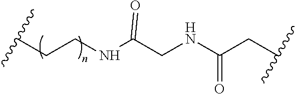 | 3 | 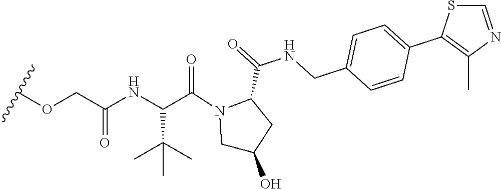 |
| 1183 | 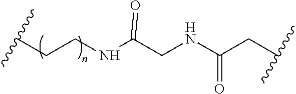 | 4 | 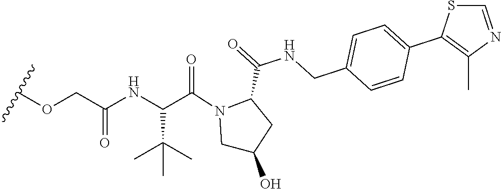 |
| 1184 | 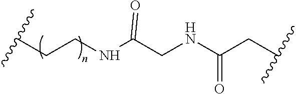 | 5 | 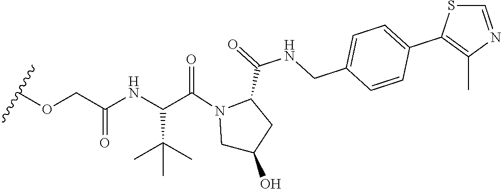 |

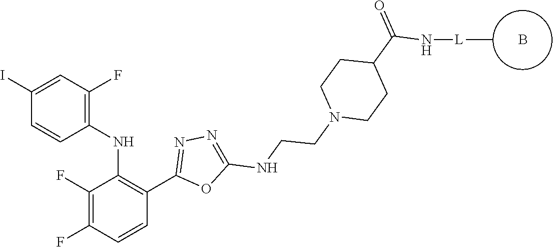

| For- | | | |
|---|---|---|---|
| mu- | | | |
| la | | | |
| No. | L | n | B |
| 1185 | 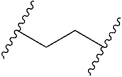 [SMILES: *CC*] | — | 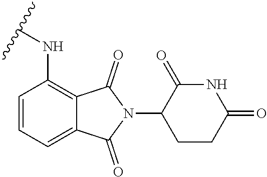 [SMILES: *Nc1cccc2c1C(=O)N(C1CCC(=O)NC1=O)C2=O] |
| 1186 | 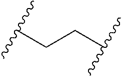 [SMILES: *CC*] | — | 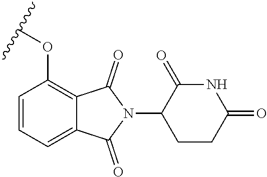 [SMILES: *Oc1cccc2c1C(=O)N(C1CCC(=O)NC1=O)C2=O] |
| 1187 | 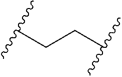 [SMILES: *CC*] | — | 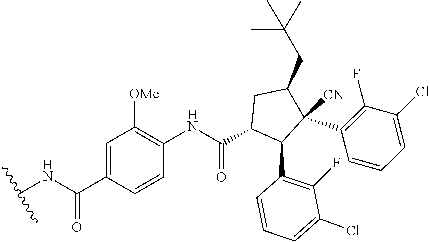 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1188 | 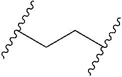 [SMILES: *CC*] | — | 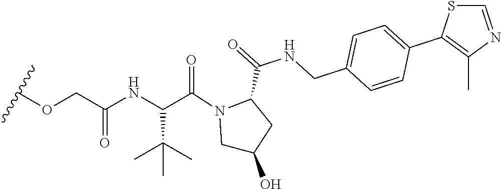 |
| 1189 | 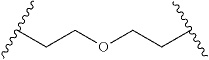 [SMILES: *CCOCC*] | — | 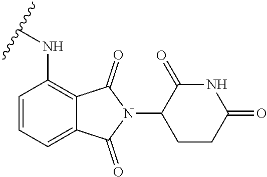 [SMILES: *Nc1cccc2c1C(=O)N(C1CCC(=O)NC1=O)C2=O] |
| 1190 | 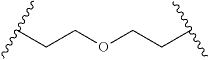 [SMILES: *CCOCC*] | — | 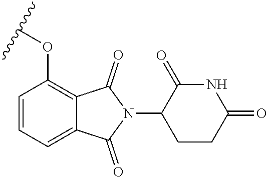 [SMILES: *Oc1cccc2c1C(=O)N(C1CCC(=O)NC1=O)C2=O] |
| 1191 | 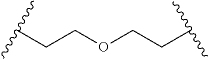 [SMILES: *CCOCC*] | — | 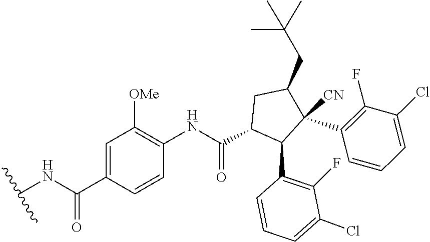 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1192 | 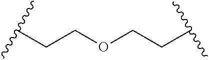 [SMILES: *CCOCC*] | — | 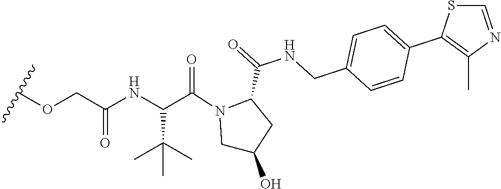 |
| 1193 | 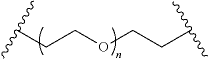 | 1 | 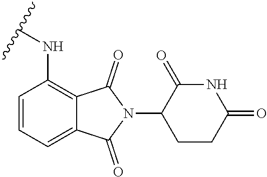 [SMILES: *Nc1cccc2c1C(=O)N(C1CCC(=O)NC1=O)C2=O] |
| 1194 | 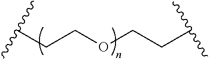 | 2 | 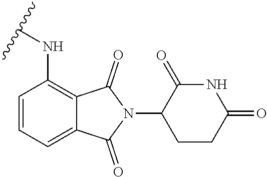 [SMILES: *Nc1cccc2c1C(=O)N(C1CCC(=O)NC1=O)C2=O] |
| 1195 | 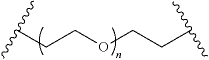 | 3 | 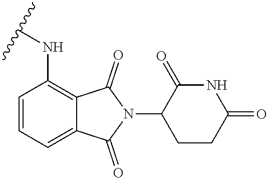 [SMILES: *Nc1cccc2c1C(=O)N(C1CCC(=O)NC1=O)C2=O] |
| 1196 | 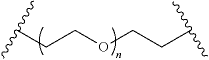 | 4 | 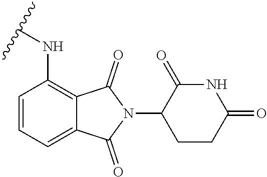 [SMILES: *Nc1cccc2c1C(=O)N(C1CCC(=O)NC1=O)C2=O] |
| 1197 | 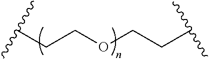 | 5 | 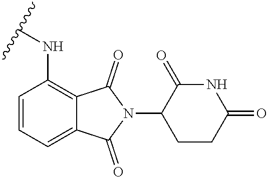 [SMILES: *Nc1cccc2c1C(=O)N(C1CCC(=O)NC1=O)C2=O] |
| 1198 | 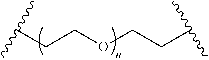 | 1 | 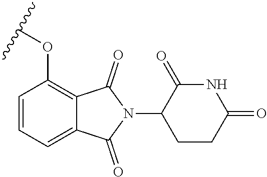 [SMILES: *Oc1cccc2c1C(=O)N(C1CCC(=O)NC1=O)C2=O] |
| 1199 | 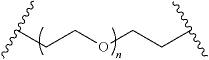 | 2 | 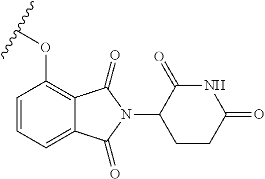 [SMILES: *Oc1cccc2c1C(=O)N(C1CCC(=O)NC1=O)C2=O] |
| 1200 | 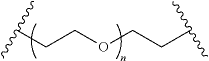 | 3 | 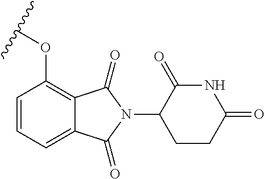 [SMILES: *Oc1cccc2c1C(=O)N(C1CCC(=O)NC1=O)C2=O] |
| 1201 | 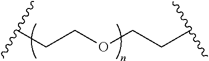 | 4 | 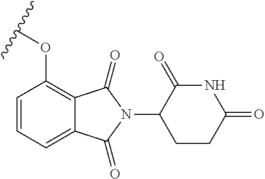 [SMILES: *Oc1cccc2c1C(=O)N(C1CCC(=O)NC1=O)C2=O] |
| 1202 | 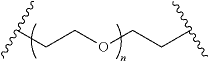 | 5 | 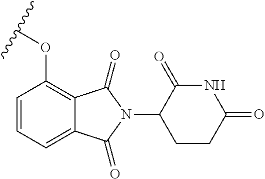 [SMILES: *Oc1cccc2c1C(=O)N(C1CCC(=O)NC1=O)C2=O] |
| 1203 | 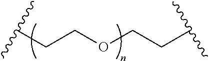 | 1 | 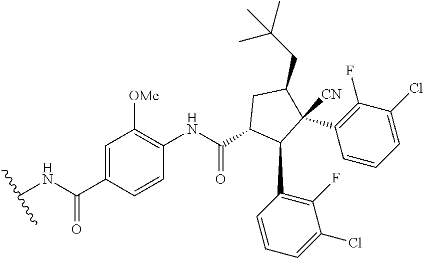 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1204 | 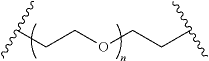 | 2 | 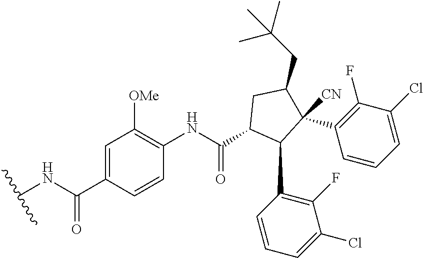 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1205 | 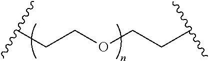 | 3 | 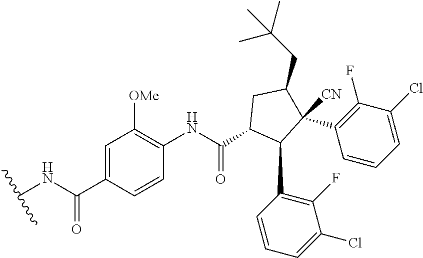 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1206 | 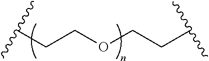 | 4 | 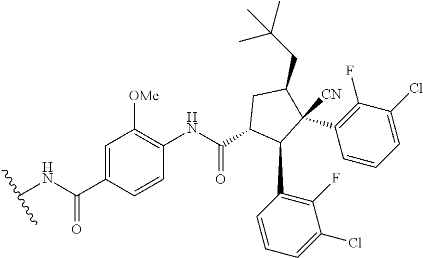 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1207 | 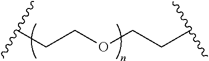 | 5 | 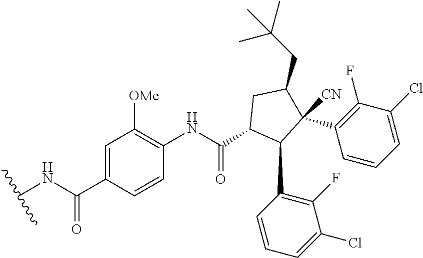 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1208 | 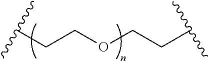 | 1 | 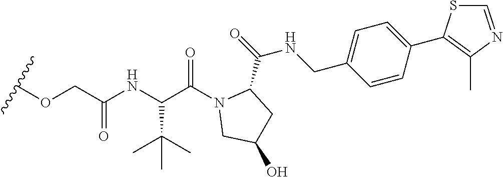 |
| 1209 | 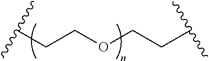 | 2 | 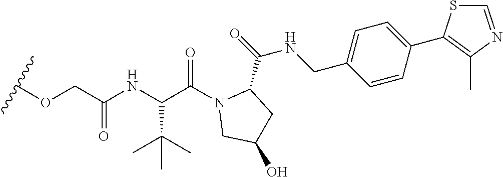 |
| 1210 | 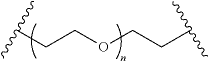 | 3 | 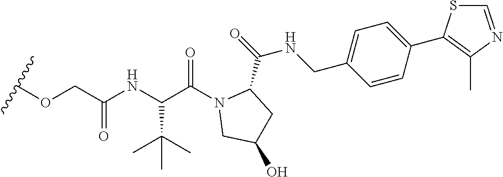 |
| 1211 | 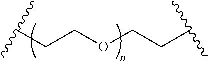 | 4 | 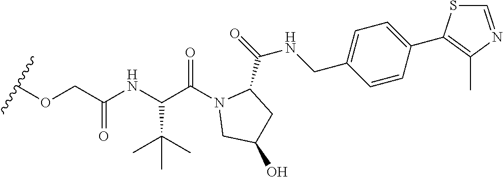 |
| 1212 | 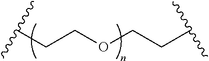 | 5 | 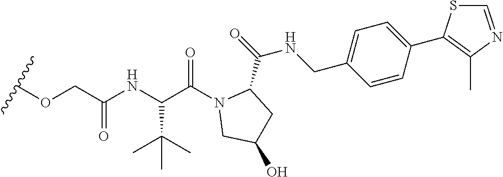 |
| 1213 | 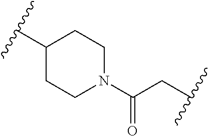 [SMILES: *CC(=O)N1CCC(*)CC1] | — | 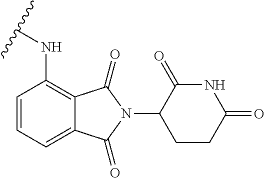 [SMILES: *Nc1cccc2c1C(=O)N(C1CCC(=O)NC1=O)C2=O] |
| 1214 | 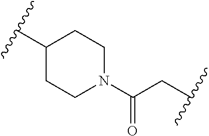 [SMILES: *CC(=O)N1CCC(*)CC1] | — | 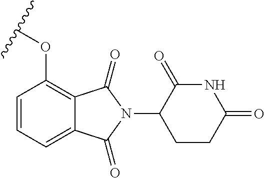 [SMILES: *Oc1cccc2c1C(=O)N(C1CCC(=O)NC1=O)C2=O] |
| 1215 | 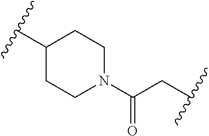 [SMILES: *CC(=O)N1CCC(*)CC1] | — | 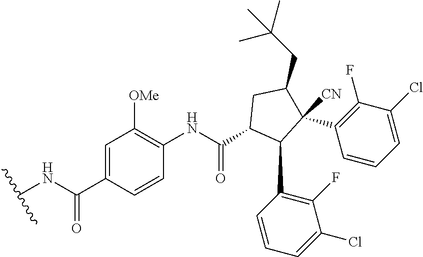 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1216 | 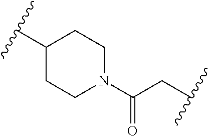 [SMILES: *CC(=O)N1CCC(*)CC1] | — | 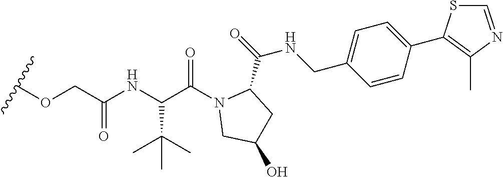 |
| 1217 | 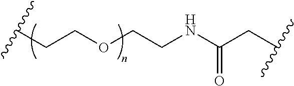 | 1 | 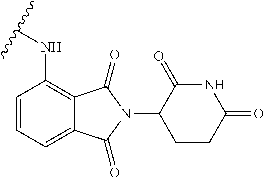 [SMILES: *Nc1cccc2c1C(=O)N(C1CCC(=O)NC1=O)C2=O] |
| 1218 | 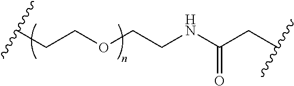 | 2 | 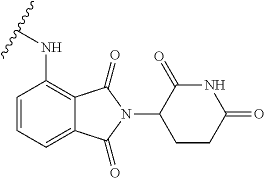 [SMILES: *Nc1cccc2c1C(=O)N(C1CCC(=O)NC1=O)C2=O] |
| 1219 | 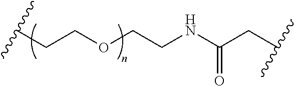 | 3 | 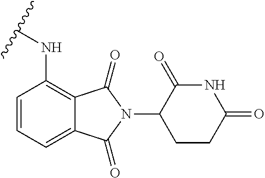 [SMILES: *Nc1cccc2c1C(=O)N(C1CCC(=O)NC1=O)C2=O] |
| 1220 | 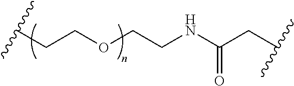 | 4 | 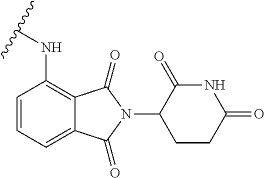 [SMILES: *Nc1cccc2c1C(=O)N(C1CCC(=O)NC1=O)C2=O] |
| 1221 | 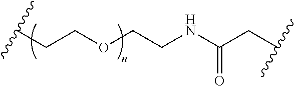 | 5 | 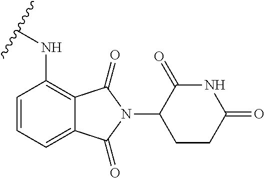 [SMILES: *Nc1cccc2c1C(=O)N(C1CCC(=O)NC1=O)C2=O] |
| 1222 | 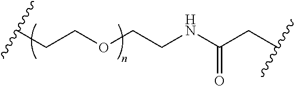 | 1 | 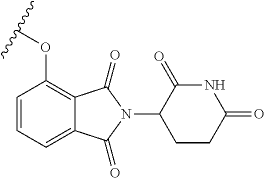 [SMILES: *Oc1cccc2c1C(=O)N(C1CCC(=O)NC1=O)C2=O] |
| 1223 | 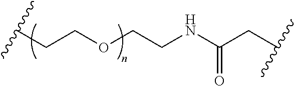 | 2 | 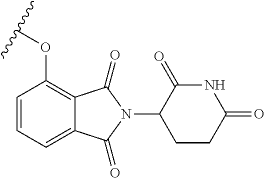 [SMILES: *Oc1cccc2c1C(=O)N(C1CCC(=O)NC1=O)C2=O] |
| 1224 | 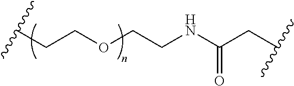 | 3 | 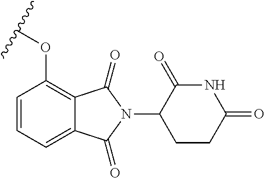 [SMILES: *Oc1cccc2c1C(=O)N(C1CCC(=O)NC1=O)C2=O] |
| 1225 | 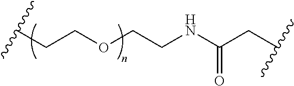 | 4 | 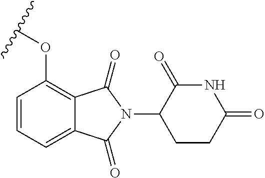 [SMILES: *Oc1cccc2c1C(=O)N(C1CCC(=O)NC1=O)C2=O] |
| 1226 | 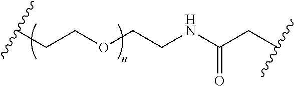 | 5 | 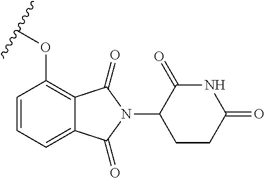 [SMILES: *Oc1cccc2c1C(=O)N(C1CCC(=O)NC1=O)C2=O] |
| 1227 | 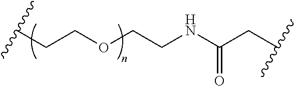 | 1 | 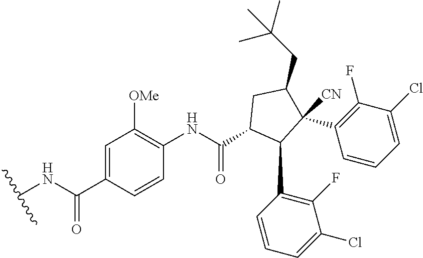 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1228 | 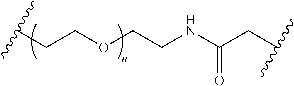 | 2 | 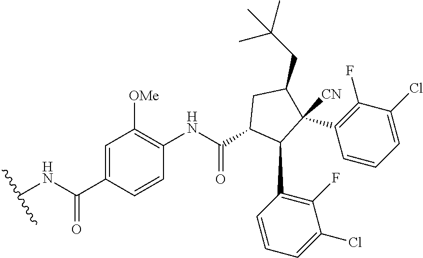 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1229 | 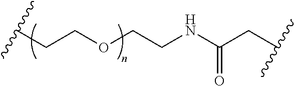 | 3 | 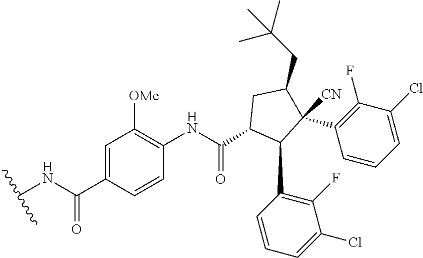 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1230 | 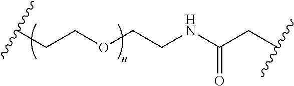 | 4 | 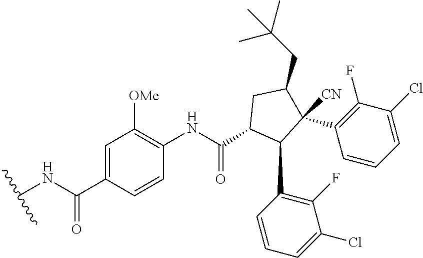 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1231 | 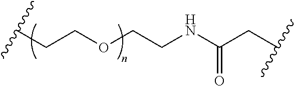 | 5 | 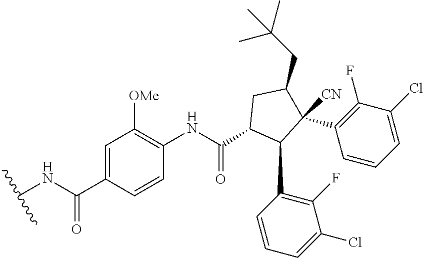 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1232 | 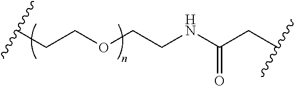 | 1 | 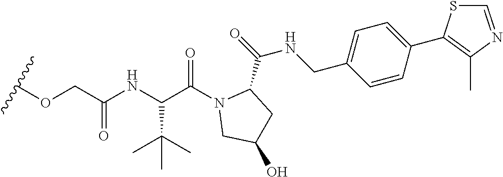 |
| 1233 | 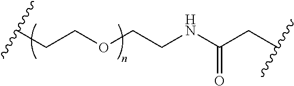 | 2 | 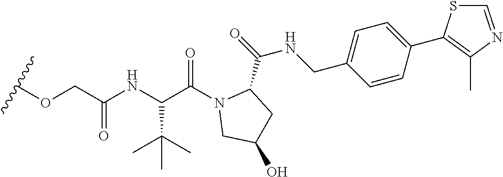 |
| 1234 | 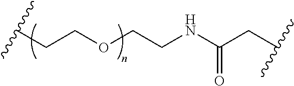 | 3 | 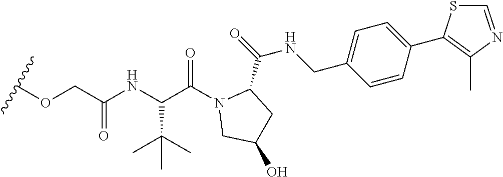 |
| 1235 | 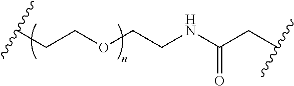 | 4 | 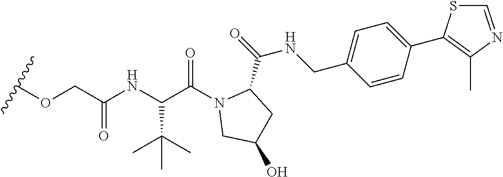 |
| 1236 | 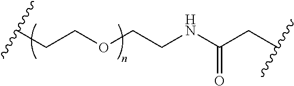 | 5 | 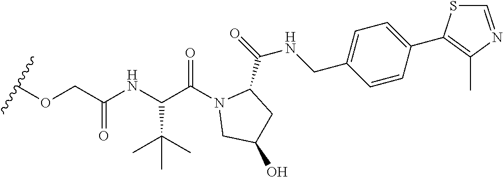 |
| 1237 | 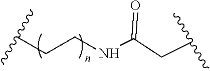 | 1 | 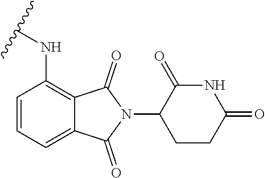 [SMILES: *Nc1cccc2c1C(=O)N(C1CCC(=O)NC1=O)C2=O] |
| 1238 | 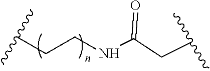 | 2 | 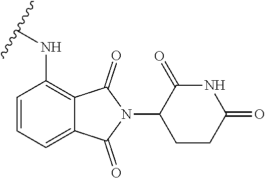 [SMILES: *Nc1cccc2c1C(=O)N(C1CCC(=O)NC1=O)C2=O] |
| 1239 | 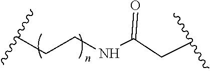 | 3 | 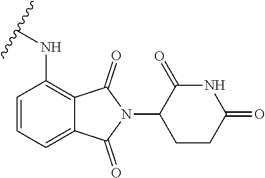 [SMILES: *Nc1cccc2c1C(=O)N(C1CCC(=O)NC1=O)C2=O] |
| 1240 | 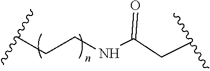 | 4 | 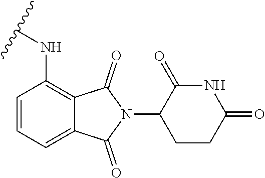 [SMILES: *Nc1cccc2c1C(=O)N(C1CCC(=O)NC1=O)C2=O] |
| 1241 | 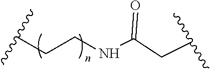 | 5 | 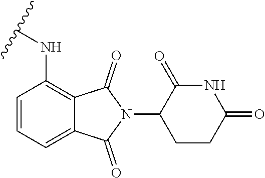 [SMILES: *Nc1cccc2c1C(=O)N(C1CCC(=O)NC1=O)C2=O] |
| 1242 | 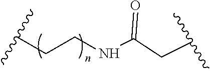 | 1 | 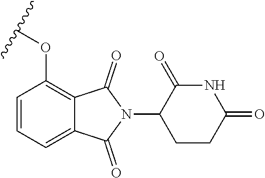 [SMILES: *Oc1cccc2c1C(=O)N(C1CCC(=O)NC1=O)C2=O] |
| 1243 | 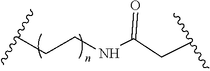 | 2 | 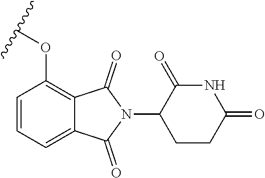 [SMILES: *Oc1cccc2c1C(=O)N(C1CCC(=O)NC1=O)C2=O] |
| 1244 | 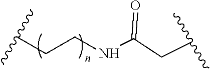 | 3 | 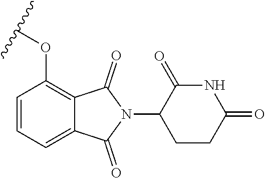 [SMILES: *Oc1cccc2c1C(=O)N(C1CCC(=O)NC1=O)C2=O] |
| 1245 | 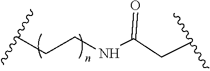 | 4 | 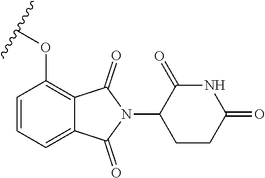 [SMILES: *Oc1cccc2c1C(=O)N(C1CCC(=O)NC1=O)C2=O] |
| 1246 | 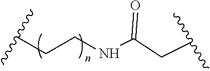 | 5 | 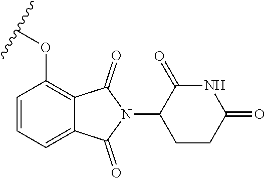 [SMILES: *Oc1cccc2c1C(=O)N(C1CCC(=O)NC1=O)C2=O] |
| 1247 | 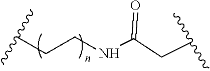 | 1 | 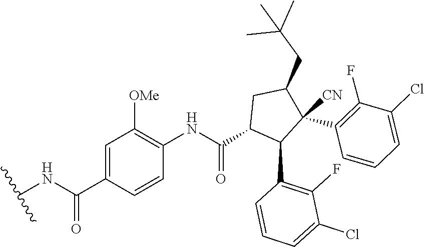 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1248 | 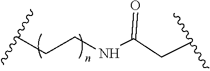 | 2 | 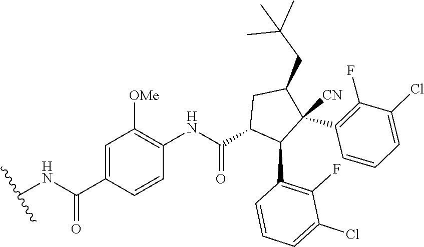 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1249 | 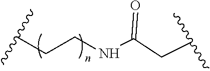 | 3 | 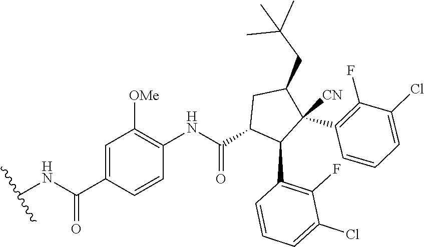 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1250 | 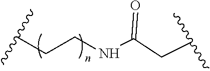 | 4 | 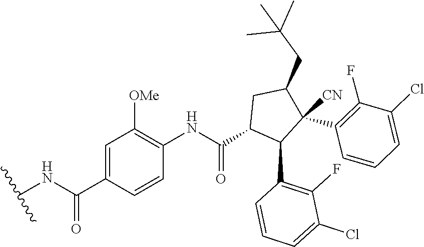 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1251 | 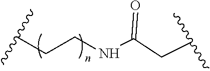 | 5 | 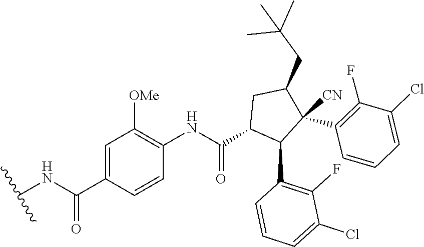 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1252 | 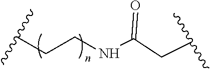 | 1 | 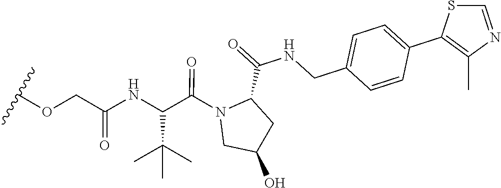 |
| 1253 | 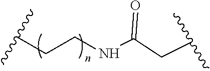 | 2 | 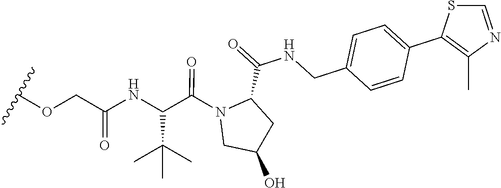 |
| 1254 | 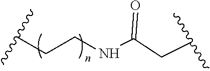 | 3 | 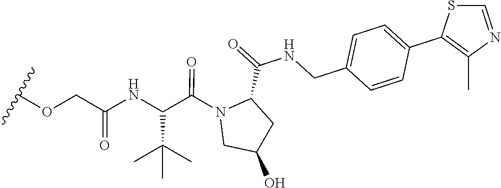 |
| 1255 | 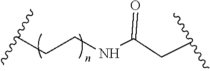 | 4 | 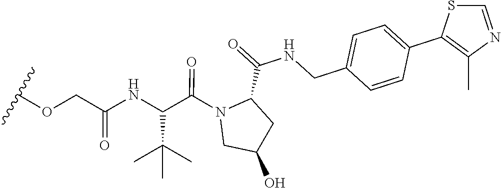 |
| 1256 | 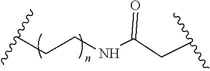 | 5 | 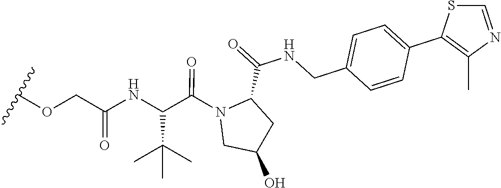 |
| 1257 | 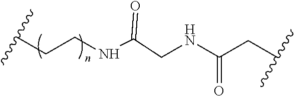 | 1 | 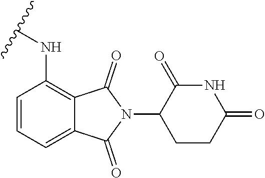 [SMILES: *Nc1cccc2c1C(=O)N(C1CCC(=O)NC1=O)C2=O] |
| 1258 | 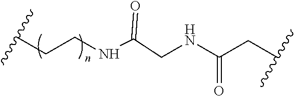 | 2 | 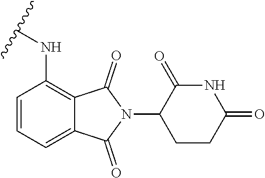 [SMILES: *Nc1cccc2c1C(=O)N(C1CCC(=O)NC1=O)C2=O] |
| 1259 | 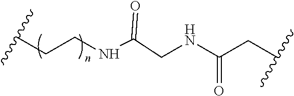 | 3 | 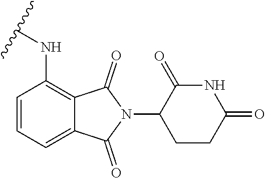 [SMILES: *Nc1cccc2c1C(=O)N(C1CCC(=O)NC1=O)C2=O] |
| 1260 | 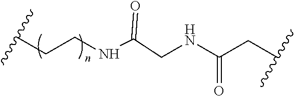 | 4 | 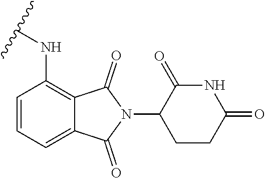 [SMILES: *Nc1cccc2c1C(=O)N(C1CCC(=O)NC1=O)C2=O] |
| 1261 | 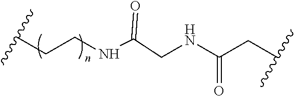 | 5 | 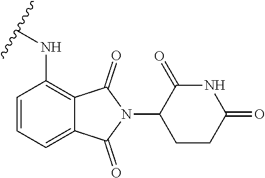 [SMILES: *Nc1cccc2c1C(=O)N(C1CCC(=O)NC1=O)C2=O] |
| 1262 | 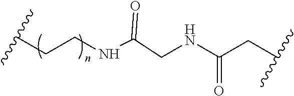 | 1 | 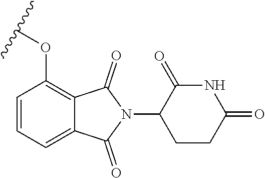 [SMILES: *Oc1cccc2c1C(=O)N(C1CCC(=O)NC1=O)C2=O] |
| 1263 | 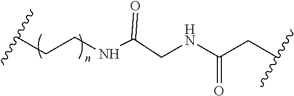 | 2 | 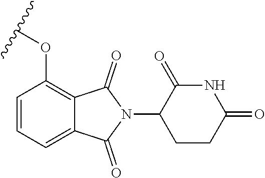 [SMILES: *Oc1cccc2c1C(=O)N(C1CCC(=O)NC1=O)C2=O] |
| 1264 | 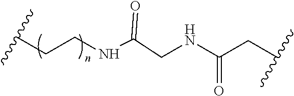 | 3 | 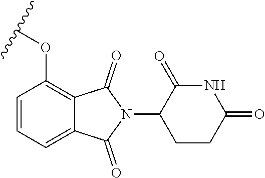 [SMILES: *Oc1cccc2c1C(=O)N(C1CCC(=O)NC1=O)C2=O] |
| 1265 | 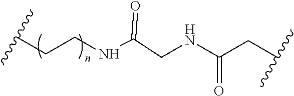 | 4 | 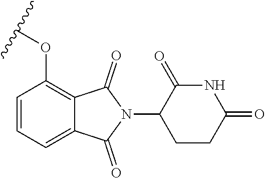 [SMILES: *Oc1cccc2c1C(=O)N(C1CCC(=O)NC1=O)C2=O] |
| 1266 | 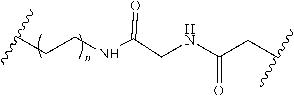 | 5 | 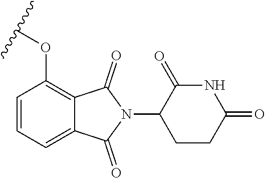 [SMILES: *Oc1cccc2c1C(=O)N(C1CCC(=O)NC1=O)C2=O] |
| 1267 | 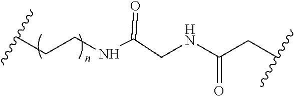 | 1 | 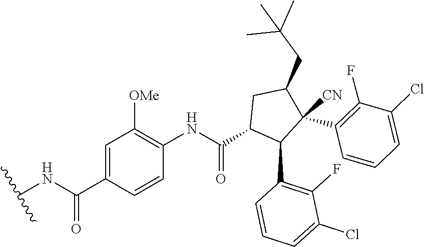 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1268 | 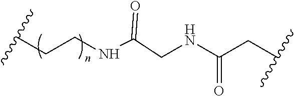 | 2 | 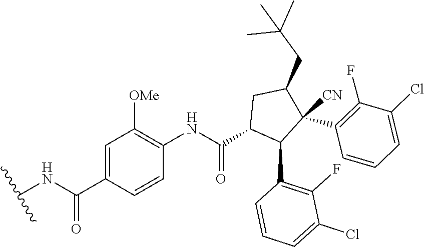 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1269 | 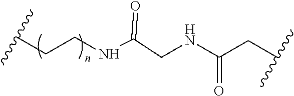 | 3 | 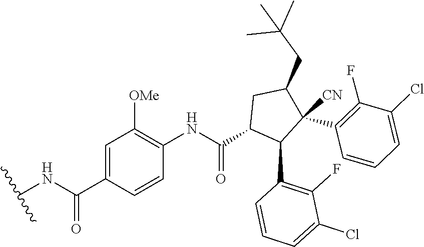 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1270 | 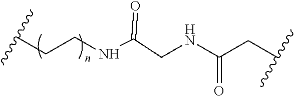 | 4 | 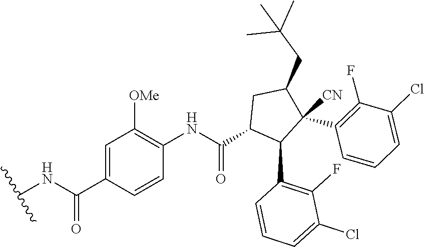 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1271 | 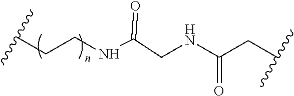 | 5 | 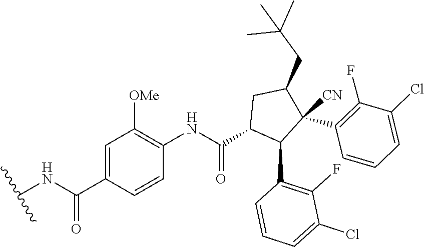 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1272 | 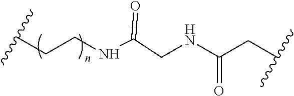 | 1 | 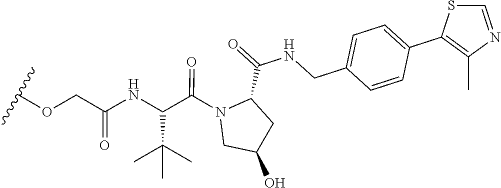 |
| 1273 | 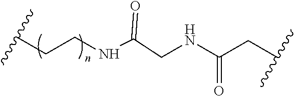 | 2 | 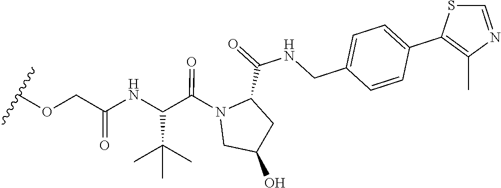 |
| 1274 | 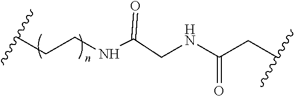 | 3 | 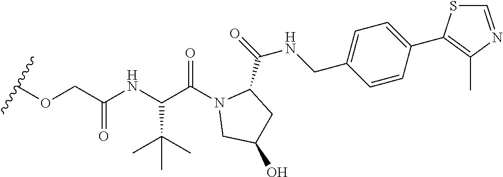 |
| 1275 | 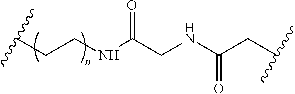 | 4 | 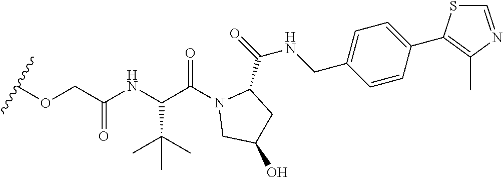 |
| 1276 | 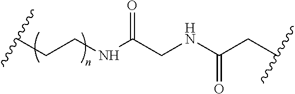 | 5 | 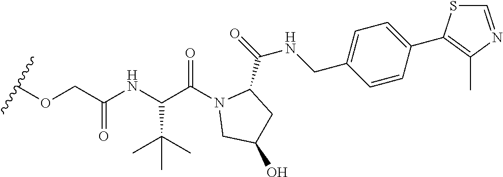 |

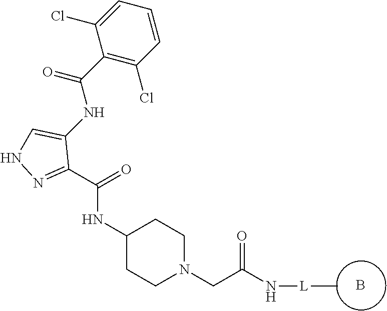

| For- | | | |
|---|---|---|---|
| mu- | | | |
| la | | | |
| No. | L | n | B |
| 1277 | 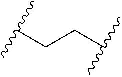 [SMILES: *CC*] | — | 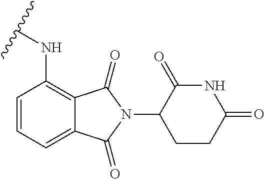 [SMILES: *Nc1cccc2c1C(=O)N(C1CCC(=O)NC1=O)C2=O] |
| 1278 | 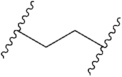 [SMILES: *CC*] | — | 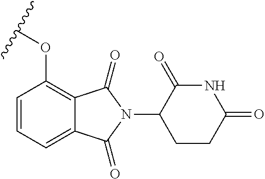 [SMILES: *Oc1cccc2c1C(=O)N(C1CCC(=O)NC1=O)C2=O] |
| 1279 | 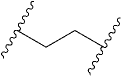 [SMILES: *CC*] | — | 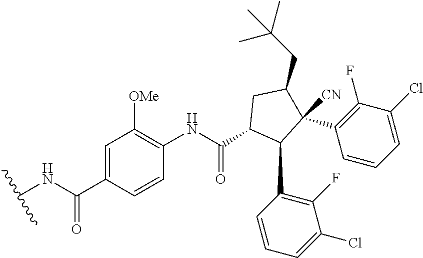 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1280 | 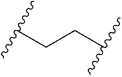 [SMILES: *CC*] | — | 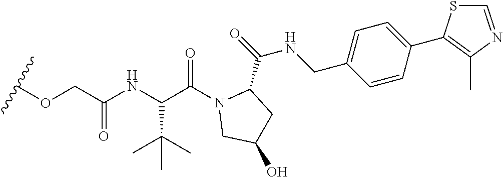 |
| 1281 | 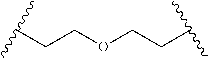 [SMILES: *CCOCC*] | — | 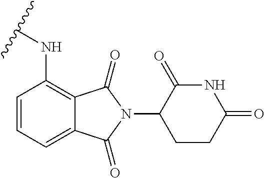 [SMILES: *Nc1cccc2c1C(=O)N(C1CCC(=O)NC1=O)C2=O] |
| 1282 | 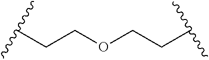 [SMILES: *CCOCC*] | — | 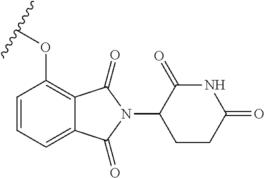 [SMILES: *Oc1cccc2c1C(=O)N(C1CCC(=O)NC1=O)C2=O] |
| 1283 | 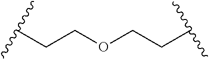 [SMILES: *CCOCC*] | — | 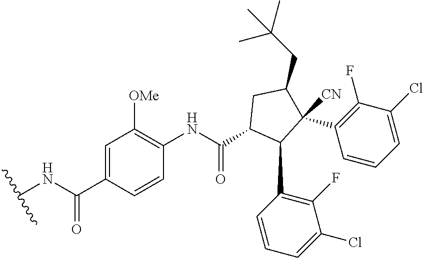 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1284 | 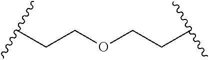 [SMILES: *CCOCC*] | — | 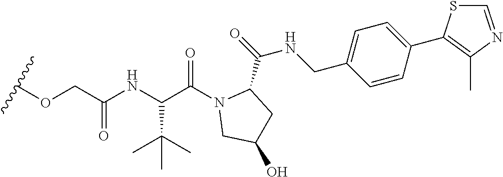 |
| 1285 | 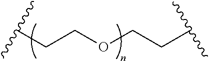 | 1 | 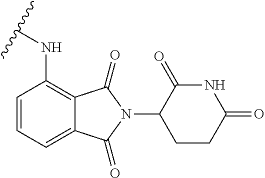 [SMILES: *Nc1cccc2c1C(=O)N(C1CCC(=O)NC1=O)C2=O] |
| 1286 | 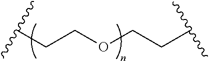 | 2 | 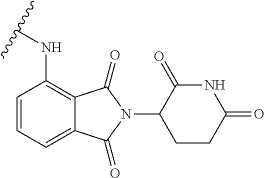 [SMILES: *Nc1cccc2c1C(=O)N(C1CCC(=O)NC1=O)C2=O] |
| 1287 | 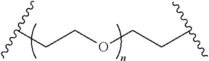 | 3 | 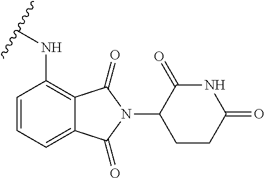 [SMILES: *Nc1cccc2c1C(=O)N(C1CCC(=O)NC1=O)C2=O] |
| 1288 | 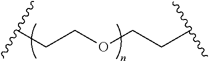 | 4 | 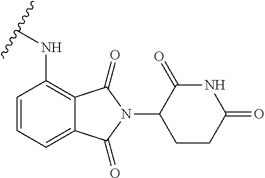 [SMILES: *Nc1cccc2c1C(=O)N(C1CCC(=O)NC1=O)C2=O] |
| 1289 | 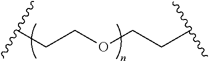 | 5 | 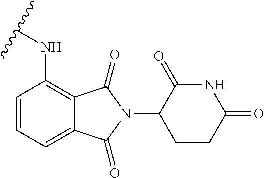 [SMILES: *Nc1cccc2c1C(=O)N(C1CCC(=O)NC1=O)C2=O] |
| 1290 | 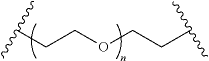 | 1 | 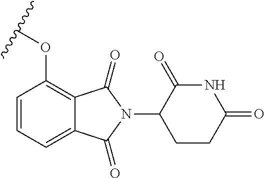 [SMILES: *Oc1cccc2c1C(=O)N(C1CCC(=O)NC1=O)C2=O] |
| 1291 | 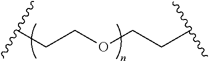 | 2 | 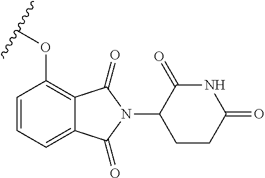 [SMILES: *Oc1cccc2c1C(=O)N(C1CCC(=O)NC1=O)C2=O] |
| 1292 | 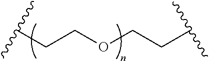 | 3 | 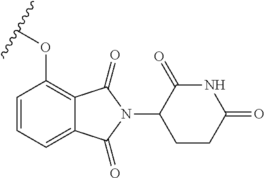 [SMILES: *Oc1cccc2c1C(=O)N(C1CCC(=O)NC1=O)C2=O] |
| 1293 | 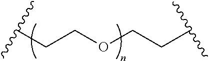 | 4 | 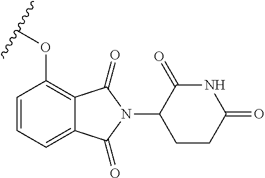 [SMILES: *Oc1cccc2c1C(=O)N(C1CCC(=O)NC1=O)C2=O] |
| 1294 | 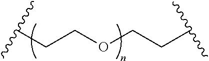 | 5 | 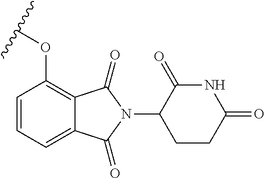 [SMILES: *Oc1cccc2c1C(=O)N(C1CCC(=O)NC1=O)C2=O] |
| 1295 | 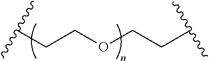 | 1 | 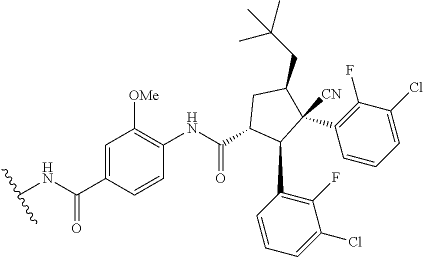 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1296 | 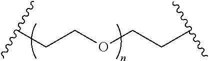 | 2 | 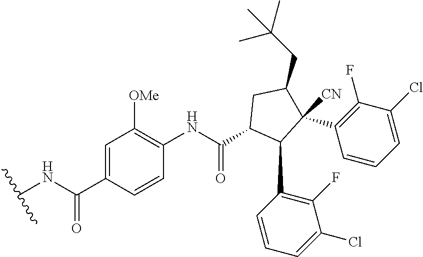 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1297 | 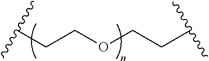 | 3 | 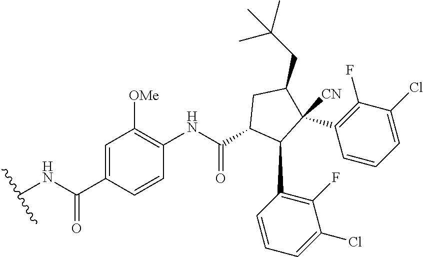 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1298 | 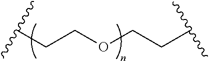 | 4 | 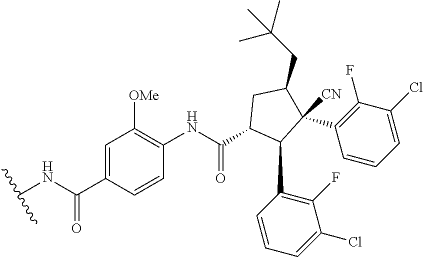 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1299 | 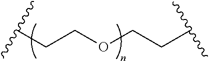 | 5 | 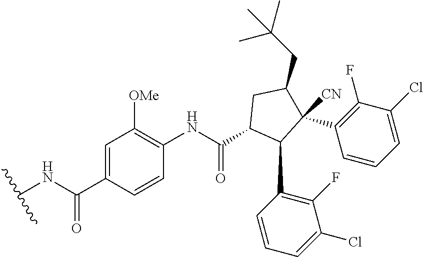 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1300 | 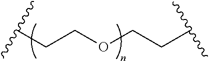 | 1 | 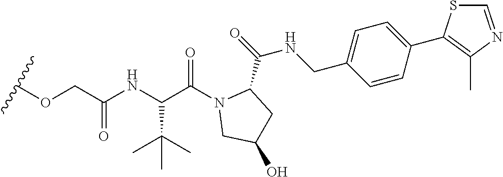 |
| 1301 | 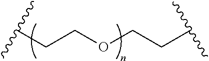 | 2 | 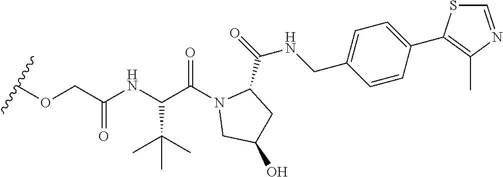 |
| 1302 | 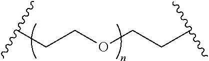 | 3 | 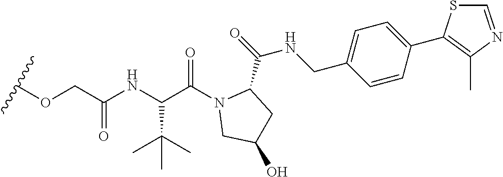 |
| 1303 | 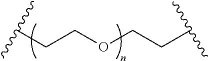 | 4 | 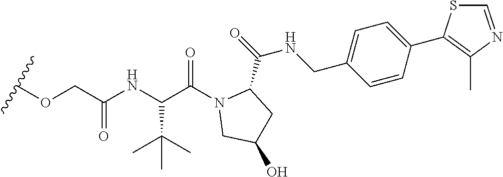 |
| 1304 | 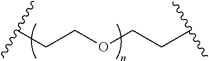 | 5 | 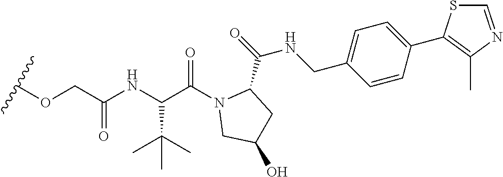 |
| 1305 | 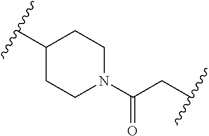 [SMILES: *CC(=O)N1CCC(*)CC1] | — | 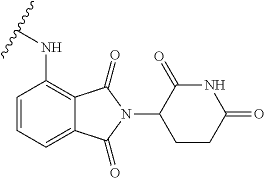 [SMILES: *Nc1cccc2c1C(=O)N(C1CCC(=O)NC1=O)C2=O] |
| 1306 | 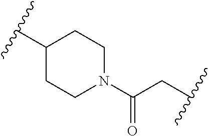 [SMILES: *CC(=O)N1CCC(*)CC1] | — | 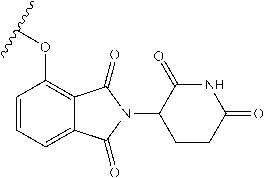 [SMILES: *Oc1cccc2c1C(=O)N(C1CCC(=O)NC1=O)C2=O] |
| 1307 | 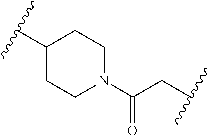 [SMILES: *CC(=O)N1CCC(*)CC1] | — | 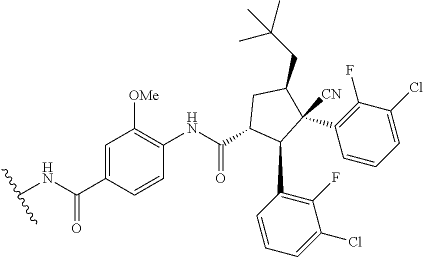 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1308 | 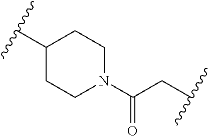 [SMILES: *CC(=O)N1CCC(*)CC1] | — | 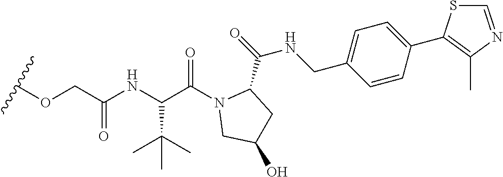 |
| 1309 | 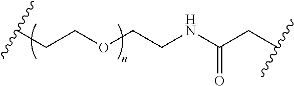 | 1 | 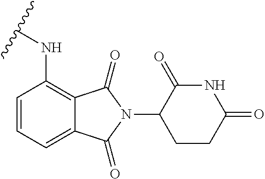 [SMILES: *Nc1cccc2c1C(=O)N(C1CCC(=O)NC1=O)C2=O] |
| 1310 | 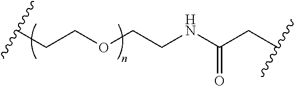 | 2 | 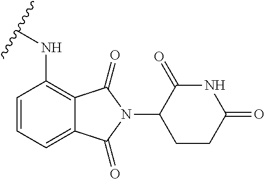 [SMILES: *Nc1cccc2c1C(=O)N(C1CCC(=O)NC1=O)C2=O] |
| 1311 | 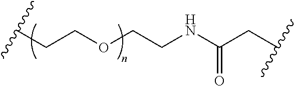 | 3 | 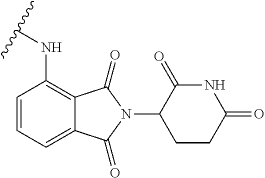 [SMILES: *Nc1cccc2c1C(=O)N(C1CCC(=O)NC1=O)C2=O] |
| 1312 | 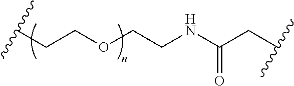 | 4 | 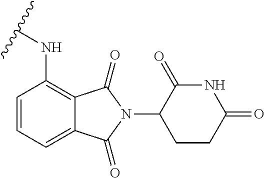 [SMILES: *Nc1cccc2c1C(=O)N(C1CCC(=O)NC1=O)C2=O] |
| 1313 | 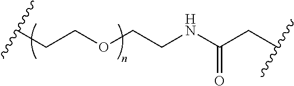 | 5 | 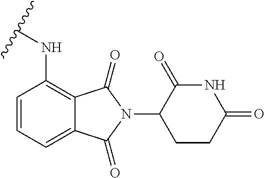 [SMILES: *Nc1cccc2c1C(=O)N(C1CCC(=O)NC1=O)C2=O] |
| 1314 | 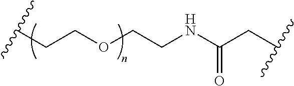 | 1 | 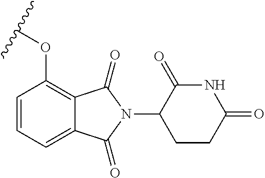 [SMILES: *Oc1cccc2c1C(=O)N(C1CCC(=O)NC1=O)C2=O] |
| 1315 | 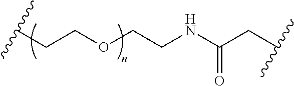 | 2 | 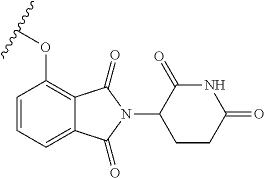 [SMILES: *Oc1cccc2c1C(=O)N(C1CCC(=O)NC1=O)C2=O] |
| 1316 | 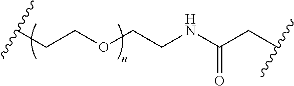 | 3 | 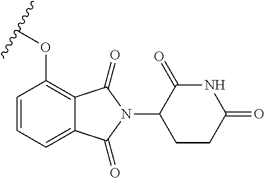 [SMILES: *Oc1cccc2c1C(=O)N(C1CCC(=O)NC1=O)C2=O] |
| 1317 | 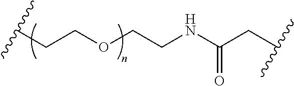 | 4 | 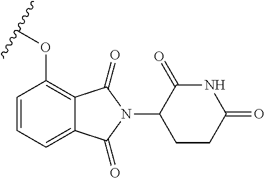 [SMILES: *Oc1cccc2c1C(=O)N(C1CCC(=O)NC1=O)C2=O] |
| 1318 | 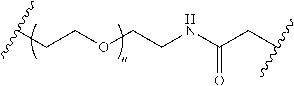 | 5 | 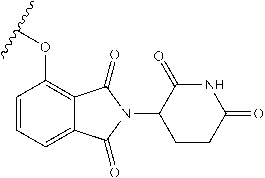 [SMILES: *Oc1cccc2c1C(=O)N(C1CCC(=O)NC1=O)C2=O] |
| 1319 | 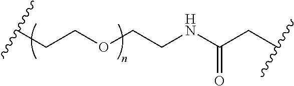 | 1 | 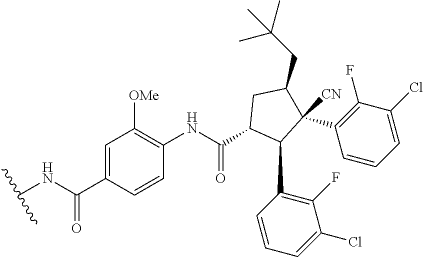 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1320 | 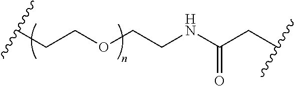 | 2 | 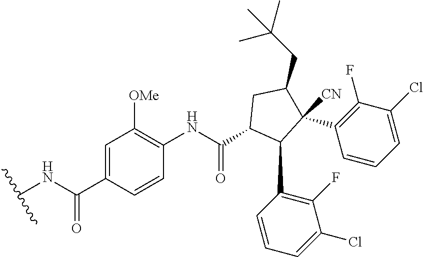 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1321 | 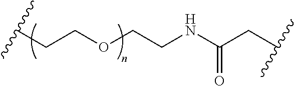 | 3 | 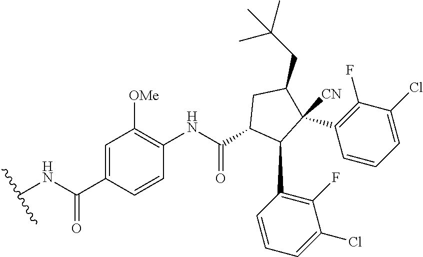 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1322 | 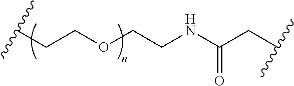 | 4 | 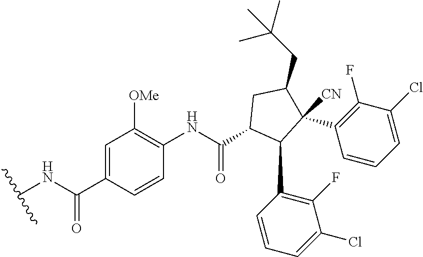 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1323 | 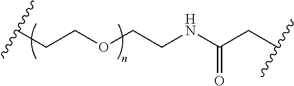 | 5 | 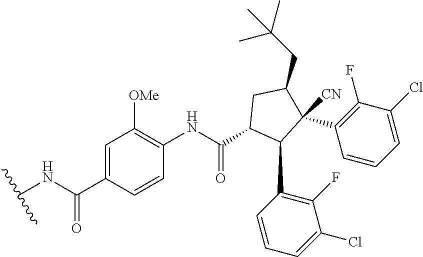 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1324 | 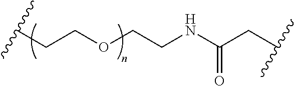 | 1 | 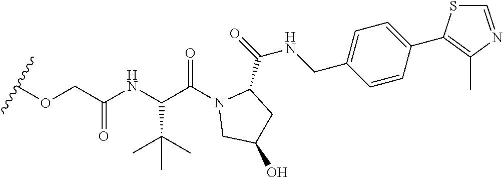 |
| 1325 | 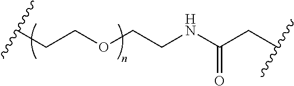 | 2 | 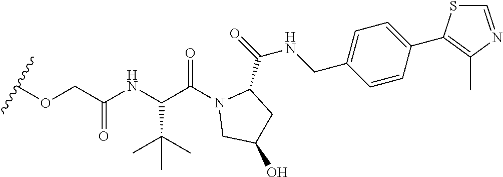 |
| 1326 | 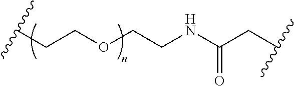 | 3 | 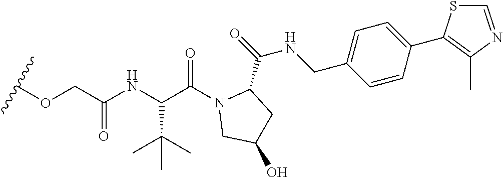 |
| 1327 | 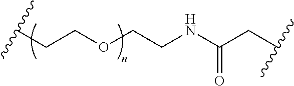 | 4 | 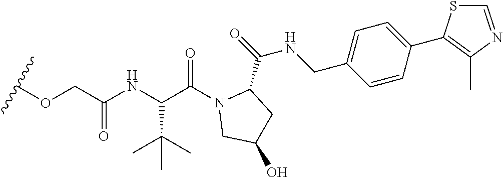 |
| 1328 | 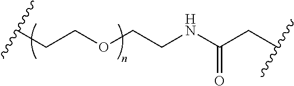 | 5 | 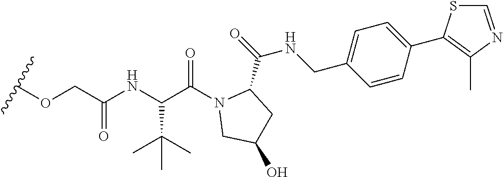 |
| 1329 | 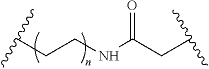 | 1 | 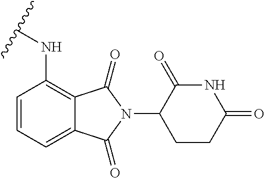 [SMILES: *Nc1cccc2c1C(=O)N(C1CCC(=O)NC1=O)C2=O] |
| 1330 | 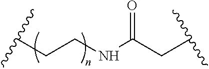 | 2 | 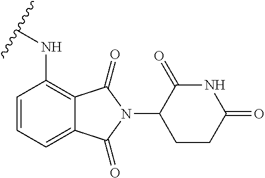 [SMILES: *Nc1cccc2c1C(=O)N(C1CCC(=O)NC1=O)C2=O] |
| 1331 | 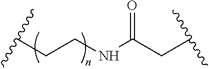 | 3 | 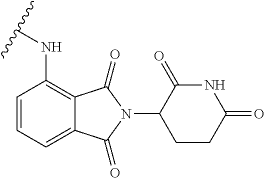 [SMILES: *Nc1cccc2c1C(=O)N(C1CCC(=O)NC1=O)C2=O] |
| 1332 | 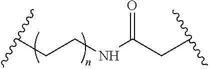 | 4 | 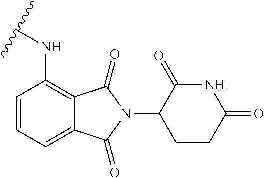 [SMILES: *Nc1cccc2c1C(=O)N(C1CCC(=O)NC1=O)C2=O] |
| 1333 | 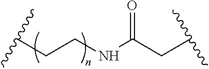 | 5 | 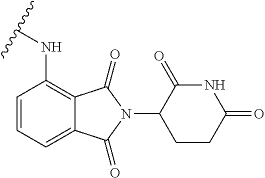 [SMILES: *Nc1cccc2c1C(=O)N(C1CCC(=O)NC1=O)C2=O] |
| 1334 | 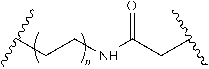 | 1 | 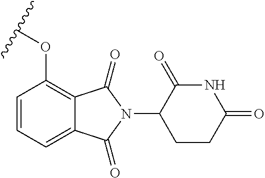 [SMILES: *Oc1cccc2c1C(=O)N(C1CCC(=O)NC1=O)C2=O] |
| 1335 | 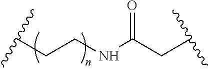 | 2 | 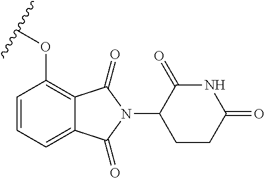 [SMILES: *Oc1cccc2c1C(=O)N(C1CCC(=O)NC1=O)C2=O] |
| 1336 | 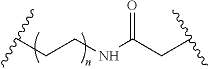 | 3 | 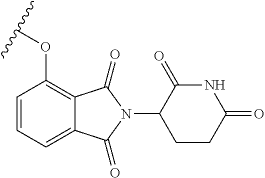 [SMILES: *Oc1cccc2c1C(=O)N(C1CCC(=O)NC1=O)C2=O] |
| 1337 | 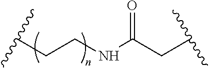 | 4 | 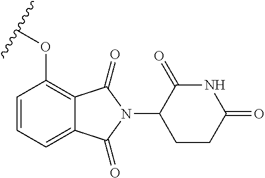 [SMILES: *Oc1cccc2c1C(=O)N(C1CCC(=O)NC1=O)C2=O] |
| 1338 | 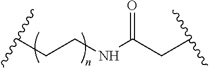 | 5 | 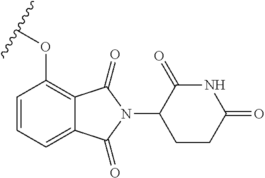 [SMILES: *Oc1cccc2c1C(=O)N(C1CCC(=O)NC1=O)C2=O] |
| 1339 | 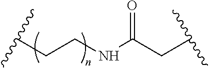 | 1 | 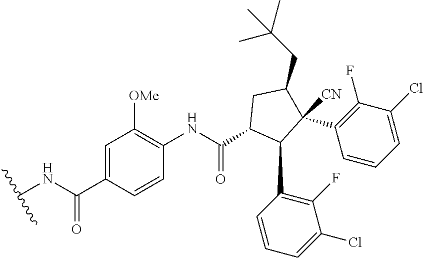 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1340 | 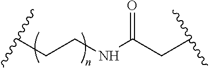 | 2 | 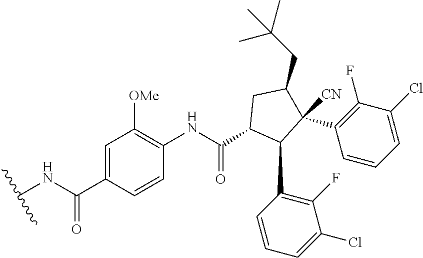 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1341 | 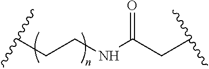 | 3 | 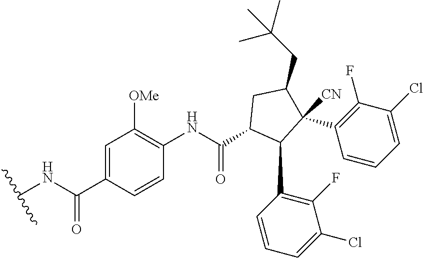 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1342 | 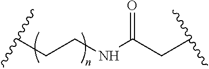 | 4 | 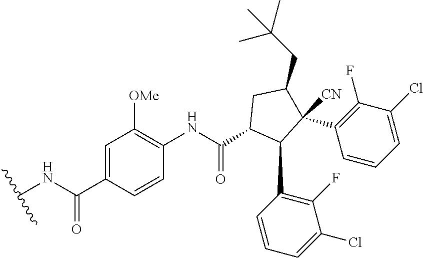 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1343 | 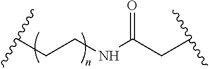 | 5 | 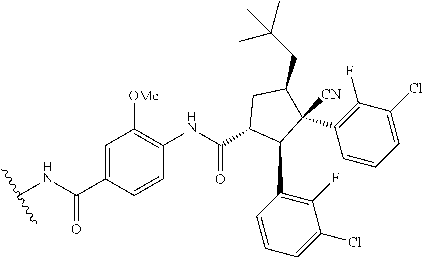 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1344 | 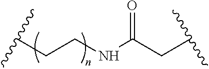 | 1 | 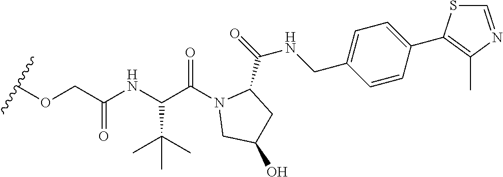 |
| 1345 | 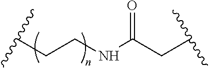 | 2 | 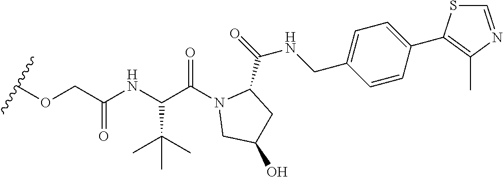 |
| 1346 | 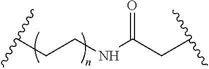 | 3 | 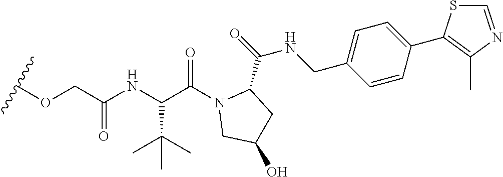 |
| 1347 | 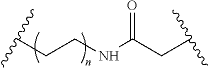 | 4 | 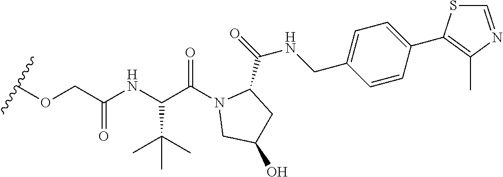 |
| 1348 | 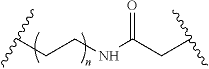 | 5 | 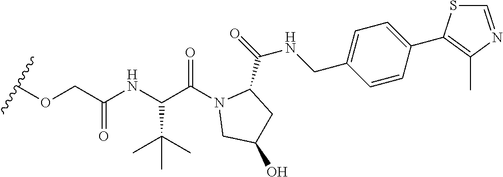 |
| 1349 | 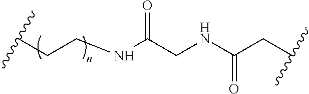 | 1 | 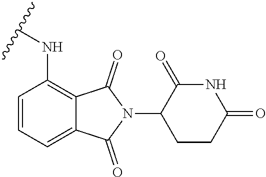 [SMILES: *Nc1cccc2c1C(=O)N(C1CCC(=O)NC1=O)C2=O] |
| 1350 | 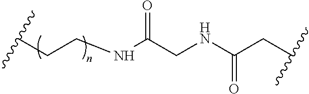 | 2 | 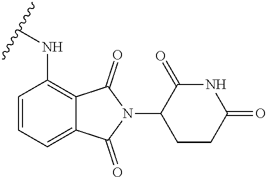 [SMILES: *Nc1cccc2c1C(=O)N(C1CCC(=O)NC1=O)C2=O] |
| 1351 | 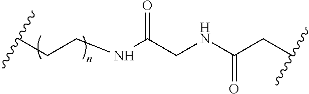 | 3 | 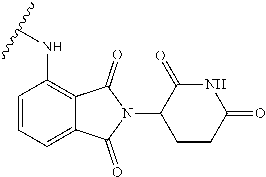 [SMILES: *Nc1cccc2c1C(=O)N(C1CCC(=O)NC1=O)C2=O] |
| 1352 | 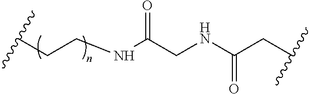 | 4 | 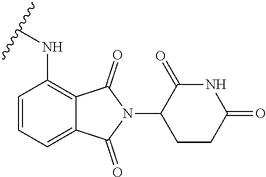 [SMILES: *Nc1cccc2c1C(=O)N(C1CCC(=O)NC1=O)C2=O] |
| 1353 | 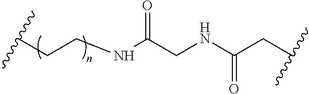 | 5 | 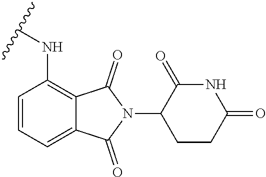 [SMILES: *Nc1cccc2c1C(=O)N(C1CCC(=O)NC1=O)C2=O] |
| 1354 | 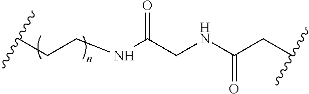 | 1 | 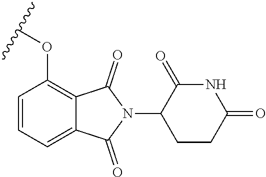 [SMILES: *Oc1cccc2c1C(=O)N(C1CCC(=O)NC1=O)C2=O] |
| 1355 | 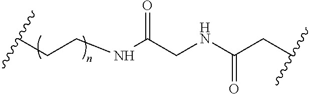 | 2 | 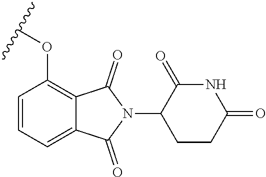 [SMILES: *Oc1cccc2c1C(=O)N(C1CCC(=O)NC1=O)C2=O] |
| 1356 | 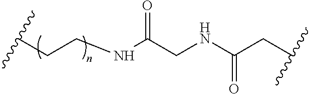 | 3 | 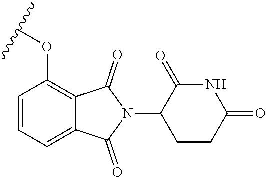 [SMILES: *Oc1cccc2c1C(=O)N(C1CCC(=O)NC1=O)C2=O] |
| 1357 | 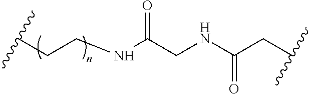 | 4 | 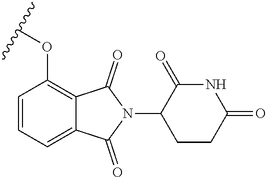 [SMILES: *Oc1cccc2c1C(=O)N(C1CCC(=O)NC1=O)C2=O] |
| 1358 | 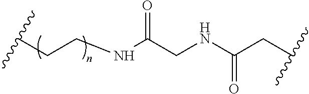 | 5 | 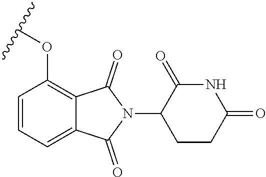 [SMILES: *Oc1cccc2c1C(=O)N(C1CCC(=O)NC1=O)C2=O] |
| 1359 | 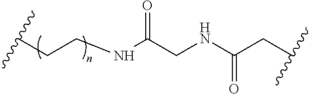 | 1 | 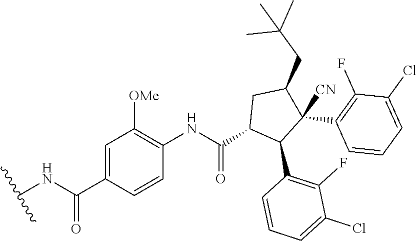 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1360 | 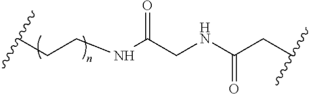 | 2 | 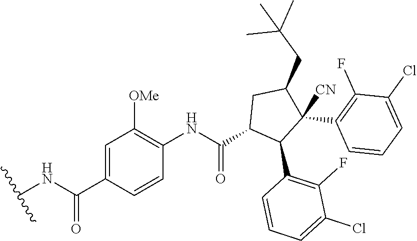 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1361 | 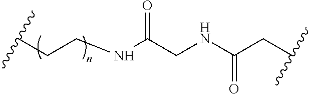 | 3 | 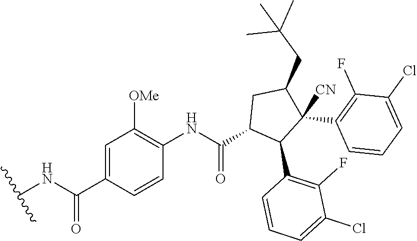 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1362 | 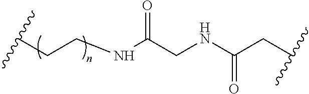 | 4 | 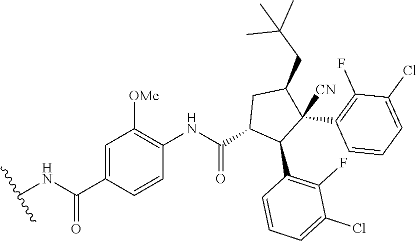 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1363 | 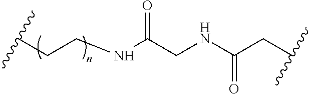 | 5 | 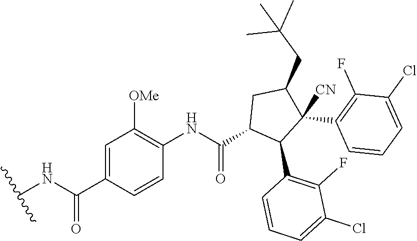 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1364 | 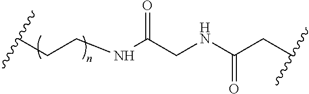 | 1 | 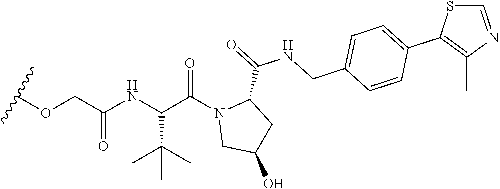 |
| 1365 | 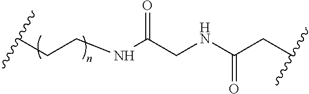 | 2 | 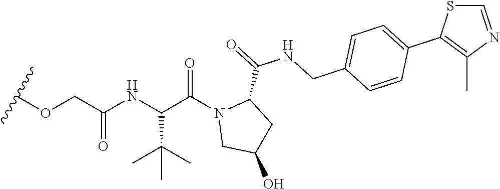 |
| 1366 | 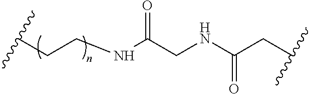 | 3 | 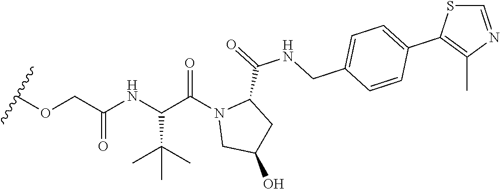 |
| 1367 | 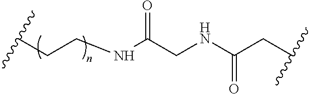 | 4 | 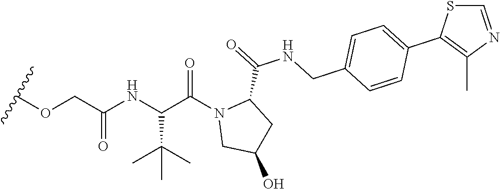 |
| 1368 | 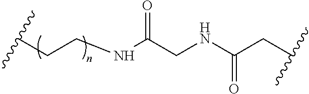 | 5 | 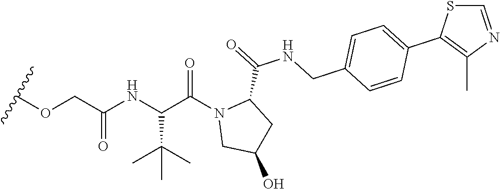 |

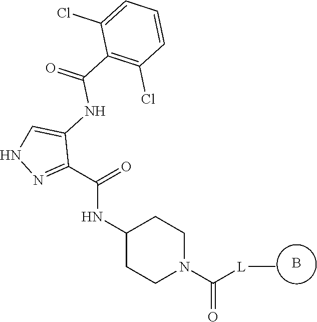

| For- | | | |
|---|---|---|---|
| mu- | | | |
| la | | | |
| No. | L | n | B |
| 1369 | 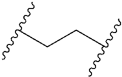 [SMILES: *CC*] | — | 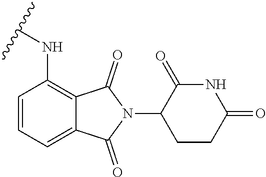 [SMILES: *Nc1cccc2c1C(=O)N(C1CCC(=O)NC1=O)C2=O] |
| 1370 | 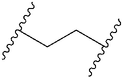 [SMILES: *CC*] | — | 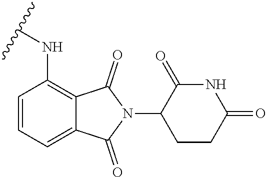 [SMILES: *Nc1cccc2c1C(=O)N(C1CCC(=O)NC1=O)C2=O] |
| 1371 | 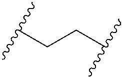 [SMILES: *CC*] | — | 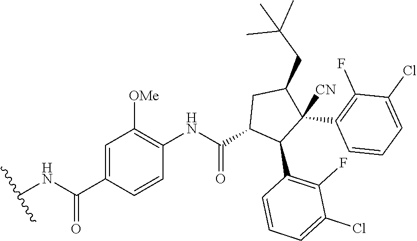 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1372 | 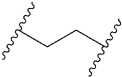 [SMILES: *CC*] | — | 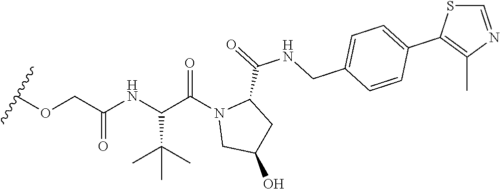 |
| 1373 | 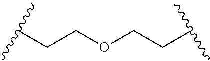 [SMILES: *CCOCC*] | — | 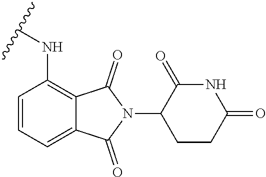 [SMILES: *Nc1cccc2c1C(=O)N(C1CCC(=O)NC1=O)C2=O] |
| 1374 | 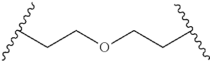 [SMILES: *CCOCC*] | — | 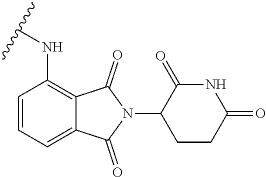 [SMILES: *Nc1cccc2c1C(=O)N(C1CCC(=O)NC1=O)C2=O] |
| 1375 | 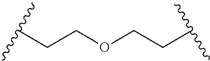 [SMILES: *CCOCC*] | — | 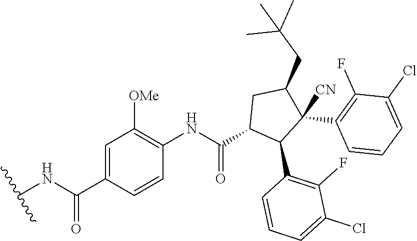 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1376 | 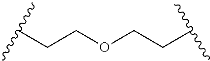 [SMILES: *CCOCC*] | — | 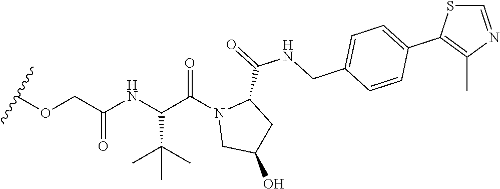 |
| 1377 | 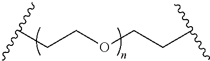 | 1 | 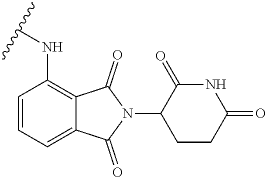 [SMILES: *Nc1cccc2c1C(=O)N(C1CCC(=O)NC1=O)C2=O] |
| 1378 | 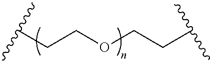 | 2 | 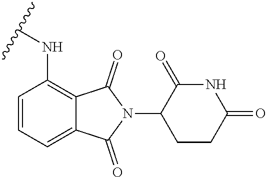 [SMILES: *Nc1cccc2c1C(=O)N(C1CCC(=O)NC1=O)C2=O] |
| 1379 | 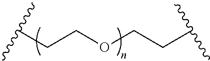 | 3 | 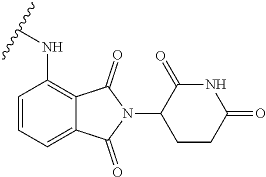 [SMILES: *Nc1cccc2c1C(=O)N(C1CCC(=O)NC1=O)C2=O] |
| 1380 | 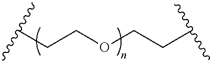 | 4 | 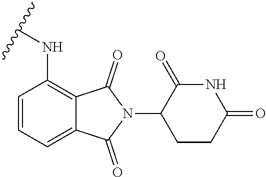 [SMILES: *Nc1cccc2c1C(=O)N(C1CCC(=O)NC1=O)C2=O] |
| 1381 | 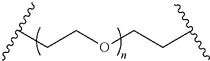 | 5 | 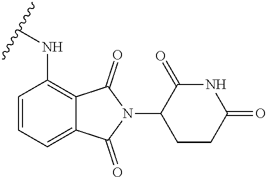 [SMILES: *Nc1cccc2c1C(=O)N(C1CCC(=O)NC1=O)C2=O] |
| 1382 | 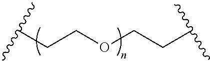 | 1 | 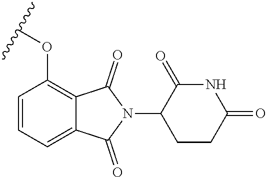 [SMILES: *Oc1cccc2c1C(=O)N(C1CCC(=O)NC1=O)C2=O] |
| 1383 | 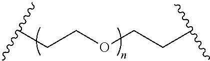 | 2 | 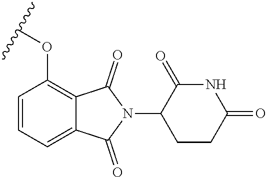 [SMILES: *Oc1cccc2c1C(=O)N(C1CCC(=O)NC1=O)C2=O] |
| 1384 | 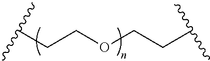 | 3 | 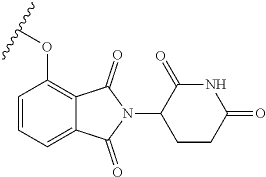 [SMILES: *Oc1cccc2c1C(=O)N(C1CCC(=O)NC1=O)C2=O] |
| 1385 | 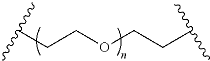 | 4 | 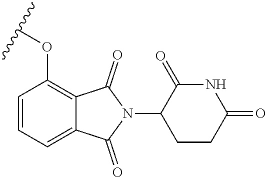 [SMILES: *Oc1cccc2c1C(=O)N(C1CCC(=O)NC1=O)C2=O] |
| 1386 | 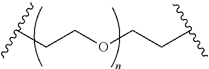 | 5 | 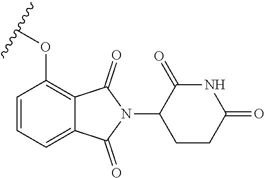 [SMILES: *Oc1cccc2c1C(=O)N(C1CCC(=O)NC1=O)C2=O] |
| 1387 | 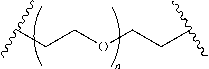 | 1 | 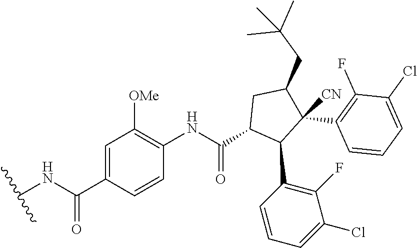 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1388 | 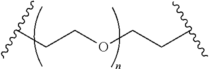 | 2 | 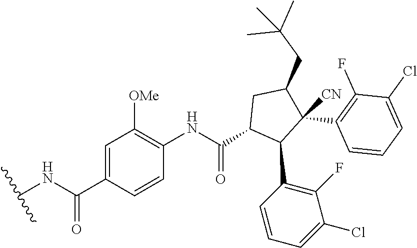 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1389 | 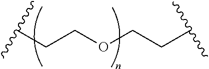 | 3 | 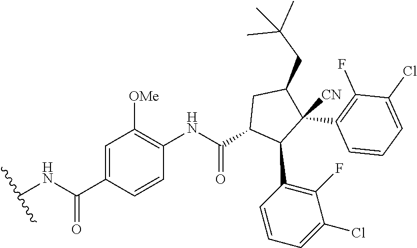 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1390 | 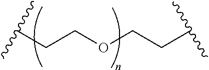 | 4 | 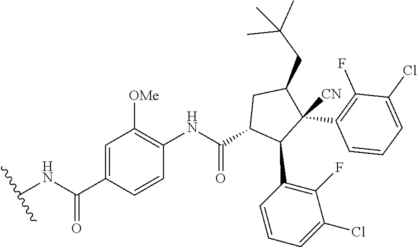 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1391 | 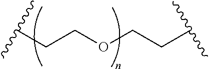 | 5 | 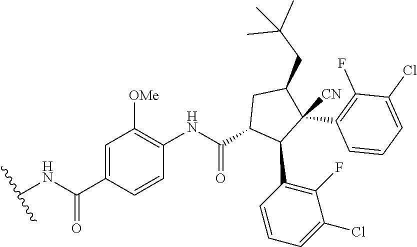 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1392 | 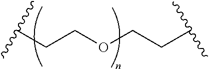 | 1 | 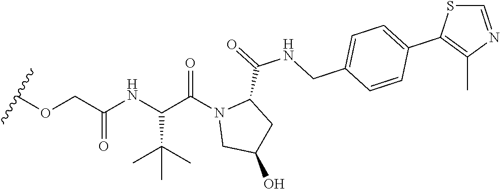 |
| 1393 | 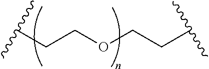 | 2 | 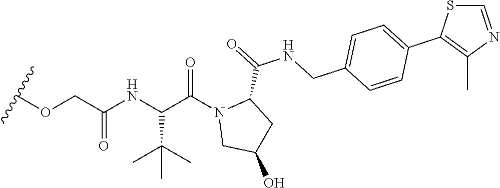 |
| 1394 | 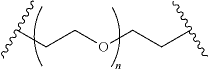 | 3 | 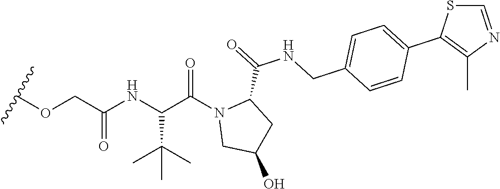 |
| 1395 | 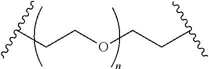 | 4 | 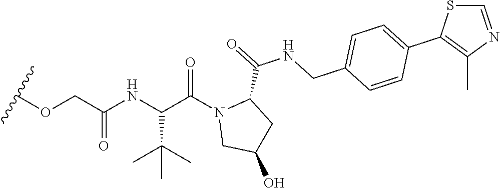 |
| 1396 | 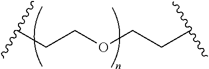 | 5 | 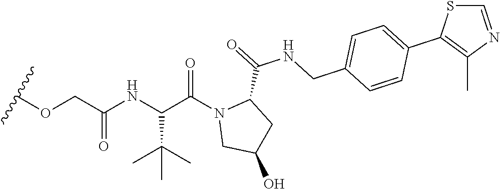 |
| 1397 | 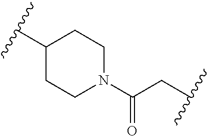 [SMILES: *CC(=O)N1CCC(*)CC1] | — | 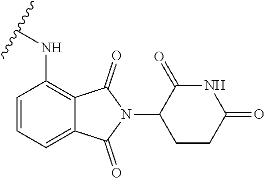 [SMILES: *Nc1cccc2c1C(=O)N(C1CCC(=O)NC1=O)C2=O] |
| 1398 | 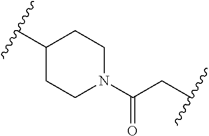 [SMILES: *CC(=O)N1CCC(*)CC1] | — | 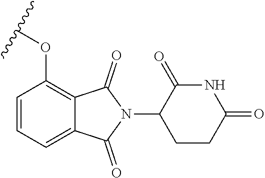 [SMILES: *Oc1cccc2c1C(=O)N(C1CCC(=O)NC1=O)C2=O] |
| 1399 | 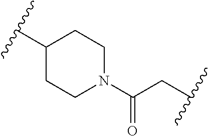 [SMILES: *CC(=O)N1CCC(*)CC1] | — | 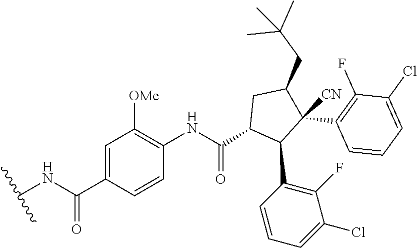 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1400 | 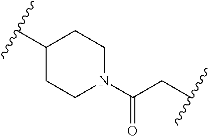 [SMILES: *CC(=O)N1CCC(*)CC1] | — | 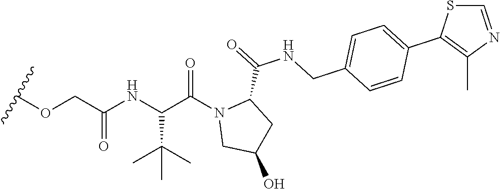 |
| 1401 | 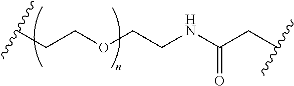 | 1 | 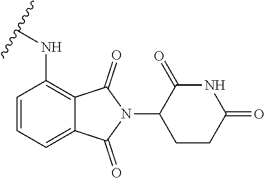 [SMILES: *Nc1cccc2c1C(=O)N(C1CCC(=O)NC1=O)C2=O] |
| 1402 | 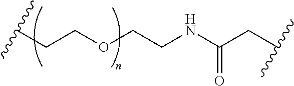 | 2 | 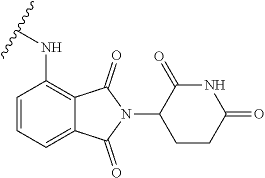 [SMILES: *Nc1cccc2c1C(=O)N(C1CCC(=O)NC1=O)C2=O] |
| 1403 | 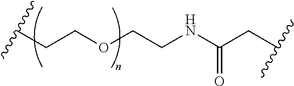 | 3 | 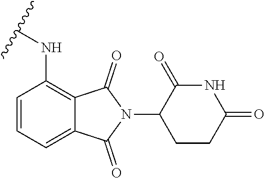 [SMILES: *Nc1cccc2c1C(=O)N(C1CCC(=O)NC1=O)C2=O] |
| 1404 | 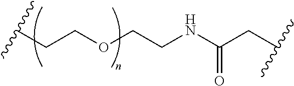 | 4 | 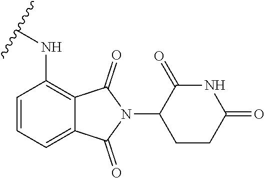 [SMILES: *Nc1cccc2c1C(=O)N(C1CCC(=O)NC1=O)C2=O] |
| 1405 | 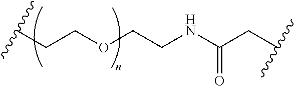 | 5 | 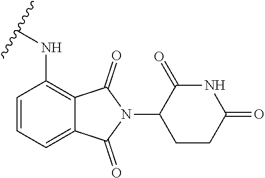 [SMILES: *Nc1cccc2c1C(=O)N(C1CCC(=O)NC1=O)C2=O] |
| 1406 | 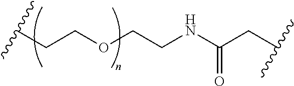 | 1 | 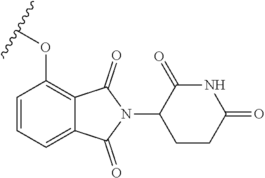 [SMILES: *Oc1cccc2c1C(=O)N(C1CCC(=O)NC1=O)C2=O] |
| 1407 | 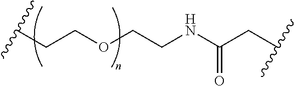 | 2 | 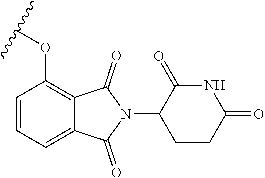 [SMILES: *Oc1cccc2c1C(=O)N(C1CCC(=O)NC1=O)C2=O] |
| 1408 | 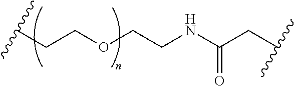 | 3 | 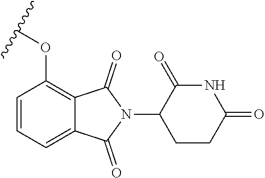 [SMILES: *Oc1cccc2c1C(=O)N(C1CCC(=O)NC1=O)C2=O] |
| 1409 | 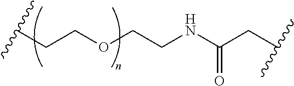 | 4 | 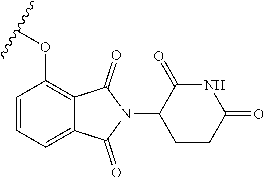 [SMILES: *Oc1cccc2c1C(=O)N(C1CCC(=O)NC1=O)C2=O] |
| 1410 | 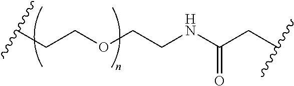 | 5 | 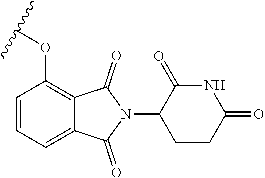 [SMILES: *Oc1cccc2c1C(=O)N(C1CCC(=O)NC1=O)C2=O] |
| 1411 | 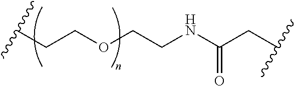 | 1 | 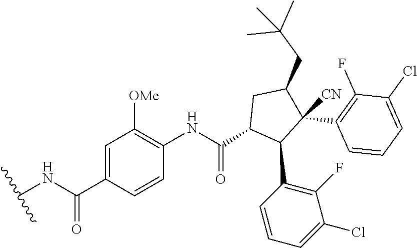 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1412 | 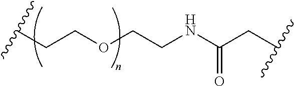 | 2 | 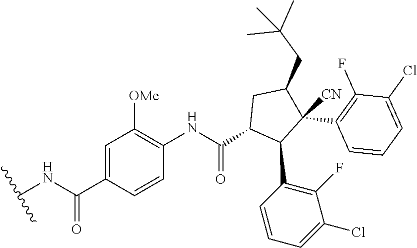 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1413 | 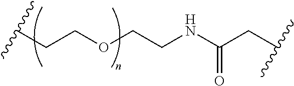 | 3 | 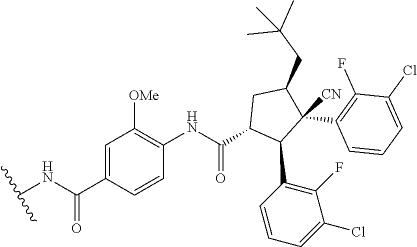 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1414 | 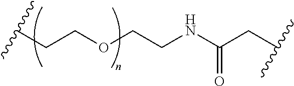 | 4 | 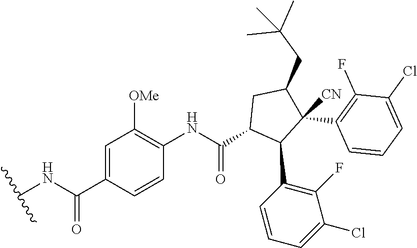 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1415 | 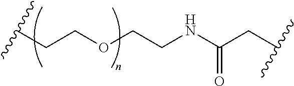 | 5 | 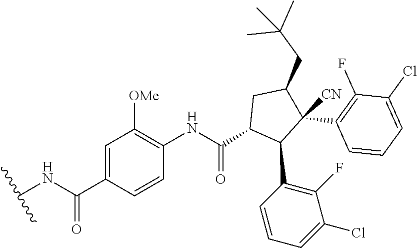 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1416 | 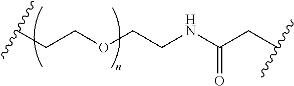 | 1 | 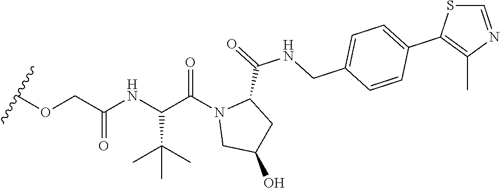 |
| 1417 | 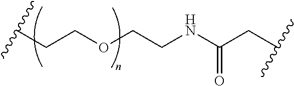 | 2 | 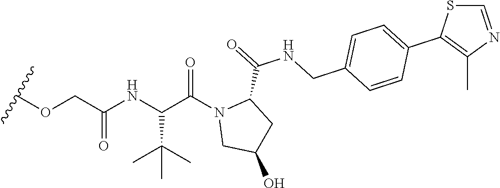 |
| 1418 | 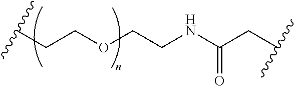 | 3 | 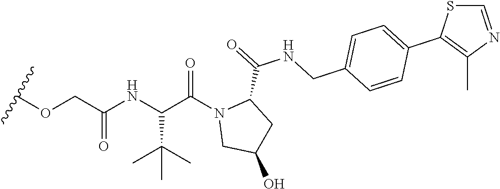 |
| 1419 | 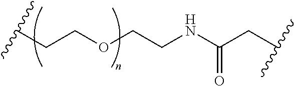 | 4 | 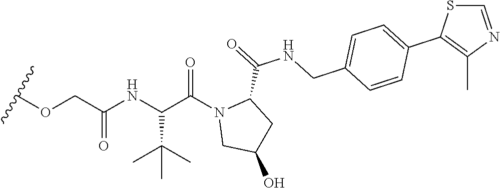 |
| 1420 | 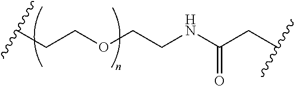 | 5 | 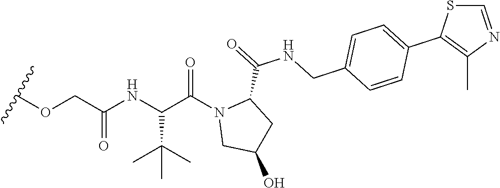 |
| 1421 | 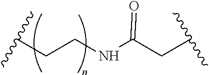 | 1 | 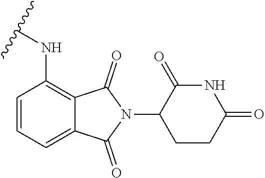 [SMILES: *Nc1cccc2c1C(=O)N(C1CCC(=O)NC1=O)C2=O] |
| 1422 | 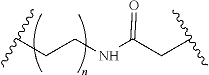 | 2 | 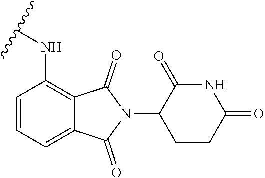 [SMILES: *Nc1cccc2c1C(=O)N(C1CCC(=O)NC1=O)C2=O] |
| 1423 | 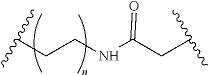 | 3 | 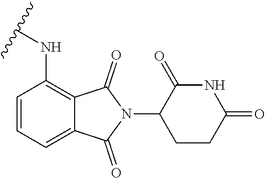 [SMILES: *Nc1cccc2c1C(=O)N(C1CCC(=O)NC1=O)C2=O] |
| 1424 | 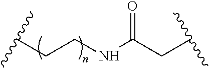 | 4 | 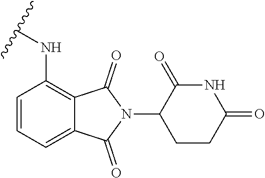 [SMILES: *Nc1cccc2c1C(=O)N(C1CCC(=O)NC1=O)C2=O] |
| 1425 | 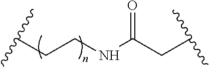 | 5 | 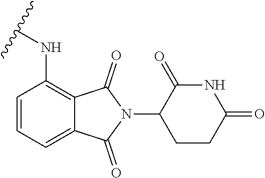 [SMILES: *Nc1cccc2c1C(=O)N(C1CCC(=O)NC1=O)C2=O] |
| 1426 | 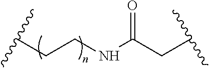 | 1 | 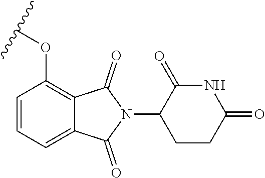 [SMILES: *Oc1cccc2c1C(=O)N(C1CCC(=O)NC1=O)C2=O] |
| 1427 | 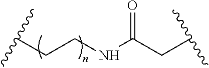 | 2 | 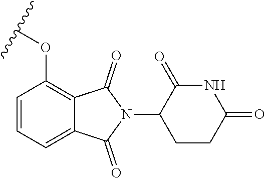 [SMILES: *Oc1cccc2c1C(=O)N(C1CCC(=O)NC1=O)C2=O] |
| 1428 | 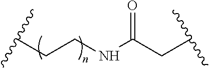 | 3 | 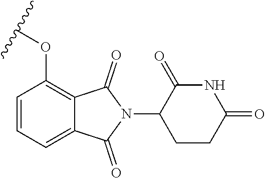 [SMILES: *Oc1cccc2c1C(=O)N(C1CCC(=O)NC1=O)C2=O] |
| 1429 | 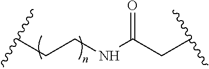 | 4 | 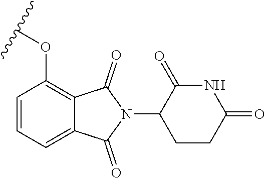 [SMILES: *Oc1cccc2c1C(=O)N(C1CCC(=O)NC1=O)C2=O] |
| 1430 | 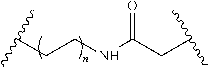 | 5 | 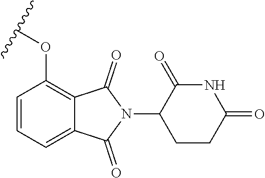 [SMILES: *Oc1cccc2c1C(=O)N(C1CCC(=O)NC1=O)C2=O] |
| 1431 | 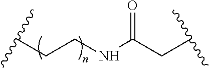 | 1 | 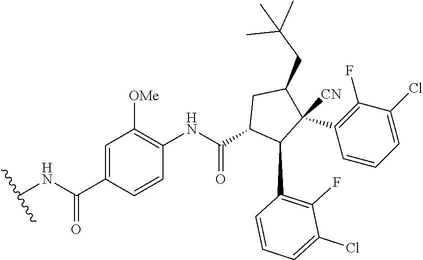 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1432 | 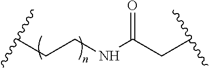 | 2 | 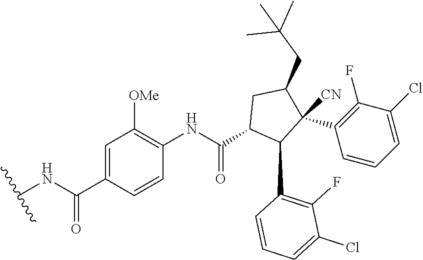 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1433 | 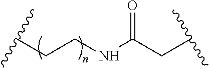 | 3 | 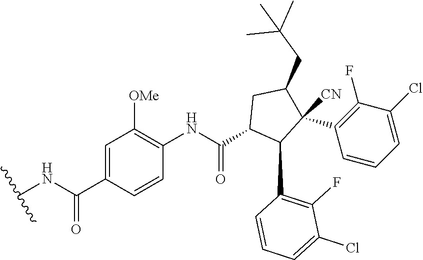 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1434 | 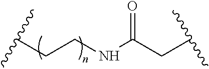 | 4 | 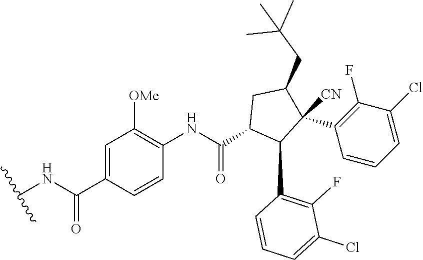 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1435 | 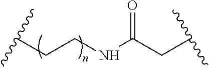 | 5 | 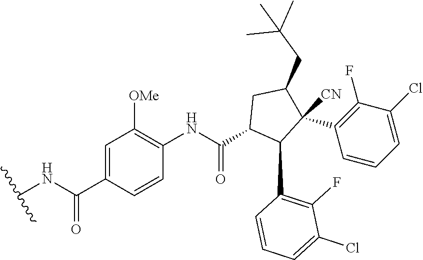 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1436 | 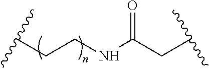 | 1 | 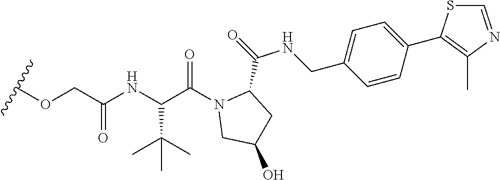 |
| 1437 | 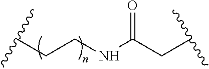 | 2 | 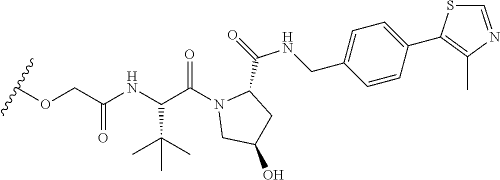 |
| 1438 | 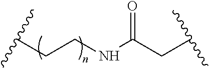 | 3 | 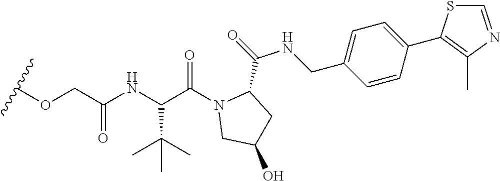 |
| 1439 | 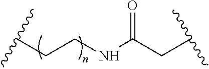 | 4 | 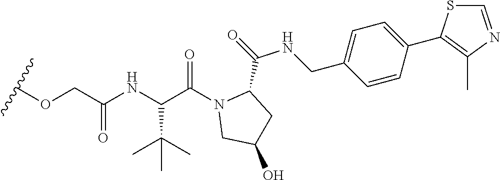 |
| 1440 | 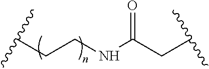 | 5 | 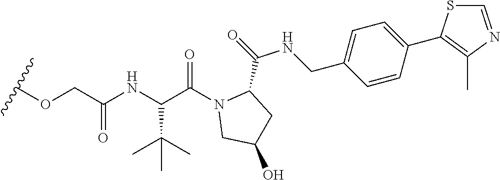 |
| 1441 | 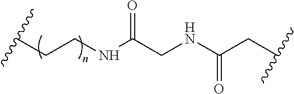 | 1 | 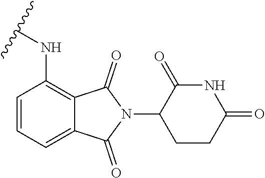 [SMILES: *Nc1cccc2c1C(=O)N(C1CCC(=O)NC1=O)C2=O] |
| 1442 | 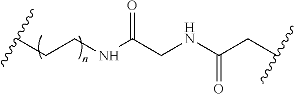 | 2 | 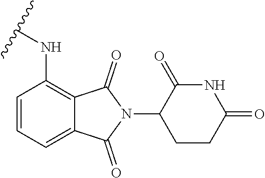 [SMILES: *Nc1cccc2c1C(=O)N(C1CCC(=O)NC1=O)C2=O] |
| 1443 | 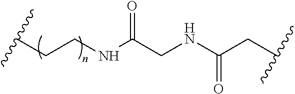 | 3 | 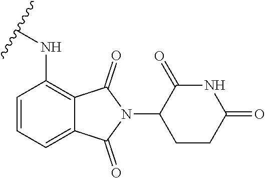 [SMILES: *Nc1cccc2c1C(=O)N(C1CCC(=O)NC1=O)C2=O] |
| 1444 | 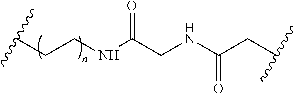 | 4 | 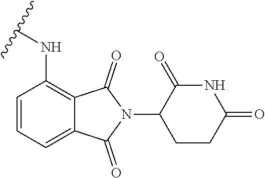 [SMILES: *Nc1cccc2c1C(=O)N(C1CCC(=O)NC1=O)C2=O] |
| 1445 | 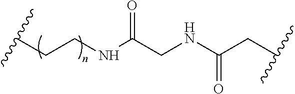 | 5 | 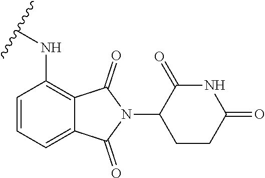 [SMILES: *Nc1cccc2c1C(=O)N(C1CCC(=O)NC1=O)C2=O] |
| 1446 | 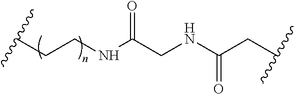 | 1 | 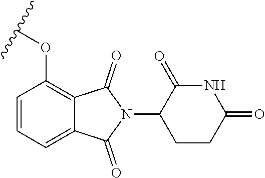 [SMILES: *Oc1cccc2c1C(=O)N(C1CCC(=O)NC1=O)C2=O] |
| 1447 | 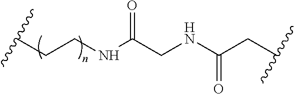 | 2 | 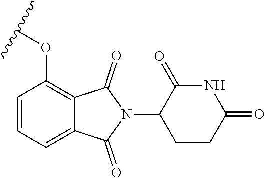 [SMILES: *Oc1cccc2c1C(=O)N(C1CCC(=O)NC1=O)C2=O] |
| 1448 | 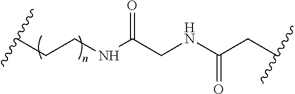 | 3 | 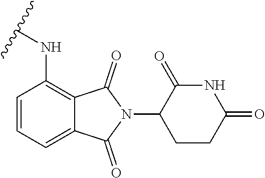 [SMILES: *Nc1cccc2c1C(=O)N(C1CCC(=O)NC1=O)C2=O] |
| 1449 | 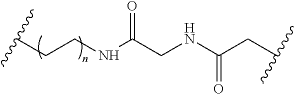 | 4 | 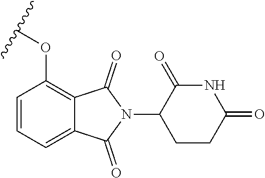 [SMILES: *Oc1cccc2c1C(=O)N(C1CCC(=O)NC1=O)C2=O] |
| 1450 | 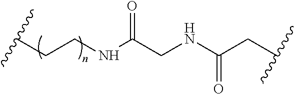 | 5 | 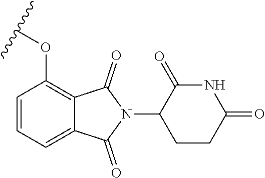 [SMILES: *Oc1cccc2c1C(=O)N(C1CCC(=O)NC1=O)C2=O] |
| 1451 | 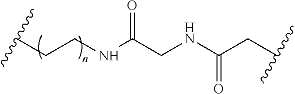 | 1 | 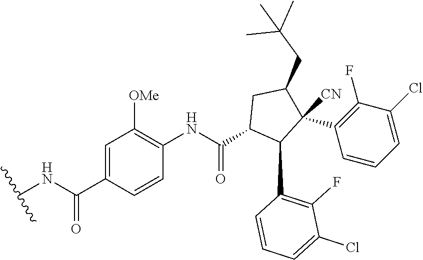 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1452 | 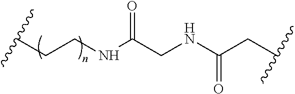 | 2 | 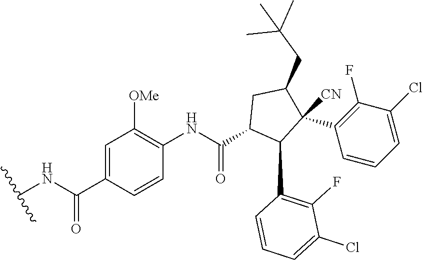 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1453 | 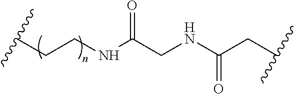 | 3 | 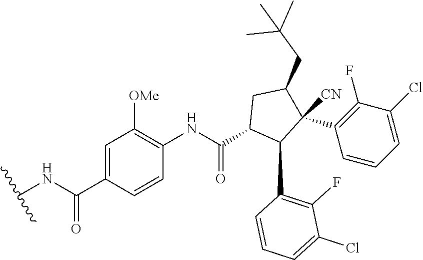 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1454 | 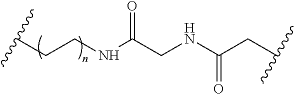 | 4 | 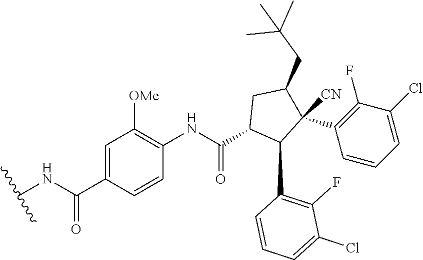 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1455 | 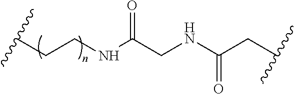 | 5 | 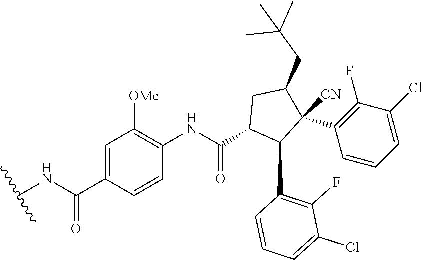 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1456 | 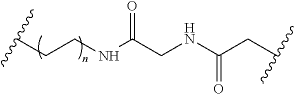 | 1 | 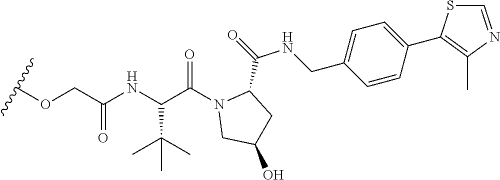 |
| 1457 | 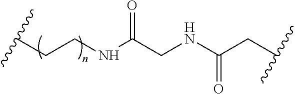 | 2 | 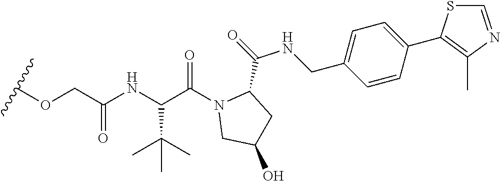 |
| 1458 | 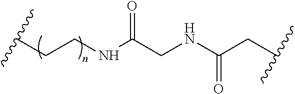 | 3 | 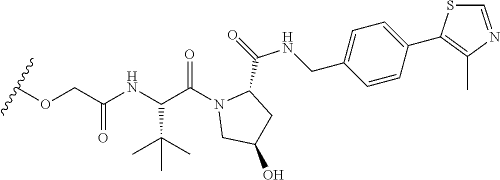 |
| 1459 | 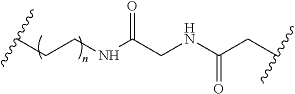 | 4 | 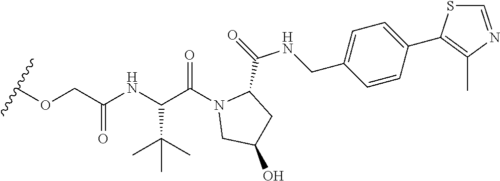 |
| 1460 | 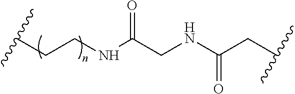 | 5 | 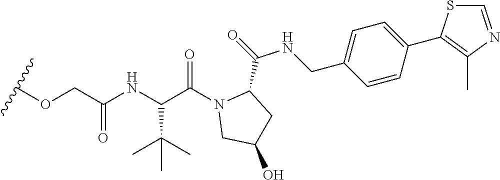 |

In some embodiments, the compound of formula (I) or formula 1001-1460 has a molecular weight not greater than about 2000 g/mol, or about 1900 g/mol, or about 1800 g/mol, or about 1700 g/mol, or about 1600 g/mol, or about 1500 g/mol, or about 1400 g/mol, or about 1300 g/mol, or about 1200 g/mol or about 1100 g/mol, or about 1000 g/mol.

### Methods of Treatment

The compounds and compositions described herein can be used in methods for treating diseases and disorders. In some embodiments, the compounds and compositions described herein can be used in methods for treating diseases associated with the upregulation of myeloid cell leukemia-1 (Mcl-1) oncoprotein. In some embodiments, the compounds and compositions described herein can be used for the treatment of hyperproliferative disorders, including those hyperproliferative disorders associated with the upregulation of Mcl-1. The compounds and compositions described herein may also be used in treating other disorders as described herein and in the following paragraphs.

In one aspect, the disclosure provides a method of treating or preventing a disease or disorder alleviated by inhibiting Mcl-1 protein activity in a patient in need of said treatment or prevention. In some embodiments, the method comprises administering a therapeutically effective amount of one or more compounds of the disclosure, or a pharmaceutically acceptable salt thereof. In one aspect, the disclosure provides a method of treating or preventing a disease or disorder alleviated by indirectly inhibiting Mcl-1 protein activity in a patient in need of said treatment or prevention. In some embodiments, the method comprises administering a therapeutically effective amount of one or more compounds of the disclosure, or a pharmaceutically acceptable salt thereof.

In some embodiments, the Mcl-1 protein activity is inhibited by the compounds of the disclosure binding to a target that downregulates and/or inhibits Mcl-1 protein activity. In some embodiments, the compounds bind to and/or inhibit one or more of CDK9, PI3K/mTOR, MEK1/2, FLT3, JAK1/2, STAT3, and ERK1/2.

In some embodiments, the disease or disorder is cancer. In some embodiments, the cancer is selected from the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like. In some embodiments, the cancer is acute myeloid leukemia (AML).

In some embodiments, the cancer is a blood cancer. In some embodiments, the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

In one aspect, the disclosure provides a method of treating or preventing acute myeloid leukemia (AML) in a patient in need of said treatment or prevention. In some embodiments, the method comprises administering a therapeutically effective amount of one or more compounds of the disclosure, or a pharmaceutically acceptable salt thereof.

In some embodiments, the hyperproliferative disorder treated by the compounds and compositions described herein includes cells having Mcl-1 protein and/or Mcl-1 related protein expression. In some embodiments, the disease treated by the compounds and compositions described herein is selected from the group consisting of myeloid leukemia, non-small cell lung cancer, pancreatic cancer, prostate cancer, and ovarian cancer.

In some embodiments, the compounds described herein may induce cell cycle arrest and/or apoptosis in cells containing functional Mcl-1 proteins. The compounds described herein may be used for sensitizing cells to additional agent(s), such as inducers of apoptosis and/or cell cycle arrest, and chemoprotection of normal cells through the induction of cell cycle arrest prior to treatment with chemotherapeutic agents.

In some embodiments, the compounds described herein may be useful for the treatment of disorders, such as those responsive to induction of apoptotic cell death, e.g., disorders characterized by dysregulation of apoptosis. In some embodiments, the compounds may be used to treat cancer that is characterized by resistance to cancer therapies (e.g., those cancer cells which are chemoresistant, radiation resistant, hormone resistant, and the like). In other embodiments, the compounds can be used to treat hyperproliferative diseases characterized by expression of functional Mcl-1 and/or Mcl-1 related proteins, which may or may not be resilient to Bcl-x_{L} inhibitors.

Efficacy of the compounds and combinations of compounds described herein treating the indicated diseases or disorders can be tested using various models known in the art, and described herein, which provide guidance for treatment of human disease.

### Pharmaceutical Compositions

In an embodiment, an active pharmaceutical ingredient or combination of active pharmaceutical ingredients, such as any of the compounds of formula (I) or formula 1001-1460, is provided as a pharmaceutically acceptable composition.

In some embodiments, the concentration of each of the active pharmaceutical ingredients provided in the pharmaceutical compositions of the disclosure, such as any of the compounds of formula (I) or formula 1001-1460, is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of each of the active pharmaceutical ingredients provided in the pharmaceutical compositions of the disclosure, such as any of the compounds of formula (I) or formula 1001-1460, is greater than 90%, 800%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the compounds of formula (I) or formula 1001-1460, is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of each of the active pharmaceutical ingredients provided in the pharmaceutical compositions of the disclosure, such as any of the compounds of formula (I) or formula 1001-1460, is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of each of the active pharmaceutical ingredients provided in the pharmaceutical compositions of the disclosure, such as any of the compounds of formula (I) or formula 1001-1460, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the disclosure, such as any of the compounds of formula (I) or formula 1001-1460, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the disclosure is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the compounds of formula (I) or formula 1001-1460 may also be used if appropriate.

In an embodiment, the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In one aspect, the disclosure provides a pharmaceutical composition comprising one or more of compounds of any one of formula (I), formula 1001-1460, or a pharmaceutically acceptable salt thereof, and a physiologically compatible carrier medium.

In one aspect, the disclosure provides a pharmaceutical composition for treating or preventing a disease or disorder alleviated by inhibiting Mcl-1 protein activity, the pharmaceutical composition comprising one or more compounds according to any one of formula (I), formula 1001-1063, or a pharmaceutically acceptable salt thereof, and a physiologically compatible carrier medium. In some embodiments, the disease or disorder is cancer.

In one aspect, the disclosure provides a pharmaceutical composition for treating or preventing a disease or disorder alleviated by indirectly inhibiting Mcl-1 protein activity, the pharmaceutical composition comprising one or more compounds according to any one of formula (I), formula 1001-1460, or a pharmaceutically acceptable salt thereof, and a physiologically compatible carrier medium. In some embodiments, the disease or disorder is cancer.

In some embodiments, the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is acute myeloid leukemia (AML).

In some embodiments, the cancer is a blood cancer. In some embodiments, the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

Furthermore, the described methods of treatment may normally include medical follow-up to determine the therapeutic or prophylactic effect brought about in the subject undergoing treatment with the compound(s) and/or composition(s) described herein.

In one aspect, the disclosure provides a pharmaceutical composition for treating or preventing from acute myeloid leukemia (AML), the pharmaceutical composition comprising one or more compounds of formula (I), formula 1001-1460, or a pharmaceutically acceptable salt thereof, and a physiologically compatible carrier medium.

Described below are non-limiting pharmaceutical compositions and methods for preparing the same.

### Pharmaceutical Compositions for Oral Administration

In an embodiment, the disclosure provides a pharmaceutical composition for oral administration containing the active pharmaceutical ingredient or combination of active pharmaceutical ingredients, such as one or more compounds of formula (I), formula 1001-1460, or a pharmaceutically acceptable salt thereof, and a pharmaceutical excipient suitable for oral administration.

In some embodiments, the disclosure provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient and optionally (iv) an effective amount of a fourth active pharmaceutical ingredient.

In some embodiments, the pharmaceutical composition may be a liquid pharmaceutical composition suitable for oral consumption. Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients.

Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art.

About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof. The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogs thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water. Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight. The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, a pharmaceutical composition is provided for injection containing an active pharmaceutical ingredient or combination of active pharmaceutical ingredients, such as one or more compounds of formula (I) or formula 1001-1460, or a pharmaceutically acceptable salt thereof, and a pharmaceutical excipient suitable for injection.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, a pharmaceutical composition is provided for transdermal delivery containing an active pharmaceutical ingredient or combination of active pharmaceutical ingredients, such as compounds of formula (I) or formula 1001-1460, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of an active pharmaceutical ingredient or combination of active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139, the entirety of which are incorporated herein by reference. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra and the compounds of formula (I) and formula 1001-1460 described herein. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions comprising one or more compounds of formula (I) and formula 1001-1460 may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety. Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The active pharmaceutical ingredient or combination of active pharmaceutical ingredients can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

The disclosure also provides kits. The kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer. In some embodiments, the disclosure provides a kit comprising a composition comprising a therapeutically effective amount of an active pharmaceutical ingredient or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the disclosure provides a kit comprising (1) a composition comprising a therapeutically effective amount of an active pharmaceutical ingredient or combination of active pharmaceutical ingredients or a pharmaceutically acceptable salt, solvate, hydrate, cocrystal, or prodrug thereof, and (2) a diagnostic test for determining whether a patient's cancer is a particular subtype of a cancer. Any of the foregoing diagnostic methods may be utilized in the kit.

The kits described above are preferably for use in the treatment of the diseases and conditions described herein. In a particular embodiment, the kits are for use in the treatment of hyperproliferative disorders.

In a particular embodiment, the kits described herein are for use in the treatment of cancer. In some embodiments, the kits described herein are for use in the treatment of a cancer selected from the group consisting of pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In particular embodiments, the kits described herein are for use in the treatment of a cancer selected from the group consisting of myeloid leukemia, non-small cell lung cancer, pancreatic cancer, prostate cancer, and ovarian cancer.

### Dosages and Dosing Regimens

The amounts of the pharmaceutical compositions administered using the methods herein, such as the dosages of compounds of formula (I) or formula 1001-1460, will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area. In some embodiments, the pharmaceutical composition comprising one or more compounds of formula (I) or formula 1001-1460 is administered in combination with venetoclax (VEN). In some embodiments, the pharmaceutical composition comprising one or more compounds of formula (I) or formula 1001-1460 is administered prior to, concurrently with, and/or after the administration of venetoclax (VEN).

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the preferred oral route, may be used as appropriate. A single dose of a pharmaceutical composition may also be used for treatment of an acute condition. In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients may continue as long as necessary. In selected embodiments, a pharmaceutical composition is administered for more than 1, 2, 3, 4, 5, 6, 7, 14, or 28 days. In some embodiments, a pharmaceutical composition is administered for less than 28, 14, 7, 6, 5, 4, 3, 2, or 1 day. In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an active pharmaceutical ingredient disclosed herein is in the range of about 0.01 mg/kg to about 200 mg/kg, or about 0.1 to 100 mg/kg, or about 1 to 50 mg/kg.

In some embodiments, an active pharmaceutical ingredient is adminstered at a dosage of 10 to 200 mg BID, including 50, 60, 70, 80, 90, 100, 150, or 200 mg BID. In some embodiments, an active pharmaceutical ingredient is adminstered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect—e.g., by dividing such larger doses into several small doses for administration throughout the day. Of course, as those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the compositions described herein further include controlled-release, sustained release, or extended-release therapeutic dosage forms for administration of the compounds described herein, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

The following clauses describe certain embodiments.

Clause 1. A compound of formula (I), or comprising a substructure of formula (I), or a pharmaceutically acceptable salt, solvate, hydrate, cocrystal, or prodrug thereof

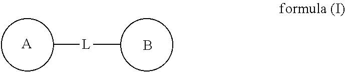

wherein in formula (I):
A comprises an Mcl-1 protein indirect inhibitor moiety;
L is a linking group; and
B comprises an E3 ubiquitin ligase ligand moiety, wherein the Mcl-1 protein inhibitor moiety thereof, and the E3 ubiquitin ligase ligand moiety thereof are each connected to L at any chemically feasible site.

Clause 2. The compound of clause 1, wherein the Mcl-1 protein indirect inhibitor moiety comprises a CDK9 inhibitor, a dual PI3K/mTOR inhibitor, a MEK1/2 inhibitor, a FLT3 inhibitor, a JAK1/2 inhibitor, a STAT3 inhibitor, an ERK1/2 inhibitor, or any substructure thereof.

Clause 3. The compound of clause 2, wherein the CDK9 inhibitor is selected from AT7519, TG02, PHA 767491, PHA-793887, PHA-848125, BAY 1143572, BAY 1112054, Cdk9 inhibitor II (CAS 140651-18-9 from Calbiochem), DRB, AZD-5438, SNS-032, dinaciclib, LY2857785, flavopiridol, purvalanol B, CDKI-71, CDKI-73, CAN508, FIT-039, CYC065, P276-00, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, shRNAs against CDK9, anti-sense mRNA against CDK9 and anti-CDK9 antibodies, and any substructure thereof.

Clause 4. The compound of clause 2, wherein the dual PI3K/mTOR inhibitor is selected from gedatolisib (PF-05212384; PKI-587), XL765, GDC-0980, BEZ235 (NVP-BEZ235), BGT226, GSK2126458, PF-04691502, and any substructure thereof.

Clause 5. The compound of clause 2, wherein the MEK1/2 inhibitor is selected from PD334581, CI-1040, AZD6244, PD318088, PD98059, RDEA119, 6-Methoxy-7-(3-morpholin-4-yl-propoxy)-4-(4-phenoxy-phenylamino)-quinoline-3-carbonitrile, 4-[3-Chloro-4-(1-methyl-1H-imidazol-2-ylsulfanyl)-phenylamino]-6-methoxy-7-(3-morpholin-4-yl-propoxy)-quinoline-3-carbonitrile, and any substructure thereof.

Clause 6. The compound of clause 2, wherein the FLT3 inhibitor is selected from midostaurin, quizartinib, crenolanib, gilteritinib, FLX-925 (AMG-925), G-749, and any substructure thereof.

Clause 7. The compound of clause 2, wherein the JAK1/2 inhibitor is selected from Ruxolitinib, Baricitinib, Tofacitinib, and any substructure thereof.

Clause 8. The compound of clause 2, wherein the STAT3 inhibitor is selected from WP1066, S3I-201, C1-C10, and any substructure thereof.

Clause 9. The compound of clause 2, wherein the ERK1/2 inhibitor is selected from WP1066AEZS-131, AEZS-136, BVD-523, SCH-722984, SCH-772984, SCH-900353 (MK-8353), and any substructure thereof.

Clause 10. The compound of clause 1, wherein the Mcl-1 protein indirect inhibitor moiety is selected from AT7519, gedatolisib (PF-05212384; PKI-587), PD334581, TG02, and any substructure thereof.

Clause 11. The compound of any one of clauses 1 and 2-9, wherein the Mcl-1 protein indirect inhibitor moiety is selected from

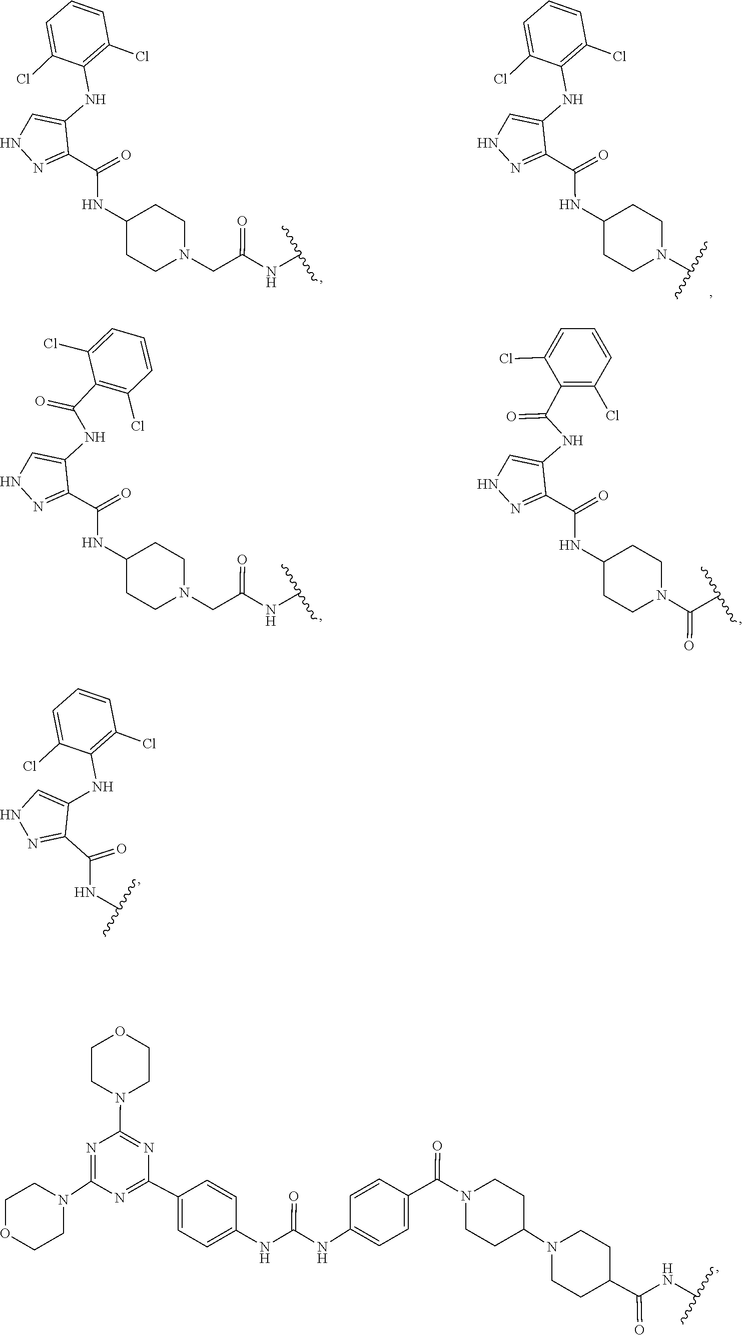 [SMILES: *NC(=O)CN1CCC(NC(=O)c2n[nH]cc2Nc2c(Cl)cccc2Cl)CC1 ; *N1CCC(NC(=O)c2n[nH]cc2Nc2c(Cl)cccc2Cl)CC1 ; *NC(=O)CN1CCC(NC(=O)c2n[nH]cc2NC(=O)c2c(Cl)cccc2Cl)CC1 ; *C(=O)N1CCC(NC(=O)c2n[nH]cc2NC(=O)c2c(Cl)cccc2Cl)CC1 ; *NC(=O)c1n[nH]cc1Nc1c(Cl)cccc1Cl ; *NC(=O)C1CCN(C2CCN(C(=O)c3ccc(NC(=O)Nc4ccc(-c5nc(N6CCOCC6)nc(N6CCOCC6)n5)cc4)cc3)CC2)CC1]

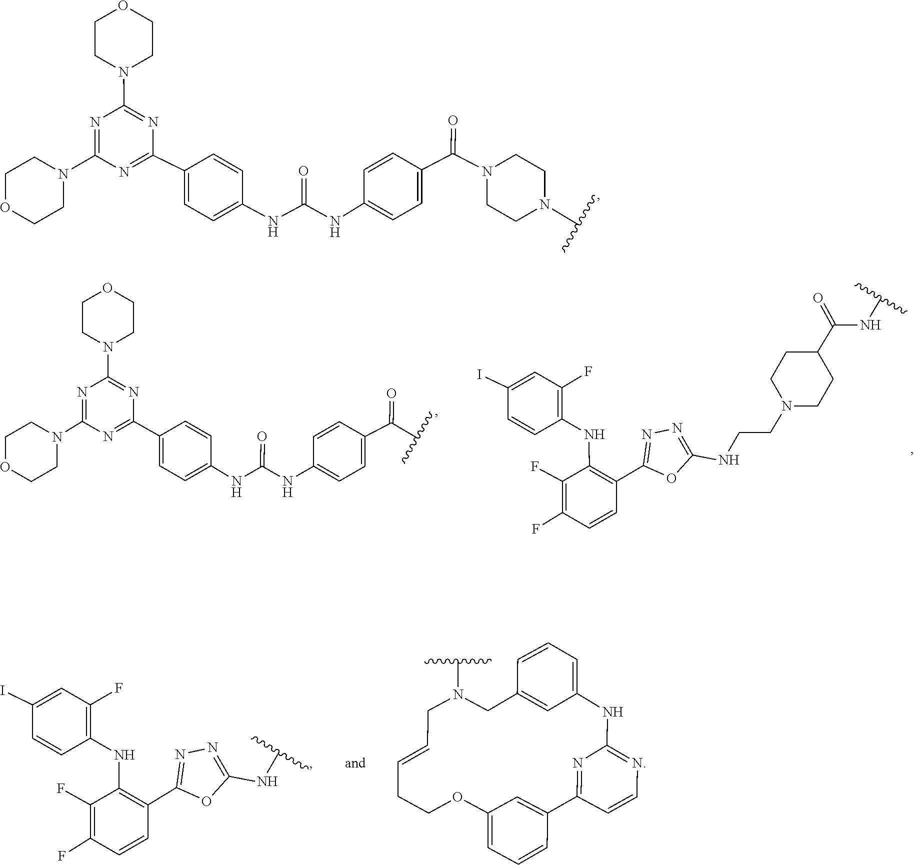 [SMILES: *N1CCN(C(=O)c2ccc(NC(=O)Nc3ccc(-c4nc(N5CCOCC5)nc(N5CCOCC5)n4)cc3)cc2)CC1 ; *C(=O)c1ccc(NC(=O)Nc2ccc(-c3nc(N4CCOCC4)nc(N4CCOCC4)n3)cc2)cc1 ; *NC(=O)C1CCN(CCNc2nnc(-c3ccc(F)c(F)c3Nc3ccc(I)cc3F)o2)CC1 ; *Nc1nnc(-c2ccc(F)c(F)c2Nc2ccc(I)cc2F)o1 ; *N1C/C=C/CCOc2cccc(c2)-c2ccnc(n2)Nc2cccc(c2)C1]

Clause 12. The compound of clause 1, wherein L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR)N(R^{b})—, —N(R^{b})S(O)_{w}—, —S(O)_{w}N(R^{b})—, —S(O).O—, —OS(O)_{w}—, —OS(O)_{w}O—, —O(O)P(OR^{b})O—, (O)P(O—)₃, —O(S)P(OR)O—, and (S)P(O—)₃, wherein w is 1 or 2, and R^{b} is independently hydrogen, optionally substituted alkyl, or optionally substituted aryl.

Clause 13. The compound of clause 12, wherein L comprises one or more linking groups selected from —C₁₋₁₀ alkyl-, —O—C₁₋₁₀ alkyl-, —O—, -cycloalkyl-, -heterocyclyl-, —C(O)—, —C(O)N(R^{b})— wherein R^{b} is hydrogen or optionally substituted alkyl, and —N(R^{b})— wherein R^{b} is optionally substituted alkyl.

Clause 14. The compound of clause 12 or 13, wherein the heterocyclyl is piperidyl or piperazinyl.

Clause 15. The compound of any one of clauses 12-14, wherein L comprises one or more linking groups selected from

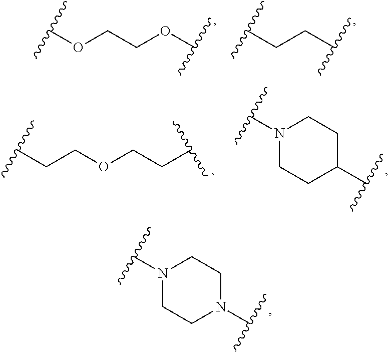

—CH₂C(O)NH—, —C(O)NH—, —C(O)CH₂—, and —OCH₂C(O)—.

Clause 16. The compound of any one of clauses 12-15, wherein L comprises one or more linking groups selected from

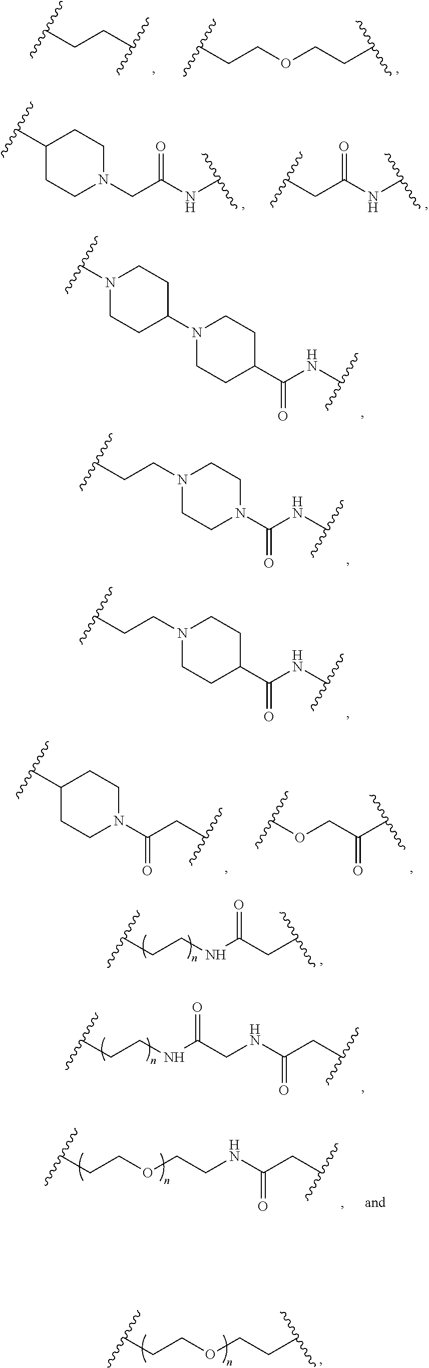

wherein n=1-5.

Clause 17. The compound of clause 1, wherein the E3 ubiquitin ligase ligand moiety comprises cereblon (CRBN) ligand, a mouse double minute 2 (MDM2) ligand, a Von Hippel-Lindau (VHL) ligand, or any substructure thereof.

Clause 18. The compound of clause 17, wherein the CRBN ligand is selected from thalidomide, lenalidomide, pomalidomide, and any substructure thereof.

Clause 19. The compound of clause 17, wherein the MDM2 ligand is selected from idasanutlin, RG7112, RG7388, MI 773/SAR 405838, AMG 232, DS-3032b, RO6839921, RO5045337, RO5503781, CGM-097, MK-8242, and any substructure thereof.

Clause 20. The compound of clause 17, wherein the VHL ligand is selected from VHL ligand 1 (VHL-1), VHL ligand 2 (VHL-2), VH032, and any substructure thereof.

Clause 21. The compound of any one of clauses 1 and 17-20, wherein the E3 ubiquitin ligase ligand moiety is selected from thalidomide, idasanutlin, VHL ligand 1 (VHL-1), and any substructure thereof.

Clause 22. The compound of clause 1, wherein the E3 ubiquitin ligase ligand moiety is selected

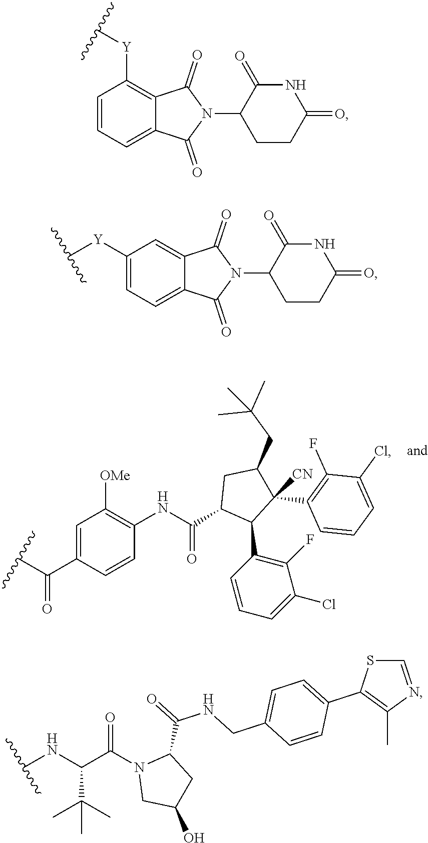

wherein Y is selected from —N(R)—, —N(H)—, and —O—, wherein R is optionally substituted alkyl.

Clause 23. The compound of clause 22, wherein the E3 ubiquitin ligase ligand moiety is selected from

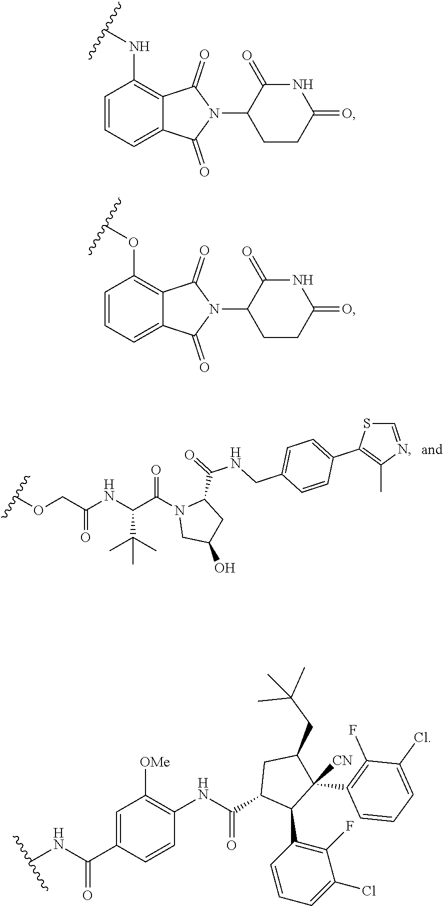 [SMILES: *Nc1cccc2c1C(=O)N(C1CCC(=O)NC1=O)C2=O ; *Oc1cccc2c1C(=O)N(C1CCC(=O)NC1=O)C2=O ; *OCC(=O)N[C@H](C(=O)N1C[C@H](O)C[C@H]1C(=O)NCc1ccc(-c2scnc2C)cc1)C(C)(C)C ; *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1]

Clause 24. The compound of any one of clauses 1-23, wherein the compound has a molecular weight not greater than about 2000 g/mol, or about 1900 g/mol, or about 1800 g/mol, or about 1700 g/mol, or about 1600 g/mol, or about 1500 g/mol, or about 1400 g/mol, or about 1300 g/mol, or about 1200 g/mol or about 1100 g/mol, or about 1000 g/mol.

Clause 25. The compound of clause 1, wherein the compound of formula (I) is a compound of any one of formula 1001-1460, or a pharmaceutically acceptable salt, solvate, hydrate, cocrystal, or prodrug thereof:

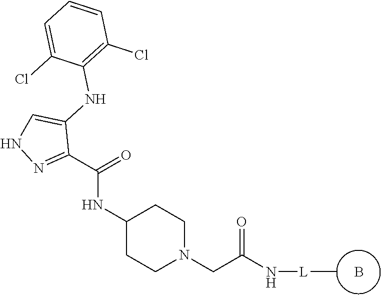

| Formula | | | |
|---|---|---|---|
| No. | L | n | B |
| 1001 | 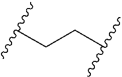 [SMILES: *CC*] | — | 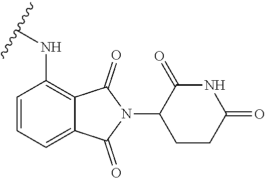 [SMILES: *Nc1cccc2c1C(=O)N(C1CCC(=O)NC1=O)C2=O] |
| 1002 | 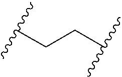 [SMILES: *CC*] | — | 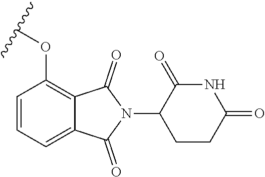 [SMILES: *Oc1cccc2c1C(=O)N(C1CCC(=O)NC1=O)C2=O] |
| 1003 | 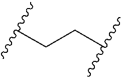 [SMILES: *CC*] | — | 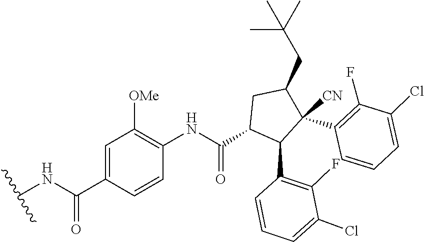 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1004 | 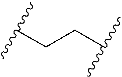 [SMILES: *CC*] | — | 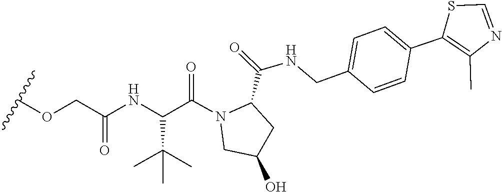 |
| 1005 | 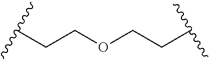 [SMILES: *CCOCC*] | — | 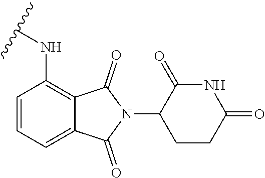 [SMILES: *Nc1cccc2c1C(=O)N(C1CCC(=O)NC1=O)C2=O] |
| 1006 | 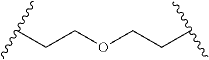 [SMILES: *CCOCC*] | — | 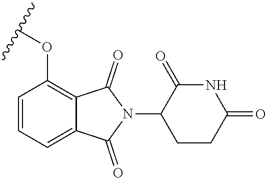 [SMILES: *Oc1cccc2c1C(=O)N(C1CCC(=O)NC1=O)C2=O] |
| 1007 | 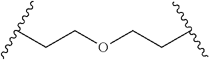 [SMILES: *CCOCC*] | — | 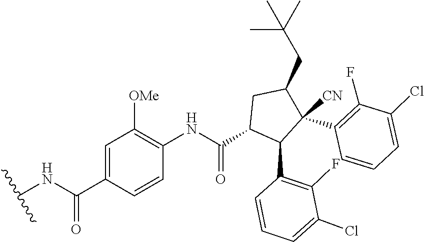 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1008 | 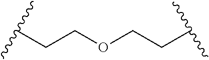 [SMILES: *CCOCC*] | — | 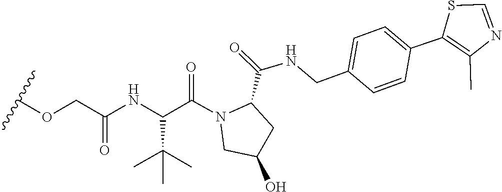 |
| 1009 | 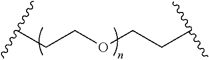 | 1 | 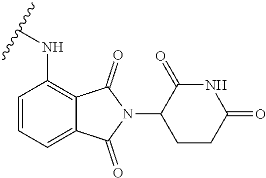 [SMILES: *Nc1cccc2c1C(=O)N(C1CCC(=O)NC1=O)C2=O] |
| 1010 | 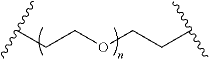 | 2 | 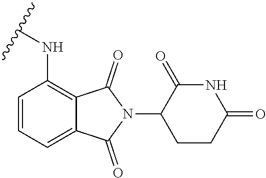 [SMILES: *Nc1cccc2c1C(=O)N(C1CCC(=O)NC1=O)C2=O] |
| 1011 | 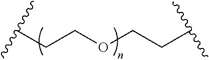 | 3 | 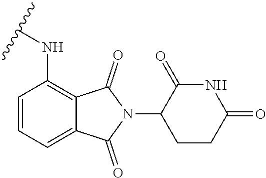 [SMILES: *Nc1cccc2c1C(=O)N(C1CCC(=O)NC1=O)C2=O] |
| 1012 | 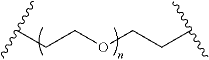 | 4 | 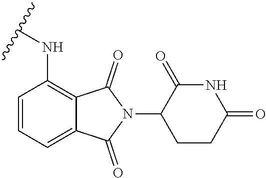 [SMILES: *Nc1cccc2c1C(=O)N(C1CCC(=O)NC1=O)C2=O] |
| 1013 | 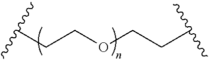 | 5 | 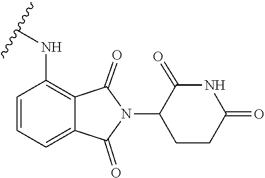 [SMILES: *Nc1cccc2c1C(=O)N(C1CCC(=O)NC1=O)C2=O] |
| 1014 | 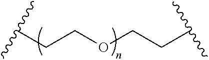 | 1 | 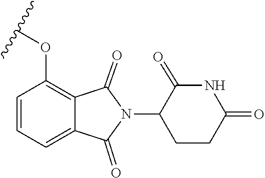 [SMILES: *Oc1cccc2c1C(=O)N(C1CCC(=O)NC1=O)C2=O] |
| 1015 | 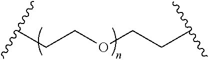 | 2 | 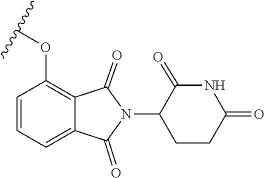 [SMILES: *Oc1cccc2c1C(=O)N(C1CCC(=O)NC1=O)C2=O] |
| 1016 | 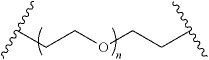 | 3 | 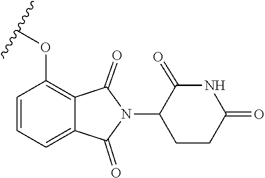 [SMILES: *Oc1cccc2c1C(=O)N(C1CCC(=O)NC1=O)C2=O] |
| 1017 | 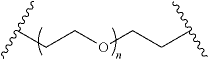 | 4 | 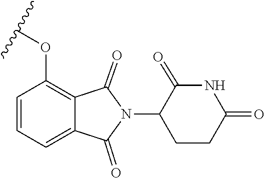 [SMILES: *Oc1cccc2c1C(=O)N(C1CCC(=O)NC1=O)C2=O] |
| 1018 | 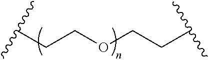 | 5 | 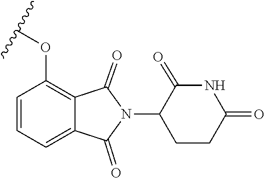 [SMILES: *Oc1cccc2c1C(=O)N(C1CCC(=O)NC1=O)C2=O] |
| 1019 | 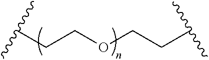 | 1 | 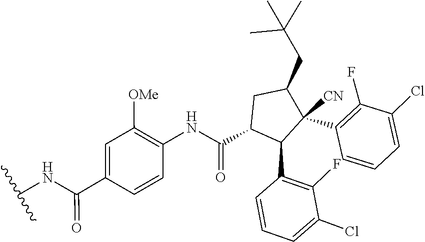 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1020 | 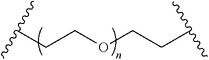 | 2 | 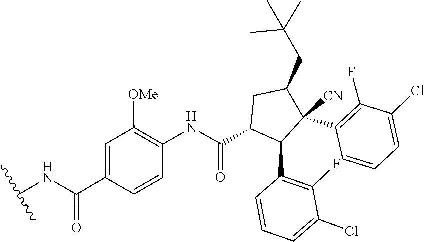 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1021 | 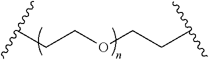 | 3 | 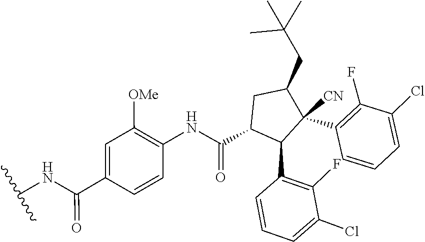 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1022 | 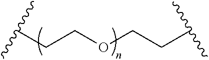 | 4 | 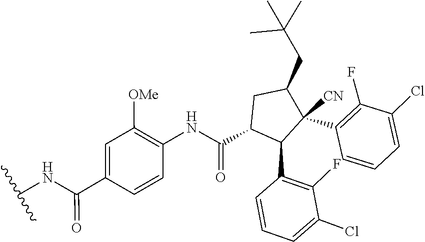 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1023 | 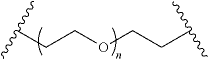 | 5 | 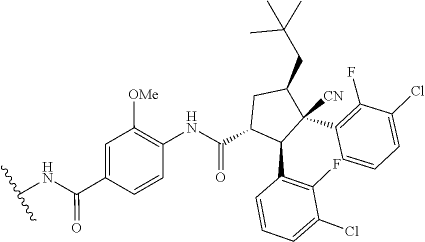 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1024 | 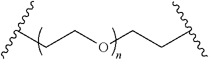 | 1 | 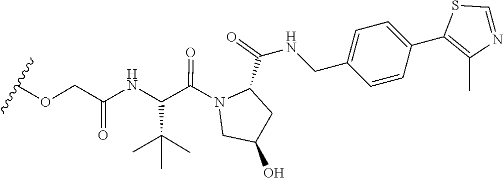 |
| 1025 | 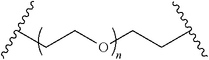 | 2 | 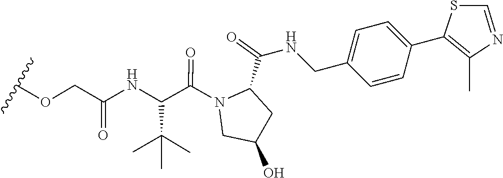 |
| 1026 | 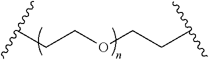 | 3 | 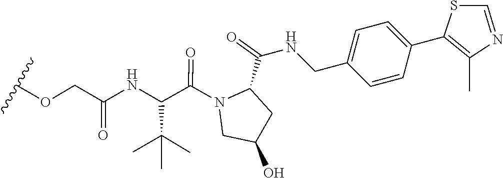 |
| 1027 | 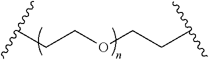 | 4 | 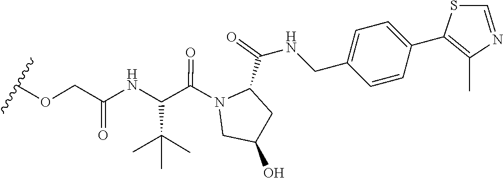 |
| 1028 | 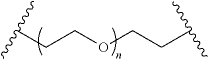 | 5 | 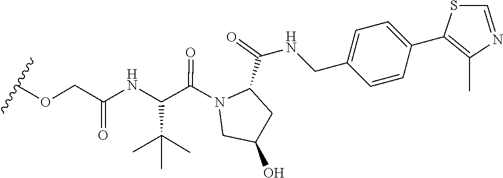 |
| 1029 | 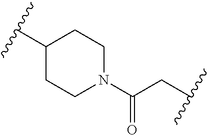 [SMILES: *CC(=O)N1CCC(*)CC1] | — | 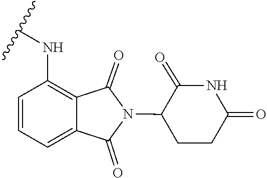 [SMILES: *Nc1cccc2c1C(=O)N(C1CCC(=O)NC1=O)C2=O] |
| 1030 | 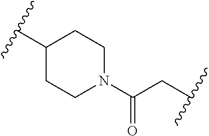 [SMILES: *CC(=O)N1CCC(*)CC1] | — | 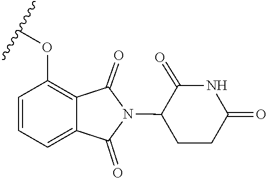 [SMILES: *Oc1cccc2c1C(=O)N(C1CCC(=O)NC1=O)C2=O] |
| 1031 | 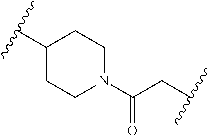 [SMILES: *CC(=O)N1CCC(*)CC1] | — | 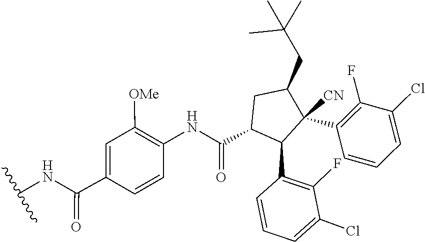 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1032 | 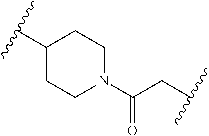 [SMILES: *CC(=O)N1CCC(*)CC1] | — | 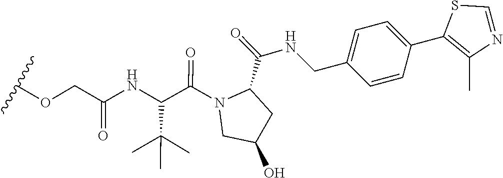 |
| 1033 | 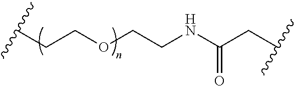 | 1 | 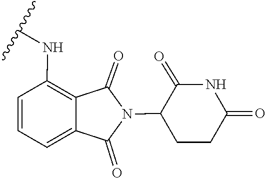 [SMILES: *Nc1cccc2c1C(=O)N(C1CCC(=O)NC1=O)C2=O] |
| 1034 | 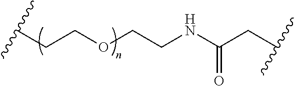 | 2 | 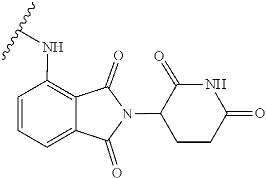 [SMILES: *Nc1cccc2c1C(=O)N(C1CCC(=O)NC1=O)C2=O] |
| 1035 | 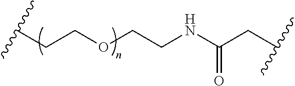 | 3 | 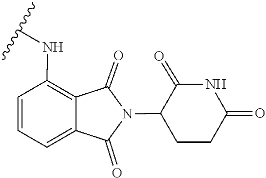 [SMILES: *Nc1cccc2c1C(=O)N(C1CCC(=O)NC1=O)C2=O] |
| 1036 | 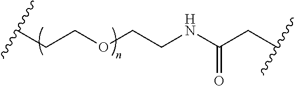 | 4 | 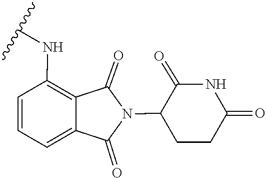 [SMILES: *Nc1cccc2c1C(=O)N(C1CCC(=O)NC1=O)C2=O] |
| 1037 | 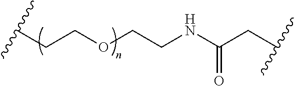 | 5 | 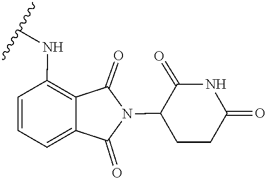 [SMILES: *Nc1cccc2c1C(=O)N(C1CCC(=O)NC1=O)C2=O] |
| 1038 | 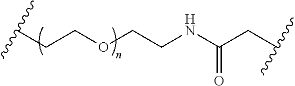 | 1 | 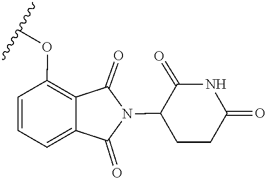 [SMILES: *Oc1cccc2c1C(=O)N(C1CCC(=O)NC1=O)C2=O] |
| 1039 | 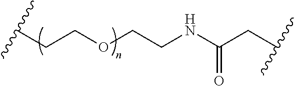 | 2 | 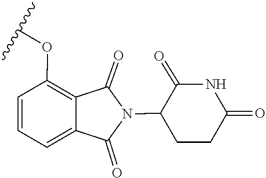 [SMILES: *Oc1cccc2c1C(=O)N(C1CCC(=O)NC1=O)C2=O] |
| 1040 | 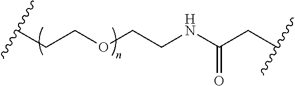 | 3 | 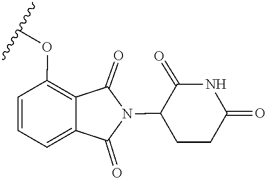 [SMILES: *Oc1cccc2c1C(=O)N(C1CCC(=O)NC1=O)C2=O] |
| 1041 | 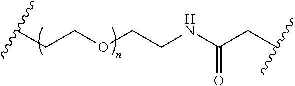 | 4 | 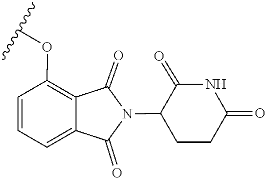 [SMILES: *Oc1cccc2c1C(=O)N(C1CCC(=O)NC1=O)C2=O] |
| 1042 | 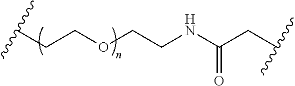 | 5 | 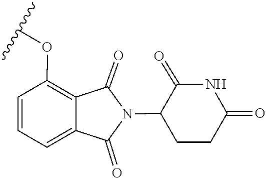 [SMILES: *Oc1cccc2c1C(=O)N(C1CCC(=O)NC1=O)C2=O] |
| 1043 | 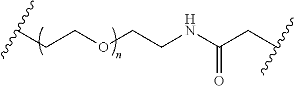 | 1 | 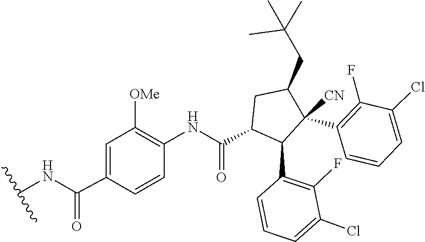 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1044 | 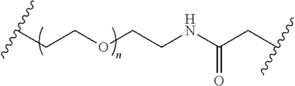 | 2 | 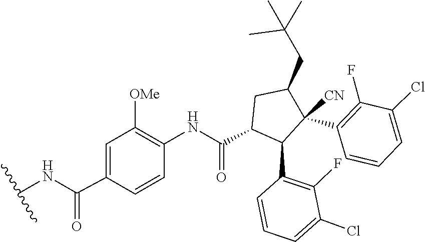 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1045 | 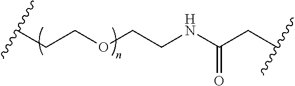 | 3 | 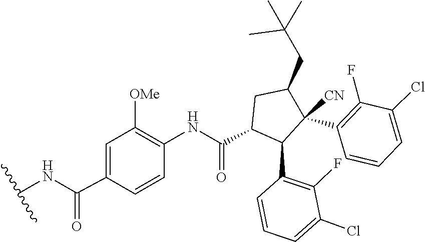 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1046 | 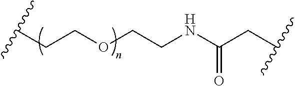 | 4 | 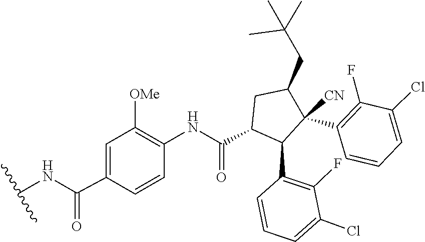 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1047 | 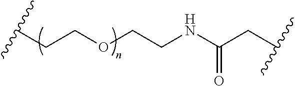 | 5 | 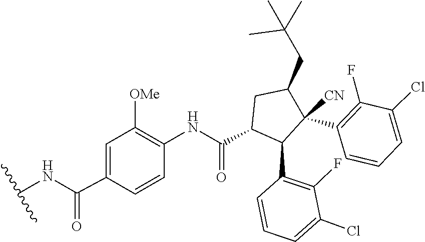 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1048 | 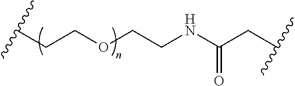 | 1 | 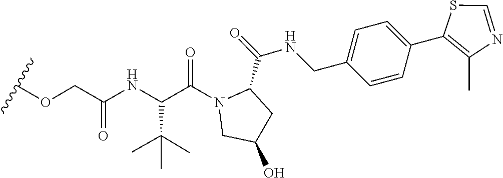 |
| 1049 | 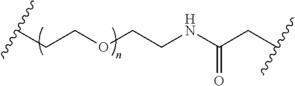 | 2 | 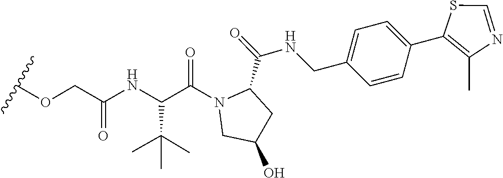 |
| 1050 | 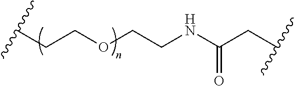 | 3 | 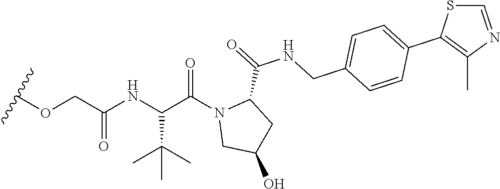 |
| 1051 | 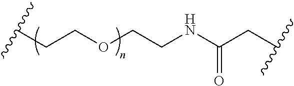 | 4 | 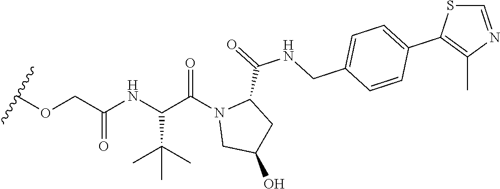 |
| 1052 | 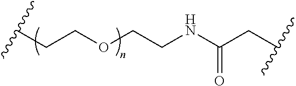 | 5 | 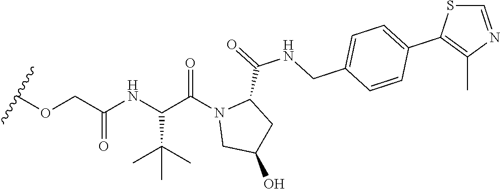 |
| 1053 | 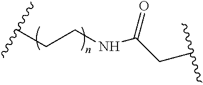 | 1 | 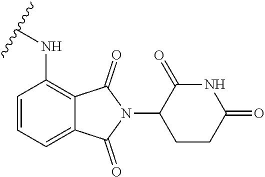 [SMILES: *Nc1cccc2c1C(=O)N(C1CCC(=O)NC1=O)C2=O] |
| 1054 | 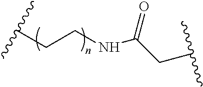 | 2 | 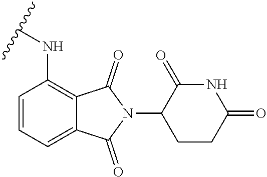 [SMILES: *Nc1cccc2c1C(=O)N(C1CCC(=O)NC1=O)C2=O] |
| 1055 | 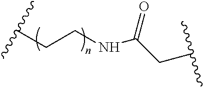 | 3 | 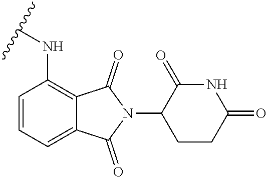 [SMILES: *Nc1cccc2c1C(=O)N(C1CCC(=O)NC1=O)C2=O] |
| 1056 | 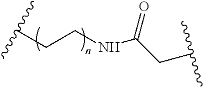 | 4 | 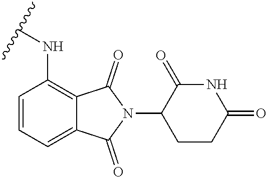 [SMILES: *Nc1cccc2c1C(=O)N(C1CCC(=O)NC1=O)C2=O] |
| 1057 | 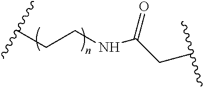 | 5 | 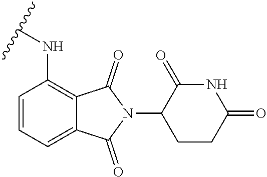 [SMILES: *Nc1cccc2c1C(=O)N(C1CCC(=O)NC1=O)C2=O] |
| 1058 | 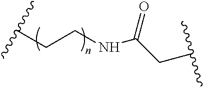 | 1 | 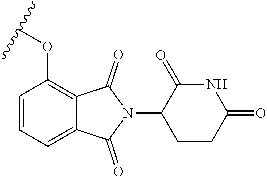 [SMILES: *Oc1cccc2c1C(=O)N(C1CCC(=O)NC1=O)C2=O] |
| 1059 | 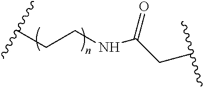 | 2 | 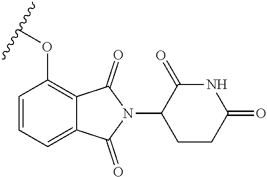 [SMILES: *Oc1cccc2c1C(=O)N(C1CCC(=O)NC1=O)C2=O] |
| 1060 | 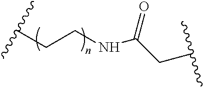 | 3 | 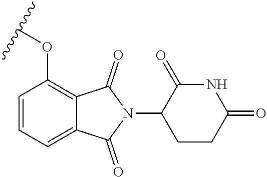 [SMILES: *Oc1cccc2c1C(=O)N(C1CCC(=O)NC1=O)C2=O] |
| 1061 | 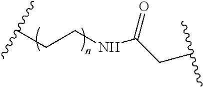 | 4 | 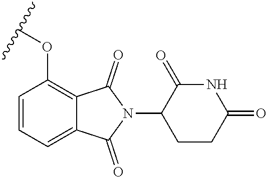 [SMILES: *Oc1cccc2c1C(=O)N(C1CCC(=O)NC1=O)C2=O] |
| 1062 | 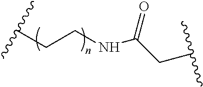 | 5 | 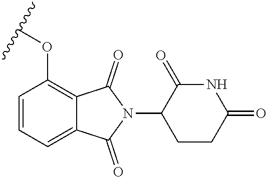 [SMILES: *Oc1cccc2c1C(=O)N(C1CCC(=O)NC1=O)C2=O] |
| 1063 | 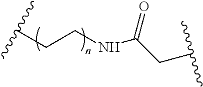 | 1 | 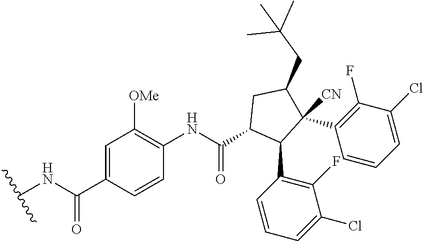 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1064 | 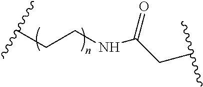 | 2 | 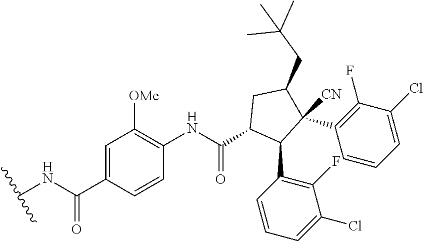 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1065 | 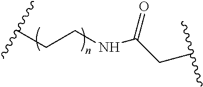 | 3 | 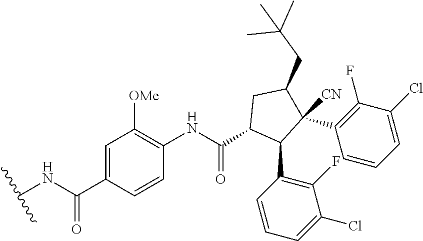 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1066 | 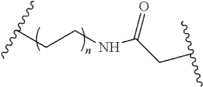 | 4 | 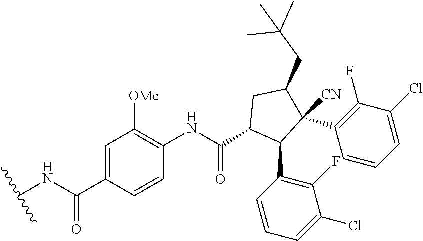 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1067 | 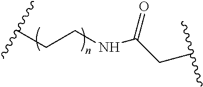 | 5 | 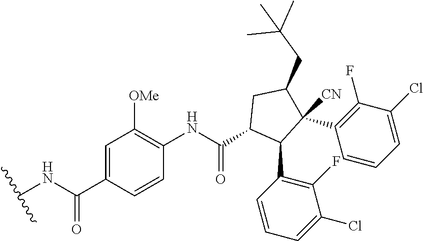 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1068 | 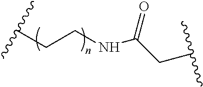 | 1 | 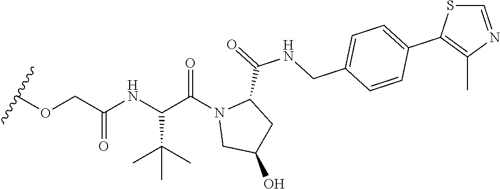 |
| 1069 | 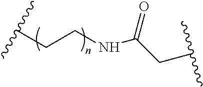 | 2 | 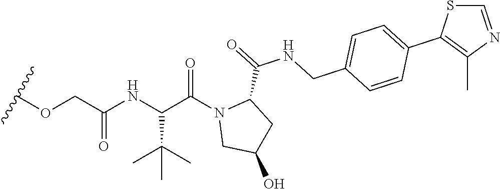 |
| 1070 | 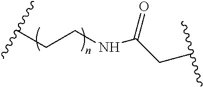 | 3 | 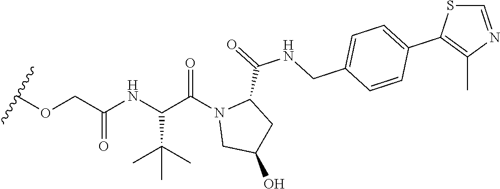 |
| 1071 | 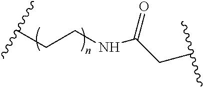 | 4 | 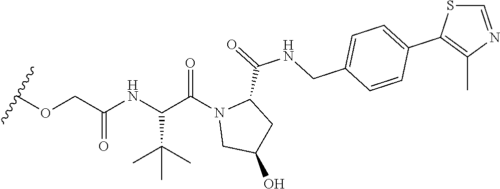 |
| 1072 | 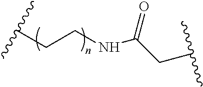 | 5 | 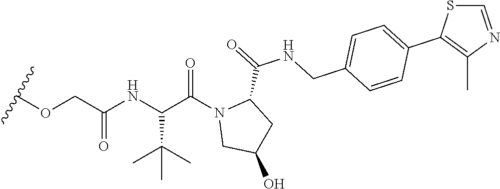 |
| 1073 | 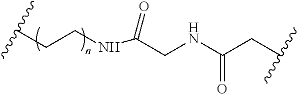 | 1 | 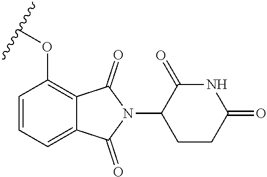 [SMILES: *Oc1cccc2c1C(=O)N(C1CCC(=O)NC1=O)C2=O] |
| 1074 | 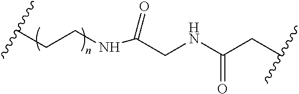 | 2 | 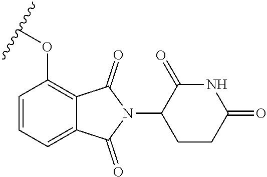 [SMILES: *Oc1cccc2c1C(=O)N(C1CCC(=O)NC1=O)C2=O] |
| 1075 | 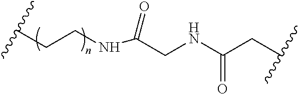 | 3 | 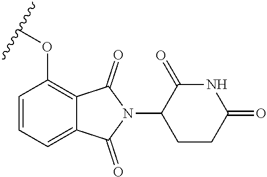 [SMILES: *Oc1cccc2c1C(=O)N(C1CCC(=O)NC1=O)C2=O] |
| 1076 | 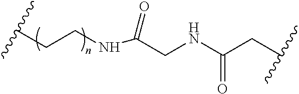 | 4 | 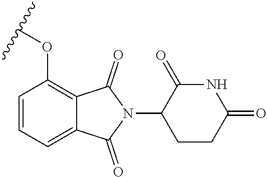 [SMILES: *Oc1cccc2c1C(=O)N(C1CCC(=O)NC1=O)C2=O] |
| 1077 | 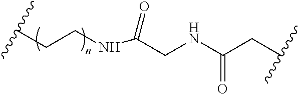 | 5 | 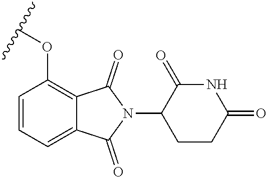 [SMILES: *Oc1cccc2c1C(=O)N(C1CCC(=O)NC1=O)C2=O] |
| 1078 | 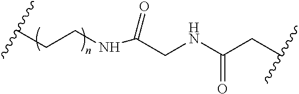 | 1 | 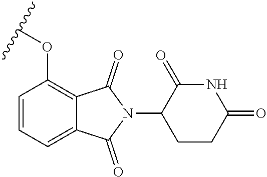 [SMILES: *Oc1cccc2c1C(=O)N(C1CCC(=O)NC1=O)C2=O] |
| 1079 | 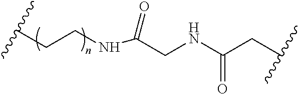 | 2 | 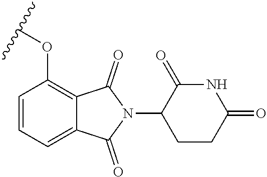 [SMILES: *Oc1cccc2c1C(=O)N(C1CCC(=O)NC1=O)C2=O] |
| 1080 | 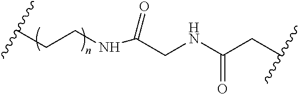 | 3 | 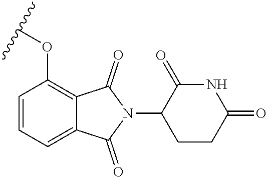 [SMILES: *Oc1cccc2c1C(=O)N(C1CCC(=O)NC1=O)C2=O] |
| 1081 | 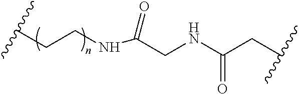 | 4 | 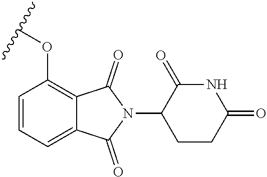 [SMILES: *Oc1cccc2c1C(=O)N(C1CCC(=O)NC1=O)C2=O] |
| 1082 | 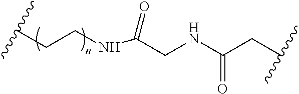 | 5 | 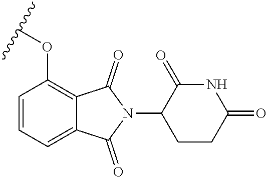 [SMILES: *Oc1cccc2c1C(=O)N(C1CCC(=O)NC1=O)C2=O] |
| 1083 | 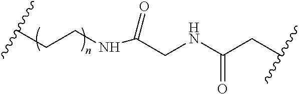 | 1 | 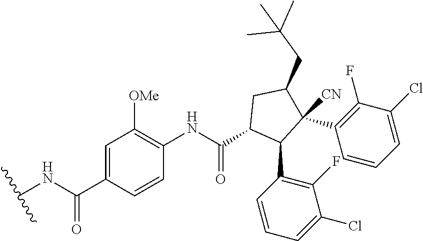 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1084 | 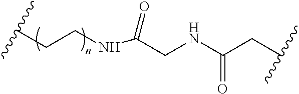 | 2 | 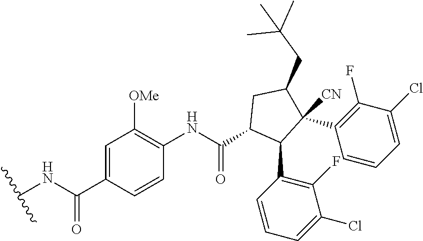 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1085 | 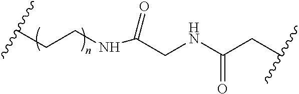 | 3 | 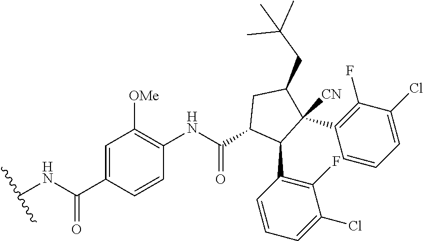 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1086 | 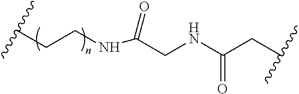 | 4 | 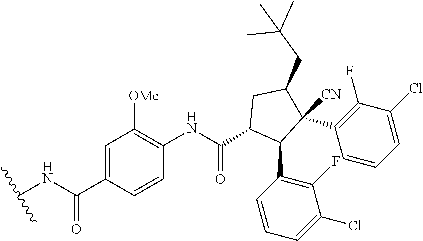 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1087 | 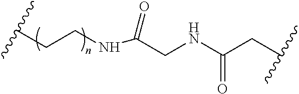 | 5 | 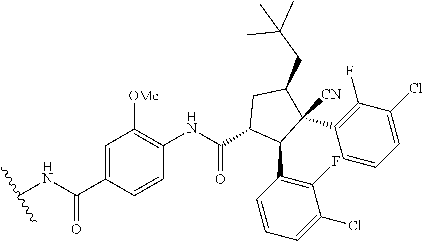 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1088 | 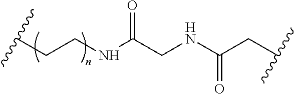 | 1 | 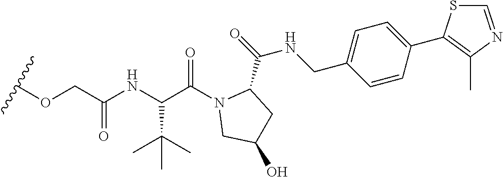 |
| 1089 | 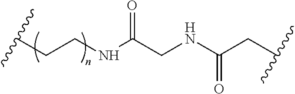 | 2 | 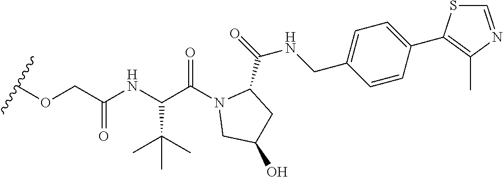 |
| 1090 | 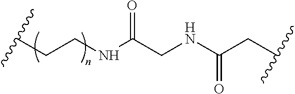 | 3 | 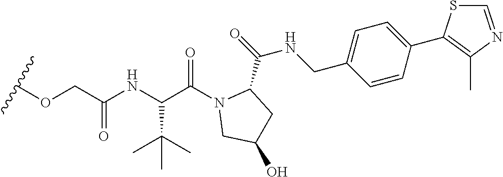 |
| 1091 | 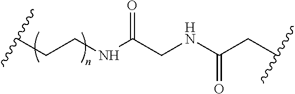 | 4 | 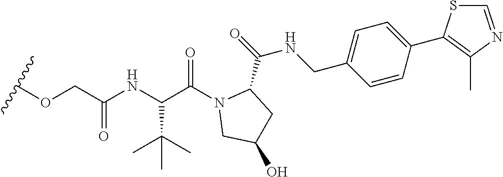 |
| 1092 | 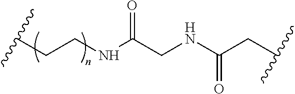 | 5 | 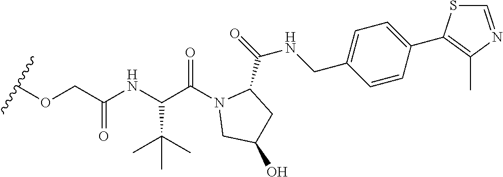 |

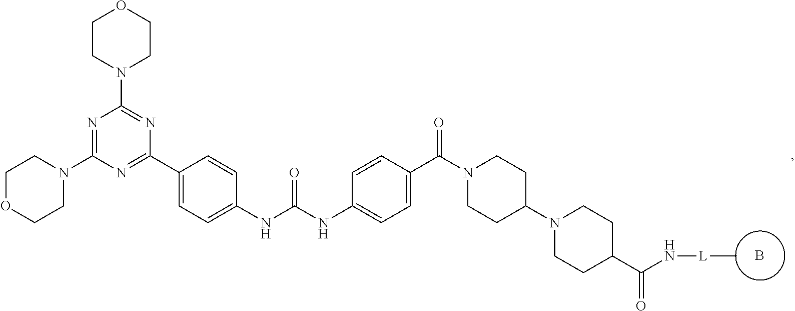

| Formula | | | |
|---|---|---|---|
| No. | L | n | B |
| 1093 | 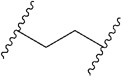 [SMILES: *CC*] | — | 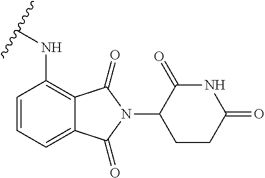 [SMILES: *Nc1cccc2c1C(=O)N(C1CCC(=O)NC1=O)C2=O] |
| 1094 | 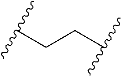 [SMILES: *CC*] | — | 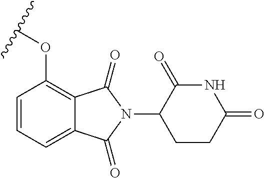 [SMILES: *Oc1cccc2c1C(=O)N(C1CCC(=O)NC1=O)C2=O] |
| 1095 | 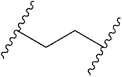 [SMILES: *CC*] | — | 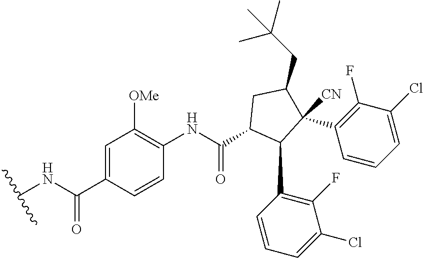 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1096 | 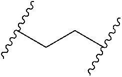 [SMILES: *CC*] | — | 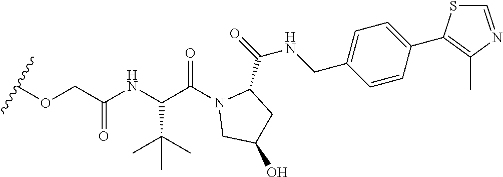 |
| 1097 | 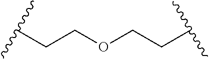 [SMILES: *CCOCC*] | — | 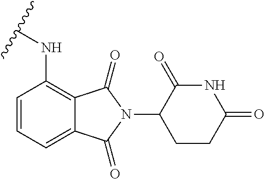 [SMILES: *Nc1cccc2c1C(=O)N(C1CCC(=O)NC1=O)C2=O] |
| 1098 | 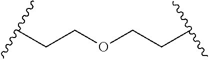 [SMILES: *CCOCC*] | — | 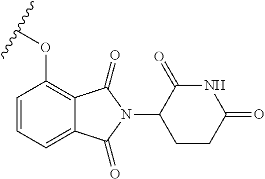 [SMILES: *Oc1cccc2c1C(=O)N(C1CCC(=O)NC1=O)C2=O] |
| 1099 | 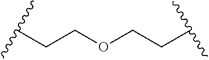 [SMILES: *CCOCC*] | — | 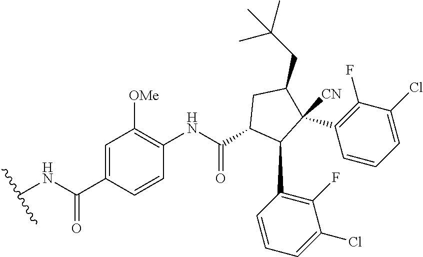 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1100 | 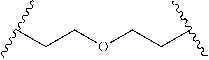 [SMILES: *CCOCC*] | — | 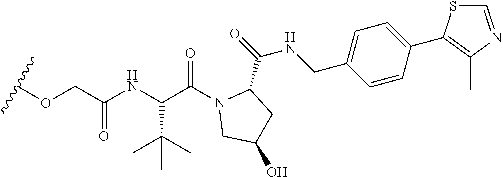 |
| 1101 | 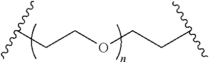 | 1 | 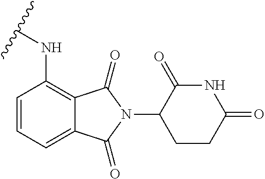 [SMILES: *Nc1cccc2c1C(=O)N(C1CCC(=O)NC1=O)C2=O] |
| 1102 | 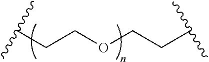 | 2 | 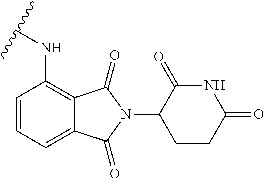 [SMILES: *Nc1cccc2c1C(=O)N(C1CCC(=O)NC1=O)C2=O] |
| 1103 | 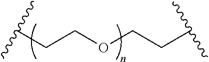 | 3 | 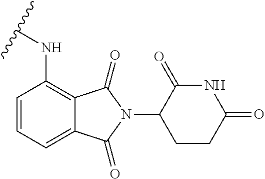 [SMILES: *Nc1cccc2c1C(=O)N(C1CCC(=O)NC1=O)C2=O] |
| 1104 | 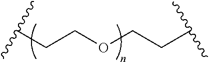 | 4 | 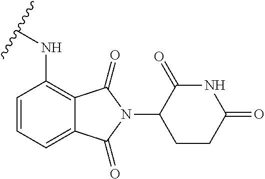 [SMILES: *Nc1cccc2c1C(=O)N(C1CCC(=O)NC1=O)C2=O] |
| 1105 | 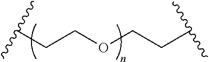 | 5 | 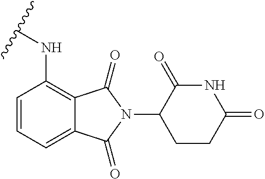 [SMILES: *Nc1cccc2c1C(=O)N(C1CCC(=O)NC1=O)C2=O] |
| 1106 | 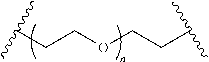 | 1 | 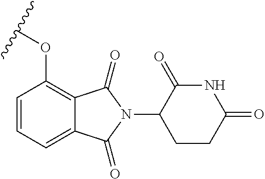 [SMILES: *Oc1cccc2c1C(=O)N(C1CCC(=O)NC1=O)C2=O] |
| 1107 | 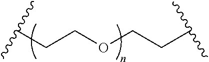 | 2 | 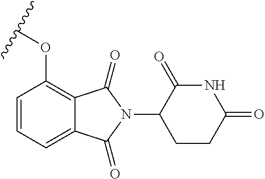 [SMILES: *Oc1cccc2c1C(=O)N(C1CCC(=O)NC1=O)C2=O] |
| 1108 | 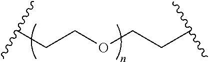 | 3 | 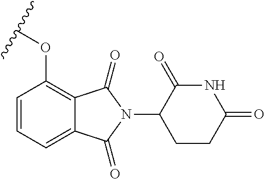 [SMILES: *Oc1cccc2c1C(=O)N(C1CCC(=O)NC1=O)C2=O] |
| 1109 | 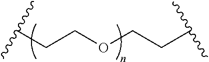 | 4 | 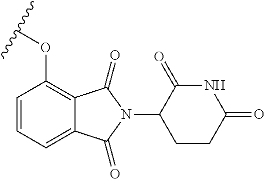 [SMILES: *Oc1cccc2c1C(=O)N(C1CCC(=O)NC1=O)C2=O] |
| 1110 | 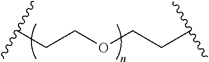 | 5 | 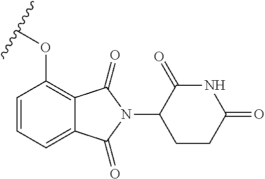 [SMILES: *Oc1cccc2c1C(=O)N(C1CCC(=O)NC1=O)C2=O] |
| 1111 | 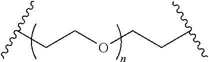 | 1 | 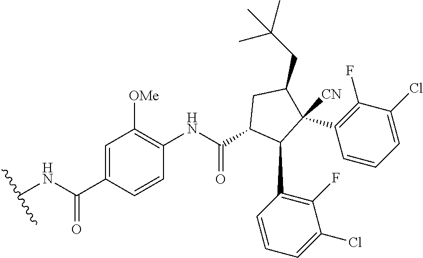 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1112 | 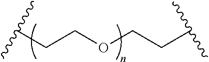 | 2 | 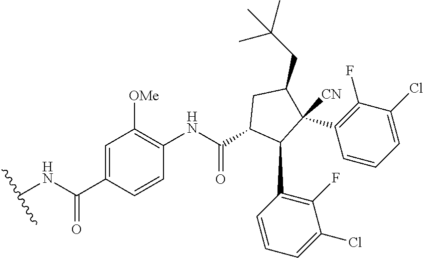 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1113 | 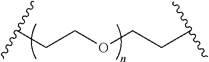 | 3 | 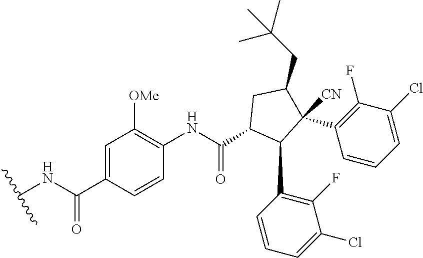 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1114 | 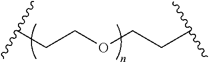 | 4 | 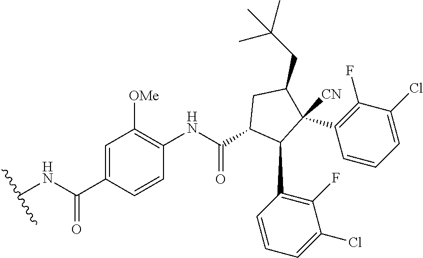 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1115 | 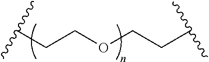 | 5 | 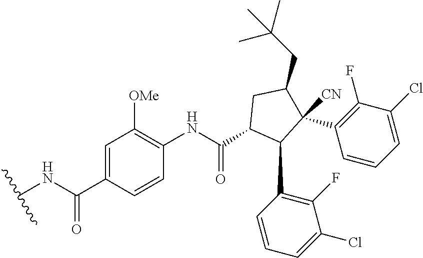 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1116 | 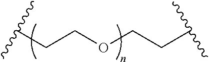 | 1 | 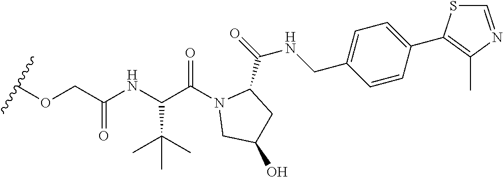 |
| 1117 | 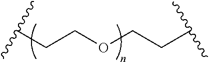 | 2 | 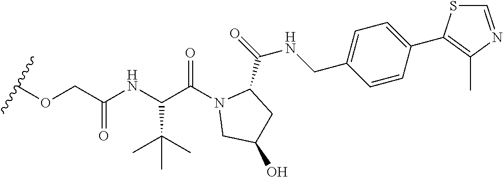 |
| 1118 | 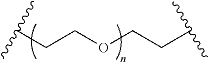 | 3 | 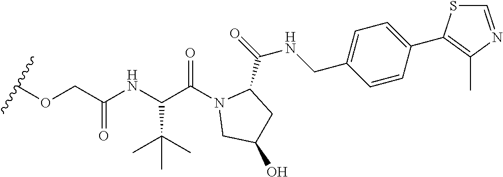 |
| 1119 | 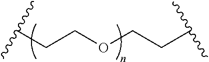 | 4 | 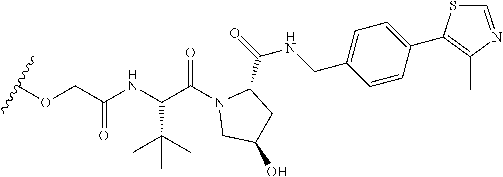 |
| 1120 | 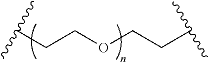 | 5 | 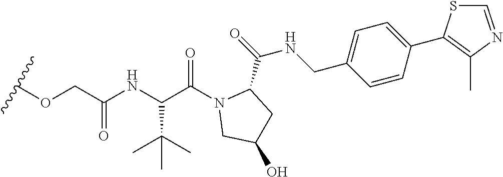 |
| 1121 | 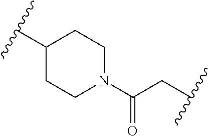 [SMILES: *CC(=O)N1CCC(*)CC1] | — | 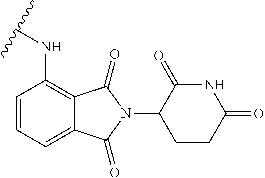 [SMILES: *Nc1cccc2c1C(=O)N(C1CCC(=O)NC1=O)C2=O] |
| 1122 | 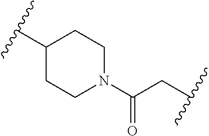 [SMILES: *CC(=O)N1CCC(*)CC1] | — | 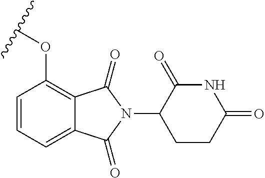 [SMILES: *Oc1cccc2c1C(=O)N(C1CCC(=O)NC1=O)C2=O] |
| 1123 | 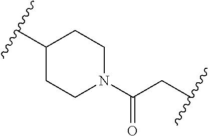 [SMILES: *CC(=O)N1CCC(*)CC1] | — | 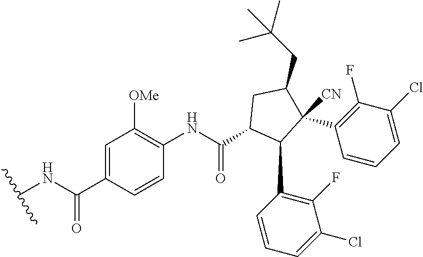 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1124 | 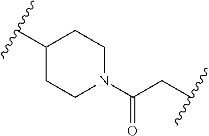 [SMILES: *CC(=O)N1CCC(*)CC1] | — | 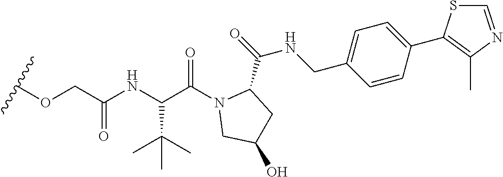 |
| 1125 | 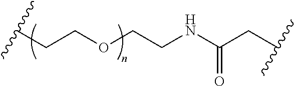 | 1 | 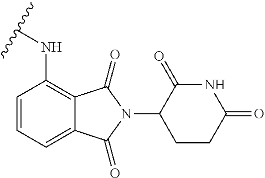 [SMILES: *Nc1cccc2c1C(=O)N(C1CCC(=O)NC1=O)C2=O] |
| 1126 | 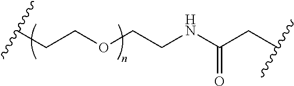 | 2 | 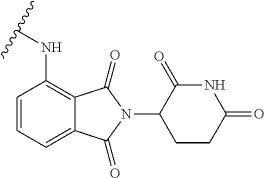 [SMILES: *Nc1cccc2c1C(=O)N(C1CCC(=O)NC1=O)C2=O] |
| 1127 | 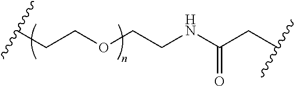 | 3 | 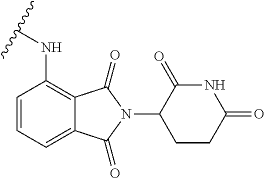 [SMILES: *Nc1cccc2c1C(=O)N(C1CCC(=O)NC1=O)C2=O] |
| 1128 | 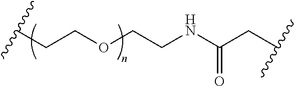 | 4 | 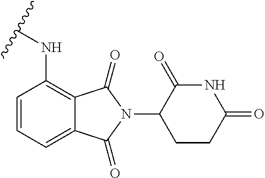 [SMILES: *Nc1cccc2c1C(=O)N(C1CCC(=O)NC1=O)C2=O] |
| 1129 | 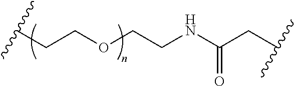 | 5 | 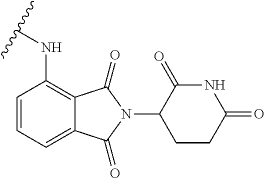 [SMILES: *Nc1cccc2c1C(=O)N(C1CCC(=O)NC1=O)C2=O] |
| 1130 | 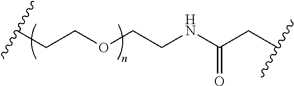 | 1 | 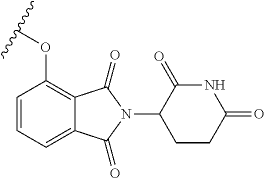 [SMILES: *Oc1cccc2c1C(=O)N(C1CCC(=O)NC1=O)C2=O] |
| 1131 | 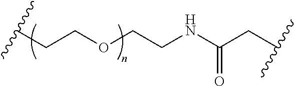 | 2 | 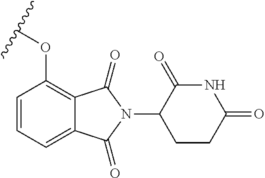 [SMILES: *Oc1cccc2c1C(=O)N(C1CCC(=O)NC1=O)C2=O] |
| 1132 | 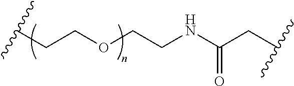 | 3 | 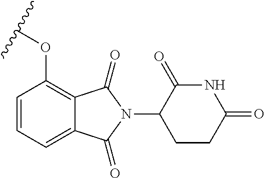 [SMILES: *Oc1cccc2c1C(=O)N(C1CCC(=O)NC1=O)C2=O] |
| 1133 | 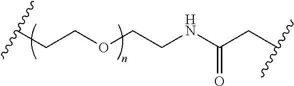 | 4 | 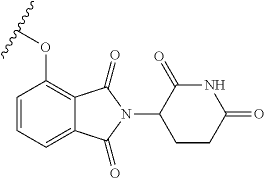 [SMILES: *Oc1cccc2c1C(=O)N(C1CCC(=O)NC1=O)C2=O] |
| 1134 | 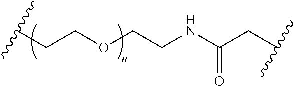 | 5 | 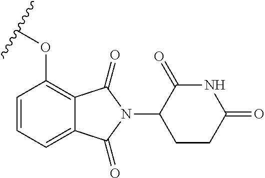 [SMILES: *Oc1cccc2c1C(=O)N(C1CCC(=O)NC1=O)C2=O] |
| 1135 | 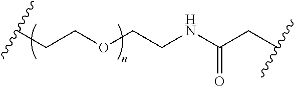 | 1 | 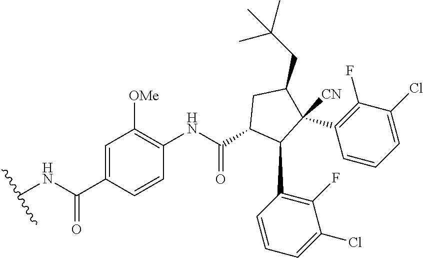 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1136 | 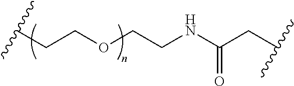 | 2 | 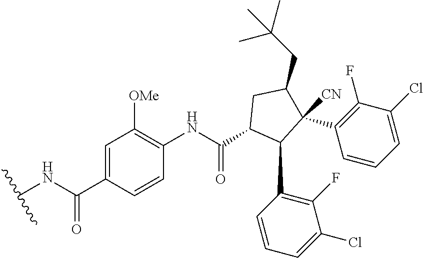 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1137 | 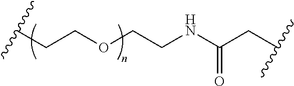 | 3 | 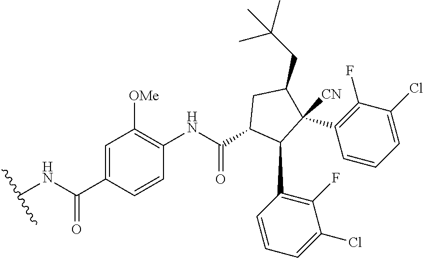 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1138 | 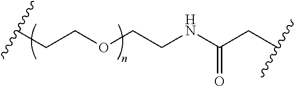 | 4 | 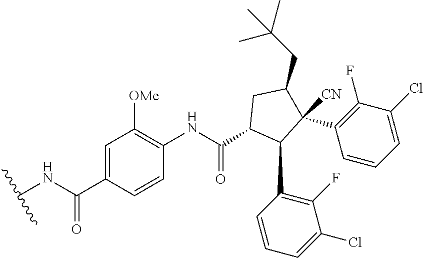 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1139 | 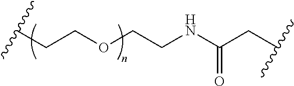 | 5 | 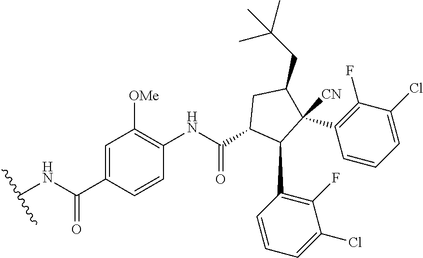 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1140 | 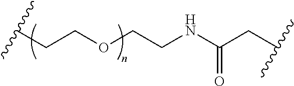 | 1 | 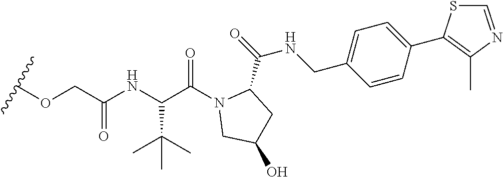 |
| 1141 | 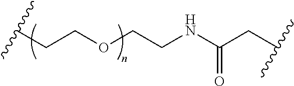 | 2 | 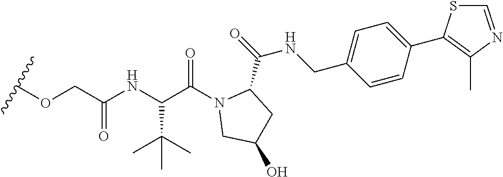 |
| 1142 | 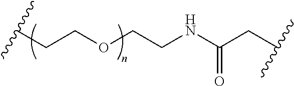 | 3 | 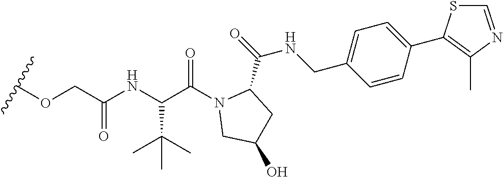 |
| 1143 | 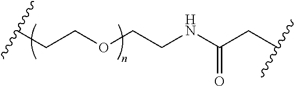 | 4 | 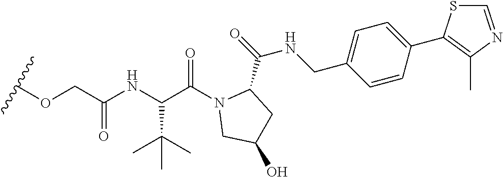 |
| 1144 | 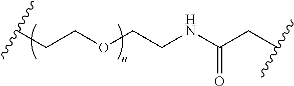 | 5 | 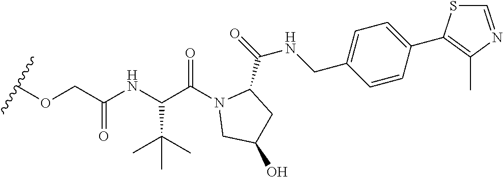 |
| 1145 | 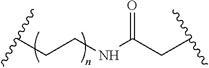 | 1 | 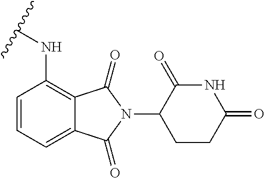 [SMILES: *Nc1cccc2c1C(=O)N(C1CCC(=O)NC1=O)C2=O] |
| 1146 | 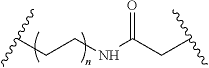 | 2 | 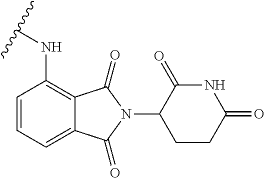 [SMILES: *Nc1cccc2c1C(=O)N(C1CCC(=O)NC1=O)C2=O] |
| 1147 | 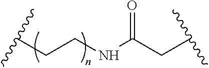 | 3 | 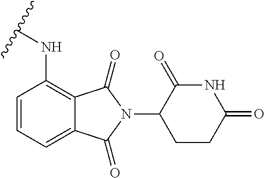 [SMILES: *Nc1cccc2c1C(=O)N(C1CCC(=O)NC1=O)C2=O] |
| 1148 | 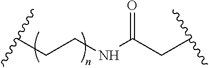 | 4 | 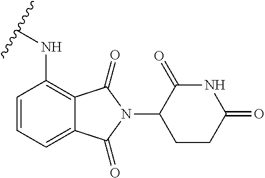 [SMILES: *Nc1cccc2c1C(=O)N(C1CCC(=O)NC1=O)C2=O] |
| 1149 | 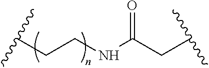 | 5 | 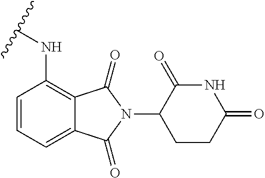 [SMILES: *Nc1cccc2c1C(=O)N(C1CCC(=O)NC1=O)C2=O] |
| 1150 | 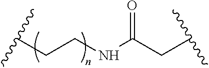 | 1 | 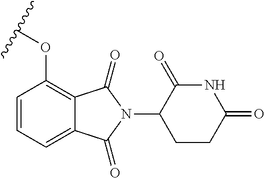 [SMILES: *Oc1cccc2c1C(=O)N(C1CCC(=O)NC1=O)C2=O] |
| 1151 | 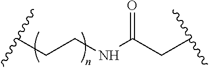 | 2 | 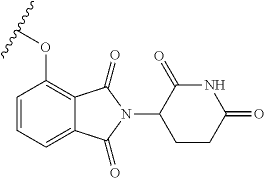 [SMILES: *Oc1cccc2c1C(=O)N(C1CCC(=O)NC1=O)C2=O] |
| 1152 | 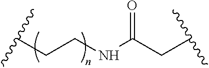 | 3 | 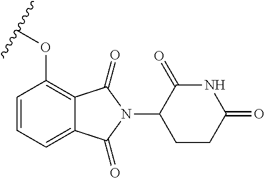 [SMILES: *Oc1cccc2c1C(=O)N(C1CCC(=O)NC1=O)C2=O] |
| 1153 | 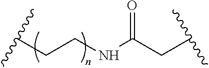 | 4 | 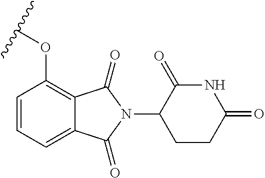 [SMILES: *Oc1cccc2c1C(=O)N(C1CCC(=O)NC1=O)C2=O] |
| 1154 | 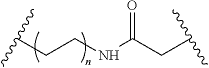 | 5 | 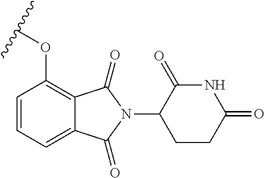 [SMILES: *Oc1cccc2c1C(=O)N(C1CCC(=O)NC1=O)C2=O] |
| 1155 | 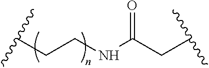 | 1 | 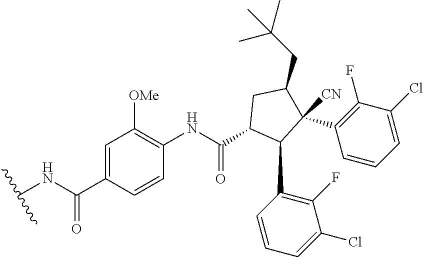 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1156 | 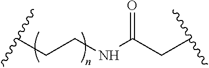 | 2 | 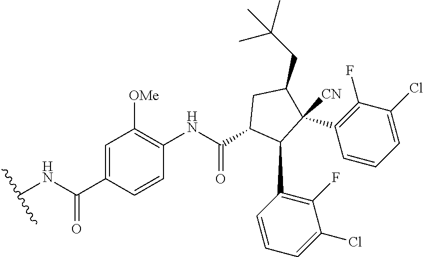 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1157 | 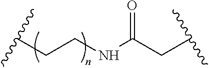 | 3 | 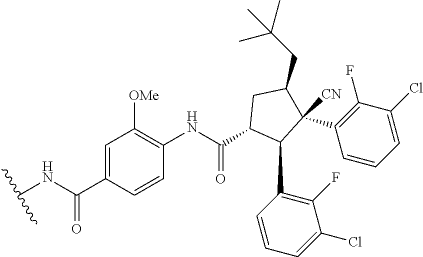 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1158 | 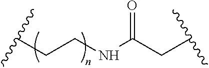 | 4 | 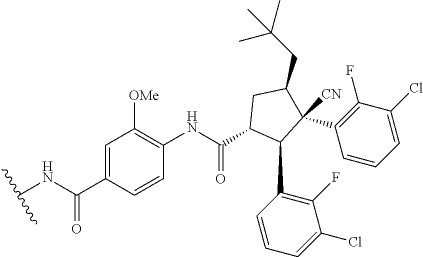 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1159 | 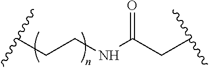 | 5 | 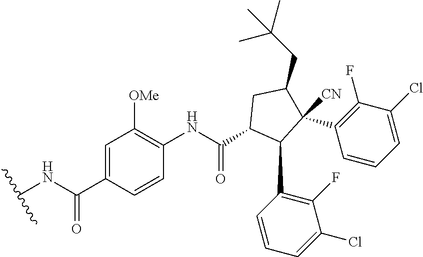 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1160 | 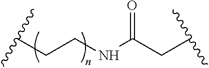 | 1 | 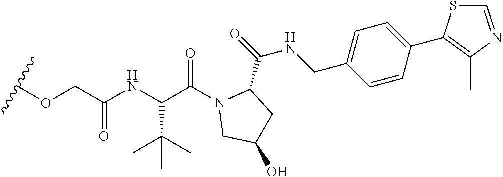 |
| 1161 | 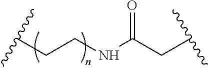 | 2 | 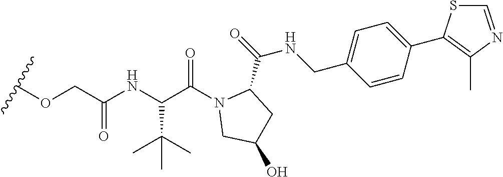 |
| 1162 | 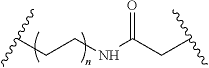 | 3 | 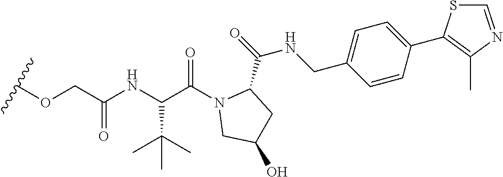 |
| 1163 | 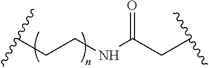 | 4 | 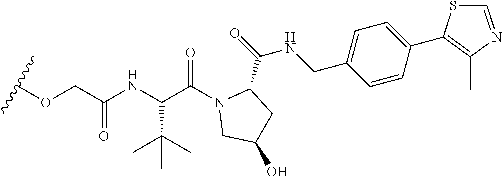 |
| 1164 | 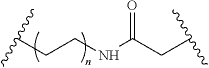 | 5 | 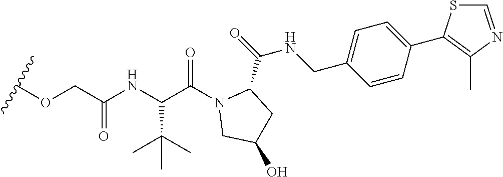 |
| 1165 | 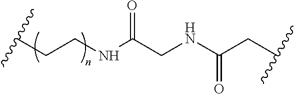 | 1 | 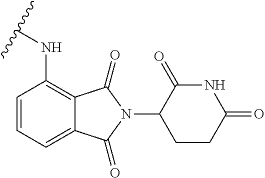 [SMILES: *Nc1cccc2c1C(=O)N(C1CCC(=O)NC1=O)C2=O] |
| 1166 | 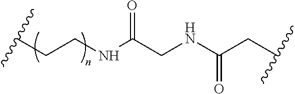 | 2 | 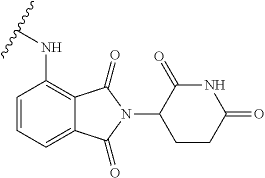 [SMILES: *Nc1cccc2c1C(=O)N(C1CCC(=O)NC1=O)C2=O] |
| 1167 | 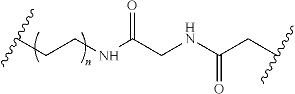 | 3 | 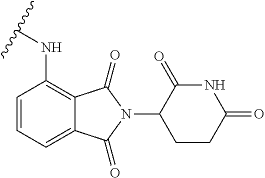 [SMILES: *Nc1cccc2c1C(=O)N(C1CCC(=O)NC1=O)C2=O] |
| 1168 | 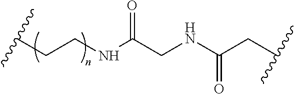 | 4 | 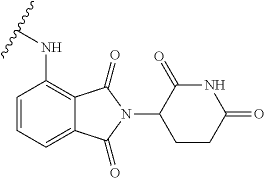 [SMILES: *Nc1cccc2c1C(=O)N(C1CCC(=O)NC1=O)C2=O] |
| 1169 | 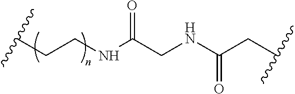 | 5 | 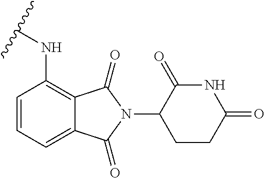 [SMILES: *Nc1cccc2c1C(=O)N(C1CCC(=O)NC1=O)C2=O] |
| 1170 | 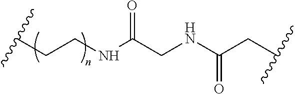 | 1 | 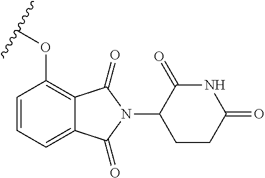 [SMILES: *Oc1cccc2c1C(=O)N(C1CCC(=O)NC1=O)C2=O] |
| 1171 | 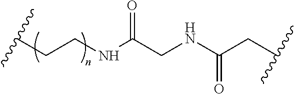 | 2 | 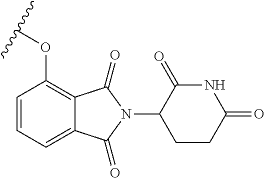 [SMILES: *Oc1cccc2c1C(=O)N(C1CCC(=O)NC1=O)C2=O] |
| 1172 | 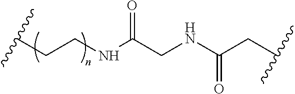 | 3 | 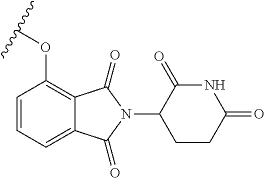 [SMILES: *Oc1cccc2c1C(=O)N(C1CCC(=O)NC1=O)C2=O] |
| 1173 | 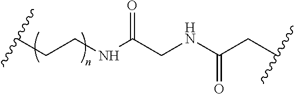 | 4 | 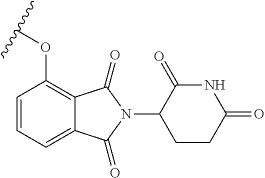 [SMILES: *Oc1cccc2c1C(=O)N(C1CCC(=O)NC1=O)C2=O] |
| 1174 | 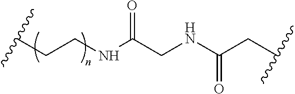 | 5 | 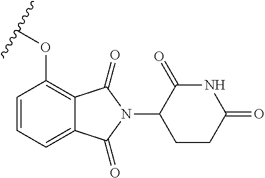 [SMILES: *Oc1cccc2c1C(=O)N(C1CCC(=O)NC1=O)C2=O] |
| 1175 | 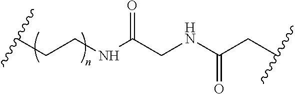 | 1 | 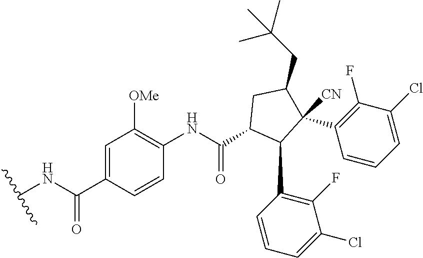 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1176 | 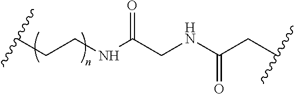 | 2 | 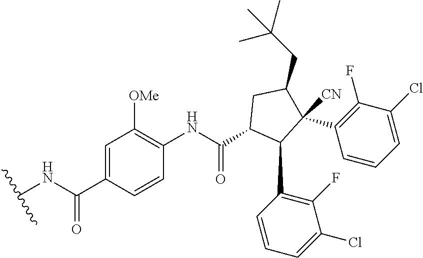 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1177 | 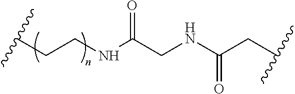 | 3 | 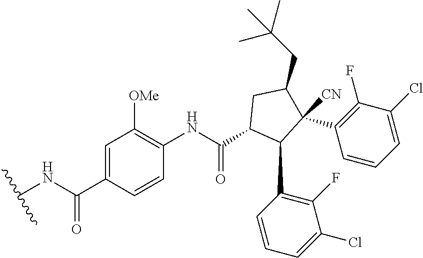 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1178 | 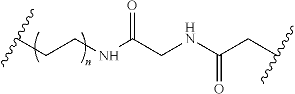 | 4 | 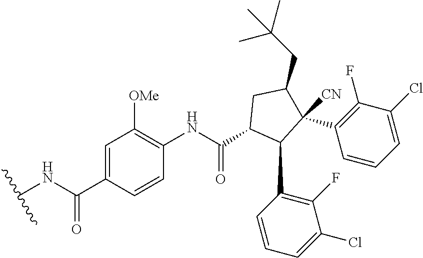 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1179 | 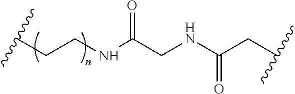 | 5 | 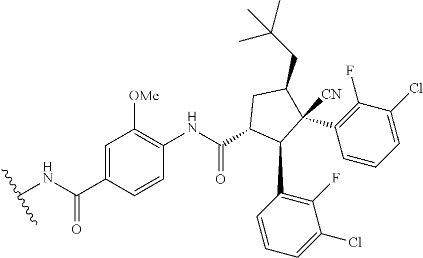 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1180 | 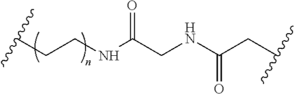 | 1 | 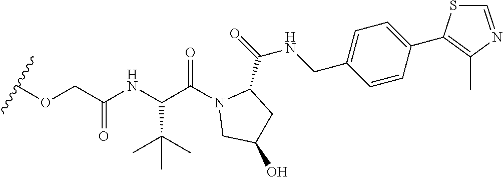 |
| 1181 | 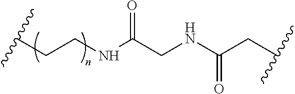 | 2 | 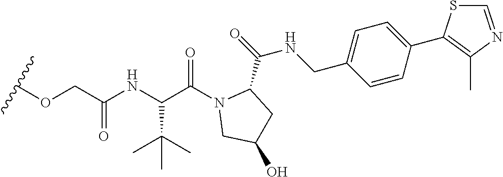 |
| 1182 | 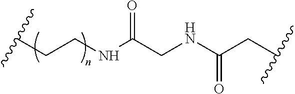 | 3 | 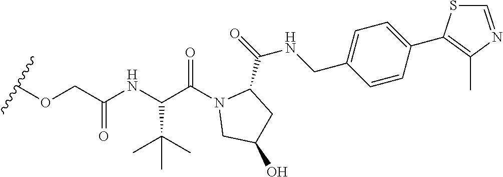 |
| 1183 | 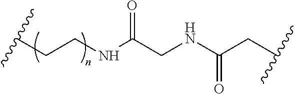 | 4 | 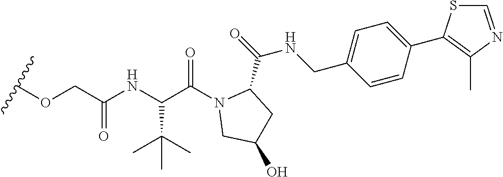 |
| 1184 | 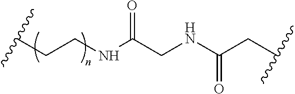 | 5 | 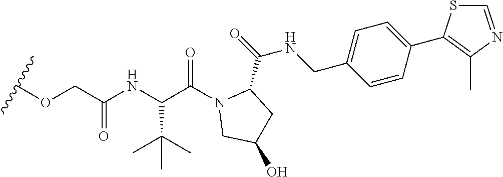 |

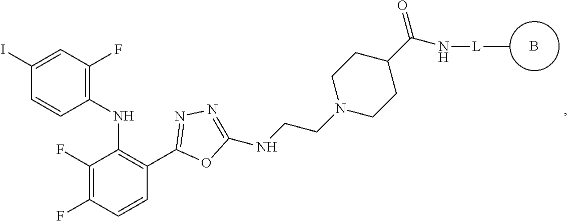

| Formula | | | |
|---|---|---|---|
| No. | L | n | B |
| 1185 | 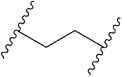 [SMILES: *CC*] | — | 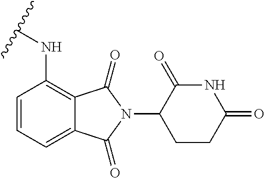 [SMILES: *Nc1cccc2c1C(=O)N(C1CCC(=O)NC1=O)C2=O] |
| 1186 | 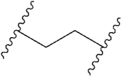 [SMILES: *CC*] | — | 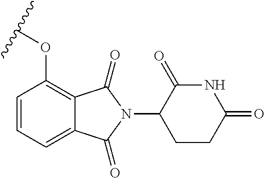 [SMILES: *Oc1cccc2c1C(=O)N(C1CCC(=O)NC1=O)C2=O] |
| 1187 | 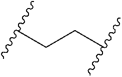 [SMILES: *CC*] | — | 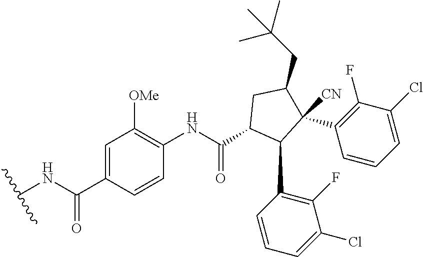 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1188 | 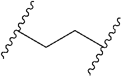 [SMILES: *CC*] | — | 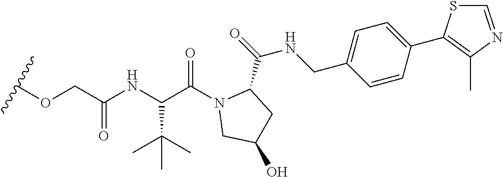 |
| 1189 | 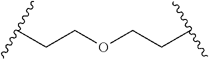 [SMILES: *CCOCC*] | — | 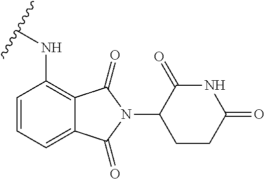 [SMILES: *Nc1cccc2c1C(=O)N(C1CCC(=O)NC1=O)C2=O] |
| 1190 | 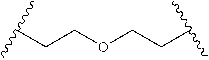 [SMILES: *CCOCC*] | — | 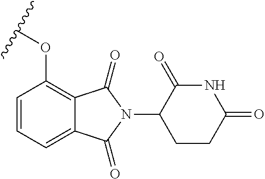 [SMILES: *Oc1cccc2c1C(=O)N(C1CCC(=O)NC1=O)C2=O] |
| 1191 | 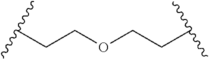 [SMILES: *CCOCC*] | — | 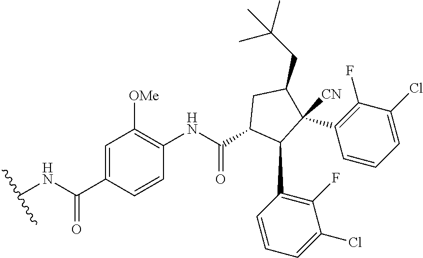 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1192 | 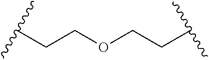 [SMILES: *CCOCC*] | — | 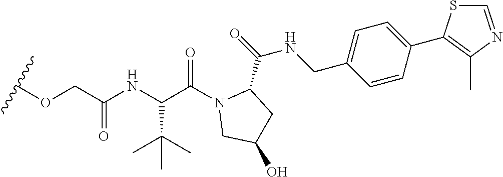 |
| 1193 | 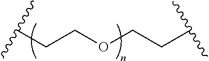 | 1 | 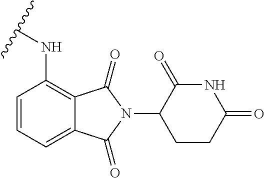 [SMILES: *Nc1cccc2c1C(=O)N(C1CCC(=O)NC1=O)C2=O] |
| 1194 | 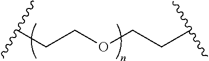 | 2 | 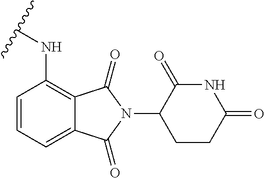 [SMILES: *Nc1cccc2c1C(=O)N(C1CCC(=O)NC1=O)C2=O] |
| 1195 | 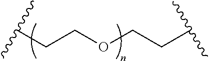 | 3 | 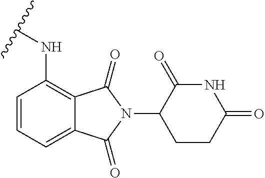 [SMILES: *Nc1cccc2c1C(=O)N(C1CCC(=O)NC1=O)C2=O] |
| 1196 | 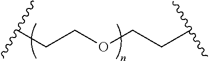 | 4 | 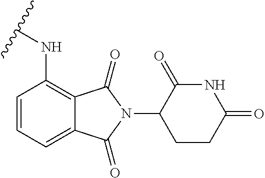 [SMILES: *Nc1cccc2c1C(=O)N(C1CCC(=O)NC1=O)C2=O] |
| 1197 | 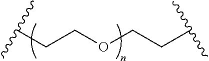 | 5 | 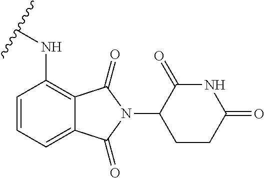 [SMILES: *Nc1cccc2c1C(=O)N(C1CCC(=O)NC1=O)C2=O] |
| 1198 | 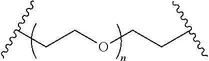 | 1 | 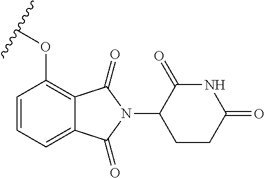 [SMILES: *Oc1cccc2c1C(=O)N(C1CCC(=O)NC1=O)C2=O] |
| 1199 | 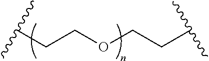 | 2 | 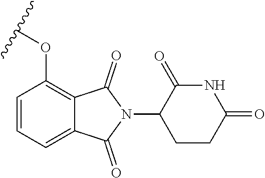 [SMILES: *Oc1cccc2c1C(=O)N(C1CCC(=O)NC1=O)C2=O] |
| 1200 | 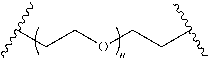 | 3 | 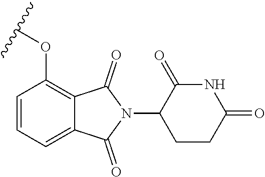 [SMILES: *Oc1cccc2c1C(=O)N(C1CCC(=O)NC1=O)C2=O] |
| 1201 | 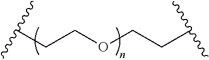 | 4 | 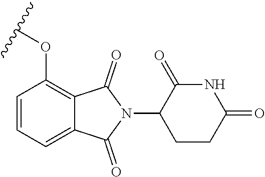 [SMILES: *Oc1cccc2c1C(=O)N(C1CCC(=O)NC1=O)C2=O] |
| 1202 | 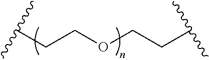 | 5 | 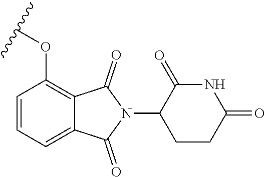 [SMILES: *Oc1cccc2c1C(=O)N(C1CCC(=O)NC1=O)C2=O] |
| 1203 | 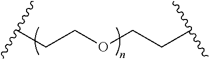 | 1 | 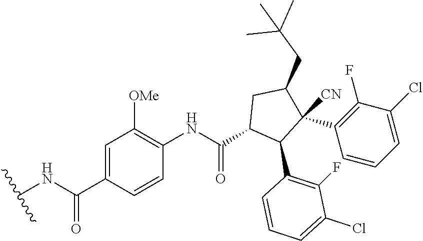 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1204 | 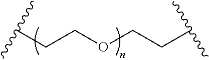 | 2 | 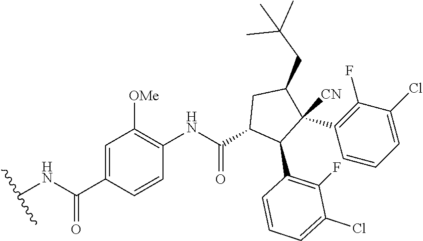 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1205 | 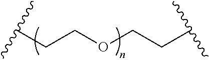 | 3 | 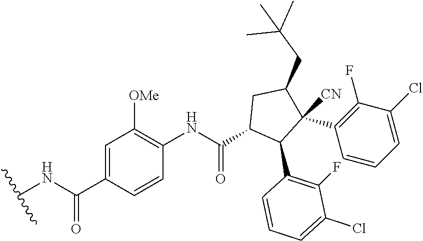 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1206 | 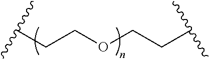 | 4 | 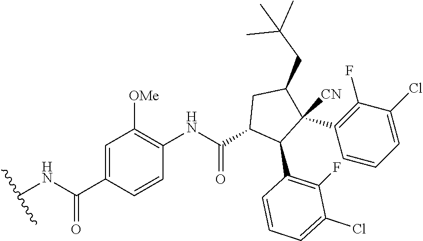 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1207 | 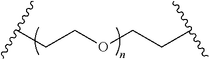 | 5 | 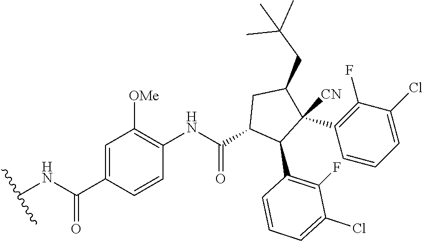 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1208 | 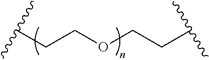 | 1 | 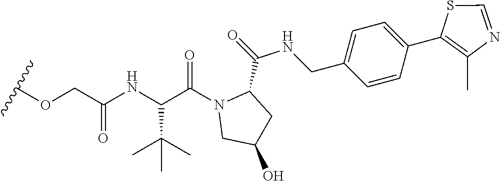 |
| 1209 | 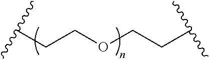 | 2 | 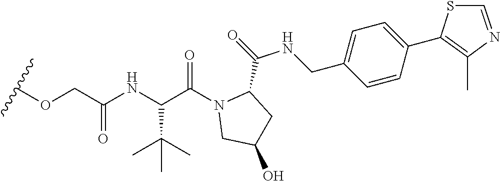 |
| 1210 | 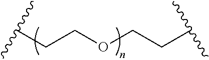 | 3 | 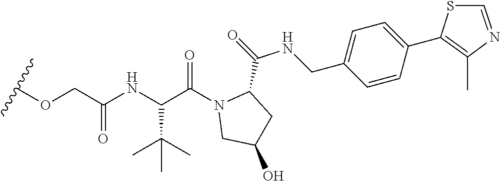 |
| 1211 | 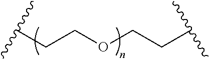 | 4 | 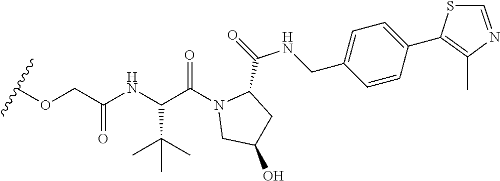 |
| 1212 | 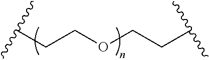 | 5 | 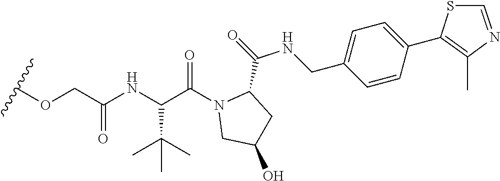 |
| 1213 | 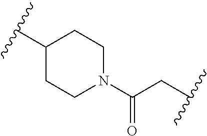 [SMILES: *CC(=O)N1CCC(*)CC1] | — | 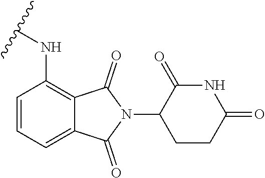 [SMILES: *Nc1cccc2c1C(=O)N(C1CCC(=O)NC1=O)C2=O] |
| 1214 | 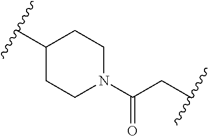 [SMILES: *CC(=O)N1CCC(*)CC1] | — | 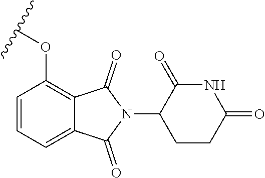 [SMILES: *Oc1cccc2c1C(=O)N(C1CCC(=O)NC1=O)C2=O] |
| 1215 | 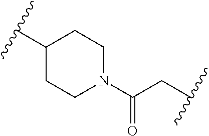 [SMILES: *CC(=O)N1CCC(*)CC1] | — | 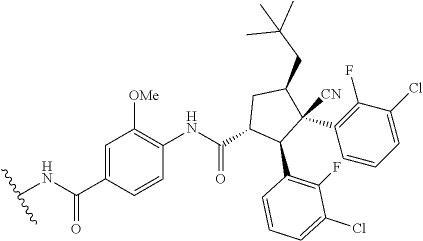 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1216 | 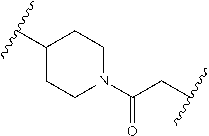 [SMILES: *CC(=O)N1CCC(*)CC1] | — | 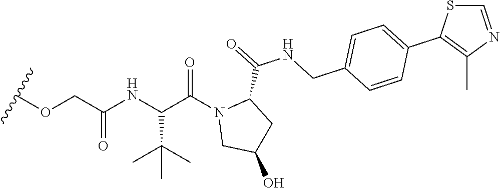 |
| 1217 | 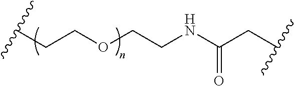 | 1 | 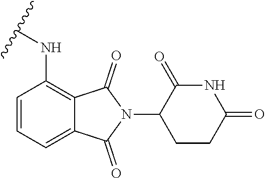 [SMILES: *Nc1cccc2c1C(=O)N(C1CCC(=O)NC1=O)C2=O] |
| 1218 | 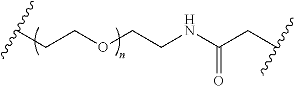 | 2 | 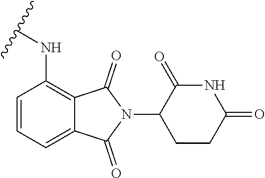 [SMILES: *Nc1cccc2c1C(=O)N(C1CCC(=O)NC1=O)C2=O] |
| 1219 | 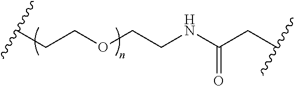 | 3 | 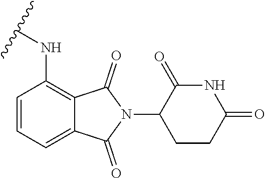 [SMILES: *Nc1cccc2c1C(=O)N(C1CCC(=O)NC1=O)C2=O] |
| 1220 | 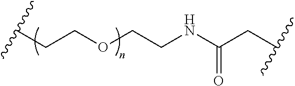 | 4 | 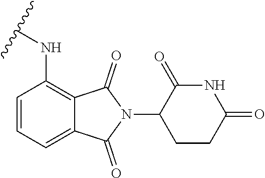 [SMILES: *Nc1cccc2c1C(=O)N(C1CCC(=O)NC1=O)C2=O] |
| 1221 | 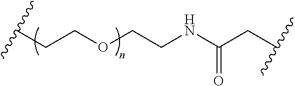 | 5 | 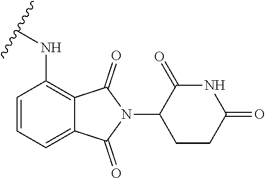 [SMILES: *Nc1cccc2c1C(=O)N(C1CCC(=O)NC1=O)C2=O] |
| 1222 | 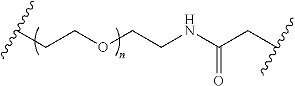 | 1 | 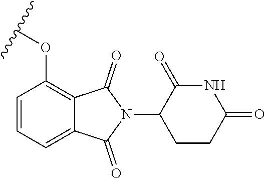 [SMILES: *Oc1cccc2c1C(=O)N(C1CCC(=O)NC1=O)C2=O] |
| 1223 | 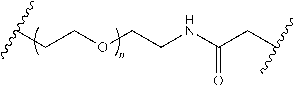 | 2 | 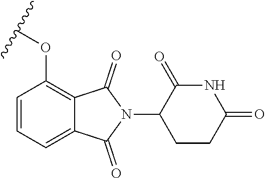 [SMILES: *Oc1cccc2c1C(=O)N(C1CCC(=O)NC1=O)C2=O] |
| 1224 | 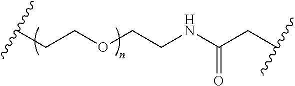 | 3 | 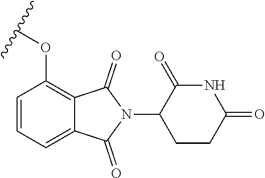 [SMILES: *Oc1cccc2c1C(=O)N(C1CCC(=O)NC1=O)C2=O] |
| 1225 | 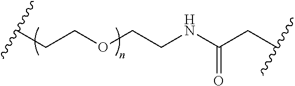 | 4 | 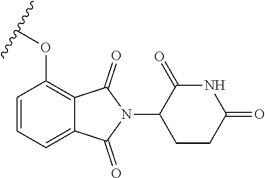 [SMILES: *Oc1cccc2c1C(=O)N(C1CCC(=O)NC1=O)C2=O] |
| 1226 | 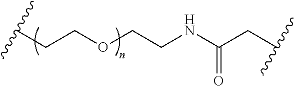 | 5 | 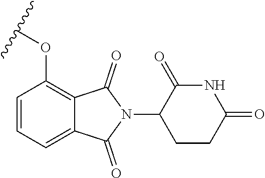 [SMILES: *Oc1cccc2c1C(=O)N(C1CCC(=O)NC1=O)C2=O] |
| 1227 | 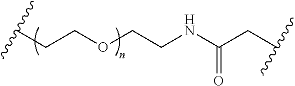 | 1 | 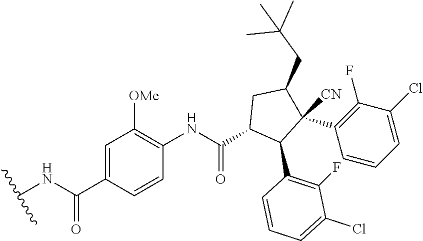 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1228 | 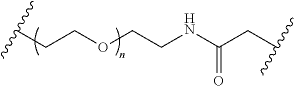 | 2 | 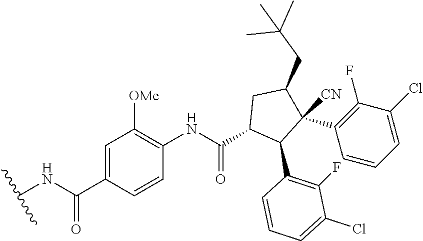 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1229 | 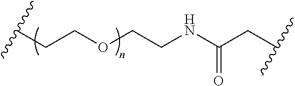 | 3 | 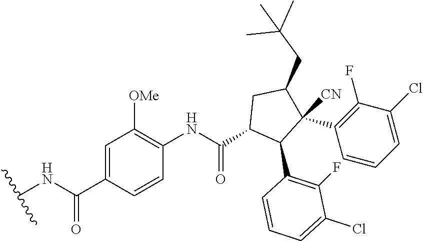 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1230 | 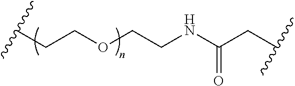 | 4 | 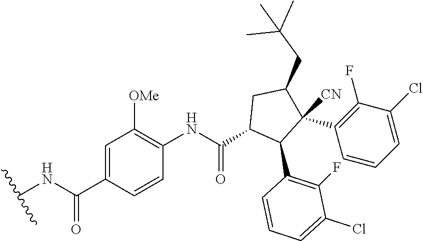 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1231 | 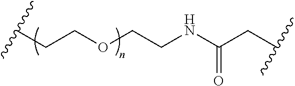 | 5 | 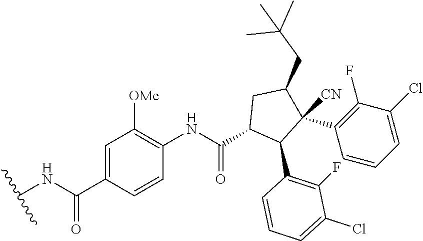 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1232 | 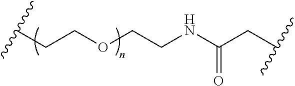 | 1 | 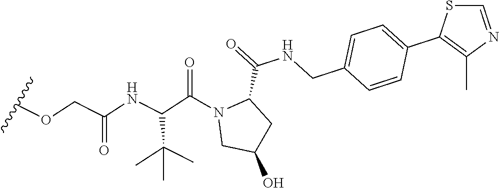 |
| 1233 | 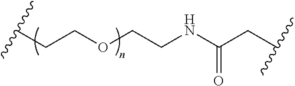 | 2 | 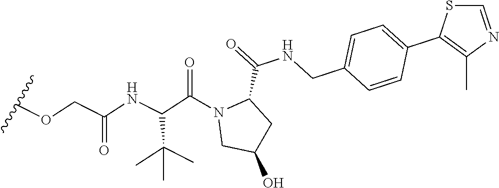 |
| 1234 | 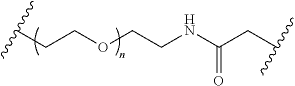 | 3 | 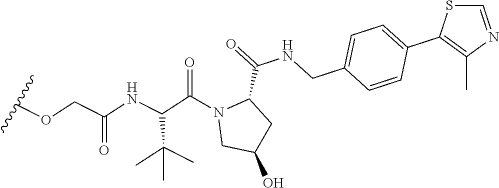 |
| 1235 | 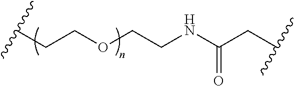 | 4 | 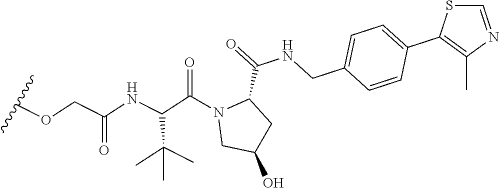 |
| 1236 | 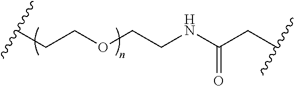 | 5 | 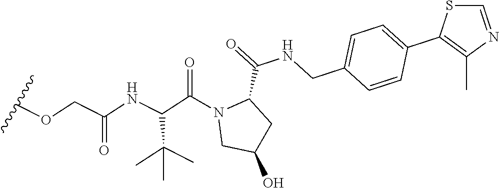 |
| 1237 | 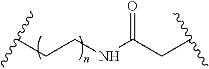 | 1 | 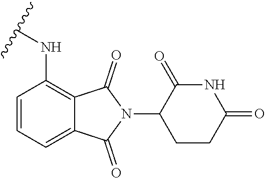 [SMILES: *Nc1cccc2c1C(=O)N(C1CCC(=O)NC1=O)C2=O] |
| 1238 | 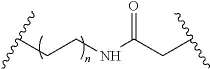 | 2 | 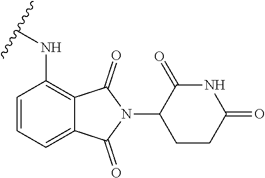 [SMILES: *Nc1cccc2c1C(=O)N(C1CCC(=O)NC1=O)C2=O] |
| 1239 | 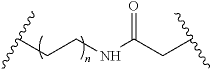 | 3 | 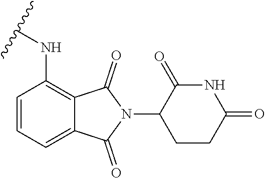 [SMILES: *Nc1cccc2c1C(=O)N(C1CCC(=O)NC1=O)C2=O] |
| 1240 | 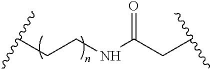 | 4 | 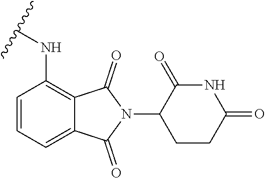 [SMILES: *Nc1cccc2c1C(=O)N(C1CCC(=O)NC1=O)C2=O] |
| 1241 | 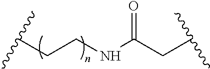 | 5 | 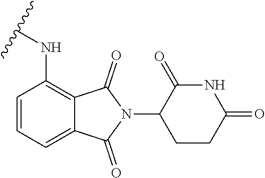 [SMILES: *Nc1cccc2c1C(=O)N(C1CCC(=O)NC1=O)C2=O] |
| 1242 | 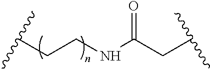 | 1 | 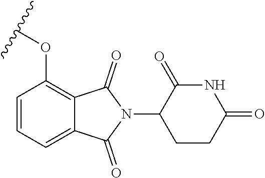 [SMILES: *Oc1cccc2c1C(=O)N(C1CCC(=O)NC1=O)C2=O] |
| 1243 | 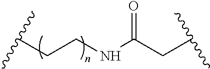 | 2 | 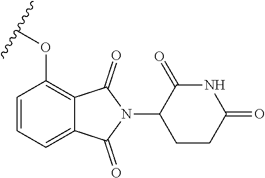 [SMILES: *Oc1cccc2c1C(=O)N(C1CCC(=O)NC1=O)C2=O] |
| 1244 | 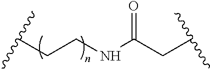 | 3 | 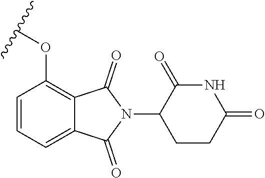 [SMILES: *Oc1cccc2c1C(=O)N(C1CCC(=O)NC1=O)C2=O] |
| 1245 | 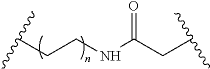 | 4 | 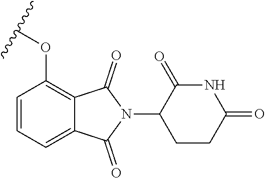 [SMILES: *Oc1cccc2c1C(=O)N(C1CCC(=O)NC1=O)C2=O] |
| 1246 | 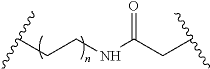 | 5 | 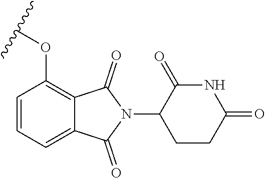 [SMILES: *Oc1cccc2c1C(=O)N(C1CCC(=O)NC1=O)C2=O] |
| 1247 | 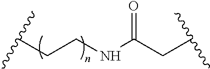 | 1 | 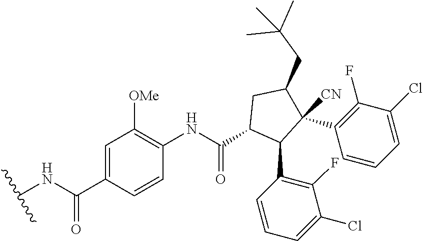 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1248 | 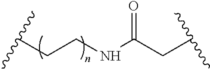 | 2 | 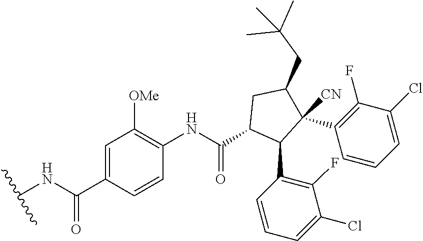 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1249 | 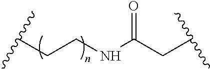 | 3 | 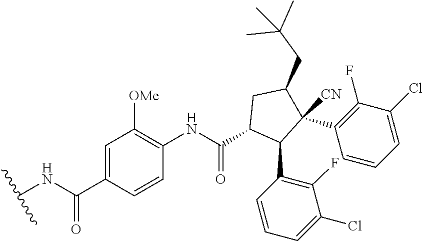 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1250 | 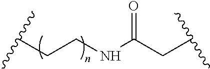 | 4 | 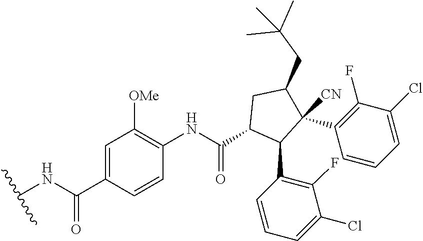 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1251 | 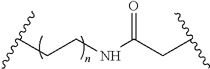 | 5 | 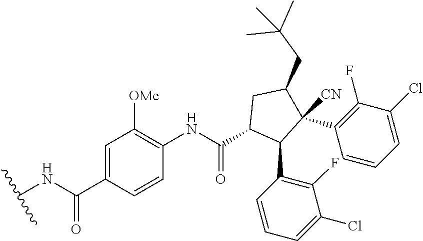 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1252 | 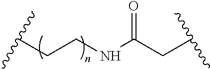 | 1 | 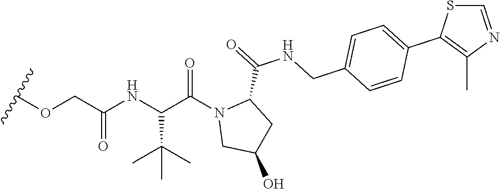 |
| 1253 | 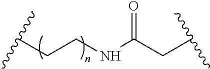 | 2 | 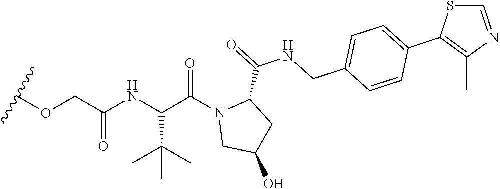 |
| 1254 | 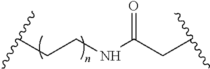 | 3 | 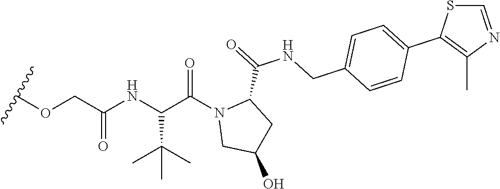 |
| 1255 | 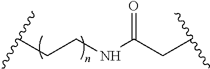 | 4 | 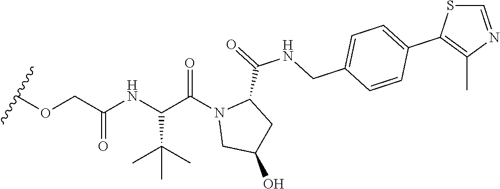 |
| 1256 | 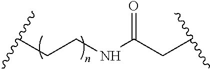 | 5 | 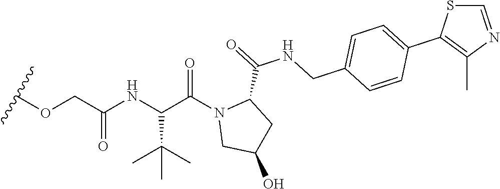 |
| 1257 | 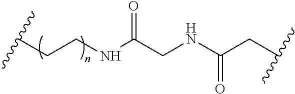 | 1 | 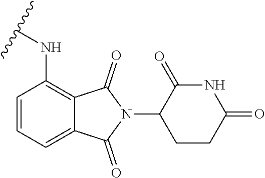 [SMILES: *Nc1cccc2c1C(=O)N(C1CCC(=O)NC1=O)C2=O] |
| 1258 | 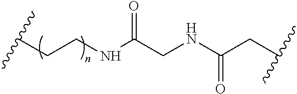 | 2 | 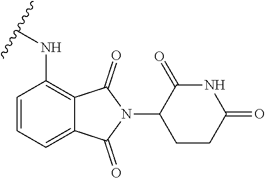 [SMILES: *Nc1cccc2c1C(=O)N(C1CCC(=O)NC1=O)C2=O] |
| 1259 | 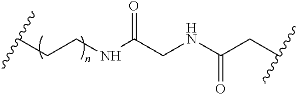 | 3 | 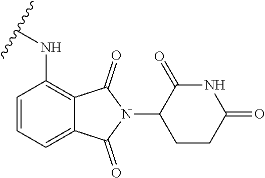 [SMILES: *Nc1cccc2c1C(=O)N(C1CCC(=O)NC1=O)C2=O] |
| 1260 | 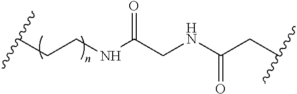 | 4 | 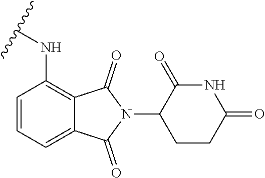 [SMILES: *Nc1cccc2c1C(=O)N(C1CCC(=O)NC1=O)C2=O] |
| 1261 | 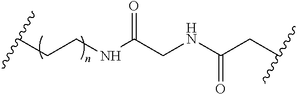 | 5 | 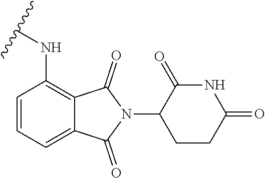 [SMILES: *Nc1cccc2c1C(=O)N(C1CCC(=O)NC1=O)C2=O] |
| 1262 | 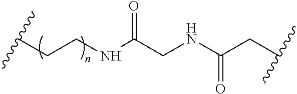 | 1 | 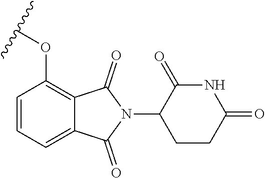 [SMILES: *Oc1cccc2c1C(=O)N(C1CCC(=O)NC1=O)C2=O] |
| 1263 | 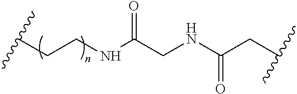 | 2 | 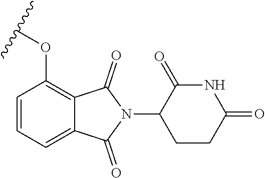 [SMILES: *Oc1cccc2c1C(=O)N(C1CCC(=O)NC1=O)C2=O] |
| 1264 | 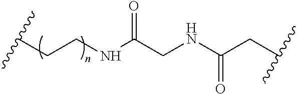 | 3 | 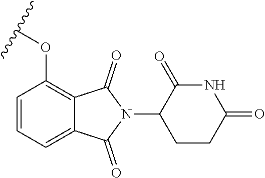 [SMILES: *Oc1cccc2c1C(=O)N(C1CCC(=O)NC1=O)C2=O] |
| 1265 | 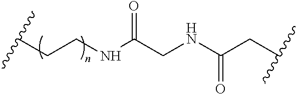 | 4 | 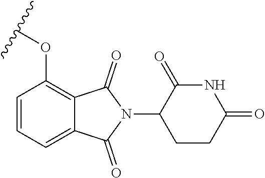 [SMILES: *Oc1cccc2c1C(=O)N(C1CCC(=O)NC1=O)C2=O] |
| 1266 | 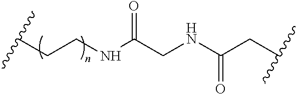 | 5 | 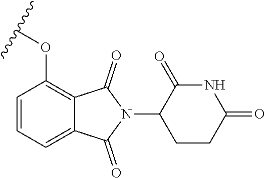 [SMILES: *Oc1cccc2c1C(=O)N(C1CCC(=O)NC1=O)C2=O] |
| 1267 | 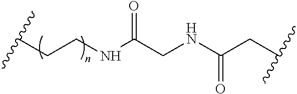 | 1 | 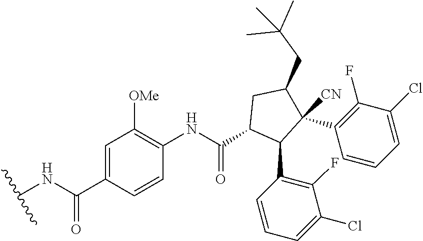 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1268 | 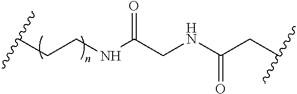 | 2 | 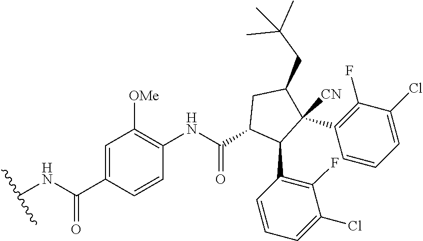 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1269 | 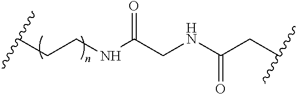 | 3 | 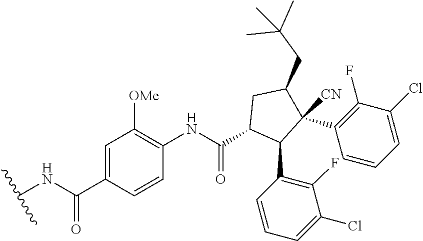 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1270 | 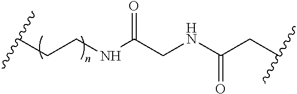 | 4 | 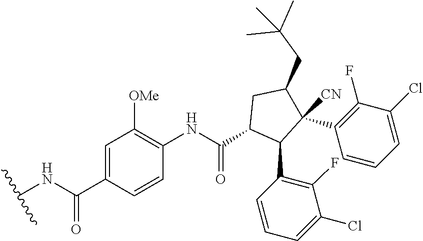 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1271 | 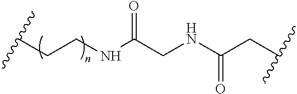 | 5 | 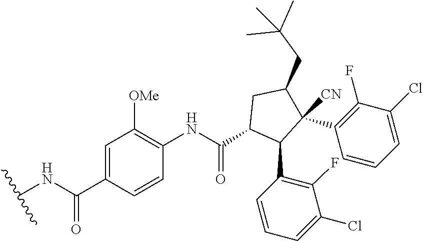 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1272 | 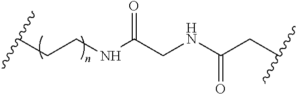 | 1 | 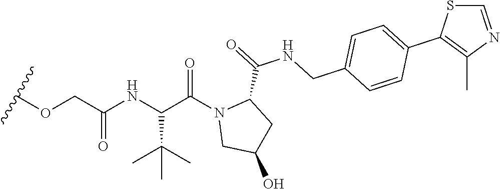 |
| 1273 | 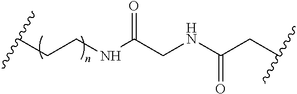 | 2 | 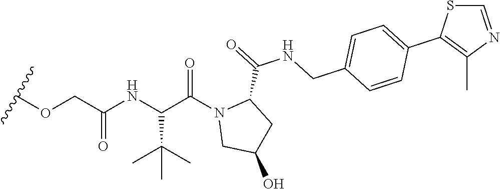 |
| 1274 | 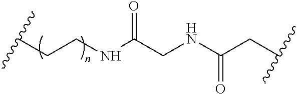 | 3 | 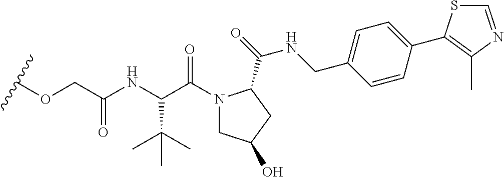 |
| 1275 | 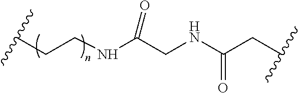 | 4 | 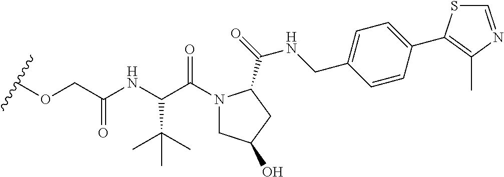 |
| 1276 | 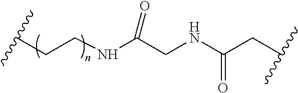 | 5 | 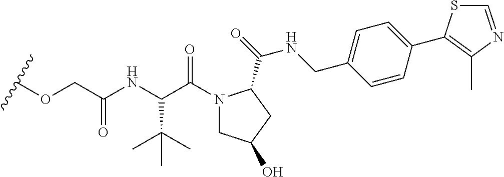 |

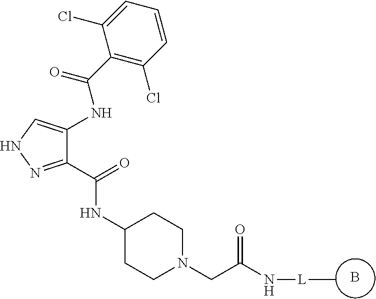

| Formula | | | |
|---|---|---|---|
| No. | L | n | B |
| 1277 | 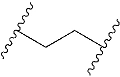 [SMILES: *CC*] | — | 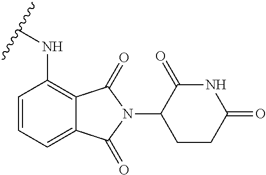 [SMILES: *Nc1cccc2c1C(=O)N(C1CCC(=O)NC1=O)C2=O] |
| 1278 | 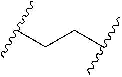 [SMILES: *CC*] | — | 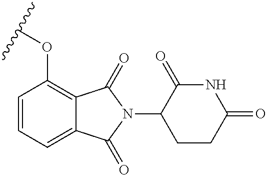 [SMILES: *Oc1cccc2c1C(=O)N(C1CCC(=O)NC1=O)C2=O] |
| 1279 | 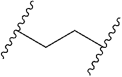 [SMILES: *CC*] | — | 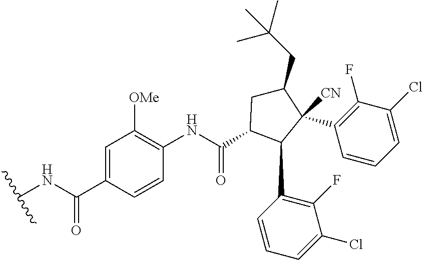 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1280 | 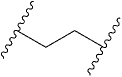 [SMILES: *CC*] | — | 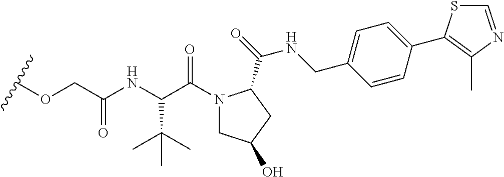 |
| 1281 | 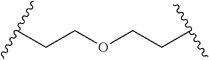 [SMILES: *CCOCC*] | — | 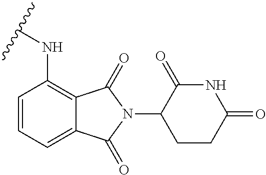 [SMILES: *Nc1cccc2c1C(=O)N(C1CCC(=O)NC1=O)C2=O] |
| 1282 | 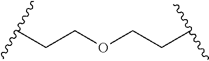 [SMILES: *CCOCC*] | — | 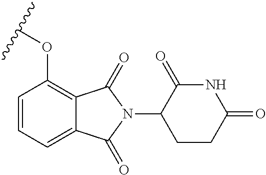 [SMILES: *Oc1cccc2c1C(=O)N(C1CCC(=O)NC1=O)C2=O] |
| 1283 | 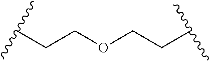 [SMILES: *CCOCC*] | — | 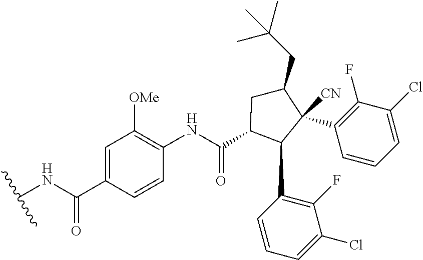 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1284 | 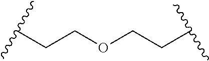 [SMILES: *CCOCC*] | — | 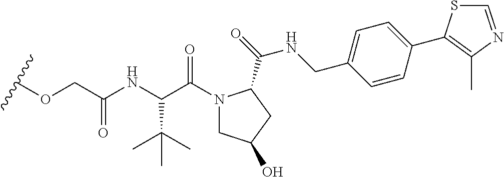 |
| 1285 | 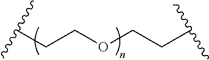 | 1 | 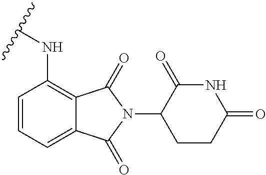 [SMILES: *Nc1cccc2c1C(=O)N(C1CCC(=O)NC1=O)C2=O] |
| 1286 | 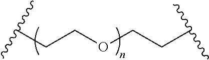 | 2 | 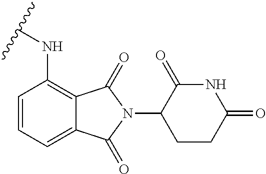 [SMILES: *Nc1cccc2c1C(=O)N(C1CCC(=O)NC1=O)C2=O] |
| 1287 | 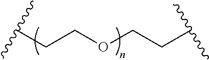 | 3 | 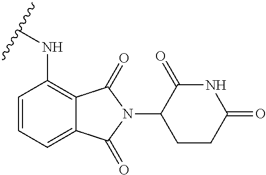 [SMILES: *Nc1cccc2c1C(=O)N(C1CCC(=O)NC1=O)C2=O] |
| 1288 | 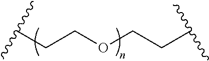 | 4 | 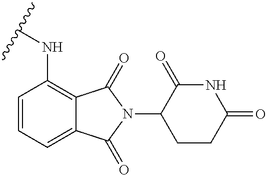 [SMILES: *Nc1cccc2c1C(=O)N(C1CCC(=O)NC1=O)C2=O] |
| 1289 | 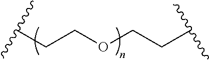 | 5 | 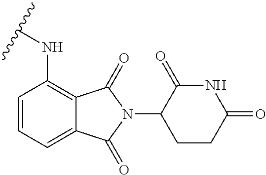 [SMILES: *Nc1cccc2c1C(=O)N(C1CCC(=O)NC1=O)C2=O] |
| 1290 | 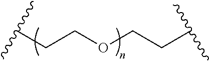 | 1 | 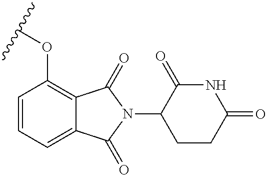 [SMILES: *Oc1cccc2c1C(=O)N(C1CCC(=O)NC1=O)C2=O] |
| 1291 | 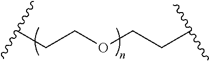 | 2 | 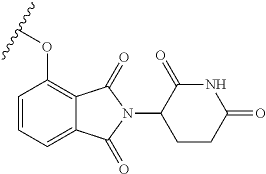 [SMILES: *Oc1cccc2c1C(=O)N(C1CCC(=O)NC1=O)C2=O] |
| 1292 | 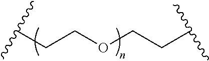 | 3 | 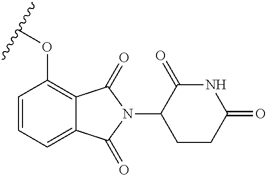 [SMILES: *Oc1cccc2c1C(=O)N(C1CCC(=O)NC1=O)C2=O] |
| 1293 | 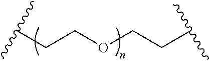 | 4 | 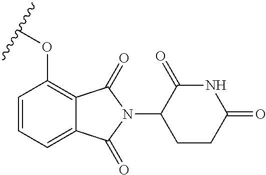 [SMILES: *Oc1cccc2c1C(=O)N(C1CCC(=O)NC1=O)C2=O] |
| 1294 | 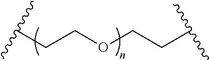 | 5 | 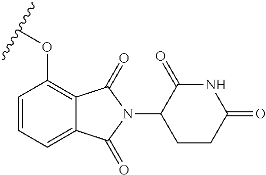 [SMILES: *Oc1cccc2c1C(=O)N(C1CCC(=O)NC1=O)C2=O] |
| 1295 | 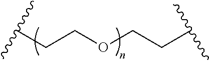 | 1 | 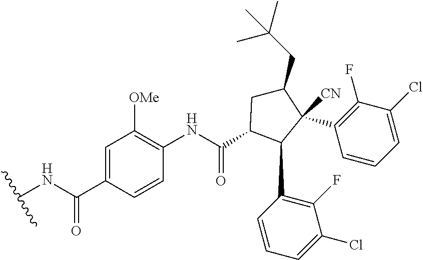 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1296 | 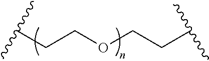 | 2 | 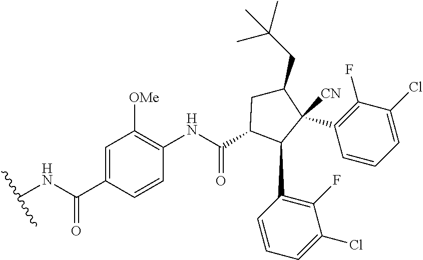 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1297 | 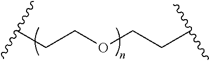 | 3 | 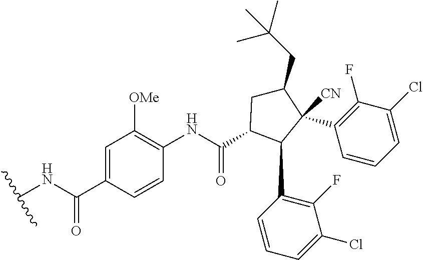 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1298 | 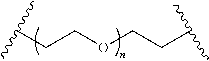 | 4 | 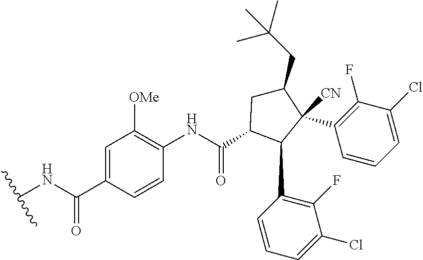 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1299 | 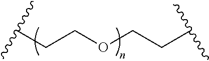 | 5 | 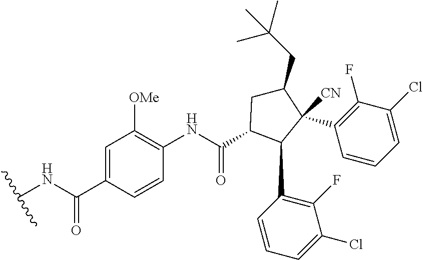 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1300 | 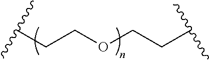 | 1 | 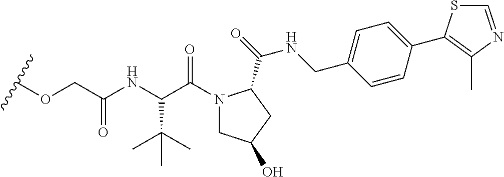 |
| 1301 | 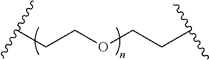 | 2 | 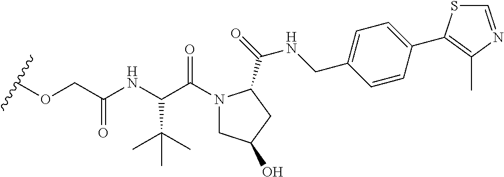 |
| 1302 | 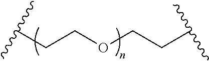 | 3 | 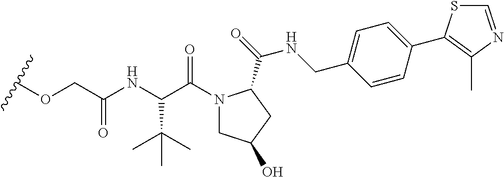 |
| 1303 | 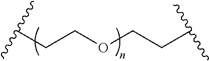 | 4 | 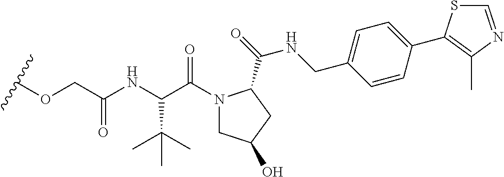 |
| 1304 | 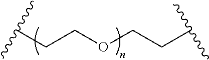 | 5 | 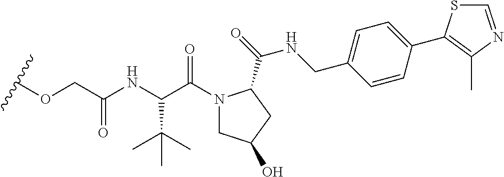 |
| 1305 | 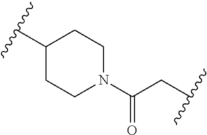 [SMILES: *CC(=O)N1CCC(*)CC1] | — | 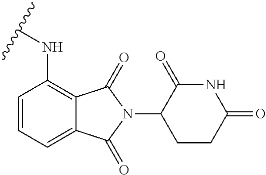 [SMILES: *Nc1cccc2c1C(=O)N(C1CCC(=O)NC1=O)C2=O] |
| 1306 | 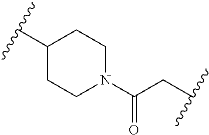 [SMILES: *CC(=O)N1CCC(*)CC1] | — | 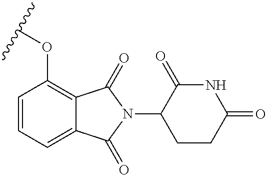 [SMILES: *Oc1cccc2c1C(=O)N(C1CCC(=O)NC1=O)C2=O] |
| 1307 | 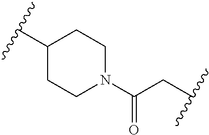 [SMILES: *CC(=O)N1CCC(*)CC1] | — | 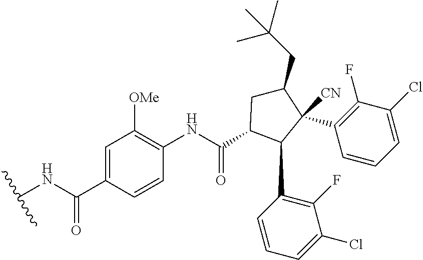 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1308 | 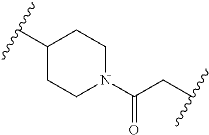 [SMILES: *CC(=O)N1CCC(*)CC1] | — | 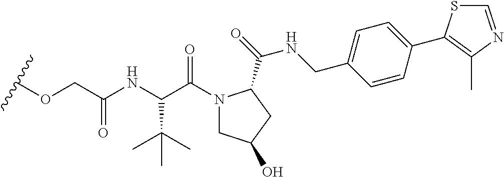 |
| 1309 | 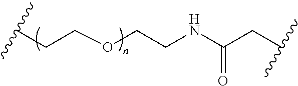 | 1 | 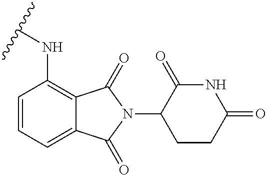 [SMILES: *Nc1cccc2c1C(=O)N(C1CCC(=O)NC1=O)C2=O] |
| 1310 | 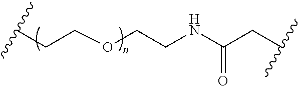 | 2 | 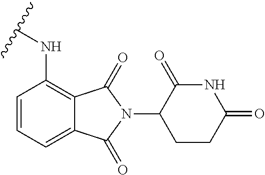 [SMILES: *Nc1cccc2c1C(=O)N(C1CCC(=O)NC1=O)C2=O] |
| 1311 | 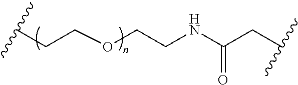 | 3 | 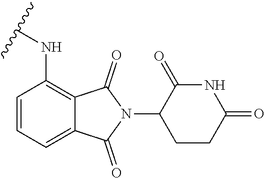 [SMILES: *Nc1cccc2c1C(=O)N(C1CCC(=O)NC1=O)C2=O] |
| 1312 | 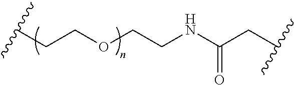 | 4 | 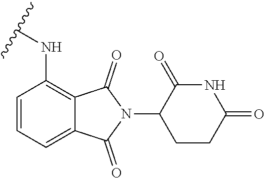 [SMILES: *Nc1cccc2c1C(=O)N(C1CCC(=O)NC1=O)C2=O] |
| 1313 | 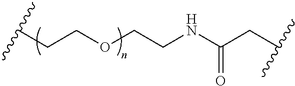 | 5 | 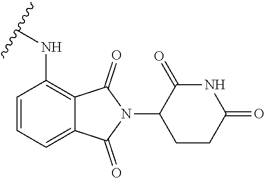 [SMILES: *Nc1cccc2c1C(=O)N(C1CCC(=O)NC1=O)C2=O] |
| 1314 | 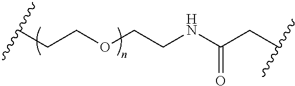 | 1 | 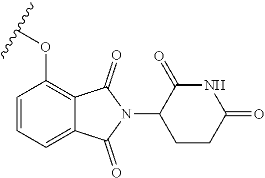 [SMILES: *Oc1cccc2c1C(=O)N(C1CCC(=O)NC1=O)C2=O] |
| 1315 | 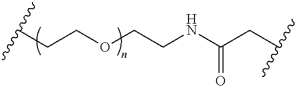 | 2 | 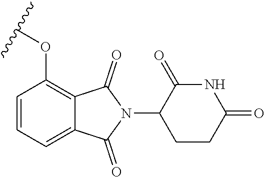 [SMILES: *Oc1cccc2c1C(=O)N(C1CCC(=O)NC1=O)C2=O] |
| 1316 | 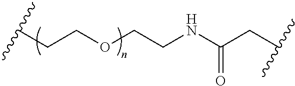 | 3 | 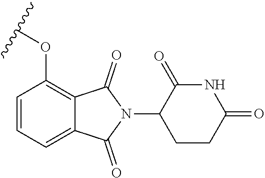 [SMILES: *Oc1cccc2c1C(=O)N(C1CCC(=O)NC1=O)C2=O] |
| 1317 | 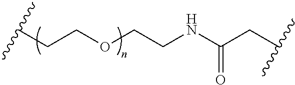 | 4 | 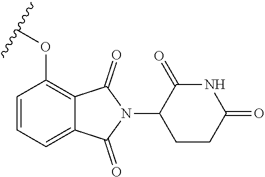 [SMILES: *Oc1cccc2c1C(=O)N(C1CCC(=O)NC1=O)C2=O] |
| 1318 | 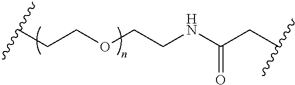 | 5 | 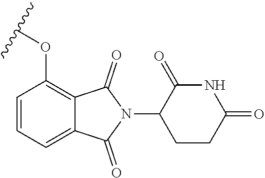 [SMILES: *Oc1cccc2c1C(=O)N(C1CCC(=O)NC1=O)C2=O] |
| 1319 | 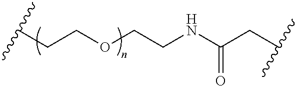 | 1 | 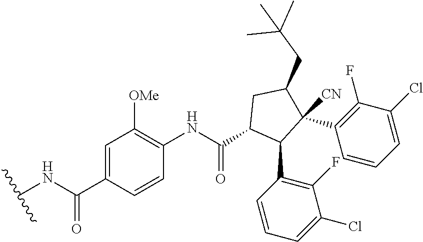 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1320 | 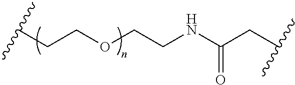 | 2 | 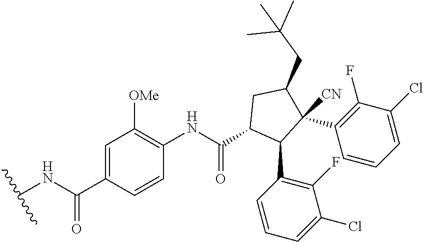 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1321 | 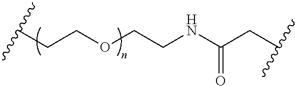 | 3 | 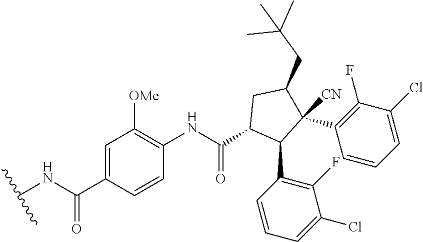 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1322 | 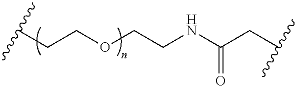 | 4 | 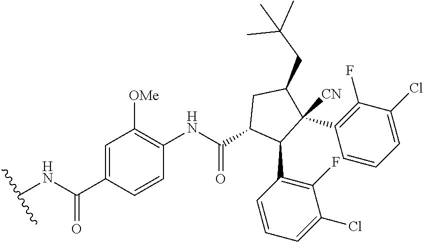 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1323 | 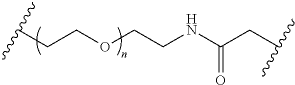 | 5 | 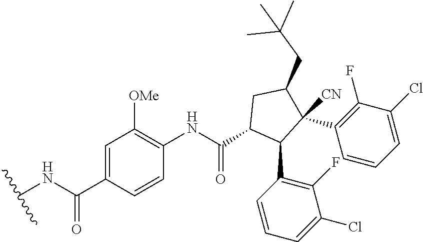 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1324 | 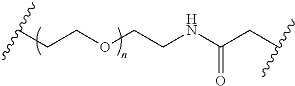 | 1 | 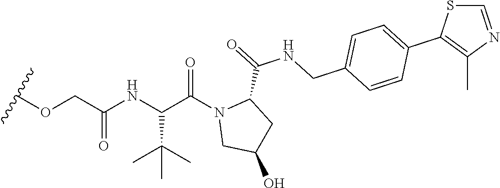 |
| 1325 | 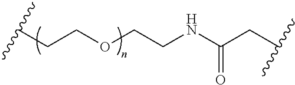 | 2 | 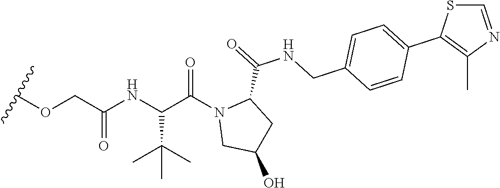 |
| 1326 | 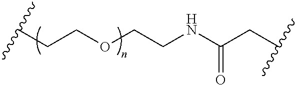 | 3 | 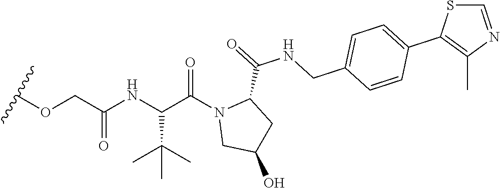 |
| 1327 | 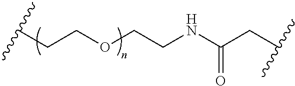 | 4 | 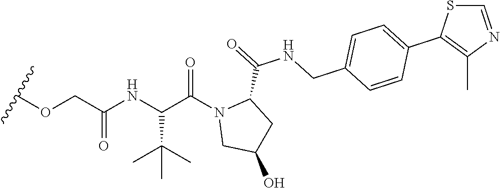 |
| 1328 | 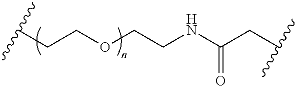 | 5 | 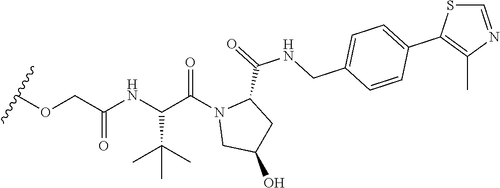 |
| 1329 | 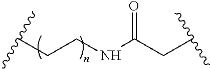 | 1 | 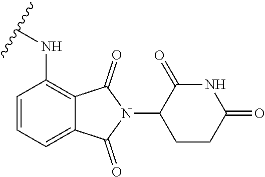 [SMILES: *Nc1cccc2c1C(=O)N(C1CCC(=O)NC1=O)C2=O] |
| 1330 | 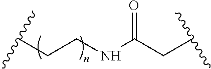 | 2 | 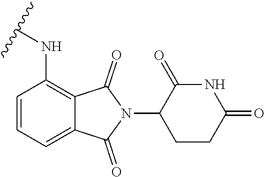 [SMILES: *Nc1cccc2c1C(=O)N(C1CCC(=O)NC1=O)C2=O] |
| 1331 | 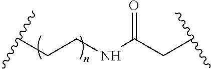 | 3 | 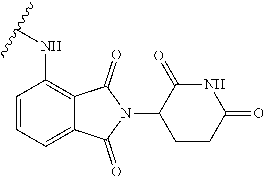 [SMILES: *Nc1cccc2c1C(=O)N(C1CCC(=O)NC1=O)C2=O] |
| 1332 | 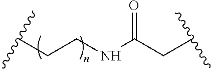 | 4 | 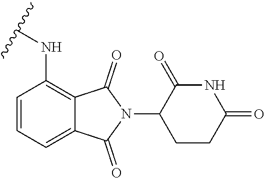 [SMILES: *Nc1cccc2c1C(=O)N(C1CCC(=O)NC1=O)C2=O] |
| 1333 | 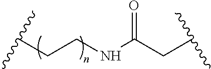 | 5 | 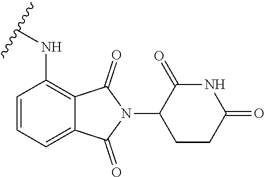 [SMILES: *Nc1cccc2c1C(=O)N(C1CCC(=O)NC1=O)C2=O] |
| 1334 | 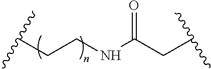 | 1 | 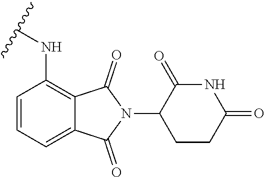 [SMILES: *Nc1cccc2c1C(=O)N(C1CCC(=O)NC1=O)C2=O] |
| 1335 | 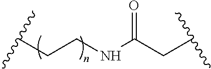 | 2 | 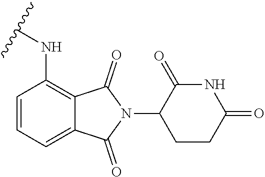 [SMILES: *Nc1cccc2c1C(=O)N(C1CCC(=O)NC1=O)C2=O] |
| 1336 | 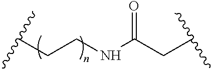 | 3 | 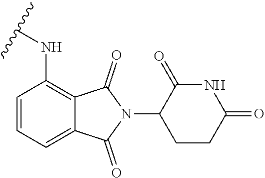 [SMILES: *Nc1cccc2c1C(=O)N(C1CCC(=O)NC1=O)C2=O] |
| 1337 | 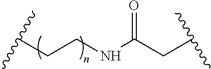 | 4 | 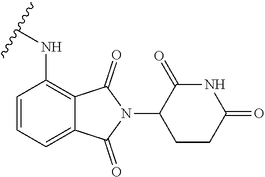 [SMILES: *Nc1cccc2c1C(=O)N(C1CCC(=O)NC1=O)C2=O] |
| 1338 | 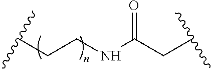 | 5 | 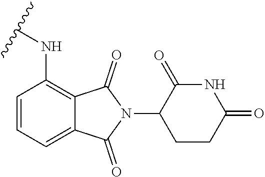 [SMILES: *Nc1cccc2c1C(=O)N(C1CCC(=O)NC1=O)C2=O] |
| 1339 | 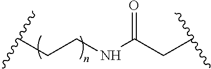 | 1 | 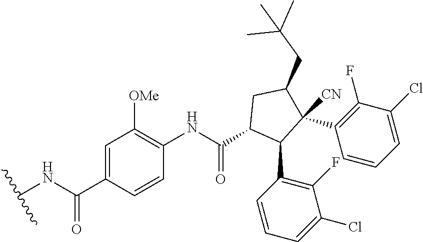 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1340 | 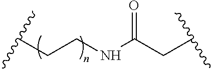 | 2 | 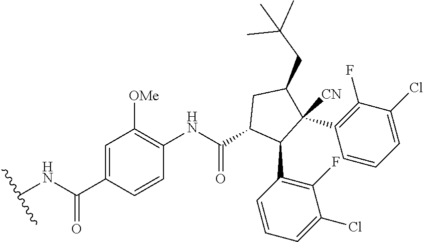 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1341 | 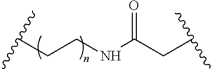 | 3 | 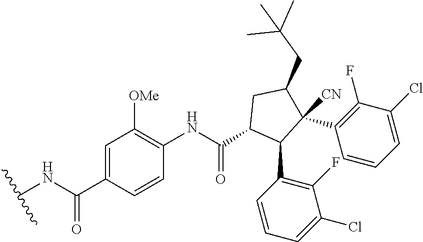 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1342 | 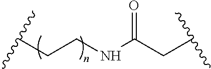 | 4 | 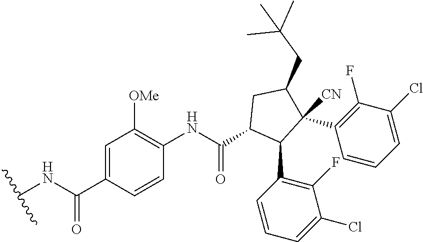 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1343 | 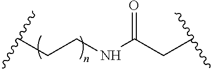 | 5 | 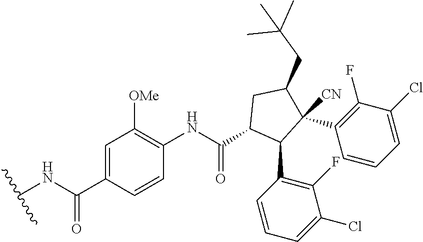 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1344 | 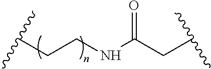 | 1 | 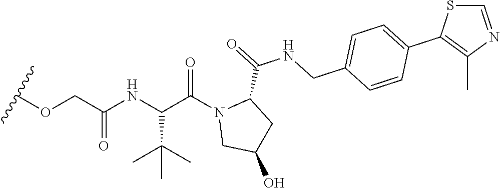 |
| 1345 | 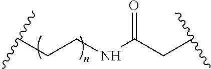 | 2 | 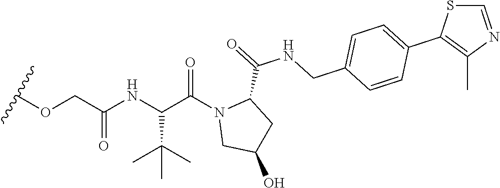 |
| 1346 | 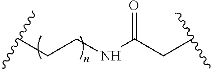 | 3 | 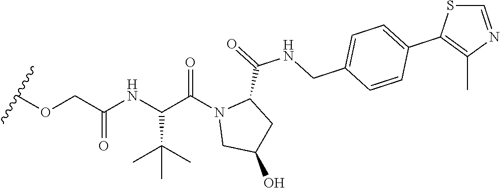 |
| 1347 | 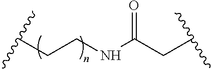 | 4 | 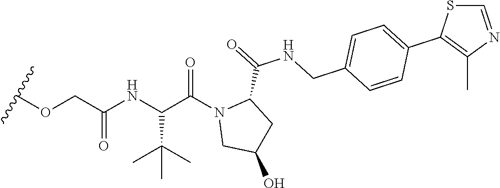 |
| 1348 | 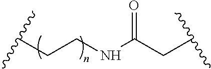 | 5 | 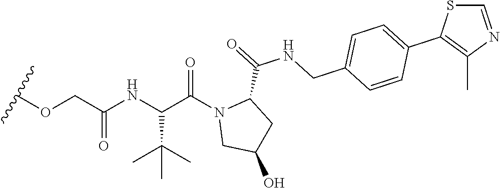 |
| 1349 | 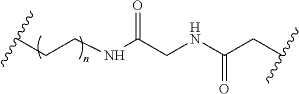 | 1 | 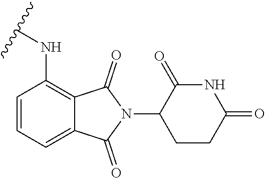 [SMILES: *Nc1cccc2c1C(=O)N(C1CCC(=O)NC1=O)C2=O] |
| 1350 | 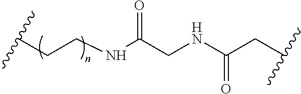 | 2 | 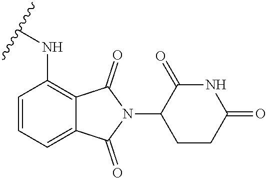 [SMILES: *Nc1cccc2c1C(=O)N(C1CCC(=O)NC1=O)C2=O] |
| 1351 | 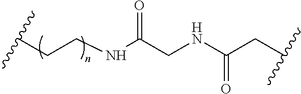 | 3 | 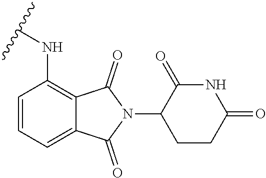 [SMILES: *Nc1cccc2c1C(=O)N(C1CCC(=O)NC1=O)C2=O] |
| 1352 | 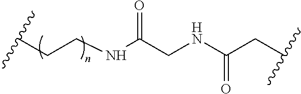 | 4 | 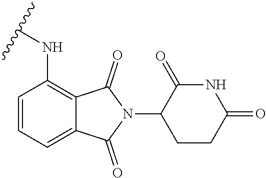 [SMILES: *Nc1cccc2c1C(=O)N(C1CCC(=O)NC1=O)C2=O] |
| 1353 | 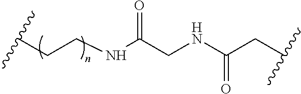 | 5 | 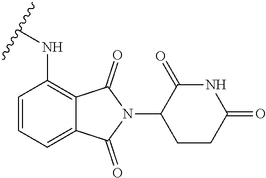 [SMILES: *Nc1cccc2c1C(=O)N(C1CCC(=O)NC1=O)C2=O] |
| 1354 | 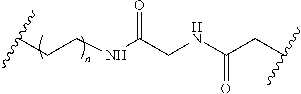 | 1 | 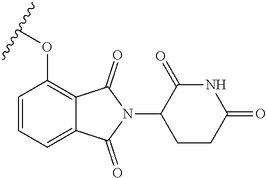 [SMILES: *Oc1cccc2c1C(=O)N(C1CCC(=O)NC1=O)C2=O] |
| 1355 | 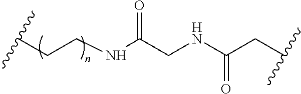 | 2 | 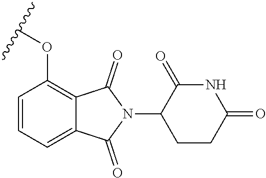 [SMILES: *Oc1cccc2c1C(=O)N(C1CCC(=O)NC1=O)C2=O] |
| 1356 | 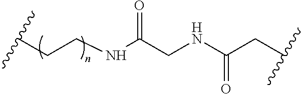 | 3 | 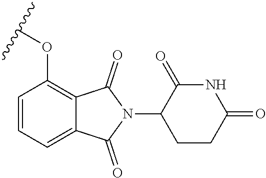 [SMILES: *Oc1cccc2c1C(=O)N(C1CCC(=O)NC1=O)C2=O] |
| 1357 | 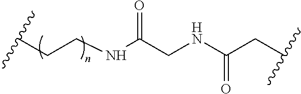 | 4 | 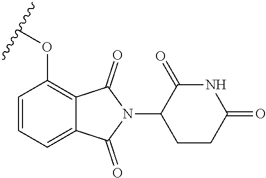 [SMILES: *Oc1cccc2c1C(=O)N(C1CCC(=O)NC1=O)C2=O] |
| 1358 | 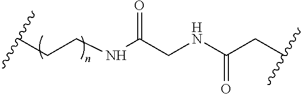 | 5 | 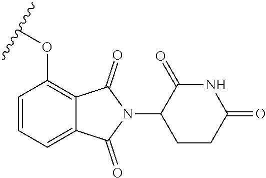 [SMILES: *Oc1cccc2c1C(=O)N(C1CCC(=O)NC1=O)C2=O] |
| 1359 | 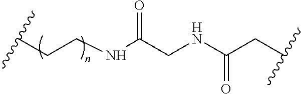 | 1 | 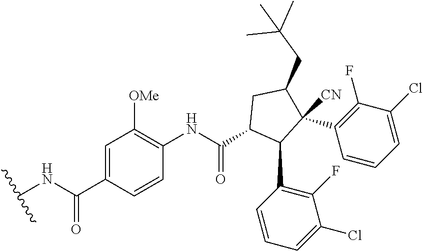 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1360 | 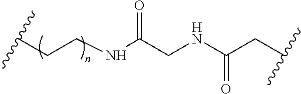 | 2 | 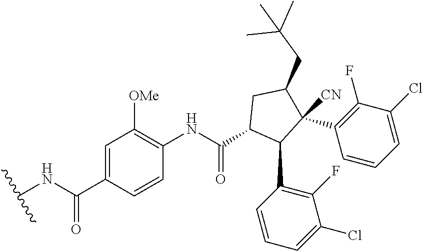 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1361 | 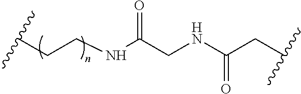 | 3 | 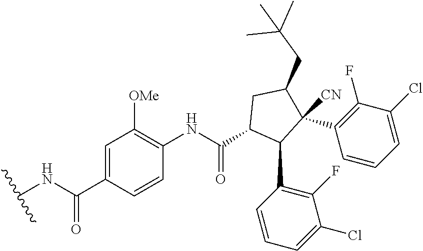 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1362 | 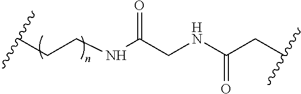 | 4 | 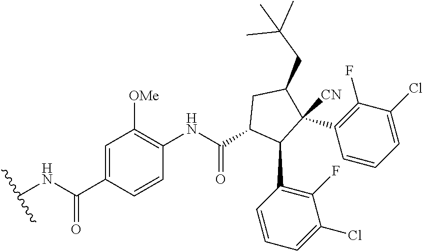 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1363 | 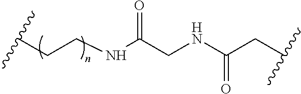 | 5 | 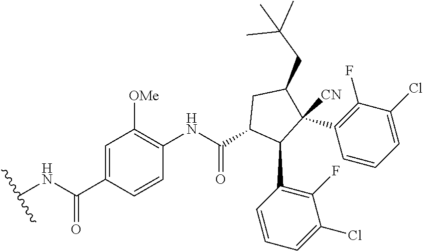 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1364 | 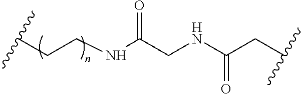 | 1 | 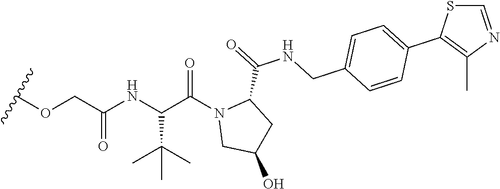 |
| 1365 | 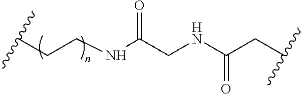 | 2 | 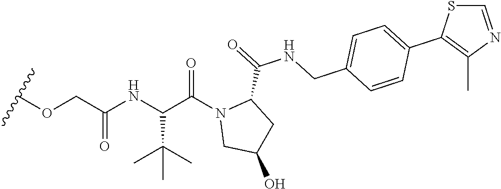 |
| 1366 | 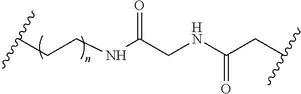 | 3 | 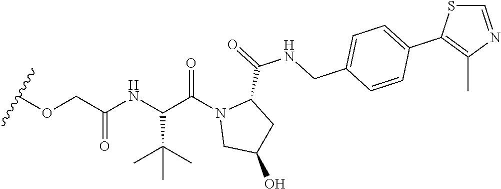 |
| 1367 | 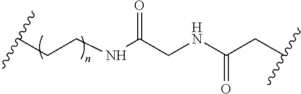 | 4 | 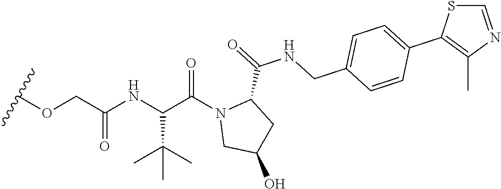 |
| 1368 | 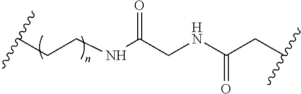 | 5 | 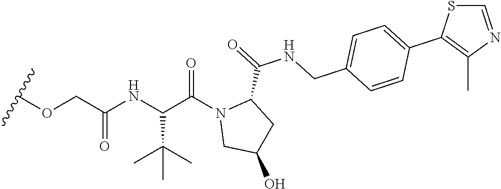 |

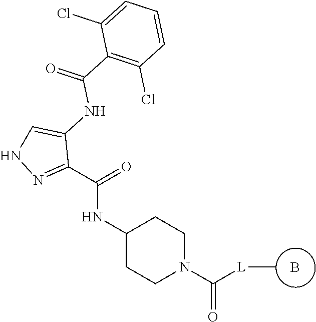

| Formula | | | |
|---|---|---|---|
| No. | L | n | B |
| 1369 | 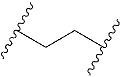 [SMILES: *CC*] | — | 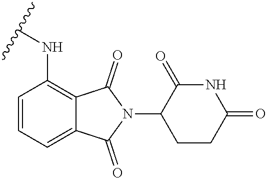 [SMILES: *Nc1cccc2c1C(=O)N(C1CCC(=O)NC1=O)C2=O] |
| 1370 | 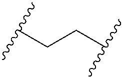 [SMILES: *CC*] | — | 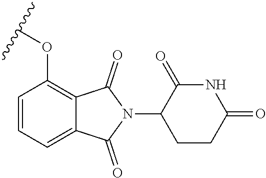 [SMILES: *Oc1cccc2c1C(=O)N(C1CCC(=O)NC1=O)C2=O] |
| 1371 | 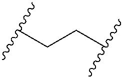 [SMILES: *CC*] | — | 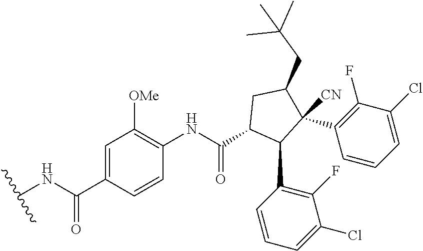 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1372 | 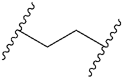 [SMILES: *CC*] | — | 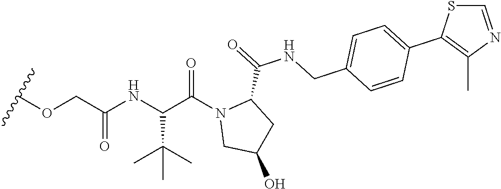 |
| 1373 | 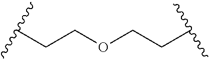 [SMILES: *CCOCC*] | — | 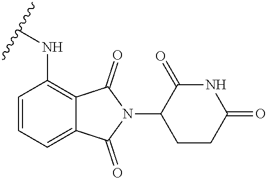 [SMILES: *Nc1cccc2c1C(=O)N(C1CCC(=O)NC1=O)C2=O] |
| 1374 | 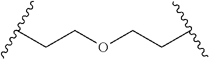 [SMILES: *CCOCC*] | — | 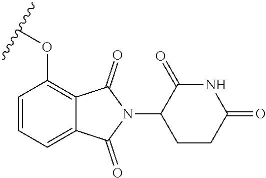 [SMILES: *Oc1cccc2c1C(=O)N(C1CCC(=O)NC1=O)C2=O] |
| 1375 | 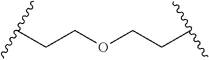 [SMILES: *CCOCC*] | — | 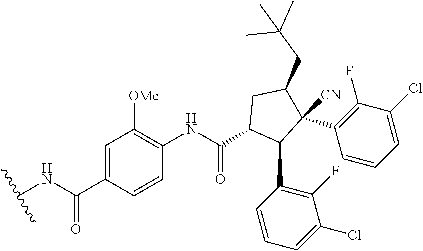 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1376 | 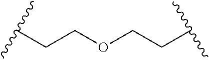 [SMILES: *CCOCC*] | — | 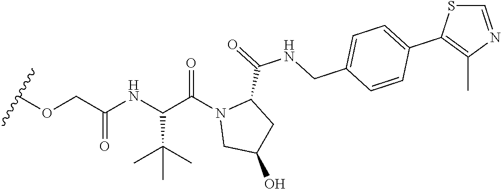 |
| 1377 | 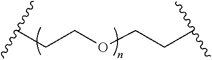 | 1 | 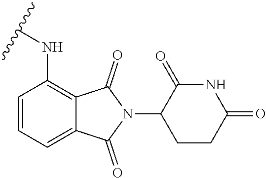 [SMILES: *Nc1cccc2c1C(=O)N(C1CCC(=O)NC1=O)C2=O] |
| 1378 | 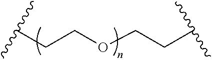 | 2 | 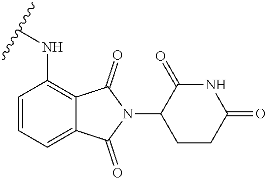 [SMILES: *Nc1cccc2c1C(=O)N(C1CCC(=O)NC1=O)C2=O] |
| 1379 | 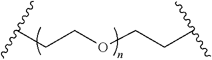 | 3 | 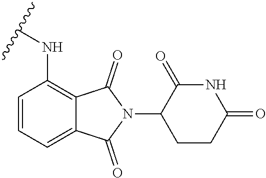 [SMILES: *Nc1cccc2c1C(=O)N(C1CCC(=O)NC1=O)C2=O] |
| 1380 | 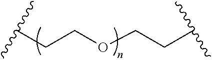 | 4 | 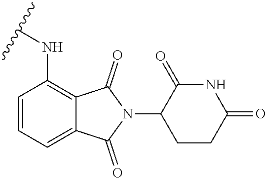 [SMILES: *Nc1cccc2c1C(=O)N(C1CCC(=O)NC1=O)C2=O] |
| 1381 | 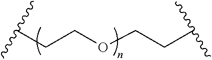 | 5 | 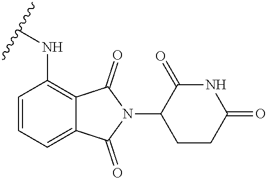 [SMILES: *Nc1cccc2c1C(=O)N(C1CCC(=O)NC1=O)C2=O] |
| 1382 | 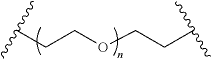 | 1 | 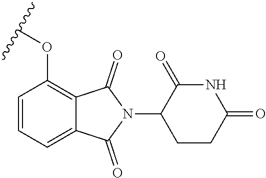 [SMILES: *Oc1cccc2c1C(=O)N(C1CCC(=O)NC1=O)C2=O] |
| 1383 | 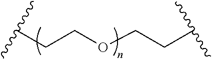 | 2 | 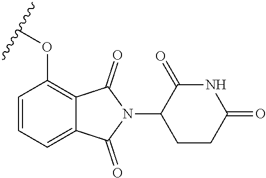 [SMILES: *Oc1cccc2c1C(=O)N(C1CCC(=O)NC1=O)C2=O] |
| 1384 | 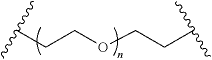 | 3 | 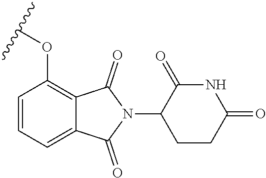 [SMILES: *Oc1cccc2c1C(=O)N(C1CCC(=O)NC1=O)C2=O] |
| 1385 | 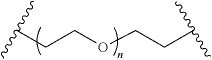 | 4 | 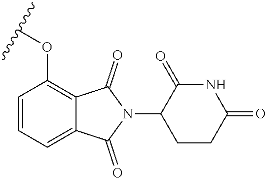 [SMILES: *Oc1cccc2c1C(=O)N(C1CCC(=O)NC1=O)C2=O] |
| 1386 | 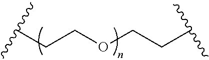 | 5 | 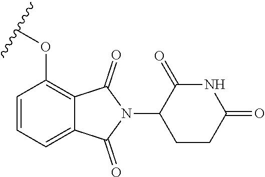 [SMILES: *Oc1cccc2c1C(=O)N(C1CCC(=O)NC1=O)C2=O] |
| 1387 | 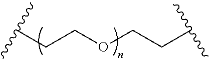 | 1 | 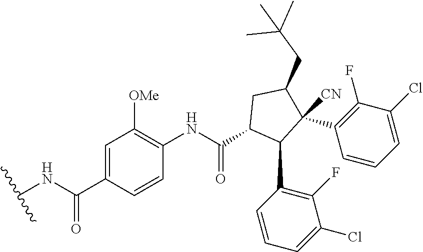 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1388 | 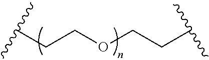 | 2 | 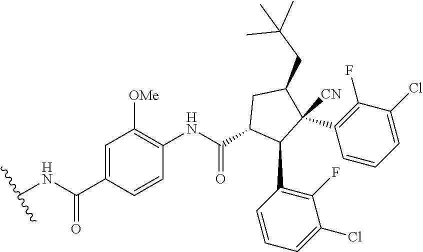 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1389 | 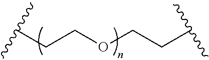 | 3 | 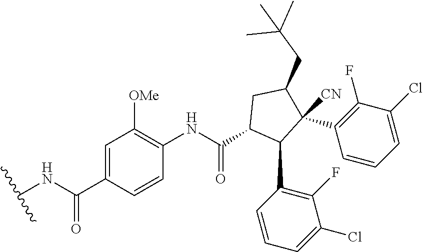 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1390 | 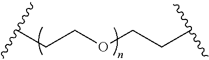 | 4 | 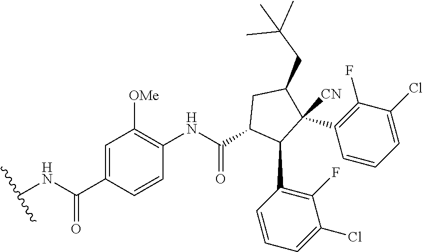 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1391 | 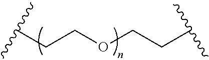 | 5 | 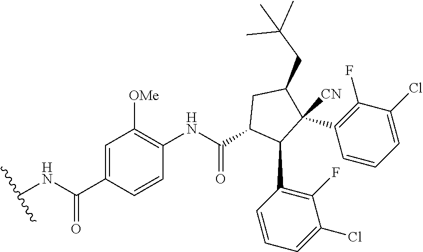 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1392 | 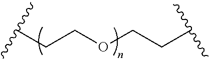 | 1 | 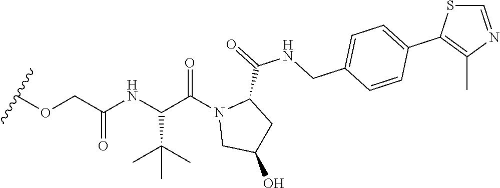 |
| 1393 | 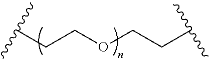 | 2 | 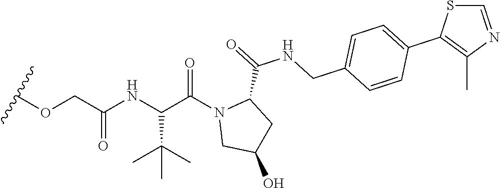 |
| 1394 | 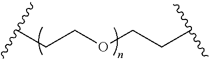 | 3 | 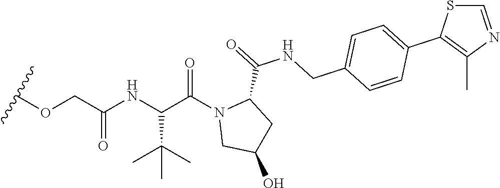 |
| 1395 | 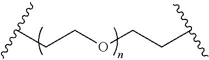 | 4 | 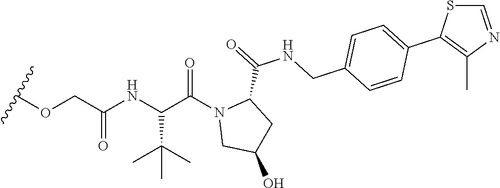 |
| 1396 | 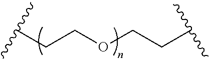 | 5 | 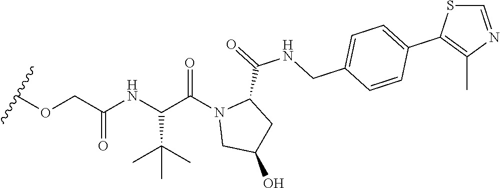 |
| 1397 | 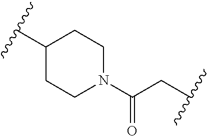 [SMILES: *CC(=O)N1CCC(*)CC1] | — | 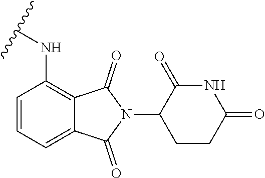 [SMILES: *Nc1cccc2c1C(=O)N(C1CCC(=O)NC1=O)C2=O] |
| 1398 | 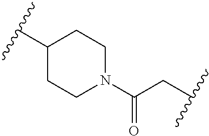 [SMILES: *CC(=O)N1CCC(*)CC1] | — | 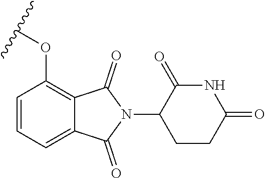 [SMILES: *Oc1cccc2c1C(=O)N(C1CCC(=O)NC1=O)C2=O] |
| 1399 | 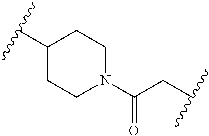 [SMILES: *CC(=O)N1CCC(*)CC1] | — | 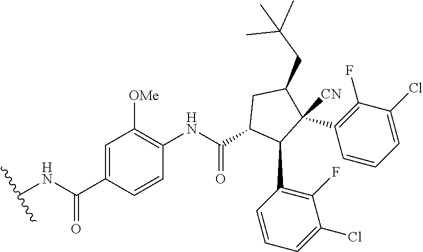 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1400 | 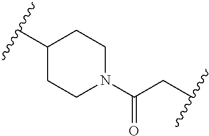 [SMILES: *CC(=O)N1CCC(*)CC1] | — | 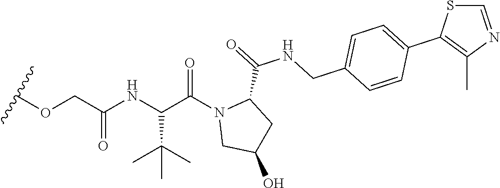 |
| 1401 | 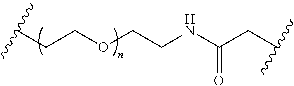 | 1 | 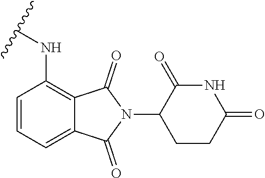 [SMILES: *Nc1cccc2c1C(=O)N(C1CCC(=O)NC1=O)C2=O] |
| 1402 | 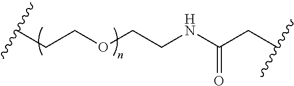 | 2 | 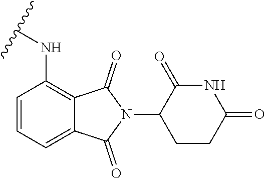 [SMILES: *Nc1cccc2c1C(=O)N(C1CCC(=O)NC1=O)C2=O] |
| 1403 | 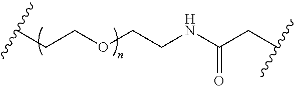 | 3 | 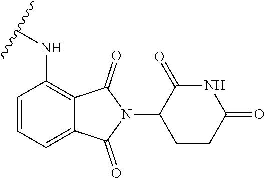 [SMILES: *Nc1cccc2c1C(=O)N(C1CCC(=O)NC1=O)C2=O] |
| 1404 | 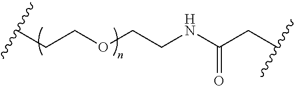 | 4 | 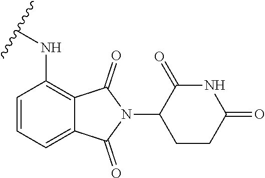 [SMILES: *Nc1cccc2c1C(=O)N(C1CCC(=O)NC1=O)C2=O] |
| 1405 | 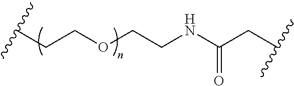 | 5 | 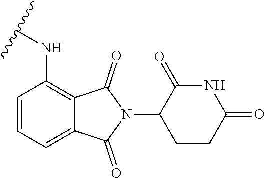 [SMILES: *Nc1cccc2c1C(=O)N(C1CCC(=O)NC1=O)C2=O] |
| 1406 | 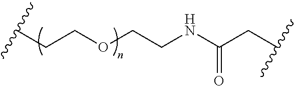 | 1 | 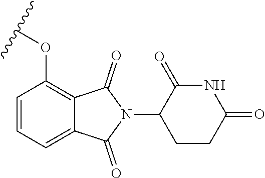 [SMILES: *Oc1cccc2c1C(=O)N(C1CCC(=O)NC1=O)C2=O] |
| 1407 | 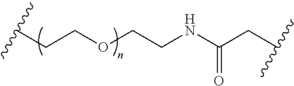 | 2 | 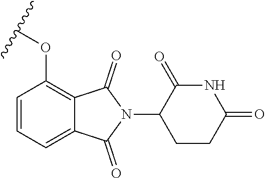 [SMILES: *Oc1cccc2c1C(=O)N(C1CCC(=O)NC1=O)C2=O] |
| 1408 | 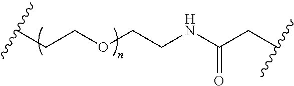 | 3 | 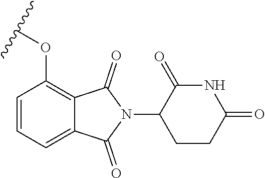 [SMILES: *Oc1cccc2c1C(=O)N(C1CCC(=O)NC1=O)C2=O] |
| 1409 | 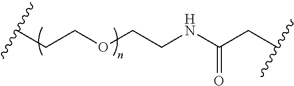 | 4 | 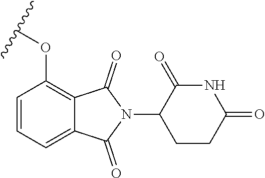 [SMILES: *Oc1cccc2c1C(=O)N(C1CCC(=O)NC1=O)C2=O] |
| 1410 | 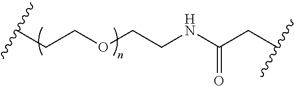 | 5 | 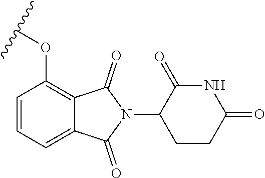 [SMILES: *Oc1cccc2c1C(=O)N(C1CCC(=O)NC1=O)C2=O] |
| 1411 | 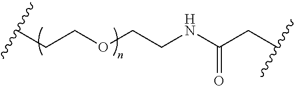 | 1 | 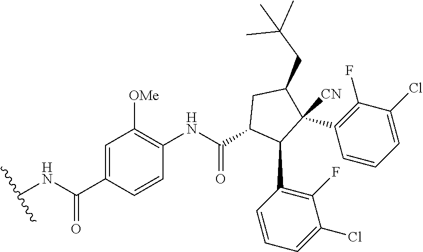 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1412 | 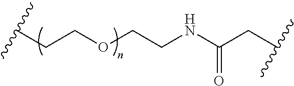 | 2 | 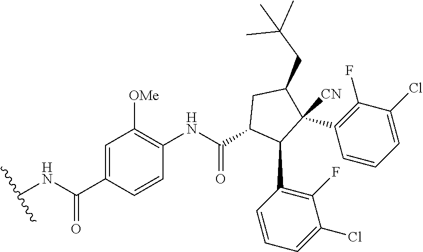 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1413 | 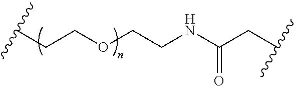 | 3 | 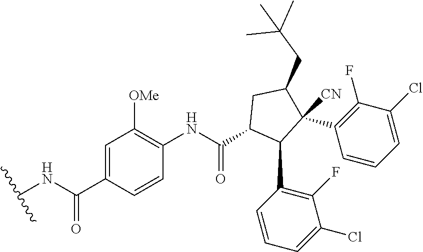 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1414 | 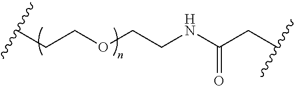 | 4 | 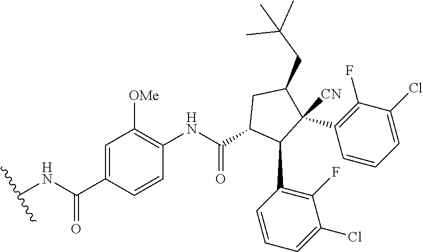 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1415 | 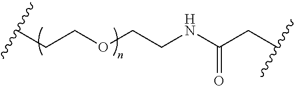 | 5 | 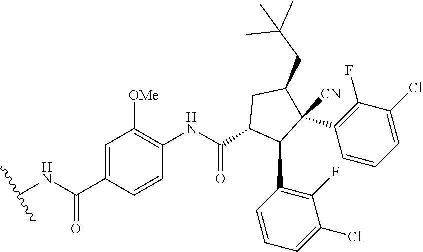 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1416 | 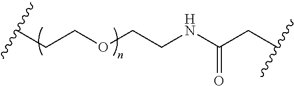 | 1 | 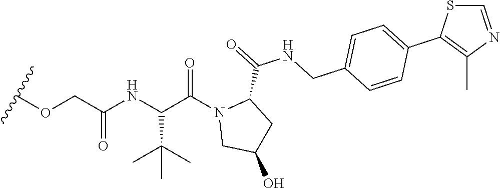 |
| 1417 | 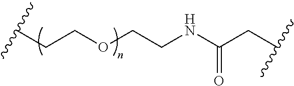 | 2 | 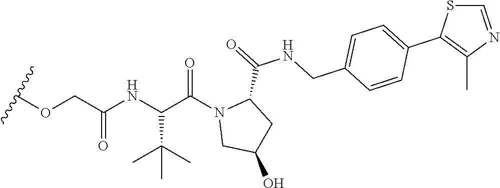 |
| 1418 | 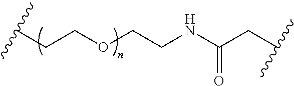 | 3 | 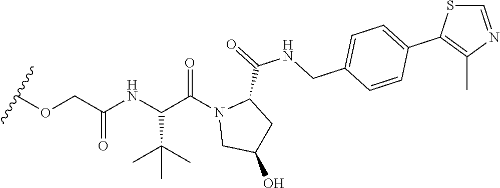 |
| 1419 | 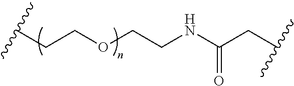 | 4 | 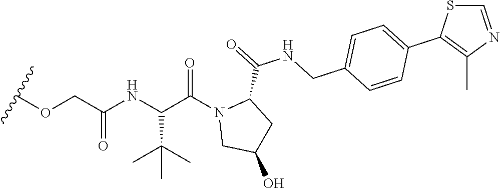 |
| 1420 | 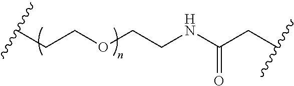 | 5 | 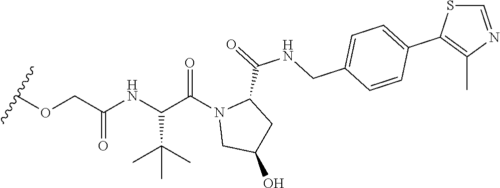 |
| 1421 | 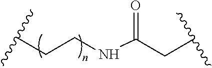 | 1 | 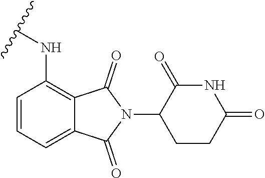 [SMILES: *Nc1cccc2c1C(=O)N(C1CCC(=O)NC1=O)C2=O] |
| 1422 | 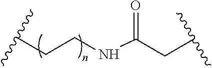 | 2 | 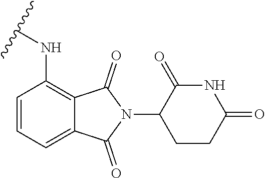 [SMILES: *Nc1cccc2c1C(=O)N(C1CCC(=O)NC1=O)C2=O] |
| 1423 | 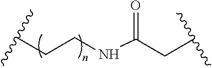 | 3 | 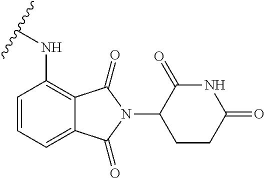 [SMILES: *Nc1cccc2c1C(=O)N(C1CCC(=O)NC1=O)C2=O] |
| 1424 | 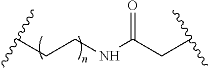 | 4 | 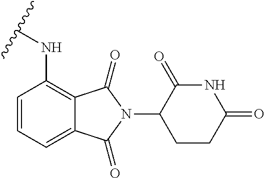 [SMILES: *Nc1cccc2c1C(=O)N(C1CCC(=O)NC1=O)C2=O] |
| 1425 | 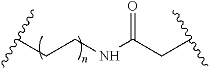 | 5 | 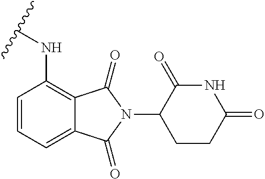 [SMILES: *Nc1cccc2c1C(=O)N(C1CCC(=O)NC1=O)C2=O] |
| 1426 | 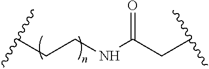 | 1 | 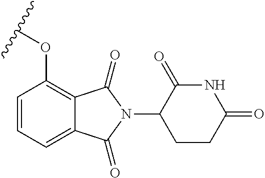 [SMILES: *Oc1cccc2c1C(=O)N(C1CCC(=O)NC1=O)C2=O] |
| 1427 | 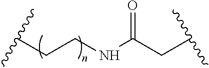 | 2 | 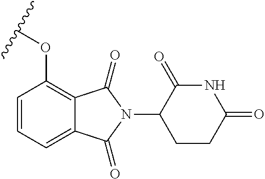 [SMILES: *Oc1cccc2c1C(=O)N(C1CCC(=O)NC1=O)C2=O] |
| 1428 | 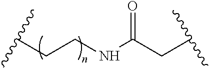 | 3 | 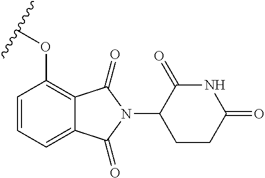 [SMILES: *Oc1cccc2c1C(=O)N(C1CCC(=O)NC1=O)C2=O] |
| 1429 | 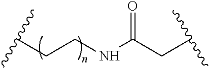 | 4 | 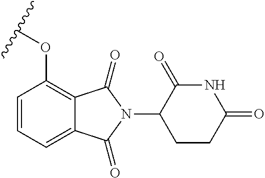 [SMILES: *Oc1cccc2c1C(=O)N(C1CCC(=O)NC1=O)C2=O] |
| 1430 | 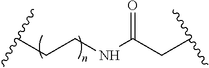 | 5 | 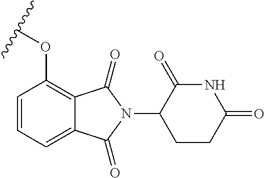 [SMILES: *Oc1cccc2c1C(=O)N(C1CCC(=O)NC1=O)C2=O] |
| 1431 | 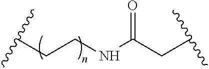 | 1 | 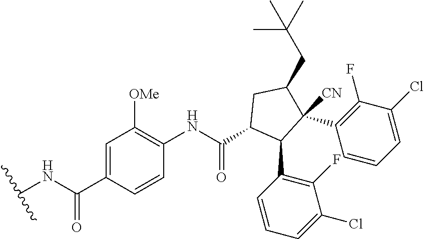 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1432 | 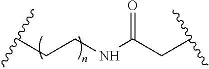 | 2 | 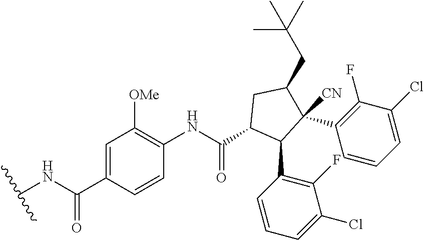 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1433 | 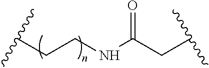 | 3 | 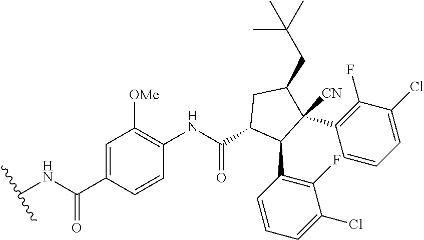 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1434 | 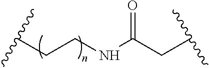 | 4 | 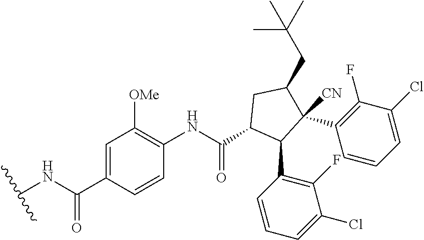 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1435 | 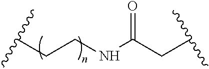 | 5 | 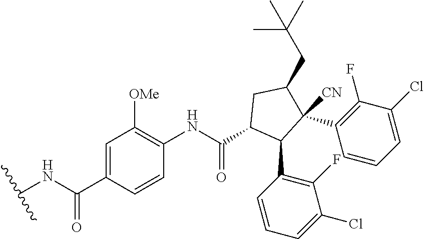 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1436 | 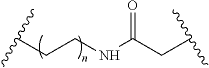 | 1 | 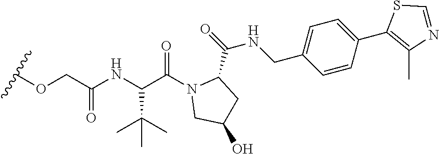 |
| 1437 | 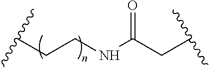 | 2 | 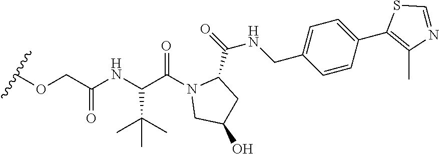 |
| 1438 | 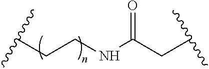 | 3 | 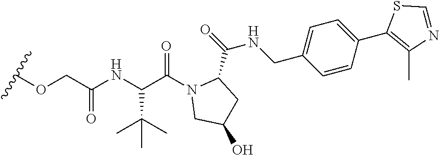 |
| 1439 | 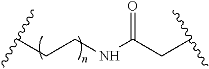 | 4 | 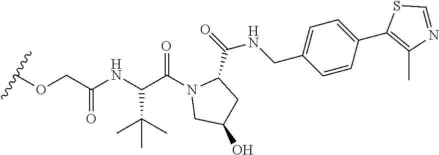 |
| 1440 | 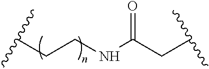 | 5 | 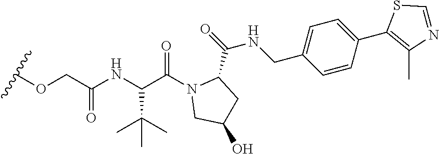 |
| 1441 | 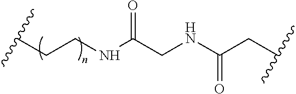 | 1 | 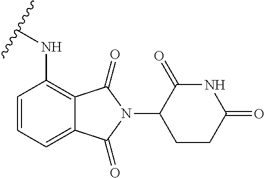 [SMILES: *Nc1cccc2c1C(=O)N(C1CCC(=O)NC1=O)C2=O] |
| 1442 | 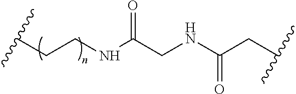 | 2 | 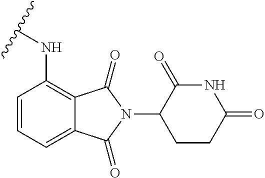 [SMILES: *Nc1cccc2c1C(=O)N(C1CCC(=O)NC1=O)C2=O] |
| 1443 | 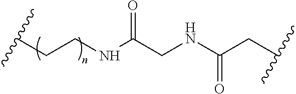 | 3 | 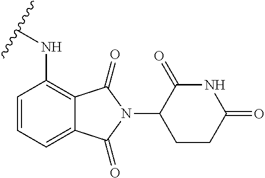 [SMILES: *Nc1cccc2c1C(=O)N(C1CCC(=O)NC1=O)C2=O] |
| 1444 | 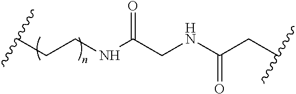 | 4 | 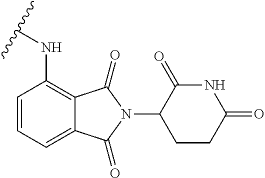 [SMILES: *Nc1cccc2c1C(=O)N(C1CCC(=O)NC1=O)C2=O] |
| 1445 | 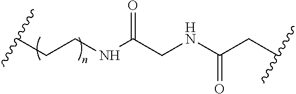 | 5 | 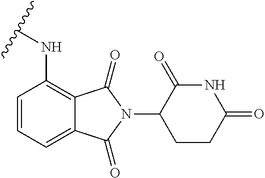 [SMILES: *Nc1cccc2c1C(=O)N(C1CCC(=O)NC1=O)C2=O] |
| 1446 | 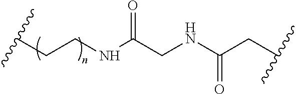 | 1 | 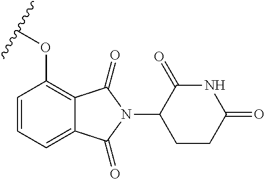 [SMILES: *Oc1cccc2c1C(=O)N(C1CCC(=O)NC1=O)C2=O] |
| 1447 | 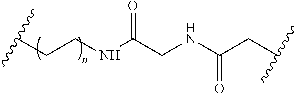 | 2 | 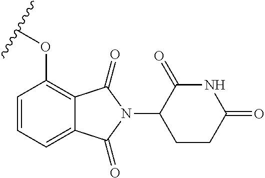 [SMILES: *Oc1cccc2c1C(=O)N(C1CCC(=O)NC1=O)C2=O] |
| 1448 | 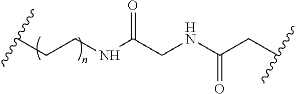 | 3 | 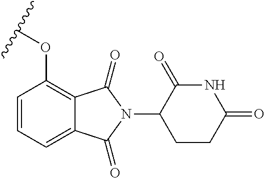 [SMILES: *Oc1cccc2c1C(=O)N(C1CCC(=O)NC1=O)C2=O] |
| 1449 | 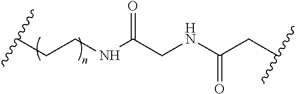 | 4 | 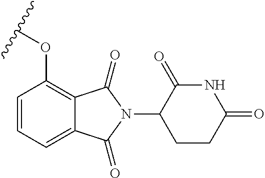 [SMILES: *Oc1cccc2c1C(=O)N(C1CCC(=O)NC1=O)C2=O] |
| 1450 | 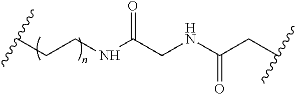 | 5 | 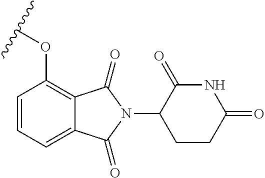 [SMILES: *Oc1cccc2c1C(=O)N(C1CCC(=O)NC1=O)C2=O] |
| 1451 | 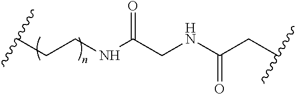 | 1 | 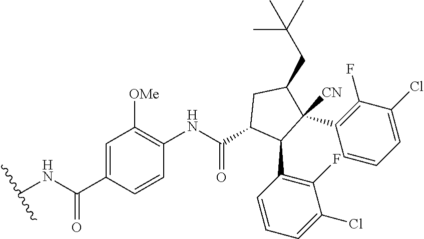 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1452 | 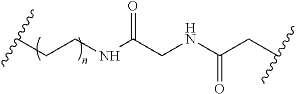 | 2 | 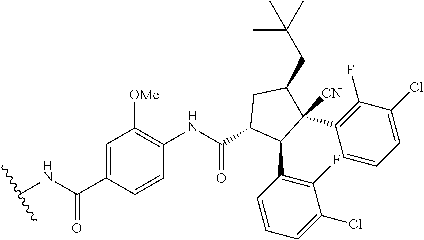 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1453 | 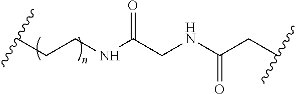 | 3 | 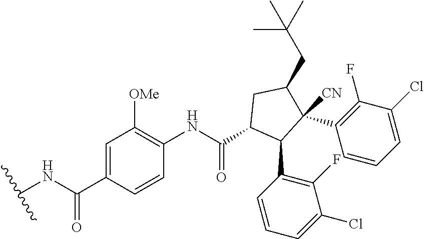 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1454 | 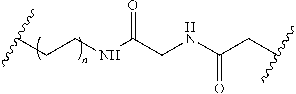 | 4 | 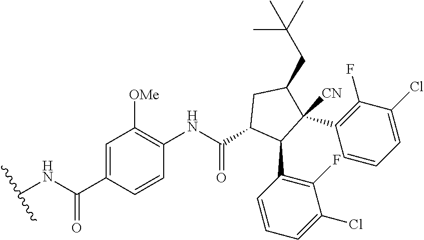 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1455 | 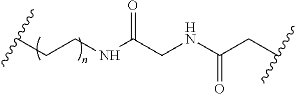 | 5 | 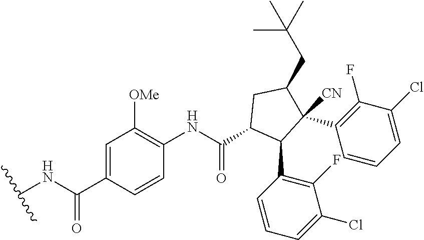 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1456 | 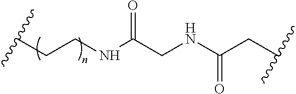 | 1 | 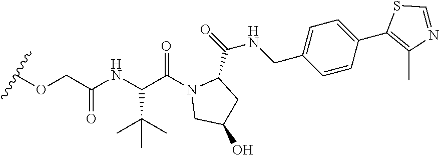 |
| 1457 | 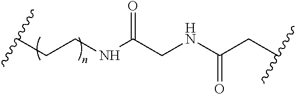 | 2 | 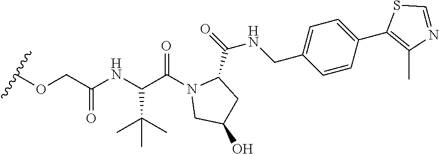 |
| 1458 | 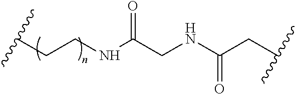 | 3 | 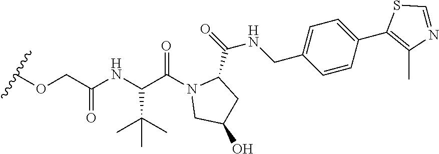 |
| 1459 | 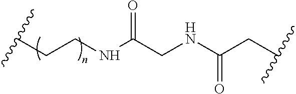 | 4 | 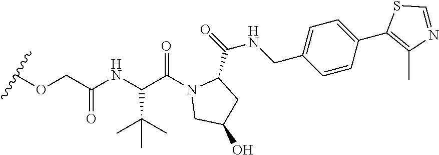 |
| 1460 | 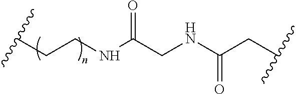 | 5 | 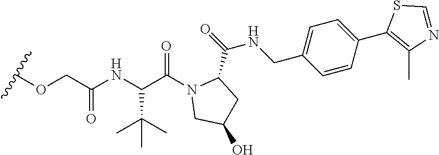 |

Clause 26. A pharmaceutical composition comprising one or more compounds of any one of clauses 1-25 or a pharmaceutically acceptable salt thereof, and a physiologically compatible carrier medium.

Clause 27. A pharmaceutical composition for treating or preventing a disease or disorder alleviated by inhibiting and/or indirectly inhibiting Mcl-1 protein activity, the pharmaceutical composition comprising one or more compounds according to any one of clauses 1-25, or a pharmaceutically acceptable salt thereof, and a physiologically compatible carrier medium.

Clause 28. The pharmaceutical composition of clause 27, wherein the disease or disorder is cancer.

Clause 29. The pharmaceutical composition of clause 28, wherein the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

Clause 30. The pharmaceutical composition of clause 28, wherein the cancer is a blood cancer.

Clause 31. The pharmaceutical composition of clause 30, wherein the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

Clause 32. The pharmaceutical composition of any one of clauses 28-31, wherein the cancer is acute myeloid leukemia (AML).

Clause 33. A pharmaceutical composition for treating or preventing acute myeloid leukemia (AML), the pharmaceutical composition comprising one or more compounds according to any one of clauses 1-25, or a pharmaceutically acceptable salt thereof, and a physiologically compatible carrier medium.

Clause 34. A method of treating or preventing a disease or disorder alleviated by inhibiting and/or indirectly inhibiting Mcl-1 protein activity in a patient in need of said treatment or prevention, the method comprising administering a therapeutically effective amount of one or more compounds of any one of clauses 1-25, or a pharmaceutically acceptable salt thereof.

Clause 35. The method of clause 34, wherein the disease or disorder is cancer.

Clause 36. The method of clause 35, wherein the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

Clause 37. The method of clause 35, wherein the cancer is a blood cancer.

Clause 38. The method of clause 37, wherein the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

Clause 39. The method of any one of clauses 35-38, wherein the cancer is acute myeloid leukemia (AML).

Clause 40. A method of treating or preventing acute myeloid leukemia (AML) in a patient in need of said treatment or prevention, the method comprising administering a therapeutically effective amount of one or more compounds of any one of clauses 1-25, or a pharmaceutically acceptable salt thereof.

The following examples describe the disclosure in further detail. These examples are provided for illustrative purposes only, and should in no way be considered as limiting the disclosure.

### EXAMPLES

### Example 1: PROTAC Compounds for Targeting MCL-1

This Example describes proteolysis targeting chimeras (PROTACs) useful for indirectly inhibiting MCL-1. This Example describes a novel strategy to inhitib MCL-1, which may circumvent the problems associated wih direct inhibition of MCL-1. There are multiple direct inhibitors of MCL-1 in clinical trials, although two have been suspended due to cardiotoxicity and one fatality.

Downregulation of MCL-1 with drug candidates that synergize with VEN. The present strategy of developing direct/competitive, sub-nanomolar MCL-1 inhibitors may never yield an approved drug, for the reasons outlined above. It is hypothesized that indirectly targeting MCL-1, using drugs proven to bind other targets in AML and as a consequence lead to downregulation of MCL-1, will reduce vulnerability to resistance anticipated with direct MCL-1 inhibitors. The conversion of such drugs into PROTACs is anticipated to confer sustained antileukemic activity from not only mechanisms attributed to the drug's direct target itself but also indirectly through MCL-1 downregulation. Lastly, since cancers require combination therapies for effective treatment, and since VEN is a highly desirable drug for antineoplastic combinations, VEN are combined with AML drugs that 1) downregulate MCL-1 indirectly and 2) have demonstrated synergy with VEN.

Since MCL-1 is a required component of the general cellular apoptosis machinery, its direct and complete inhibition may never prove safe. Competitive inhibition of MCL-1 activity is further challenged by the apparent requirement for exquisite affinity in vitro to realize efficacy in vivo, which predisposes to drug resistance. Therefore, in an innovative strategy to inhibit MCL-1, it is hypothesized that PROTACs that downregulate MCL-1 indirectly (hereafter, “indirect MCL-1 PROTACs”) may bypass some of the negative aspects of direct MCL-1 inhibitors. Since MCL-1 upregulation is a major mechanism of resistance to VEN, these new indirect MCL-1 PROTACs are evaluated alone and in combination with VEN.

Indirect MCL-1 PROTACs: As described herein, PROTACs are developed targeting proteins that are involved in the regulation of MCL-1 expression. No such “indirect MCL-1 PROTACs” purposefully designed to downregulate MCL-1 are known.

MCL-1 downregulation is sought via three orthogonal regulatory levels: The CDK9 inhibitor AT7519, the dual PI3K/mTOR inhibitor gedatolisib, and the MEK1 inhibitor PD334581 are converted into PROTACs to interfere with MCL-1 transcription, translation, and post-translation, respectively. Significantly, inhibitors of these protein targets have demonstrated synergy with VEN. The above-designated inhibitors were selected by considering multiple criteria, including: 1) activity in AML clinical trials or advanced preclinical studies, 2) relatively low molecular weight (MW), 3) a solvent-exposed suitable grafting point, 4) synergism with VEN and 5) not been previously converted into a PROTAC.

Use of 3 different E3 ligase ligands: To more fully explore the effects of indirect MCL-1 PROTACs, three different E3 ligase ligands with chemistry attractive for ligation are utilized -thalidomide (CRBN ligand), idasanutlin (MDM2 ligand) and the VHL1 peptide (von Hippel-Lindau ligand).

Combination of an indirect MCL-1 PROTAC with VEN in AML: Since upregulation of MCL-1 is a major mechanism of resistance to VEN, combinations of indirect MCL-1 PROTACs with VEN are explored.

Indirect MCL-1 PROTACs are also evaluated in state-of-the-art AML models including xenografts.

Design and Synthesize PROTACs that Downregulate MCL-1 Indirectly

Of >100 human E3 ligases identified, only three have been utilized in PROTACs: cereblon (CRBN), murine double minute 2 (MDM2) and von Hippel-Lindau (VHL). Each of these can be selectively targeted with thalidomide, idasanutlin and VHL ligand 1, respectively. While there has been concern that PROTACs exhibit large MWs, there is now strong evidence that this is not a major problem. For example, Arvinas's lead PROTAC ARV-110 (structure undisclosed but patents suggest MW>1000) is in Phase 1 trial for prostate cancer, and the BCL-xL PROTAC DT2216 (MW=1540) has activity in mouse cancer models. In some embodiments, the molecular weights (MWs) of PROTACs disclosed herein are well below 1540.

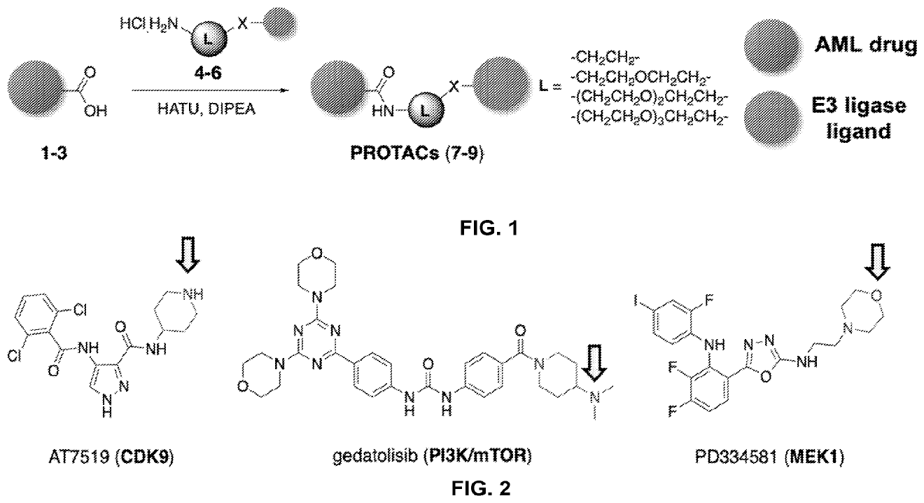

The general strategy for this proposal is encapsulated in FIG. 1, which illustrates that the indirect MCL-1 PROTACs (7-9) are prepared by coupling selected existing AML drug candidates (1-3, blue) to E3 ligase ligands (thalidomide, idasanutlin and VHL ligand, green) bearing four different linker lengths (4-6, gray) with increasing numbers of PEG units because PROTACs can be sensitive to linker length. Such linkers also assist in promoting solubility and are routinely deployed in PROTACs. Since three AML drug candidates re exploited in this proposal along with three E3 ligase ligands, this combinatorial approach provides 36 different PROTACs. To streamline chemical syntheses, a highly convergent approach is used in which the linker/E3 ligase ligand conjugates are prepared first. Conjugation to the AML drug candidates is done through solvent-exposed regions to minimize impact on target binding of the parent drug components. Drug candidates identified in FIG. 1 were selected based on multiple criteria including orthogonal mechanisms at which they regulate MCL-1 protein levels, availability of co-crystal structures, conjugatable amines or carboxylic acids (or the ability to introduce them), successful or ongoing clinical trials, reported synergy with VEN, commercial availability and/or ease of synthesis, and relatively low MW; those candidates matching the most criteria were chosen.

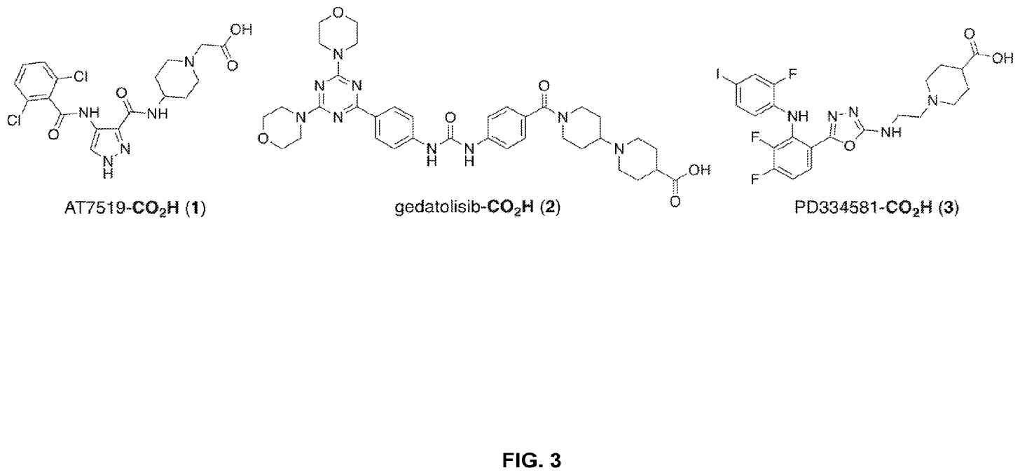

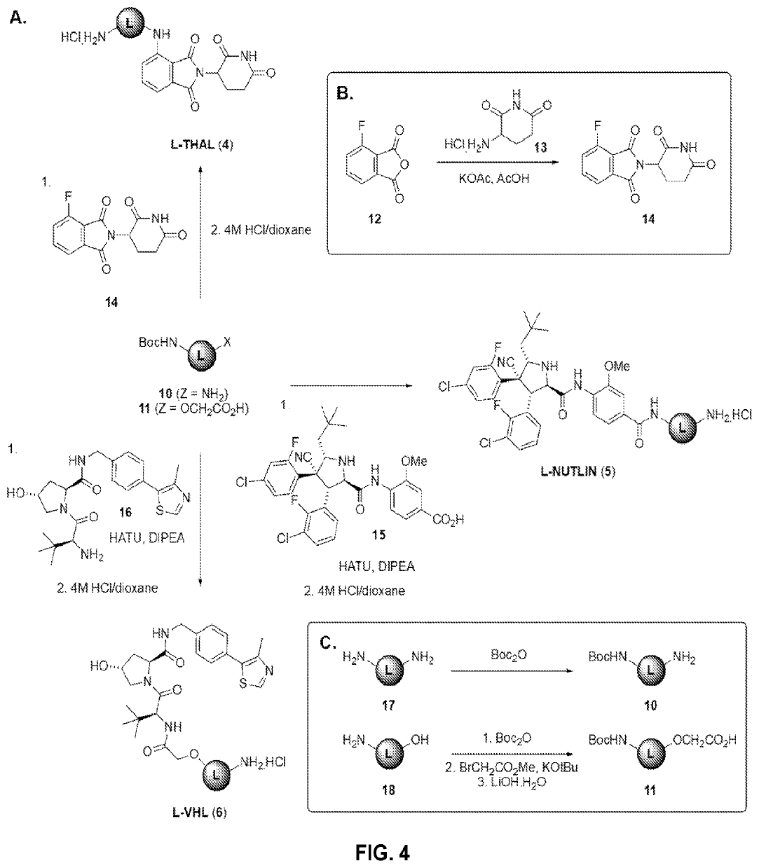

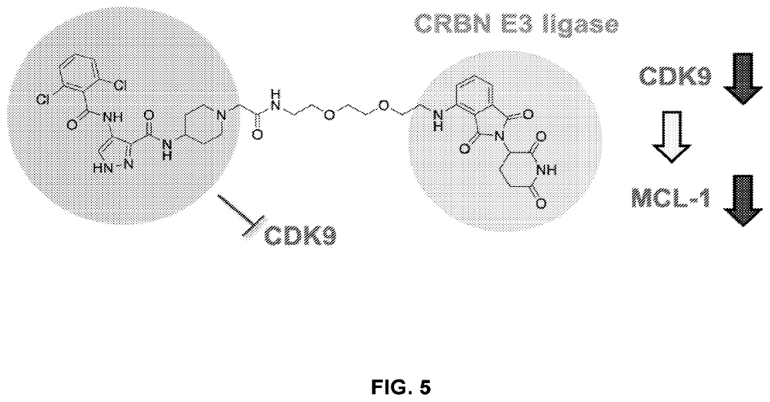

As shown in FIG. 2, the solvent-exposed, i.e. non-target binding, domains of AT7519, gedatolisib and PD334581 are highlighted in blue, and yellow arrows indicate where the molecules are modified to include a carboxylic acid to permit the coupling described in FIG. 1. The modified drug candidates are depicted in FIG. 3; crucially, the basic, solubilizing tertiary amines have been retained. AT7519 is commercially available and affordable, so AT7519-CO₂H (1) is synthesized in only two steps. Gedatolisib-CO₂H (2) and PD334581-CO₂H (3) are synthesized de novo, but their syntheses are short (6 and 5 steps, respectively) and are executed by simple manipulations of the published syntheses for the parent compounds. Meanwhile, the E3 ligase ligand linker conjugates are prepared per FIG. 4 using previously reported methods. L-THAL (4) represents the linker/thalidomide conjugate to prepare a cereblon-targeting PROTAC, L-NUTLIN (5) represents the linker/thalidomide conjugate to prepare an MDM2-targeting PROTAC, and L-VHL (6) represents the linker/VHL1 peptide conjugate to prepare a VHL-targeting PROTAC. All proposed PROTACs are prepared in two steps from the appropriate linker (10 or 11) of FIG. 4, selection of which is made based on if an amine or carboxylic acid is required to couple to the E3 ligase ligand. L-THALs (4) is prepared by a nucleophilic aromatic substitution of linkers 10 with thalidomide derivative 14, followed by acidic deprotection of the Boc group. L-NUTLINs (5) is synthesized by a HATU-mediated coupling of linkers 10 to commercially available idasanutlin 15. L-VHLs (6) are prepared by a HATU-mediated coupling of linkers 11 to commercially available VHL1 ligand 16. The secondary amine of idasanutlin is not expected to be sufficiently sterically hindered such that it will interfere in this chemistry. E3 ligase ligand/linker conjugates 4-6 are then coupled to the carboxylic acid-modified AML drug candidates 1-3 per FIG. 1, demonstrating the significant convergence of our synthetic chemistry approach. The MW ranges of the indirect MCL-1 PROTACs are as follows: AT7519=738-1227; gedatolisib=998-1487; PD334581=851-1340. All of these MWs are less than 1540, which is the MW the BCL-xL PROTAC DT2216, which is active in vivo. Moreover, several of our PROTACs have MWs less than that of FDA-approved VEN (MW=868). In addition, each PROTAC bears at least one ionizable tertiary amine to promote solubility. Lastly, the linkers are based on PEG units, which further enhances PROTAC solubilities. A total of 36 novel PROTAC candidates are synthesized in for further evaluation.

### Evaluate the Antileukemic Efficacy and Tolerability of the Indirect MCL-1 PROTACs in Vitro and in Human AML Xenograft Models

In vitro evaluations. All 36 PROTACs are evaluated in the relevant kinase assays, and those with Kᵢ values >10-fold weaker than the parent drug are not advanced further. In a non-limiting example, although PROTACs can be effective with weaker binding ligands, significantly less potent and presumably less selective candidates are not advanced further in the study. Next, the IC₅₀s of lead indirect MCL-1 PROTACs are determined by alamarBlue cell viability assays of four human AML cell lines.

Both MOLM14 and MV4;11 AML cell lines are relatively sensitive to VEN in vitro; harbor a FLT3-ITD mutation plus an MLL rearrangement, two relatively common poor-prognosis AML mutations (both cells lines also contain additional mutations); and are dependent on MCL-1 and BCL-2, and express relatively high levels of both. In addition, both MOLM14 and MV4;11 are sensitive to CDK9 inhibitors, PI3K/mTOR inhibitors and MEK inhibitors. Furthermore, MOLM14 cells were lentivirally-transduced to overexpress MCL-1 (designated MOLM14-MCL-1-OE), and showed multiple single-cell subclones to overexpress MCL-1 mRNA and to have reduced sensitivity to the selective MCL-1 inhibitor S63845; MV4;11-MCL-1-OE cloned cell lines are similarily prepared and characterized. Three repeats of each IC₅₀ determination assay are performed for each compound against each of these four AML cell lines. Multiple compounds are tested against all four AML cell lines at once, using alamarBlue microplate assays previously described. Specifically, cell lines are treated with vehicle (negative control), PROTAC alone, parent (non-PROTAC) drug alone, VEN alone, the combination of parent drug+VEN, and the combination of PROTAC+VEN, each at multiple concentrations, and IC₅₀s in the AML cell lines determined. PROTACs with IC₅₀s<1 mM that are superior to VEN or the PROTAC alone, synergistic with VEN (Chou-Talalay analysis), and, in combination with VEN, superior to VEN+the corresponding parent drug are identified. Western blotting is conducted after treatment of cells with the active PROTACs to determine if dose-dependent reduction of MCL-1 and CDK9 or PI3K/mTOR or MEK proteins is observed.

Method: OLM14 AML cell were lentivirally transduced to overexpress MCL1. Multiple human leukemia cell lines were immunoblotted for the 3 major BCL-2 family member anti-apoptotic proteins (BCL-2, MCL-1, BCL-xL), demonstrating that the MOLM14 and MV4;11 AML cell lines contain substantial basal levels of BCL-2 and MCL-1 proteins. MOLM14 AML cells were transduced with lentiviral expression vector (LV) pWCC43 backbone containing EF1a—driven GFP and Ubiquitin (Ub)-driven MCL1, or control empty LV without MCL1, or control LV containing Luc in place of MCL1, and then single-cell subcloned. MCL1 mRNA is overexpressed in 9/9 tested MOLM14 MCL1 OE cloned cell lines with no substantial changes in mRNA expression of 2 control genes (DDIT3, BCL2). In 48 h alamarBlue cell viability assays, all tested MOLM14 MCL1 OE cloned cell lines are more resistant to the MCL-1 inhibitor S6845 and the BCL-2 inhibitor VEN compared to control cell lines; similar analysis, as well as immunoblotting, is carried out for the above-mentioned additional MOLM14 MCL1 OE cell lines, in order to characterize a set of clones with a range of MCL1 OE from within the (patho)physiologic range to markedly supra-(patho)physiologic.

Prior to final selection of two indirect MCL-1 PROTAC candidates for in vivo testing, results of the alamarBlue assays for the top identified compounds as discussed above are confirmed by Annexin flow cytometric assays to quantify apoptosis, since apoptotic cytotoxicity is expected. Selection criteria for the two selected PROTACs includes binding affinity, on-target cytotoxicity, potency against VEN-sensitive and VEN-resistant AML cells, targeted protein reduction, and ideally orthogonal MCL-1 downregulation mechanisms. To evaluate in vitro hematopoietic toxicity, primary human CD34⁺ hematopoietic stem-progenitor cells from healthy adult donors are exposed to each of the two most potent indirect MCL-1 PROTACs (with combination and control groups as above), then the ability of treated cells to generate hematopoietic colonies is quantitated.

In vivo evaluation of the two most potent indirect MCL-1 PROTACs begins with dose escalation toxicity experiments to determine the maximal tolerated dose (MTD) in non-leukemia-bearing NOD/Rag^{KO}/Il2gamma^{KO} (NRG) mice administered each of the two selected indirect MCL-1 PROTACs PO (gavage) and assessed for lethal toxicity within 7 d, using a traditional 3+3 design. Next, these two compounds are tested at their MTD(s) in a 5 d schedule that utilized to evaluate VEN-based combinations against a luciferase (Luc)-labelled MOLM14 human AML cell line xenograft model. NRG mice are infused with Luc-labelled MOLM14 AML cells on day -10. After Xenogen quantification of baseline total body luminescence on d0, mice are placed into experimental groups balanced for AML burden. Each experiment to evaluate the two selected indirect MCL-1 PROTACs in vivo involves 10 groups of 5 MOLM14 AML-bearing mice treated with: (1) vehicle, (2-3) lead PROTACs, (4-5) corresponding parent drugs, (6) VEN, (7-8) corresponding parent drugs+VEN and (9-10) lead PROTACs+VEN. PROTAC and VEN are administered at the same molar concentration as the novel PROTACs; additional control groups are needed if the MTDs of the two PROTACs differ. MOLM14 AML-bearing NRG mice are dosed daily PO on dl-5 and d21-25, assessed for AML burden by Xenogen imaging on d7, d14, and d21, and monitored until clinical signs dictate need for euthanasia (projected average 10 weeks). Primary response endpoints are AML response quantitation (fold-change in AML burden on d7/14/21 vs d0 for each mouse) via imaging. Comparisons of quantitative imaging results are confirmed by Kaplan-Meier survival analyses. In addition, the two selected PROTACs are similarly tested against a Luc-labelled MV4;11 cell line xenograft model. The goal is to identify two novel indirect MCL-1 PROTACs that, in combination with VEN, each result in enhanced reduction of MOLM14 and MV4;11 AML burden relative to VEN or the PROTAC alone, and also to the combination of VEN+the respective parent (non-PROTAC) drug. If the experiments above indicate that the more drug-resistant MOLM14-MCL-1-OE and MV4;11-MCL-1-OE cells lines are advantageous for comparing activity of the indirect MCL-1 PROTACs vs. controls in vitro, this/these genetically-modified cell line(s) is used, rather than unmodified MOLM14 and MV4;11, for the xenograft experiments.

Therapeutic window: Mouse body weights is measured weekly, as a general indicator of drug toxicity. To assess hematopoietic toxicity in vivo, two additional groups of 5 (non-AML-bearing) NRG mice are treated with each of the two chosen PROTACs or the above controls, and on d10 of treatment cycle #2, these mice are bled for complete blood counts (CBCs) and euthanized.

Statistics, sample size: Results of pilot experiment using the MOLM14 AML xenograft model and two 5 d PO treatment cycles (5 mice/group) provide an empiric estimate of needed mice/group. VEN alone at the established standard dose had no detectable effect vs treatment with vehicle, on either AML burden over time or survival (adjusted p=⁰0.7²² at d21 and 1.0 at d84, respectively). Sorafenib (SOR; inhibits FLT3 and other kinases) alone at an established dose, reduced AML burden only moderately (adjusted p=⁰0.0⁹1 at d21). Co-administration of VEN+SOR resulted in deeper, longer AML burden reductions (adjusted p<0.05 at d21) and prolonged survivals (p=0.0⁴⁸ at d84), vs vehicle or either drug alone. If one or both of the two selected indirect MCL-1 PROTACs in combination with VEN is at least as effective as co-administered VEN+SOR, 5 mice/group are expected to be sufficient to demonstrate statistical superiority over VEN or the partner alone in reducing AML burden and possibly prolonging survival. Addition of a third drug (either PEGylated L-asparaginase (ASP) or an artemisinin derivative (ART) further reduced AML burden (adjusted p<0.001) and prolonged survival (p<0.01 at d84), compared to vehicle or monotherapy (VEN or SOR). If either or both of the two selected PROTACs in combination with VEN is at least as effective as co-administered VEN+SOR+these third drugs, 5 mice/group are expected to be sufficient to demonstrate statistical superiority over vehicle or monotherapy (VEN or SOR) in prolonging survival. Required mouse numbers are estimated based on inferences from these preliminary results; a similar or better efficacy for novel compounds, based on potential superiority of PROTACs over competitive inhibitors, is hypothesized. Thus, the proposed sample size (5 mice/group) is expected to provide sufficient power for statistical analyses (at p<0.05 level; α=0.05, β=0.1), which is estimated with the standard deviation of AML burden over time monitored and the expected drug efficacy differences. Moreover, each in vivo experiment is repeated twice (n=10 mice/group total). Statistical analyses are conducted using GraphPad software. AML burden is quantified using log transformed ratio of luminescence at d7/14/21 vs d0 for each mouse (fold-change); groups are compared using the appropriate parametric (ANOVA; t-test, etc) or non-parametric alternative as necessary (Wilcoxon rank sum, Kruskal-Wallis). For time-to-event endpoints, Kaplan-Meier survival of groups are compared using the log-rank test. p<0.05 is considered statistically different. This approach (including mouse numbers) has been successful for development of a novel three-drug combination that includes VEN.

Two of the parent drug candidates are synthesized but the syntheses are published and short. The E3 ligase ligands and linkers are prepared separately, using routine published chemistry, and then conjugated to the parent drugs using known coupling chemistry. The indirect MCL-1 PROTAC candidates are evaluated in the kinase assays as they are synthesized. In vitro and in vivo evaluations of the top compounds are then as well as compound scale-up for in vivo work. Method: AML xenografts were treated with venetoclax monotherapy or combinations. NRG mice were transplanted IV with Luc-labelled MOLM14 AML cells. On d0, after Xenogen quantification of baseline total body luminescence (AML burden), groups of 5 AML-bearing mice were treated on days 1-5, per a previously established MTD POx5d schedule, with VEN (150 mg/kg/d POx5), as monotherapy or co-administered with SOR (30 mg/kg/d POx5), ART (50 mg/kg/d POx5), and/or ASP (2001U/kg IP single dose). A second identical drug cycle was administered on d21-25. On d7, d14 and d21, each mouse was Xenogen imaged for total body luminescence, fold-change AML burden from d0 was calculated for each mouse, and mean (+/−SD) fold-change for each experimental group plotted. Statistically significant differences (p<0.05) in AML burden were found between vehicle and VEN+SOR, VEN+SOR+ASP, VEN+SOR+ART, respectively, at d7, d14, and d21. SOR monotherapy caused significant difference in AML burden only at d7 and d14, as compared to Vehicle. Kaplan-Meier survivals were examined. Statistically significant differences (p<0.05) in survived animal number were found between the combination treatments and Vehicle or monotherapy (VEN or SOR).

Monotherapy with ART or ASP had minimal activity as well, similar to VEN monotherapy and Vehicle. In some embodiments, parent drug components of the indirect MCL-1 PROTACs retain a solubilizing tertiary amine. In a non-limiting example, an additional ionizable tertiary amine is introduced into the linker L. In a non-limiting example, if the indirect MCL-1 PROTACs are not effective degraders of their target proteins, then longer linkers (one and two additional PEG (CH₂CH₂O) units) are employed, as PROTACs can be sensitive to linker length. In a non-limiting example, analogous PROTACs of TG02, an anti-leukemic, potent, multi-kinase inhibitor of CDKs, JAK2 and FLT3, are prepared.

### Example 2: CDK9 Inhibition and Cell Viability Activity Assays

### Testing of CDK9 Inhibition

CDK9 activity assays were performed by Nanosyn using a microfluidics mobility shift platform. In this assay, a fluorescently labeled substrate is incubated with the enzyme in the presence of test compounds. Samples are run through microfluidic chips, separating them based on differences in charge; this separates the more positive substrate from the more negative product. Enzyme activity is measured through comparison of the fluorescence from substrate and product peaks. The results are shown in Table 1.

### Testing Cell Viability

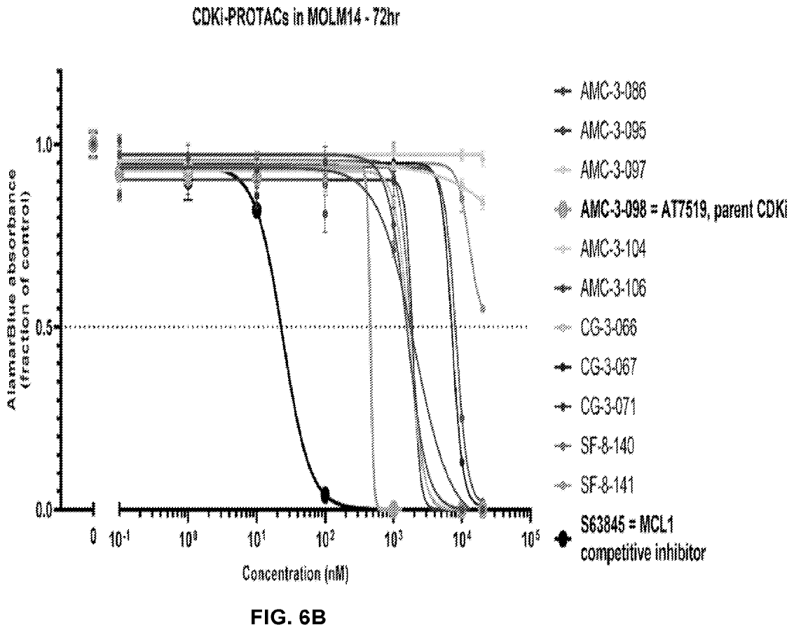

Cell viability of MOLM14 cells was measured in presence of test compounds via alamarBlue assay. This is a colorimetric assay that uses resazurin, a reduction-oxidation indicator. After incubation, resazurin is added to the test wells. The reduced form of resazurin is resorufin, which is pink and highly fluorescent. This can be measured through absorbance or fluorescence and is proportional to the amount of living cells. The results are shown in Table 1 and FIGS. 6A-6B.

TABLE 1
CDK9 Inhibition and Cell Viability Assays

| | | | | Cell | Cell |
|---|---|---|---|---|---|
| | | Molec- | | Viability | Viability |
| Com- | | ular | CDK9 | (MOLM14 | (MOLM14 |
| pound | | Weight | IC₅₀ | IC₅₀-nM) | IC₅₀-nM) |
| Name | Structure | (g/mol) | (nM) | 48 hr | 72 hr |
| AMC- 3-098 (AT7519) | 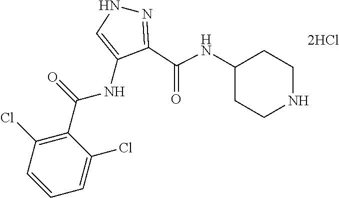 [SMILES: O=C(NC1CCNCC1)c1n[nH]cc1NC(=O)c1c(Cl)cccc1Cl] | 455.16 | 2.06 | 177.5 | 460.3 |
| AMC- 3-095 | 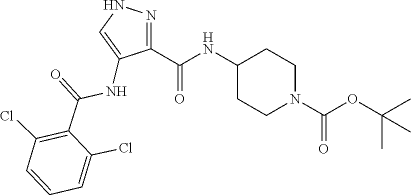 [SMILES: CC(C)(C)OC(=O)N1CCC(NC(=O)c2n[nH]cc2NC(=O)c2c(Cl)cccc2Cl)CC1] | 482.36 | 11.3 | 1679 | 2012 |
| AMC- 3-086 | 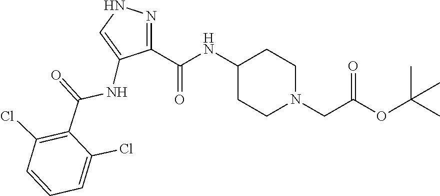 [SMILES: CC(C)(C)OC(=O)CN1CCC(NC(=O)c2n[nH]cc2NC(=O)c2c(Cl)cccc2Cl)CC1] | 496.39 | 11.0 | 1559 | 1586 |
| AMC- 3-097 | 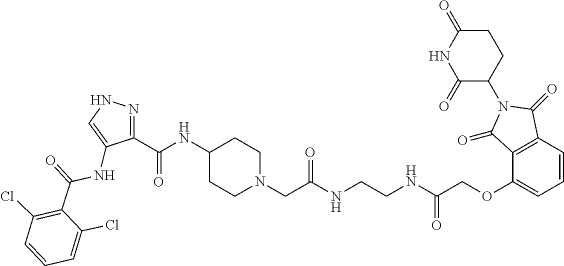 [SMILES: O=C(COc1cccc2c1C(=O)N(C1CCC(=O)NC1=O)C2=O)NCCNC(=O)CN1CCC(NC(=O)c2n[nH]cc2NC(=O)c2c(Cl)cccc2Cl)CC1] | 796.62 | 3.96 | ND | ND |
| AMC- 3-104 | 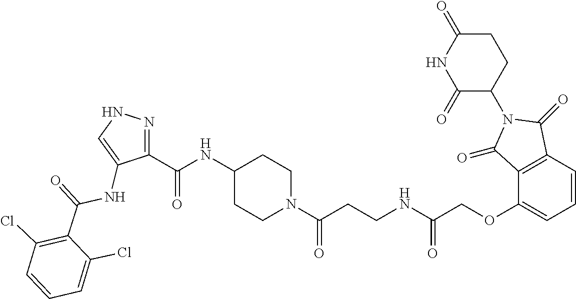 [SMILES: O=C(COc1cccc2c1C(=O)N(C1CCC(=O)NC1=O)C2=O)NCCC(=O)N1CCC(NC(=O)c2n[nH]cc2NC(=O)c2c(Cl)cccc2Cl)CC1] | 767.58 | 2.82 | ND | ND |
| AMC- 3-106 | 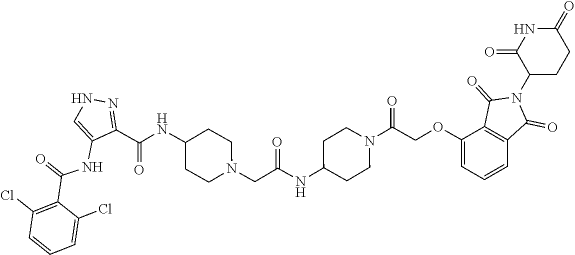 [SMILES: O=C1CCC(N2C(=O)c3cccc(OCC(=O)N4CCC(NC(=O)CN5CCC(NC(=O)c6n[nH]cc6NC(=O)c6c(Cl)cccc6Cl)CC5)CC4)c3C2=O)C(=O)N1] | 836.68 | 3.04 | 6067 | 8167 |
| CG- 3-066 | 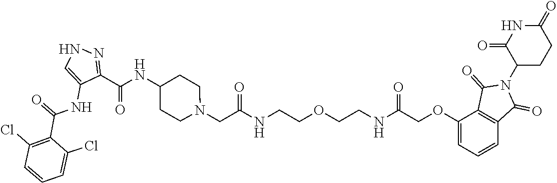 [SMILES: O=C(COc1cccc2c1C(=O)N(C1CCC(=O)NC1=O)C2=O)NCCOCCNC(=O)CN1CCC(NC(=O)c2n[nH]cc2NC(=O)c2c(Cl)cccc2Cl)CC1] | 840.67 | 3.32 | 1973 | 1690 |
| CG- 3-067 | 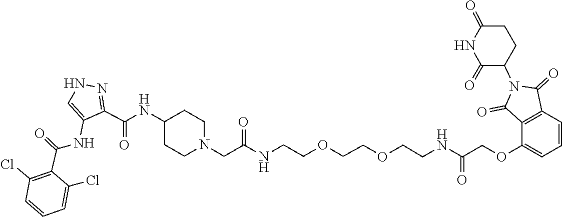 [SMILES: O=C(COc1cccc2c1C(=O)N(C1CCC(=O)NC1=O)C2=O)NCCOCCOCCNC(=O)CN1CCC(NC(=O)c2n[nH]cc2NC(=O)c2c(Cl)cccc2Cl)CC1] | 884.73 | 3.89 | 4317 | 7151 |
| CG- 3-071 | 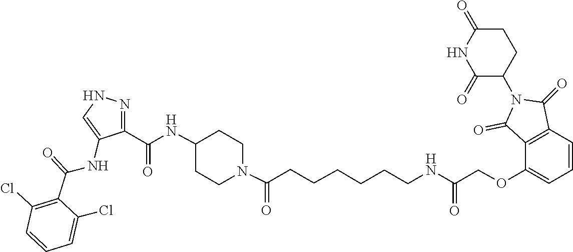 [SMILES: O=C(COc1cccc2c1C(=O)N(C1CCC(=O)NC1=O)C2=O)NCCCCCCC(=O)N1CCC(NC(=O)c2n[nH]cc2NC(=O)c2c(Cl)cccc2Cl)CC1] | 823.69 | 2.85 | 1713 | 1911 |
| SF- 8-140 | 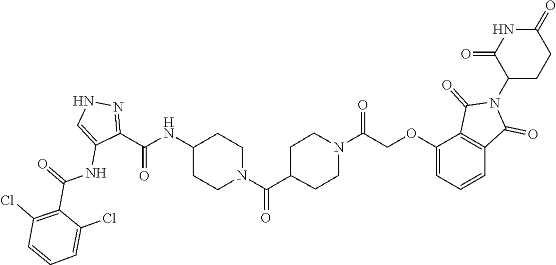 [SMILES: O=C1CCC(N2C(=O)c3cccc(OCC(=O)N4CCC(C(=O)N5CCC(NC(=O)c6n[nH]cc6NC(=O)c6c(Cl)cccc6Cl)CC5)CC4)c3C2=O)C(=O)N1] | 807.64 | 1.66 | 2998 | 1821 |
| SF- 8-141 | 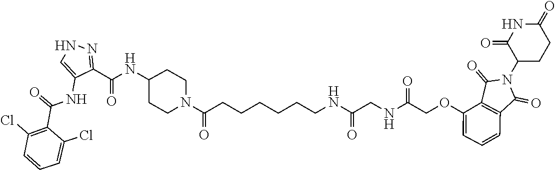 [SMILES: O=C(CNC(=O)COc1cccc2c1C(=O)N(C1CCC(=O)NC1=O)C2=O)NCCCCCCC(=O)N1CCC(NC(=O)c2n[nH]cc2NC(=O)c2c(Cl)cccc2Cl)CC1] | 880.74 | 4.49 | ND | ND |

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claims. All compounds, compositions, formulations, and methods described herein that embody the present disclosure can, in alternate embodiments, be more specifically defined by any of the transitional terms “comprising,” “consisting essentially of,” and “consisting of.”

### REFERENCES

1. Dohner H, Estey E H, Amadori S, Appelbaum F R, Buchner T, Burnett A K, Dombret H, Fenaux P, Grimwade D, Larson R A, Lo-Coco F, Naoe T, Niederwieser D, Ossenkoppele G J, Sanz M A, Sierra J, Tallman M S, Lowenberg B, Bloomfield C D, European LeukemiaNet. Diagnosis and management of acute myeloid leukemia in adults: recommendations from an international expert panel, on behalf of the European LeukemiaNet. Blood. 2010 Jan. 21; 115(3):453-474. PMID: 19880497
2. https://www.cancer.org/cancer/acute-myeloid-leukemia/about/key-statistics.
3. Oran B, Weisdorf D J. Survival for older patients with acute myeloid leukemia: a population-based study. Haematologica. 2012 December; 97(12):1916-1924. PMCID: PMC3590098
4. DiNardo C D, Jonas B A, Pullarkat V, Thirman M J, Garcia J S, Wei A H, Konopleva M, Dohner H, Letai A, Fenaux P, Koller E, Havelange V, Leber B, Esteve J, Wang J, Pejsa V, Hajek R, Porkka K, Illes A, Lavie D, Lemoli R M, Yamamoto K, Yoon S-S, Jang J-H, Yeh S-P, Turgut M, Hong W-J, Zhou Y, Potluri J, Pratz K W. Azacitidine and Venetoclax in Previously Untreated Acute Myeloid Leukemia. N Engl J Med. 2020 Aug. 13; 383(7):617-629. PMID: 32786187
5. Pollyea D A, Pratz K, Letai A, Jonas B A, Wei A H, Pullarkat V, Konopleva M, Thirman M J, Arellano M, Becker P S, Chyla B, Hong W-J, Jiang Q, Potluri J, DiNardo C D. Venetoclax with azacitidine or decitabine in patients with newly diagnosed acute myeloid leukemia: Long term follow-up from a phase 1b study. Am J Hematol. 2021 Feb. 1; 96(2):208-217. PMID: 33119898
6. Kavanagh S, Murphy T, Law A, Yehudai D, Ho J M, Chan S, Schimmer A D. Emerging therapies for acute myeloid leukemia: translating biology into the clinic. JCI Insight. 2017 Sep. 21; 2(18). PMCID: PMC5621868
7. Carter J L, Hege K, Yang J, Kalpage H A, Su Y, Edwards H, Huttemann M, Taub J W, Ge Y. Targeting multiple signaling pathways: the new approach to acute myeloid leukemia therapy. Signal Transduction and Targeted Therapy. Nature Publishing Group; 2020 Dec. 18;5(1):1-29.
8. DiNardo C D, Pratz K W, Letai A, Jonas B A, Wei A H, Thirman M, Arellano M, Frattini M G, Kantarjian H, Popovic R, Chyla B, Xu T, Dunbar M, Agarwal S K, Humerickhouse R, Mabry M, Potluri J, Konopleva M, Pollyea D A. Safety and preliminary efficacy of venetoclax with decitabine or azacitidine in elderly patients with previously untreated acute myeloid leukaemia: a non-randomised, open-label, phase 1b study. Lancet Oncol. 2018 February; 19(2):216-228. PMID: 29339097
9. Lin K H, Winter P S, Xie A, Roth C, Martz C A, Stein E M, Anderson G R, Tingley J P, Wood K C. Targeting MCL-1/BCL-XL Forestalls the Acquisition of Resistance to ABT-199 in Acute Myeloid Leukemia. Sci Rep [Internet]. 2016 Jun. 10 [cited 2021 Feb. 7];6. Available from: https://www.ncbi.nlm.nih.gov/pmc/articles/PMC4901329/PMCID: PMC4901329
10. Wang Q, Wan J, Zhang W, Hao S. MCL-1 or BCL-xL-dependent resistance to the BCL-2 antagonist (ABT-199) can be overcome by specific inhibitor as single agents and in combination with ABT-199 in acute myeloid leukemia cells. Leuk Lymphoma. 2019 September; 60(9):2170-2180. PMID: 30626241
11. Perini G F, Ribeiro G N, Pinto Neto J V, Campos L T, Hamerschlak N. BCL-2 as therapeutic target for hematological malignancies. J Hematol Oncol. 2018 May 11; 11(1):65. PMCID: PMC5946445
12. Adams J M, Cory S. The BCL-2 arbiters of apoptosis and their growing role as cancer targets. Cell Death Differ. 2018 January; 25(1):27-36. PMCID: PMC5729526
13. Kale J, Osterlund E J, Andrews D W. BCL-2 family proteins: changing partners in the dance towards death. Cell Death Differ. 2018 January; 25(1):65-80. PMCID: PMC5729540
14. Souers A J, Leverson J D, Boghaert E R, Ackler S L, Catron N D, Chen J, Dayton B D, Ding H, Enschede S H, Fairbrother W J, Huang D C S, Hymowitz S G, Jin S, Khaw S L, Kovar P J, Lam L T, Lee J, Maecker H L, Marsh K C, Mason K D, Mitten M J, Nimmer P M, Oleksijew A, Park C H, Park C-M, Phillips D C, Roberts A W, Sampath D, Seymour J F, Smith M L, Sullivan G M, Tahir S K, Tse C, Wendt M D, Xiao Y, Xue J C, Zhang H, Humerickhouse R A, Rosenberg S H, Elmore S W. ABT-199, a potent and selective BCL-2 inhibitor, achieves antitumor activity while sparing platelets. Nat Med. 2013 February; 19(2):202-208. PMID: 23291630
15. Wei A H, Tiong I S. Midostaurin, enasidenib, CPX-351, gemtuzumab ozogamicin, and venetoclax bring new hope to AML. Blood. 2017 Dec. 7; 130(23):2469-2474. PMID: 29051180
16. Yap J L, Chen L, Lanning M E, Fletcher S. Expanding the Cancer Arsenal with Targeted Therapies: Disarmament of the Antiapoptotic Bcl-2 Proteins by Small Molecules. J Med Chem. 2017 Feb. 9; 60(3):821-838. PMID: 27749061
17. Juarez-Salcedo L M, Desai V, Dalia S. Venetoclax: evidence to date and clinical potential. Drugs Context. 2019; 8:212574. PMCID: PMC6788387
18. Konopleva M, Pollyea D A, Potluri J, Chyla B, Hogdal L, Busman T, McKeegan E, Salem A H, Zhu M, Ricker J L, Blum W, DiNardo C D, Kadia T, Dunbar M, Kirby R, Falotico N, Leverson J, Humerickhouse R, Mabry M, Stone R, Kantarjian H, Letai A. Efficacy and Biological Correlates of Response in a Phase II Study of Venetoclax Monotherapy in Patients with Acute Myelogenous Leukemia. Cancer Discov. 2016 October; 6(10):1106-1117. PMCID: PMC5436271
19. Kaufmann S H, Karp J E, Svingen P A, Krajewski S, Burke P J, Gore S D, Reed J C. Elevated expression of the apoptotic regulator Mcl-1 at the time of leukemic relapse. Blood. 1998 Feb. 1; 91(3):991-1000. PMID: 9446661
20. Leverson J D, Cojocari D. Hematologic Tumor Cell Resistance to the BCL-2 Inhibitor Venetoclax: A Product of Its Microenvironment? Front Oncol. 2018; 8:458. PMCID: PMC6204401
21. Luedtke D A, Niu X, Pan Y, Zhao J, Liu S, Edwards H, Chen K, Lin H, Taub J W, Ge Y. Inhibition of Mcl-1 enhances cell death induced by the Bcl-2-selective inhibitor ABT-199 in acute myeloid leukemia cells. Signal Transduct Target Ther. 2017; 2:17012. PMCID: PMC5661618
22. Tahir S K, Smith M L, Hessler P, Rapp L R, Idler K B, Park C H, Leverson J D, Lam L T. Potential mechanisms of resistance to venetoclax and strategies to circumvent it. BMC Cancer. 2017 Jun. 2; 17(1):399. PMCID: PMC5457565
23. Wei A H, Roberts A W, Spencer A, Rosenberg A S, Siegel D, Walter R B, Caenepeel S, Hughes P, McIver Z, Mezzi K, Morrow P K, Stein A. Targeting MCL-1 in hematologic malignancies: Rationale and progress. Blood Rev. 2020 November; 44:100672. PMCID: PMC7442684
24. Bolomsky A, Vogler M, Kose M C, Heckman C A, Ehx G, Ludwig H, Caers J. MCL-1 inhibitors, fast-lane development of a new class of anti-cancer agents. J Hematol Oncol [Internet]. 2020 Dec. 11 [cited 2021 Feb. 7];13. Available from: https://www.ncbi.nlm.nih.gov/pmc/articles/PMC7731749/PMCID: PMC7731749
25. Fletcher S. MCL-1 inhibitors—where are we now (2019)? Expert Opin Ther Pat. 2019 November; 29(11):909-919. PMID: 31566022
26. Xiang W, Yang C-Y, Bai L. MCL-1 inhibition in cancer treatment. Onco Targets Ther. 2018 Oct. 23; 11:7301-7314. PMCID: PMC6205821
27. Leverson J D, Zhang H, Chen J, Tahir S K, Phillips D C, Xue J, Nimmer P, Jin S, Smith M, Xiao Y, Kovar P, Tanaka A, Bruncko M, Sheppard G S, Wang L, Gierke S, Kategaya L, Anderson D J, Wong C, Eastham-Anderson J, Ludlam M J C, Sampath D, Fairbrother W J, Wertz I, Rosenberg S H, Tse C, Elmore S W, Souers A J. Potent and selective small-molecule MCL-1 inhibitors demonstrate on-target cancer cell killing activity as single agents and in combination with ABT-263 (navitoclax). Cell Death & Disease. Nature Publishing Group; 2015 January; 6(1):e1590-e1590.
28. Opferman J T, Iwasaki H, Ong C C, Suh H, Mizuno S, Akashi K, Korsmeyer S J. Obligate role of anti-apoptotic MCL-1 in the survival of hematopoietic stem cells. Science. 2005 Feb. 18; 307(5712):1101-1104. PMID: 15718471
29. Wang X, Bathina M, Lynch J, Koss B, Calabrese C, Frase S, Schuetz J D, Rehg J E, Opferman J T. Deletion of MCL-1 causes lethal cardiac failure and mitochondrial dysfunction. Genes Dev. 2013 Jun. 15; 27(12):1351-1364. PMCID: PMC3701191
30. Vick B, Weber A, Urbanik T, Maass T, Teufel A, Krammer P H, Opferman J T, Schuchmann M, Galle P R, Schulze-Bergkamen H. Knockout of myeloid cell leukemia-1 induces liver damage and increases apoptosis susceptibility of murine hepatocytes. Hepatology. 2009 February; 49(2):627-636. PMCID: PMC2753874
31. Rinkenberger J L, Homing S, Klocke B, Roth K, Korsmeyer S J. Mcl-1 deficiency results in peri-implantation embryonic lethality. Genes Dev. 2000 Jan. 1; 14(1):23-27. PMCID: PMC316347
32. Thomas R L, Roberts D J, Kubli D A, Lee Y, Quinsay M N, Owens J B, Fischer K M, Sussman M A, Miyamoto S, Gustafsson A B. Loss of MCL-1 leads to impaired autophagy and rapid development of heart failure. Genes Dev. 2013 Jun. 15; 27(12):1365-1377. PMCID: PMC3701192
33. https://clinicaltrials.gov/ct2/show/NCT03797261.
34. https://www.evaluate.com/vantage/articles/news/snippets/amgen-halts-trials-cardiac-toxicity.
35. Tron A E, Belmonte M A, Adam A, Aquila B M, Boise L H, Chiarparin E, Cidado J, Embrey K J, Gangl E, Gibbons F D, Gregory G P, Hargreaves D, Hendricks J A, Johannes J W, Johnstone R W, Kazmirski S L, Kettle J G, Lamb M L, Matulis S M, Nooka A K, Packer M J, Peng B, Rawlins P B, Robbins D W, Schuller A G, Su N, Yang W, Ye Q, Zheng X, Secrist J P, Clark E A, Wilson D M, Fawell S E, Hird A W. Discovery of Mcl-1-specific inhibitor AZD5991 and preclinical activity in multiple myeloma and acute myeloid leukemia. Nature Communications. Nature Publishing Group; 2018 Dec. 17;9(1):5341.
36. https://clinicaltrials.gov/ct2/show/NCT03218683.
37. Gadd M S, Testa A, Lucas X, Chan K-H, Chen W, Lamont D J, Zengerle M, Ciulli A. Structural basis of PROTAC cooperative recognition for selective protein degradation. Nat Chem Biol. 2017 May; 13(5):514-521. PMCID: PMC5392356
38. Paiva S-L, Crews C M. Targeted protein degradation: elements of PROTAC design. Curr Opin Chem Biol. 2019 June; 50:111-119. PMCID: PMC6930012
39. Zou Y, Ma D, Wang Y. The PROTAC technology in drug development. Cell Biochem Funct. 2019 January; 37(1):21-30. PMCID: PMC6590639
40. Li X, Song Y. Proteolysis-targeting chimera (PROTAC) for targeted protein degradation and cancer therapy. Journal of Hematology & Oncology. 2020 May 13;13(1):50.
41. Buhimschi A D, Armstrong H A, Toure M, Jaime-Figueroa S, Chen T L, Lehman A M, Woyach J A, Johnson A J, Byrd J C, Crews C M. Targeting the C481S Ibrutinib-Resistance Mutation in Bruton's Tyrosine Kinase Using PROTAC-Mediated Degradation. Biochemistry. 2018 Jul. 3; 57(26):3564-3575. PMID: 29851337
42. Squires M S, Feltell R E, Wallis N G, Lewis E J, Smith D-M, Cross D M, Lyons J F, Thompson N T. Biological characterization of AT7519, a small-molecule inhibitor of cyclin-dependent kinases, in human tumor cell lines. Mol Cancer Ther. American Association for Cancer Research; 2009 Feb. 1;8(2):324-332. PMID: 19174555
43. Venkatesan A M, Dehnhardt C M, Delos Santos E, Chen Z, Dos Santos O, Ayral-Kaloustian S, Khafizova G, Brooijmans N, Mallon R, Hollander I, Feldberg L, Lucas J, Yu K, Gibbons J, Abraham R T, Chaudhary I, Mansour T S. Bis(morpholino-1,3,5-triazine) derivatives: potent adenosine 5′-triphosphate competitive phosphatidylinositol-3-kinase/mammalian target of rapamycin inhibitors: discovery of compound 26 (PKI-587), a highly efficacious dual inhibitor. J Med Chem. 2010 Mar. 25; 53(6):2636-2645. PMID: 20166697
44. Zhang J, Yang P L, Gray N S. Targeting cancer with small molecule kinase inhibitors. Nat Rev Cancer. 2009 January; 9(1):28-39. PMID: 19104514
45. Bogenberger J, Whatcott C, Hansen N, Delman D, Shi C-X, Kim W, Haws H, Soh K, Lee Y S, Peterson P, Siddiqui-Jain A, Weitman S, Stewart K, Bearss D, Mesa R, Warner S, Tibes R. Combined venetoclax and alvocidib in acute myeloid leukemia. Oncotarget. 2017 Dec. 5; 8(63):107206-107222. PMCID: PMC5739808
46. Jiang H, Lwin T, Zhao X, Ren Y, Li G, Moscinski L, Shah B, Tao J. Venetoclax as a single agent and in combination with PI3K-MTOR1/2 kinase inhibitors against ibrutinib sensitive and resistant mantle cell lymphoma. Br J Haematol. 2019 January; 184(2):298-302. PMCID: PMC6066460
47. Pollyea D A, Amaya M, Strati P, Konopleva M Y. Venetoclax for AML: changing the treatment paradigm. Blood Adv. 2019 Dec. 23; 3(24):4326-4335. PMCID: PMC6929394
48. Lu J, Qian Y, Altieri M, Dong H, Wang J, Raina K, Hines J, Winkler J D, Crew A P, Coleman K, Crews C M. Hijacking the E3 Ubiquitin Ligase Cereblon to Efficiently Target BRD4. Chem Biol. 2015 Jun. 18; 22(6):755-763. PMCID: PMC4475452
49. Hines J, Lartigue S, Dong H, Qian Y, Crews C M. MDM2-Recruiting PROTAC Offers Superior, Synergistic Antiproliferative Activity via Simultaneous Degradation of BRD4 and Stabilization of p53. Cancer Res. 2019 Jan. 1; 79(1):251-262. PMCID: PMC6318015
50. Buckley D L, Van Molle I, Gareiss P C, Tae H S, Michel J, Noblin D J, Jorgensen W L, Ciulli A, Crews C M. Targeting the von Hippel-Lindau E3 ubiquitin ligase using small molecules to disrupt the VHI/HIF-1α interaction. J Am Chem Soc. 2012 Mar. 14; 134(10):4465-4468. PMCID: PMC3448299
51. https://clinicaltrials.gov/ct2/show/NCT03888612.
52. He Y, Koch R, Budamagunta V, Zhang P, Zhang X, Khan S, Thummuri D, Ortiz Y T, Zhang X, Lv D, Wiegand J S, Li W, Palmer A C, Zheng G, Weinstock D M, Zhou D. DT2216-a Bcl-xL-specific degrader is highly active against Bcl-xL-dependent T cell lymphomas. J Hematol Oncol. 2020 Jul. 16; 13(1):95. PMCID: PMC7364785
53. Khan S, Zhang X, Lv D, Zhang Q, He Y, Zhang P, Liu X, Thummuri D, Yuan Y, Wiegand J S, Pei J, Zhang W, Sharma A, McCurdy C R, Kuruvilla V M, Baran N, Ferrando A A, Kim Y-M, Rogojina A, Houghton P J, Huang G, Hromas R, Konopleva M, Zheng G, Zhou D. A selective BCL-XL PROTAC degrader achieves safe and potent antitumor activity. Nat Med. 2019 December; 25(12):1938-1947. PMCID: PMC6898785
54. Boffo S, Damato A, Alfano L, Giordano A. CDK9 inhibitors in acute myeloid leukemia. Journal of Experimental & Clinical Cancer Research. 2018 Feb. 23; 37(1):36.
55. Phillips D C, Jin S, Gregory G P, Zhang Q, Xue J, Zhao X, Chen J, Tong Y, Zhang H, Smith M, Tahir S K, Clark R F, Penning T D, Devlin J R, Shorn J, Hsi E D, Albert D H, Konopleva M, Johnstone R W, Leverson J D, Souers A J. A novel CDK9 inhibitor increases the efficacy of venetoclax (ABT-199) in multiple models of hematologic malignancies. Leukemia. 2020 June; 34(6):1646-1657. PMCID: PMC7266741
56. Dey J, Deckwerth T L, Kerwin W S, Casalini J R, Merrell A J, Grenley M O, Burns C, Ditzler S H, Dixon C P, Beirne E, Gillespie K C, Kleinman E F, Klinghoffer R A. Voruciclib, a clinical stage oral CDK9 inhibitor, represses MCL-1 and sensitizes high-risk Diffuse Large B-cell Lymphoma to BCL2 inhibition. Sci Rep. 2017 Dec. 21; 7(1):18007. PMCID: PMC5740070
57. Kour S, Rana S, Contreras J I, King H M, Robb C M, Sonawane Y A, Bendjennat M, Crawford A J, Barger C J, Kizhake S, Luo X, Hollingsworth M A, Natarajan A. CDK5 Inhibitor Downregulates Mcl-1 and Sensitizes Pancreatic Cancer Cell Lines to Navitoclax. Mol Pharmacol. American Society for Pharmacology and Experimental Therapeutics; 2019 Oct. 1;96(4):419-429. PMID: 31467029
58. Wyatt P G, Woodhead A J, Berdini V, Boulstridge J A, Carr M G, Cross D M, Davis D J, Devine L A, Early T R, Feltell R E, Lewis E J, McMenamin R L, Navarro E F, O'Brien M A, O'Reilly M, Reule M, Saxty G, Seavers L C A, Smith D-M, Squires M S, Trewartha G, Walker M T, Woolford A J-A. Identification of N-(4-Piperidinyl)-4-(2,6-dichlorobenzoylamino)-1H-pyrazole-3-carboxamide (AT7519), a Novel Cyclin Dependent Kinase Inhibitor Using Fragment-Based X-Ray Crystallography and Structure Based Drug Design. J Med Chem. American Chemical Society; 2008 Aug. 1;51(16):4986-4999.
59. Zhou F, Chen L, Cao C, Yu J, Luo X, Zhou P, Zhao L, Du W, Cheng J, Xie Y, Chen Y. Development of selective mono or dual PROTAC degrader probe of CDK isoforms. European Journal of Medicinal Chemistry. 2020 Feb. 1; 187:111952.
60. Nepstad I, Hatfield K J, Grenningsater I S, Reikvam H. The PI3K-Akt-mTOR Signaling Pathway in Human Acute Myeloid Leukemia (AML) Cells. Int J Mol Sci. 2020 Apr. 21; 21(8). PMCID: PMC7215987
61. Mills J R, Hippo Y, Robert F, Chen S M H, Malina A, Lin C-J, Trojahn U, Wendel H-G, Charest A, Bronson R T, Kogan S C, Nadon R, Housman D E, Lowe S W, Pelletier J. mTORC1 promotes survival through translational control of Mcl-1. Proc Natl Acad Sci USA. 2008 Aug. 5; 105(31):10853-10858. PMCID: PMC2504845
62. Hua H, Kong Q, Zhang H, Wang J, Luo T, Jiang Y. Targeting mTOR for cancer therapy. Journal of Hematology & Oncology. 2019 Jul. 5; 12(1):71.
63. Vargaftig J, Farhat H, Ades L, Briaux A, Benoist C, Turbiez I, Vey N, Glaisner S, Callens C. Phase 2 Trial of Single Agent Gedatolisib (PF-05212384), a Dual PI3K/mTOR Inhibitor, for Adverse Prognosis and Relapse/Refractory AML: Clinical and Transcriptomic Results. Blood. 2018 Nov. 29; 132(Supplement 1):5233-5233.
64. Yap J L, Worlikar S, MacKerell A D, Shapiro P, Fletcher S. Small-molecule inhibitors of the ERK signaling pathway: Towards novel anticancer therapeutics. Chem Med Chem. 2011 Jan. 3; 6(1):38-48. PMCID: PMC3477473
65. Domina A M, Vrana J A, Gregory M A, Hann S R, Craig R W. MCL1 is phosphorylated in the PEST region and stabilized upon ERK activation in viable cells, and at additional sites with cytotoxic okadaic acid or taxol. Oncogene. 2004 Jul. 8; 23(31):5301-5315. PMID: 15241487
66. Vollmer S, Cunoosamy D, Lv H, Feng H, Li X, Nan Z, Yang W, Perry M W D. Design, Synthesis, and Biological Evaluation of MEK PROTACs. J Med Chem. 2020 Jan. 9; 63(1):157-162. PMID: 31804822
67. Liu X, Zhu G, Li L, Liu Y, Wang F, Song X, Mao Y. New and Practical Synthesis of Gedatolisib. Org Process Res Dev. American Chemical Society; 2018 Jan. 19;22(1):62-66.
68. Sharon D, Cathelin S, Mirali S, Di Trani J M, Yanofsky D J, Keon K A, Rubinstein J L, Schimmer A D, Ketela T, Chan S M. Inhibition of mitochondrial translation overcomes venetoclax resistance in AML through activation of the integrated stress response. Sci Transl Med. 2019 Oct. 30; 11(516). PMID: 31666400
69. Seipel K, Marques MAT, Sidler C, Mueller B U, Pabst T. MDM2- and FLT3-inhibitors in the treatment of FLT3-ITD acute myeloid leukemia, specificity and efficacy of NVP-HDM201 and midostaurin. Haematologica. 2018 November; 103(11):1862-1872. PMCID: PMC6278968
70. Anstee N S, Bilardi R A, Ng A P, Xu Z, Robati M, Vandenberg C J, Cory S. Impact of elevated anti-apoptotic MCL-1 and BCL-2 on the development and treatment of MLL-AF9 AML in mice. Cell Death Differ. 2019 July; 26(7):1316-1331. PMCID: PMC6748137
71. Pan R, Hogdal L J, Benito J M, Bucci D, Han L, Borthakur G, Cortes J, DeAngelo D J, Debose L, Mu H, Dohner H, Gaidzik V I, Galinsky I, Golfman L S, Haferlach T, Harutyunyan K G, Hu J, Leverson J D, Marcucci G, Muschen M, Newman R, Park E, Ruvolo P P, Ruvolo V, Ryan J, Schindela S, Zweidler-McKay P, Stone R M, Kantarjian H, Andreeff M, Konopleva M, Letai A G. Selective BCL-2 inhibition by ABT-199 causes on-target cell death in acute myeloid leukemia. Cancer Discov. 2014 March; 4(3):362-375. PMCID: PMC3975047
72. Lindblad O, Cordero E, Puissant A, Macaulay L, Ramos A, Kabir N N, Sun J, Vallon-Christersson J, Haraldsson K, Hemann M T, Borg A, Levander F, Stegmaier K, Pietras K, Ronnstrand L, Kazi J U. Aberrant activation of the PI3K/mTOR pathway promotes resistance to sorafenib in AML. Oncogene. 2016 Sep. 29; 35(39):5119-5131. PMCID: PMC5399143
73. Seipel K, Marques MAT, Sidler C, Mueller B U, Pabst T. The Cellular p53 Inhibitor MDM2 and the Growth Factor Receptor FLT3 as Biomarkers for Treatment Responses to the MDM2-Inhibitor Idasanutlin and the MEK1 Inhibitor Cobimetinib in Acute Myeloid Leukemia. Cancers (Basel). 2018 May 31; 10(6). PMCID: PMC6025168
74. Moses B S, McCullough S, Fox J M, Mott B T, Bentzen S M, Kim M, Tyner J W, Lapidus R G, Emadi A, Rudek M A, Kingsbury T J, Civin C I. Antileukemic efficacy of a potent artemisinin combined with sorafenib and venetoclax. Blood Adv. 2021 Feb. 9; 5(3):711-724. PMCID: PMC7876886
75. Fox J M, Moynihan J R, Mott B T, Mazzone J R, Anders N M, Brown P A, Rudek M A, Liu J O, Arav-Boger R, Posner G H, Civin C I, Chen X. Artemisinin-derived dimer ART-838 potently inhibited human acute leukemias, persisted in vivo, and synergized with antileukemic drugs. Oncotarget. 2016 Feb. 9; 7(6):7268-7279. PMCID: PMC4872784
76. Goh K C, Novotny-Diermayr V, Hart S, Ong L C, Loh Y K, Cheong A, Tan Y C, Hu C, Jayaraman R, William A D, Sun E T, Dymock B W, Ong K H, Ethirajulu K, Burrows F, Wood J M. TG02, a novel oral multi-kinase inhibitor of CDKs, JAK2 and FLT3 with potent anti-leukemic properties. Leukemia. 2012 February; 26(2):236-243. PMID: 21860433

It is claimed:

1. A compound of formula (I), or comprising a substructure of formula (I), or a pharmaceutically acceptable salt, solvate, hydrate, cocrystal, or prodrug thereof 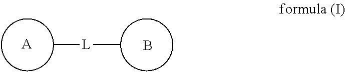
wherein in formula (I):
A comprises an Mcl-1 protein indirect inhibitor moiety;
L is a linking group; and
B comprises an E3 ubiquitin ligase ligand moiety, wherein the Mcl-1 protein inhibitor moiety thereof, and the E3 ubiquitin ligase ligand moiety thereof are each connected to L at any chemically feasible site.

2. The compound of claim 1, wherein the Mcl-1 protein indirect inhibitor moiety comprises a CDK9 inhibitor, a dual PI3K/mTOR inhibitor, a MEK1/2 inhibitor, a FLT3 inhibitor, a JAK1/2 inhibitor, a STAT3 inhibitor, an ERK1/2 inhibitor, or any substructure thereof.

3. The compound of claim 2, wherein the CDK9 inhibitor is selected from AT7519, TG02, PHA 767491, PHA-793887, PHA-848125, BAY 1143572, BAY 1112054, Cdk9 inhibitor II (CAS 140651-18-9 from Calbiochem), DRB, AZD-5438, SNS-032, dinaciclib, LY2857785, flavopiridol, purvalanol B, CDKI-71, CDKI-73, CAN508, FIT-039, CYC065, P276-00, 3,4-dimethyl-5-[2-(4-piperazin-1-yl-phenylamino)-pyrimidin-4-yl]-3H-thiazol-2-one, wogonin, apigenin, chrysin, luteolin, 4-methyl-5-[2-(3-nitroanilino)pyrimidin-4-yl]-1,3-thiazol-2-amine, shRNAs against CDK9, anti-sense mRNA against CDK9 and anti-CDK9 antibodies, and any substructure thereof.

4. The compound of claim 2, wherein the dual PI3K/mTOR inhibitor is selected from gedatolisib (PF-05212384; PKI-587), XL765, GDC-0980, BEZ235 (NVP-BEZ235), BGT226, GSK2126458, PF-04691502, and any substructure thereof.

5. The compound of claim 2, wherein the MEK1/2 inhibitor is selected from PD334581, CI-1040, AZD6244, PD318088, PD98059, RDEA119, 6-Methoxy-7-(3-morpholin-4-yl-propoxy)-4-(4-phenoxy-phenylamino)-quinoline-3-carbonitrile, 4-[3-Chloro-4-(1-methyl-1H-imidazol-2-ylsulfanyl)-phenylamino]-6-methoxy-7-(3-morpholin-4-yl-propoxy)-quinoline-3-carbonitrile, and any substructure thereof.

6. The compound of claim 2, wherein the FLT3 inhibitor is selected from midostaurin, quizartinib, crenolanib, gilteritinib, FLX-925 (AMG-925), G-749, and any substructure thereof.

7. The compound of claim 2, wherein the JAK1/2 inhibitor is selected from Ruxolitinib, Baricitinib, Tofacitinib, and any substructure thereof.

8. The compound of claim 2, wherein the STAT3 inhibitor is selected from WP1066, S31-201, C1-C10, and any substructure thereof.

9. The compound of claim 2, wherein the ERK1/2 inhibitor is selected from WP1066AEZS-131, AEZS-136, BVD-523, SCH-722984, SCH-772984, SCH-900353 (MK-8353), and any substructure thereof.

10. The compound of claim 1, wherein the Mcl-1 protein indirect inhibitor moiety is selected from AT7519, gedatolisib (PF-05212384; PKI-587), PD334581, TG02, and any substructure thereof.

11. The compound of claim 1, wherein the Mcl-1 protein indirect inhibitor moiety is selected from

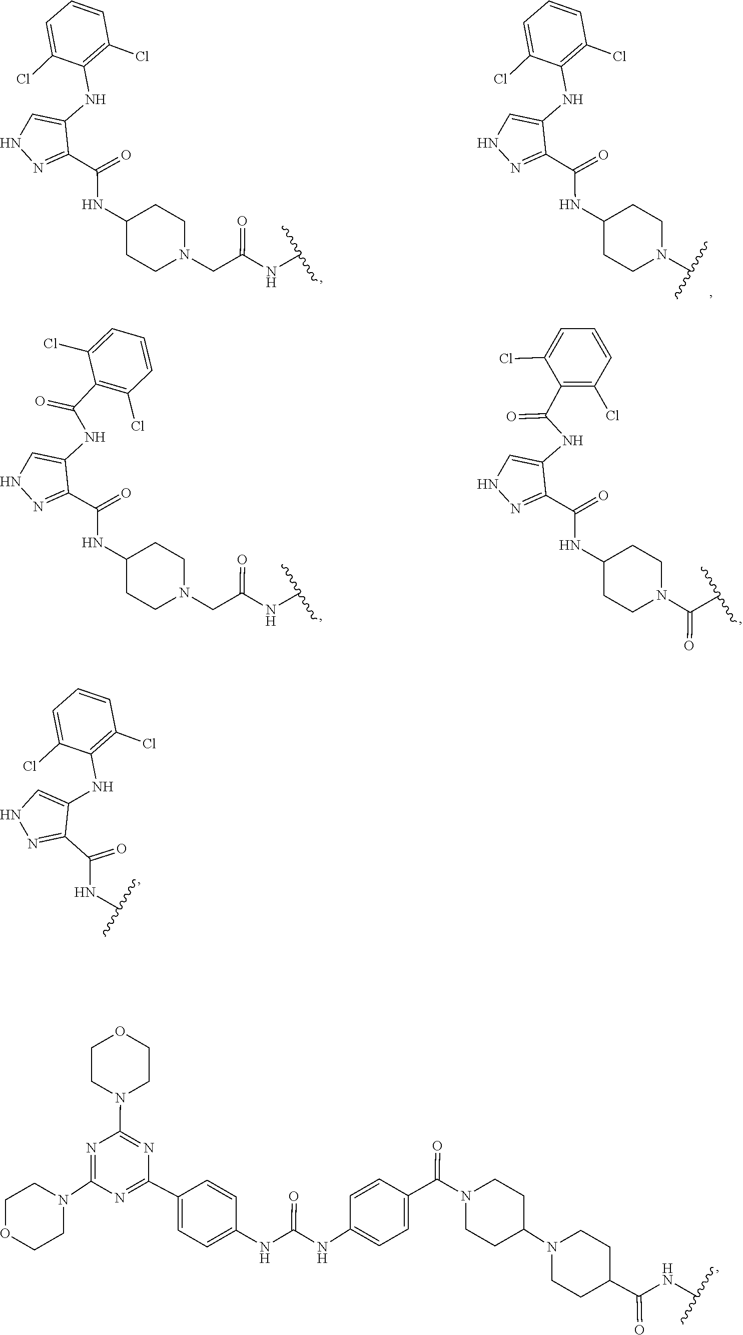 [SMILES: *NC(=O)CN1CCC(NC(=O)c2n[nH]cc2Nc2c(Cl)cccc2Cl)CC1 ; *N1CCC(NC(=O)c2n[nH]cc2Nc2c(Cl)cccc2Cl)CC1 ; *NC(=O)CN1CCC(NC(=O)c2n[nH]cc2NC(=O)c2c(Cl)cccc2Cl)CC1 ; *C(=O)N1CCC(NC(=O)c2n[nH]cc2NC(=O)c2c(Cl)cccc2Cl)CC1 ; *NC(=O)c1n[nH]cc1Nc1c(Cl)cccc1Cl ; *NC(=O)C1CCN(C2CCN(C(=O)c3ccc(NC(=O)Nc4ccc(-c5nc(N6CCOCC6)nc(N6CCOCC6)n5)cc4)cc3)CC2)CC1]

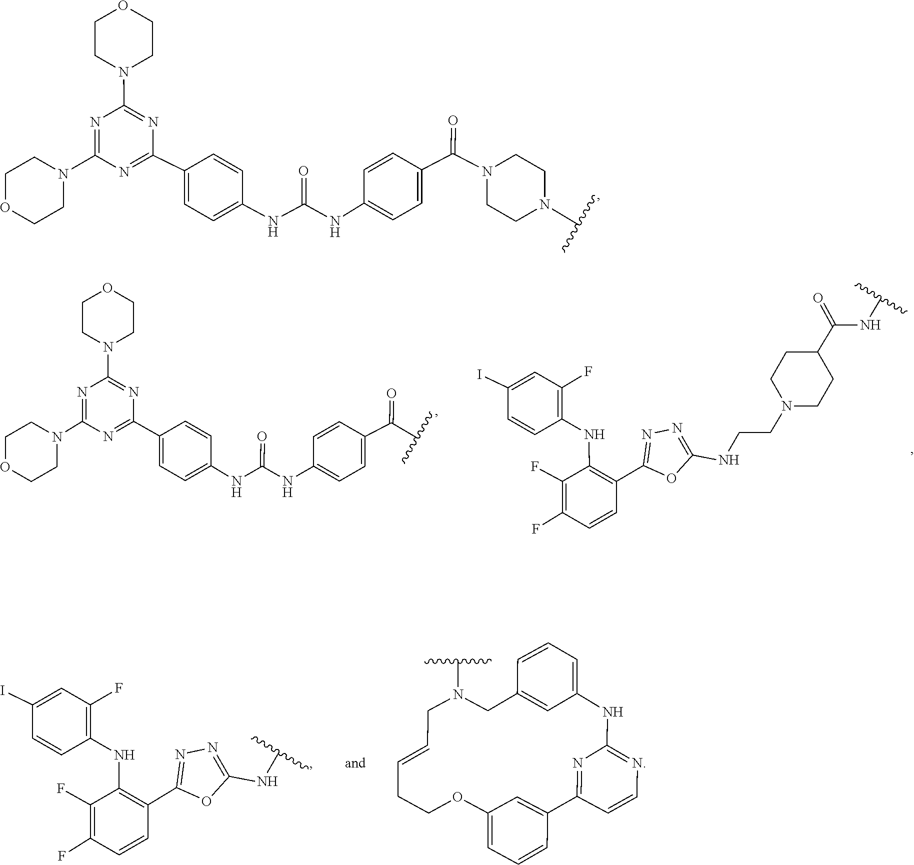 [SMILES: *N1CCN(C(=O)c2ccc(NC(=O)Nc3ccc(-c4nc(N5CCOCC5)nc(N5CCOCC5)n4)cc3)cc2)CC1 ; *C(=O)c1ccc(NC(=O)Nc2ccc(-c3nc(N4CCOCC4)nc(N4CCOCC4)n3)cc2)cc1 ; *NC(=O)C1CCN(CCNc2nnc(-c3ccc(F)c(F)c3Nc3ccc(I)cc3F)o2)CC1 ; *Nc1nnc(-c2ccc(F)c(F)c2Nc2ccc(I)cc2F)o1 ; *N1C/C=C/CCOc2cccc(c2)-c2ccnc(n2)Nc2cccc(c2)C1]

12. The compound of claim 1, wherein L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, -cycloalkyl-, -heterocyclyl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR)N(R^{b})—, —N(R^{b})S(O)_{w}—, —S(O)N(R^{b})—, —S(O)_{w}O—, —OS(O)_{w}—, —OS(O)_{w}O—, —O(O)P(OR^{b})O—, (O)P(O—)₃, —O(S)P(OR)O—, and (S)P(O—)₃, wherein w is 1 or 2, and R^{b} is independently hydrogen, optionally substituted alkyl, or optionally substituted aryl.

13. The compound of claim 12, wherein L comprises one or more linking groups selected from —C₁₋₁₀ alkyl-, —O—C₁₋₁₀ alkyl-, —O—, -cycloalkyl-, -heterocyclyl-, —C(O)—, —C(O)N(R^{b})— wherein R^{b} is hydrogen or optionally substituted alkyl, and —N(R^{b})— wherein R^{b} is optionally substituted alkyl.

14. The compound of claim 12 or 13, wherein the heterocyclyl is piperidyl or piperazinyl.

15. The compound of any one of claims 12-14, wherein L comprises one or more linking groups selected from 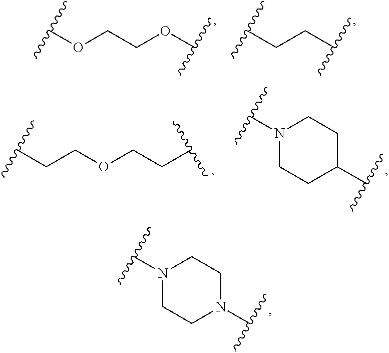
—CH₂C(O)NH—, —C(O)NH—, —C(O)CH₂—, and —OCH₂C(O)—.

16. The compound of any one of claims 12-15, wherein L comprises one or more linking groups selected from 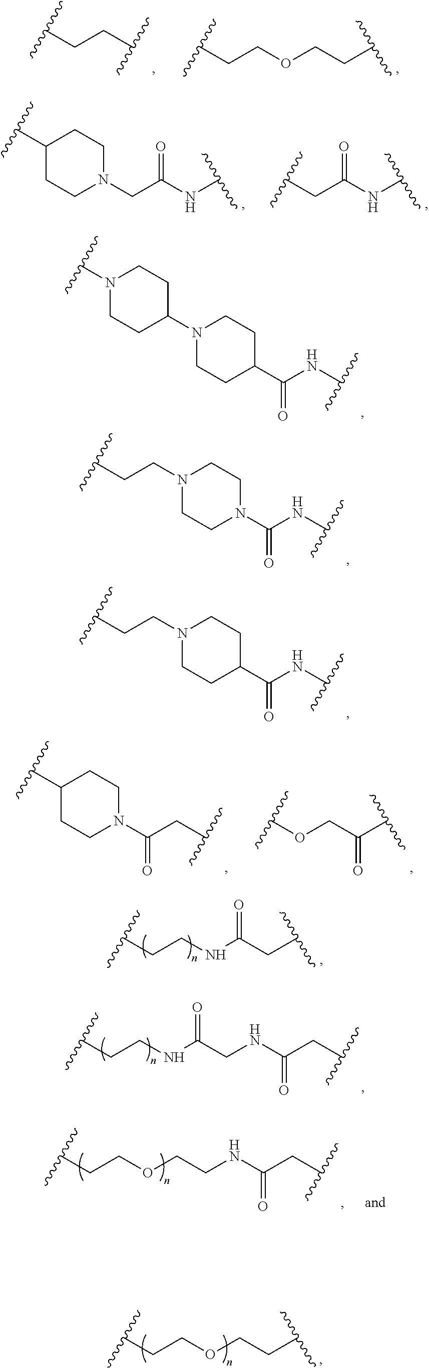
wherein n=1-5.

17. The compound of claim 1, wherein the E3 ubiquitin ligase ligand moiety comprises cereblon (CRBN) ligand, a mouse double minute 2 (MDM2) ligand, a Von Hippel-Lindau (VHL) ligand, or any substructure thereof.

18. The compound of claim 17, wherein the CRBN ligand is selected from thalidomide, lenalidomide, pomalidomide, and any substructure thereof.

19. The compound of claim 17, wherein the MDM2 ligand is selected from idasanutlin, RG7112, RG7388, MI 773/SAR 405838, AMG 232, DS-3032b, RO6839921, RO5045337, RO5503781, CGM-097, MK-8242, and any substructure thereof.

20. The compound of claim 17, wherein the VHL ligand is selected from VHL ligand 1 (VHL-1), VHL ligand 2 (VHL-2), VH032, and any substructure thereof.

21. The compound of claim 1, wherein the E3 ubiquitin ligase ligand moiety is selected from thalidomide, idasanutlin, VHL ligand 1 (VHL-1), and any substructure thereof.

22. The compound of claim 1, wherein the E3 ubiquitin ligase ligand moiety is selected from 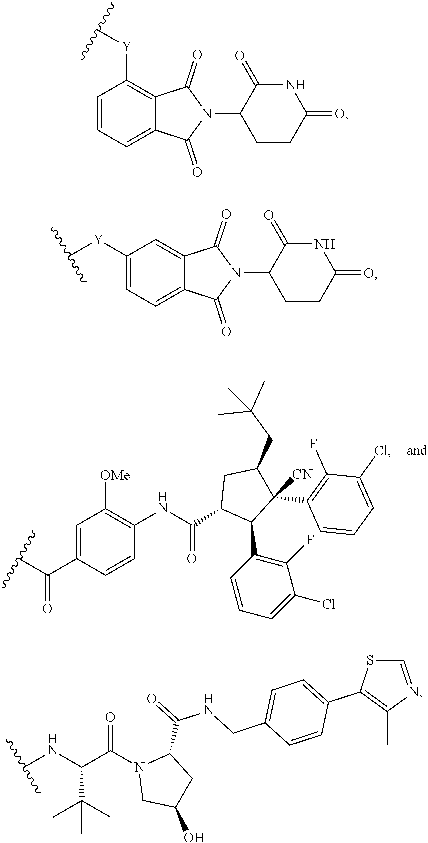
wherein Y is selected from —N(R)—, —N(H)—, and —O—, wherein R is optionally substituted alkyl.

23. The compound of claim 22, wherein the E3 ubiquitin ligase ligand moiety is selected from

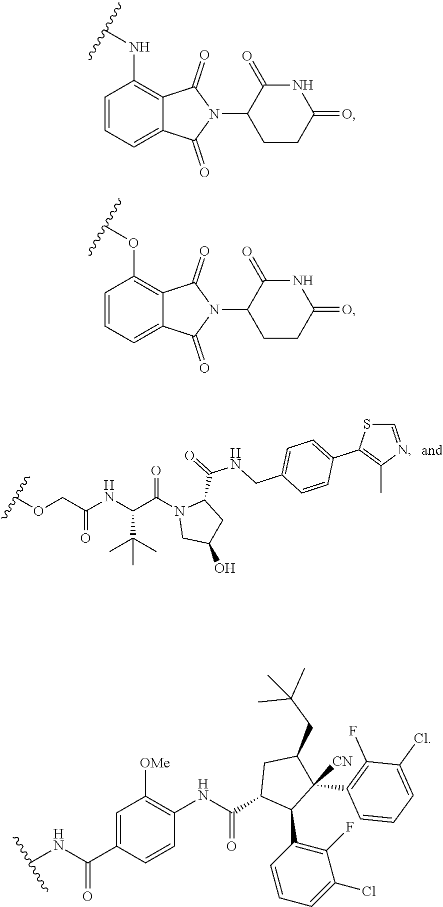 [SMILES: *Nc1cccc2c1C(=O)N(C1CCC(=O)NC1=O)C2=O ; *Oc1cccc2c1C(=O)N(C1CCC(=O)NC1=O)C2=O ; *OCC(=O)N[C@H](C(=O)N1C[C@H](O)C[C@H]1C(=O)NCc1ccc(-c2scnc2C)cc1)C(C)(C)C ; *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1]

24. The compound of any one of claims 1-23, wherein the compound has a molecular weight not greater than about 2000 g/mol, or about 1900 g/mol, or about 1800 g/mol, or about 1700 g/mol, or about 1600 g/mol, or about 1500 g/mol, or about 1400 g/mol, or about 1300 g/mol, or about 1200 g/mol or about 1100 g/mol, or about 1000 g/mol.

25. The compound of claim 1, wherein the compound of formula (I) is a compound of any one of formula 1001-1460, or a pharmaceutically acceptable salt, solvate, hydrate, cocrystal, or prodrug thereof:

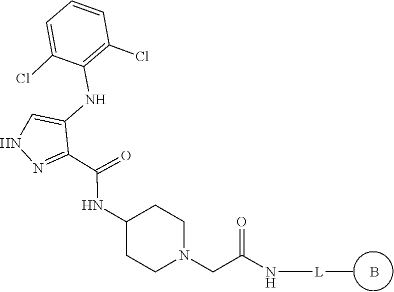

| Formula | | | |
|---|---|---|---|
| No. | L | n | B |
| 1001 | 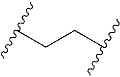 [SMILES: *CC*] | — | 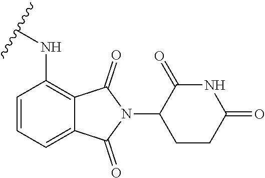 [SMILES: *Nc1cccc2c1C(=O)N(C1CCC(=O)NC1=O)C2=O] |
| 1002 | 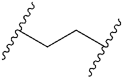 [SMILES: *CC*] | — | 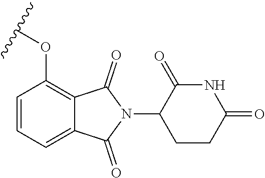 [SMILES: *Oc1cccc2c1C(=O)N(C1CCC(=O)NC1=O)C2=O] |
| 1003 | 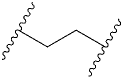 [SMILES: *CC*] | — | 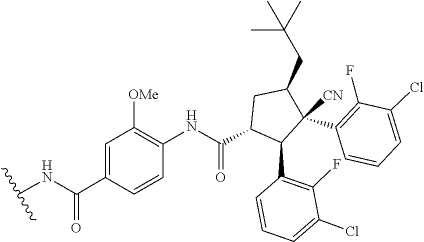 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1004 | 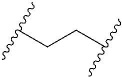 [SMILES: *CC*] | — | 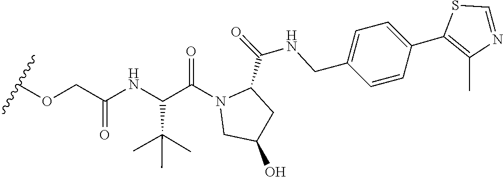 |
| 1005 | 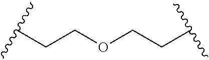 [SMILES: *CCOCC*] | — | 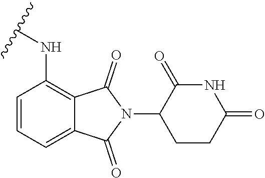 [SMILES: *Nc1cccc2c1C(=O)N(C1CCC(=O)NC1=O)C2=O] |
| 1006 | 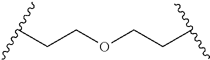 [SMILES: *CCOCC*] | — | 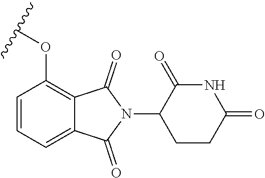 [SMILES: *Oc1cccc2c1C(=O)N(C1CCC(=O)NC1=O)C2=O] |
| 1007 | 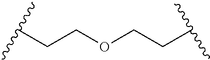 [SMILES: *CCOCC*] | — | 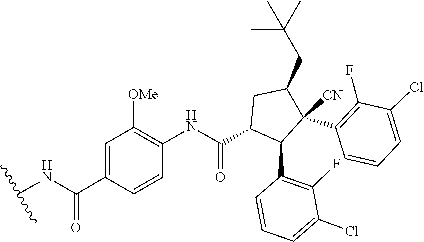 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1008 | 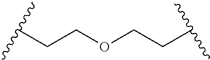 [SMILES: *CCOCC*] | — | 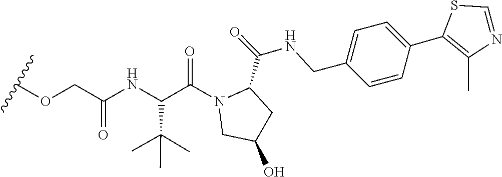 |
| 1009 | 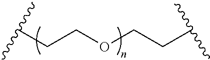 | 1 | 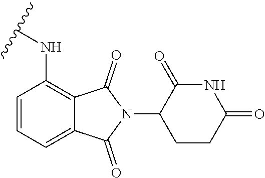 [SMILES: *Nc1cccc2c1C(=O)N(C1CCC(=O)NC1=O)C2=O] |
| 1010 | 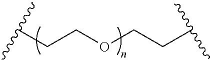 | 2 | 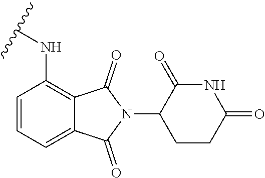 [SMILES: *Nc1cccc2c1C(=O)N(C1CCC(=O)NC1=O)C2=O] |
| 1011 | 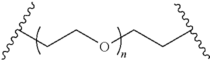 | 3 | 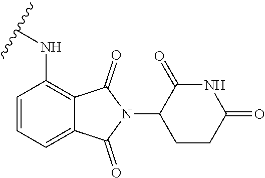 [SMILES: *Nc1cccc2c1C(=O)N(C1CCC(=O)NC1=O)C2=O] |
| 1012 | 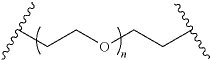 | 4 | 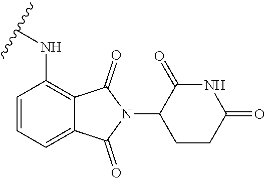 [SMILES: *Nc1cccc2c1C(=O)N(C1CCC(=O)NC1=O)C2=O] |
| 1013 | 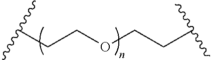 | 5 | 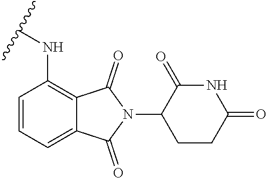 [SMILES: *Nc1cccc2c1C(=O)N(C1CCC(=O)NC1=O)C2=O] |
| 1014 | 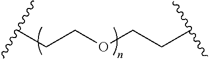 | 1 | 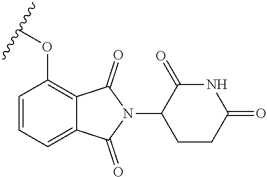 [SMILES: *Oc1cccc2c1C(=O)N(C1CCC(=O)NC1=O)C2=O] |
| 1015 | 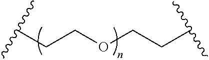 | 2 | 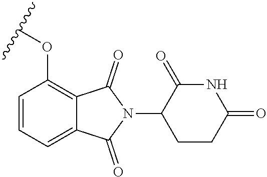 [SMILES: *Oc1cccc2c1C(=O)N(C1CCC(=O)NC1=O)C2=O] |
| 1016 | 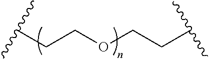 | 3 | 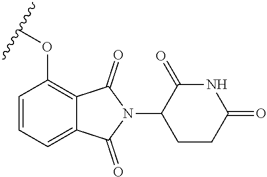 [SMILES: *Oc1cccc2c1C(=O)N(C1CCC(=O)NC1=O)C2=O] |
| 1017 | 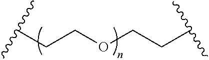 | 4 | 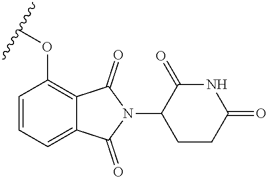 [SMILES: *Oc1cccc2c1C(=O)N(C1CCC(=O)NC1=O)C2=O] |
| 1018 | 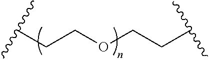 | 5 | 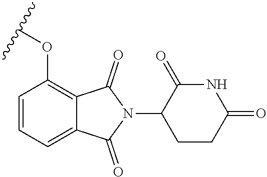 [SMILES: *Oc1cccc2c1C(=O)N(C1CCC(=O)NC1=O)C2=O] |
| 1019 | 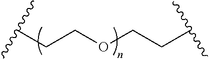 | 1 | 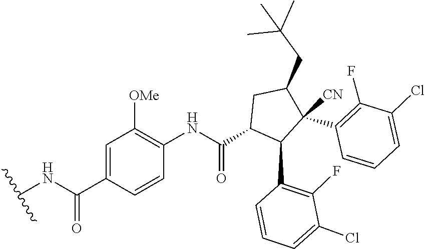 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1020 | 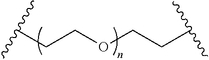 | 2 | 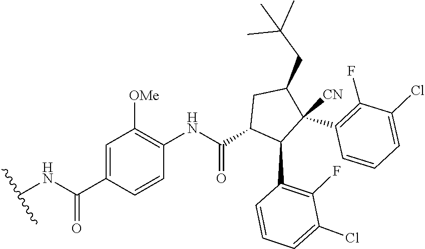 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1021 | 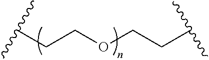 | 3 | 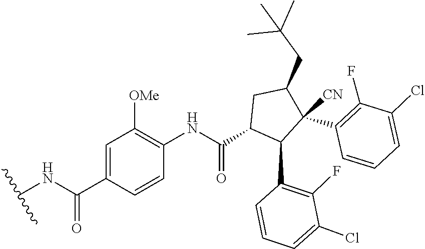 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1022 | 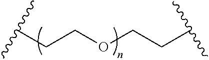 | 4 | 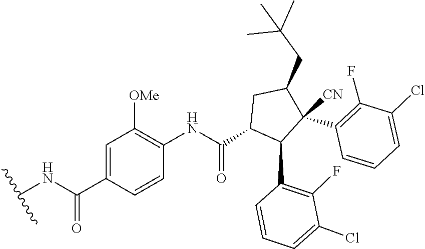 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1023 | 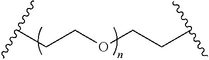 | 5 | 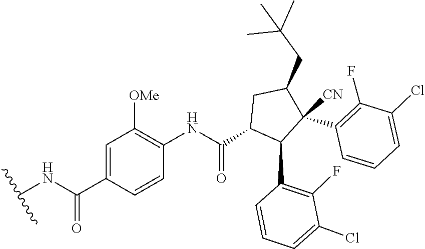 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1024 | 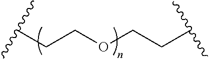 | 1 | 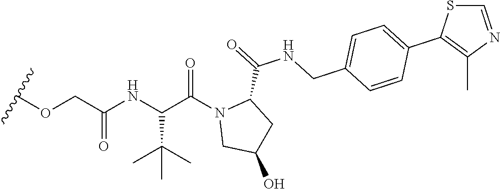 |
| 1025 | 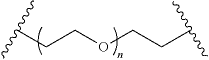 | 2 | 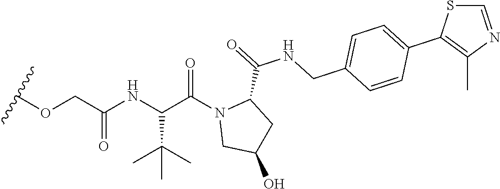 |
| 1026 | 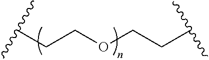 | 3 | 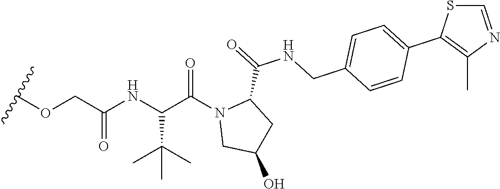 |
| 1027 | 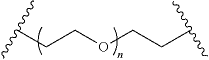 | 4 | 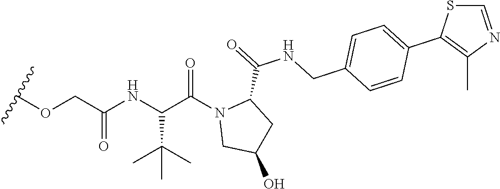 |
| 1028 | 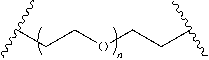 | 5 | 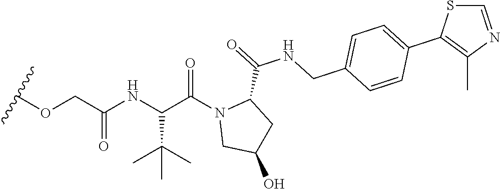 |
| 1029 | 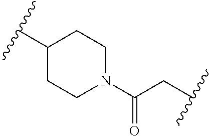 [SMILES: *CC(=O)N1CCC(*)CC1] | — | 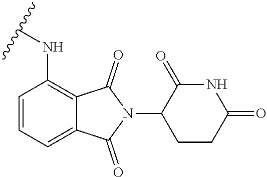 [SMILES: *Nc1cccc2c1C(=O)N(C1CCC(=O)NC1=O)C2=O] |
| 1030 | 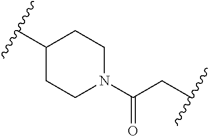 [SMILES: *CC(=O)N1CCC(*)CC1] | — | 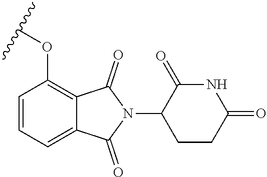 [SMILES: *Oc1cccc2c1C(=O)N(C1CCC(=O)NC1=O)C2=O] |
| 1031 | 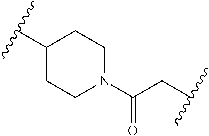 [SMILES: *CC(=O)N1CCC(*)CC1] | — | 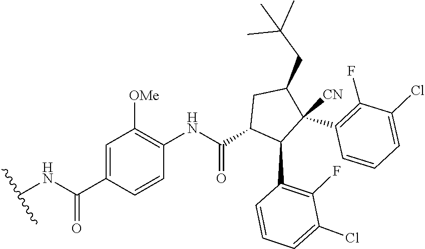 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1032 | 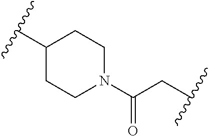 [SMILES: *CC(=O)N1CCC(*)CC1] | — | 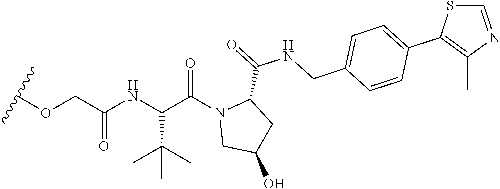 |
| 1033 | 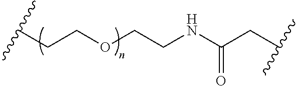 | 1 | 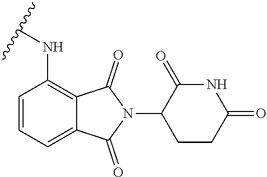 [SMILES: *Nc1cccc2c1C(=O)N(C1CCC(=O)NC1=O)C2=O] |
| 1034 | 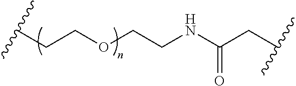 | 2 | 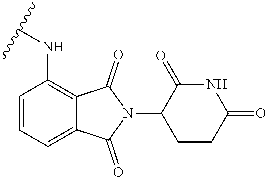 [SMILES: *Nc1cccc2c1C(=O)N(C1CCC(=O)NC1=O)C2=O] |
| 1035 | 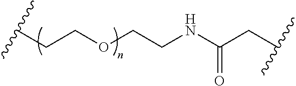 | 3 | 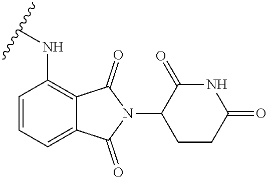 [SMILES: *Nc1cccc2c1C(=O)N(C1CCC(=O)NC1=O)C2=O] |
| 1036 | 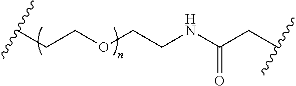 | 4 | 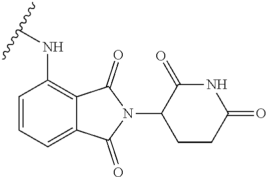 [SMILES: *Nc1cccc2c1C(=O)N(C1CCC(=O)NC1=O)C2=O] |
| 1037 | 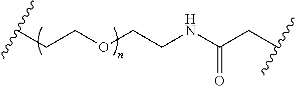 | 5 | 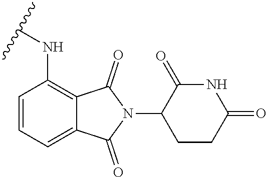 [SMILES: *Nc1cccc2c1C(=O)N(C1CCC(=O)NC1=O)C2=O] |
| 1038 | 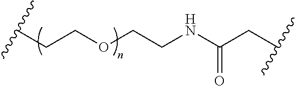 | 1 | 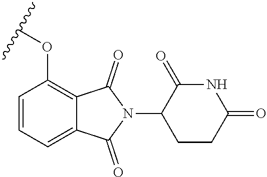 [SMILES: *Oc1cccc2c1C(=O)N(C1CCC(=O)NC1=O)C2=O] |
| 1039 | 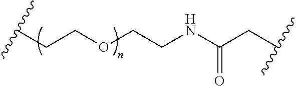 | 2 | 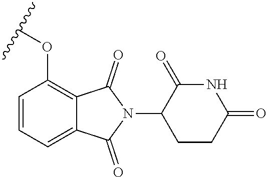 [SMILES: *Oc1cccc2c1C(=O)N(C1CCC(=O)NC1=O)C2=O] |
| 1040 | 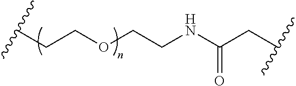 | 3 | 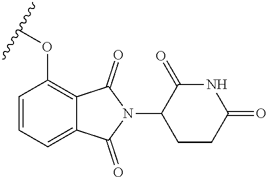 [SMILES: *Oc1cccc2c1C(=O)N(C1CCC(=O)NC1=O)C2=O] |
| 1041 | 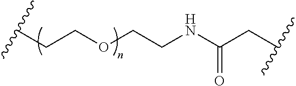 | 4 | 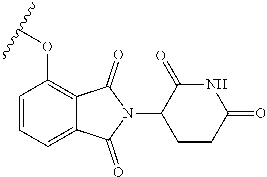 [SMILES: *Oc1cccc2c1C(=O)N(C1CCC(=O)NC1=O)C2=O] |
| 1042 | 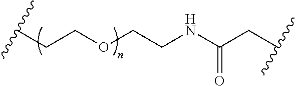 | 5 | 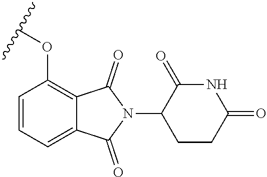 [SMILES: *Oc1cccc2c1C(=O)N(C1CCC(=O)NC1=O)C2=O] |
| 1043 | 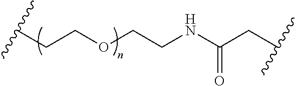 | 1 | 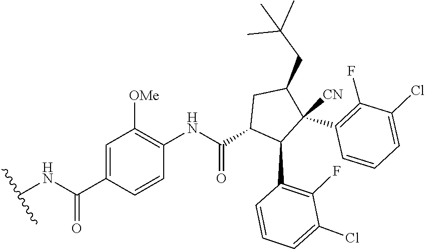 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1044 | 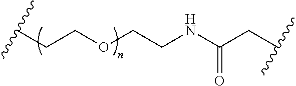 | 2 | 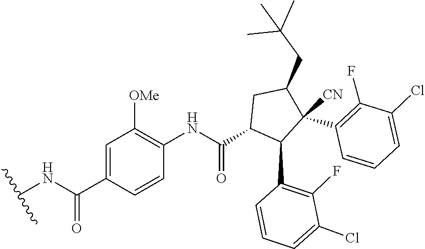 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1045 | 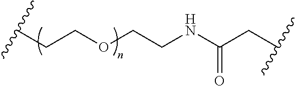 | 3 | 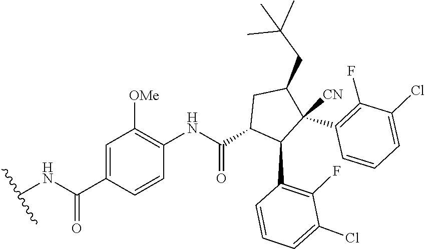 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1046 | 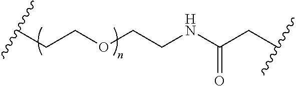 | 4 | 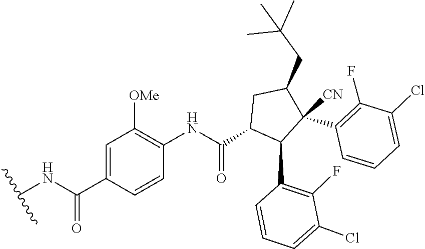 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1047 | 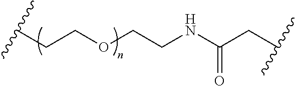 | 5 | 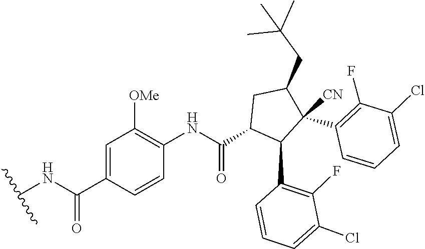 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1048 | 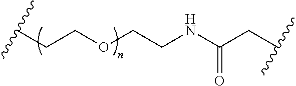 | 1 | 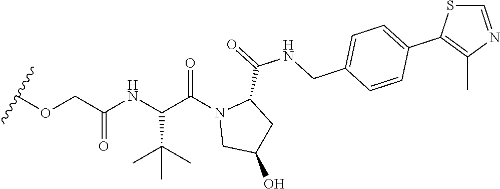 |
| 1049 | 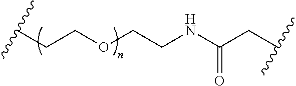 | 2 | 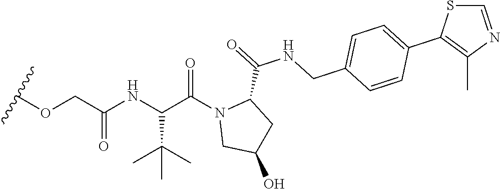 |
| 1050 | 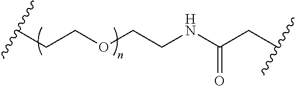 | 3 | 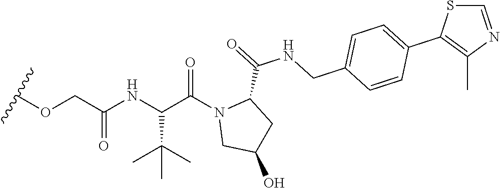 |
| 1051 | 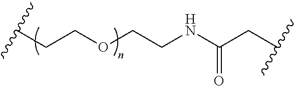 | 4 | 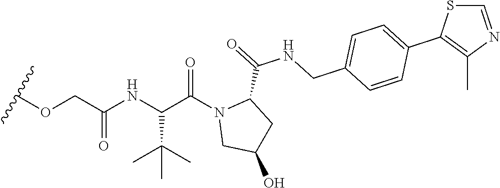 |
| 1052 | 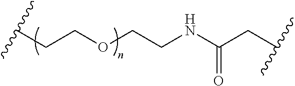 | 5 | 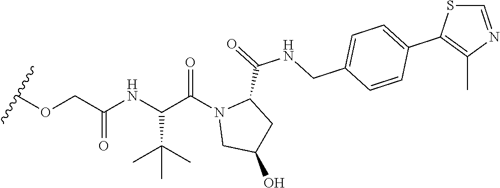 |
| 1053 | 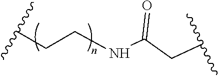 | 1 | 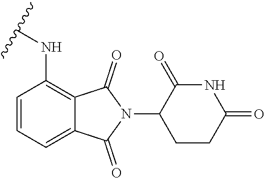 [SMILES: *Nc1cccc2c1C(=O)N(C1CCC(=O)NC1=O)C2=O] |
| 1054 | 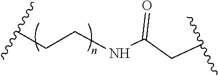 | 2 | 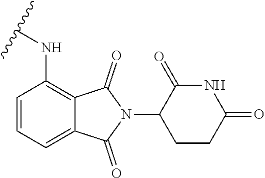 [SMILES: *Nc1cccc2c1C(=O)N(C1CCC(=O)NC1=O)C2=O] |
| 1055 | 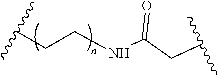 | 3 | 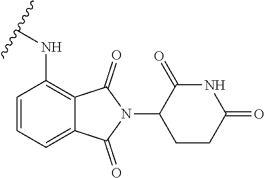 [SMILES: *Nc1cccc2c1C(=O)N(C1CCC(=O)NC1=O)C2=O] |
| 1056 | 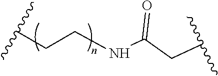 | 4 | 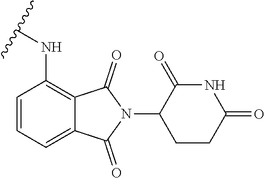 [SMILES: *Nc1cccc2c1C(=O)N(C1CCC(=O)NC1=O)C2=O] |
| 1057 | 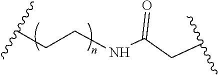 | 5 | 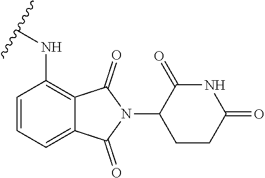 [SMILES: *Nc1cccc2c1C(=O)N(C1CCC(=O)NC1=O)C2=O] |
| 1058 | 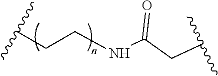 | 1 | 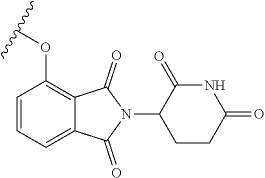 [SMILES: *Oc1cccc2c1C(=O)N(C1CCC(=O)NC1=O)C2=O] |
| 1059 | 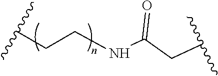 | 2 | 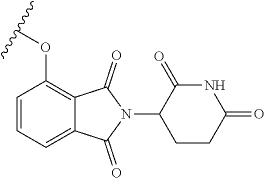 [SMILES: *Oc1cccc2c1C(=O)N(C1CCC(=O)NC1=O)C2=O] |
| 1060 | 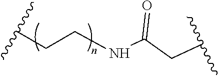 | 3 | 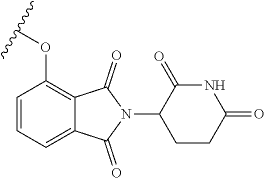 [SMILES: *Oc1cccc2c1C(=O)N(C1CCC(=O)NC1=O)C2=O] |
| 1061 | 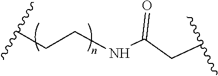 | 4 | 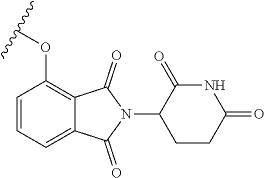 [SMILES: *Oc1cccc2c1C(=O)N(C1CCC(=O)NC1=O)C2=O] |
| 1062 | 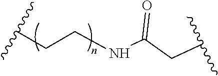 | 5 | 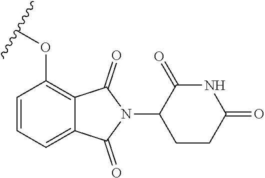 [SMILES: *Oc1cccc2c1C(=O)N(C1CCC(=O)NC1=O)C2=O] |
| 1063 | 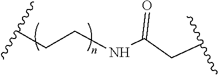 | 1 | 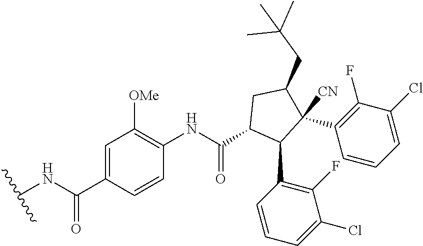 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1064 | 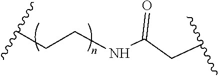 | 2 | 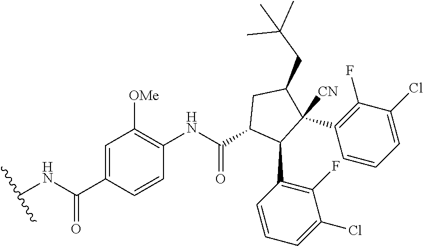 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1065 | 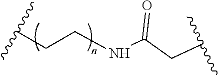 | 3 | 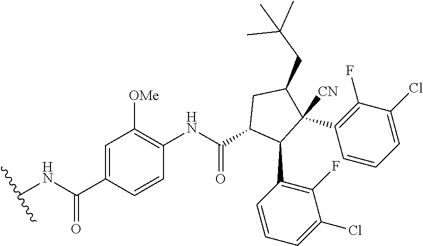 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1066 | 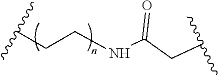 | 4 | 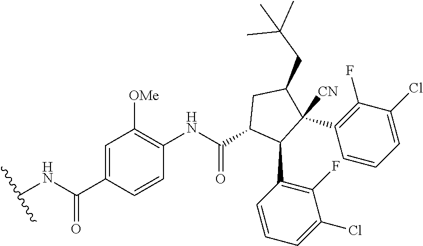 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1067 | 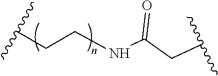 | 5 | 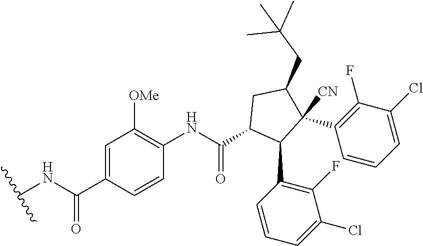 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1068 | 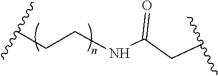 | 1 | 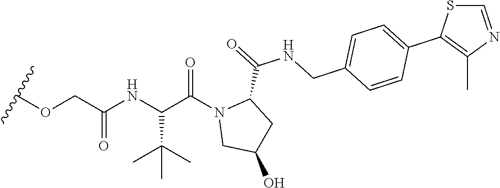 |
| 1069 | 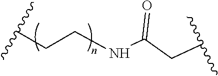 | 2 | 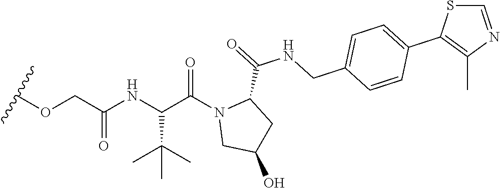 |
| 1070 | 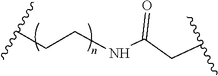 | 3 | 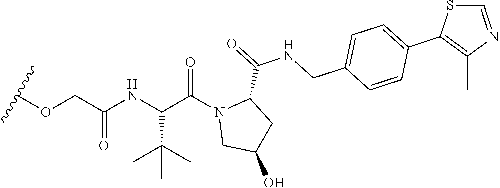 |
| 1071 | 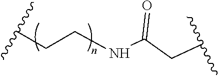 | 4 | 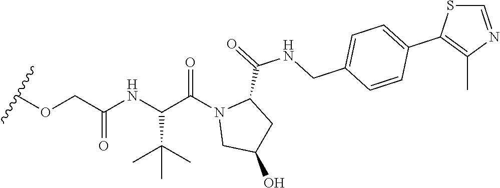 |
| 1072 | 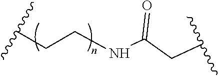 | 5 | 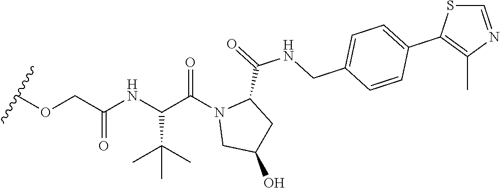 |
| 1073 | 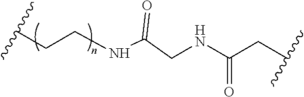 | 1 | 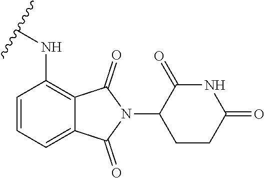 [SMILES: *Nc1cccc2c1C(=O)N(C1CCC(=O)NC1=O)C2=O] |
| 1074 | 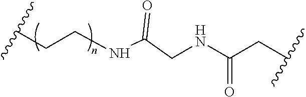 | 2 | 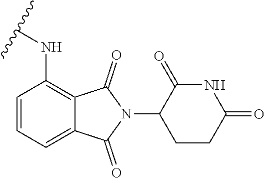 [SMILES: *Nc1cccc2c1C(=O)N(C1CCC(=O)NC1=O)C2=O] |
| 1075 | 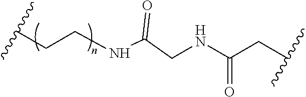 | 3 | 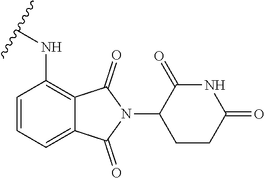 [SMILES: *Nc1cccc2c1C(=O)N(C1CCC(=O)NC1=O)C2=O] |
| 1076 | 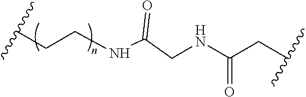 | 4 | 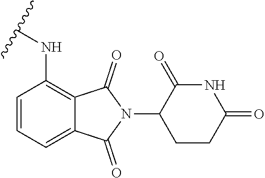 [SMILES: *Nc1cccc2c1C(=O)N(C1CCC(=O)NC1=O)C2=O] |
| 1077 | 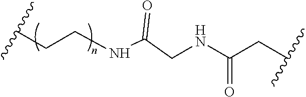 | 5 | 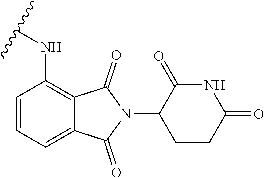 [SMILES: *Nc1cccc2c1C(=O)N(C1CCC(=O)NC1=O)C2=O] |
| 1078 | 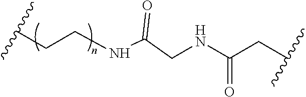 | 1 | 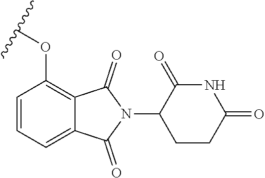 [SMILES: *Oc1cccc2c1C(=O)N(C1CCC(=O)NC1=O)C2=O] |
| 1079 | 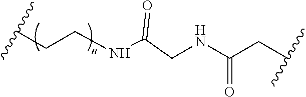 | 2 | 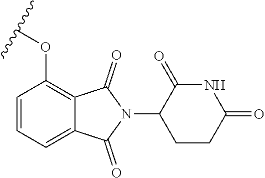 [SMILES: *Oc1cccc2c1C(=O)N(C1CCC(=O)NC1=O)C2=O] |
| 1080 | 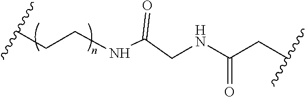 | 3 | 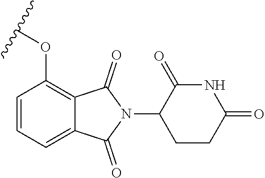 [SMILES: *Oc1cccc2c1C(=O)N(C1CCC(=O)NC1=O)C2=O] |
| 1081 | 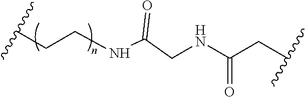 | 4 | 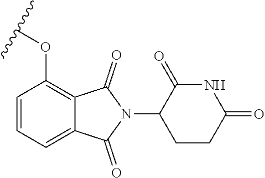 [SMILES: *Oc1cccc2c1C(=O)N(C1CCC(=O)NC1=O)C2=O] |
| 1082 | 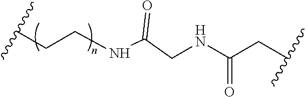 | 5 | 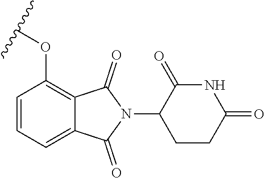 [SMILES: *Oc1cccc2c1C(=O)N(C1CCC(=O)NC1=O)C2=O] |
| 1083 | 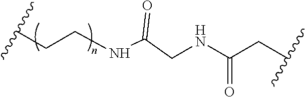 | 1 | 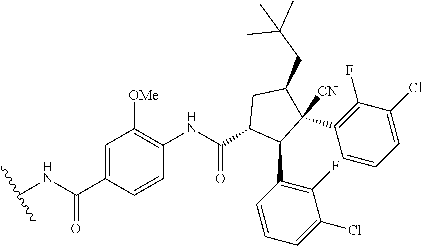 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1084 | 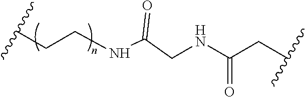 | 2 | 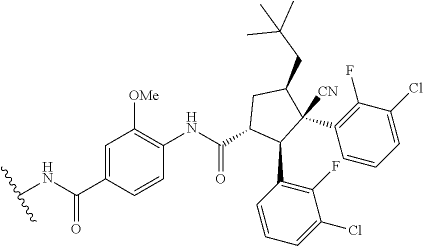 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1085 | 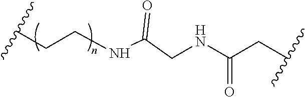 | 3 | 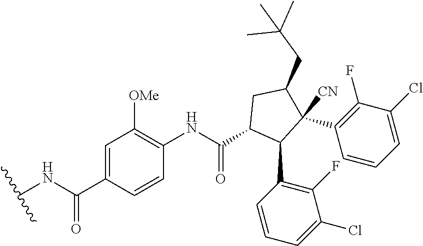 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1086 | 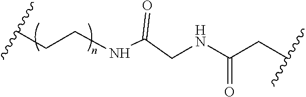 | 4 | 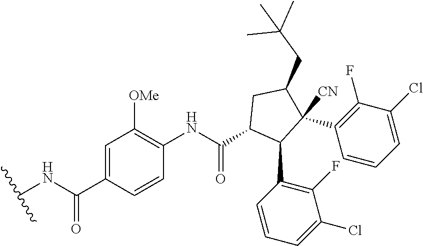 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1087 | 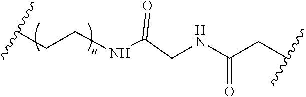 | 5 | 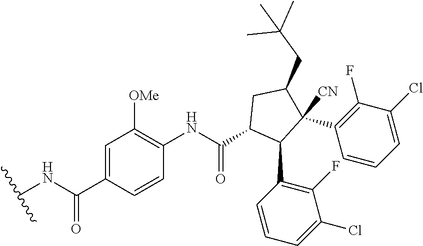 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1088 | 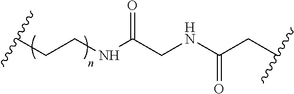 | 1 | 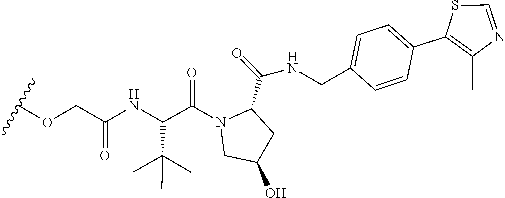 |
| 1089 | 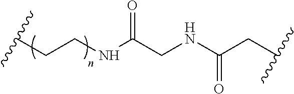 | 2 | 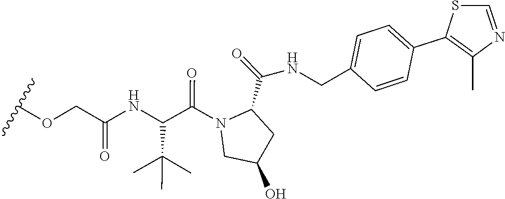 |
| 1090 | 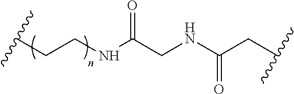 | 3 | 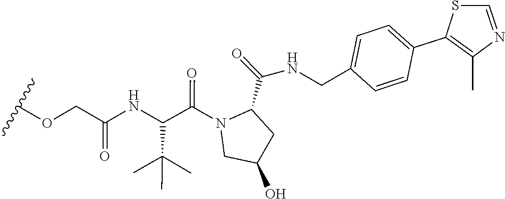 |
| 1091 | 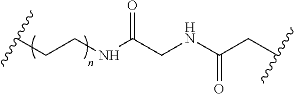 | 4 | 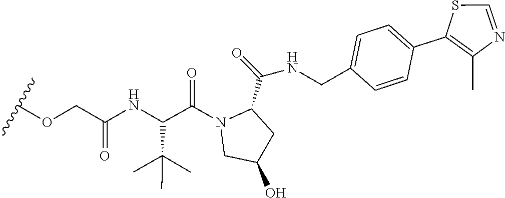 |
| 1092 | 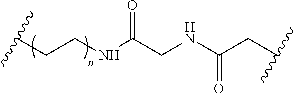 | 5 | 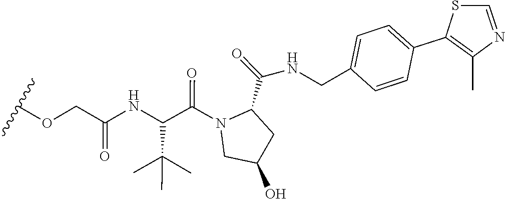 |

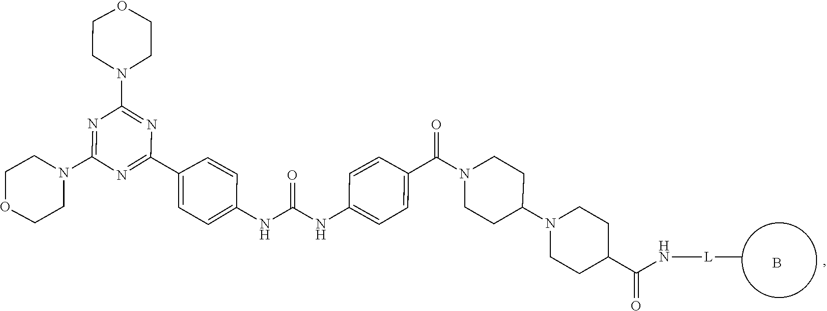
| Formula | | | |
|---|---|---|---|
| No. | L | n | B |
| 1093 | 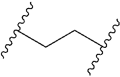 [SMILES: *CC*] | — | 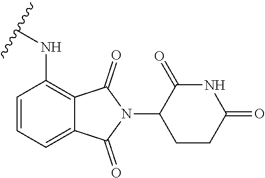 [SMILES: *Nc1cccc2c1C(=O)N(C1CCC(=O)NC1=O)C2=O] |
| 1094 | 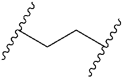 [SMILES: *CC*] | — | 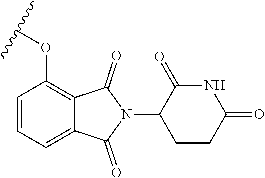 [SMILES: *Oc1cccc2c1C(=O)N(C1CCC(=O)NC1=O)C2=O] |
| 1095 | 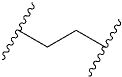 [SMILES: *CC*] | — | 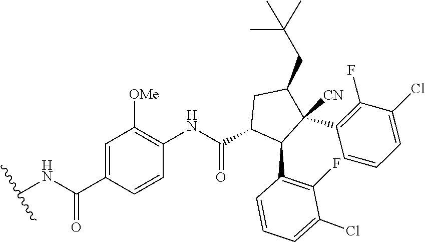 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1096 | 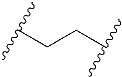 [SMILES: *CC*] | — | 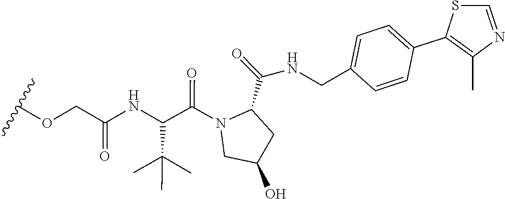 |
| 1097 | 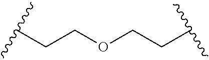 [SMILES: *CCOCC*] | — | 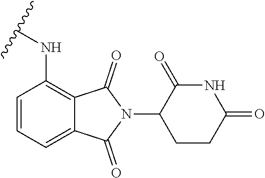 [SMILES: *Nc1cccc2c1C(=O)N(C1CCC(=O)NC1=O)C2=O] |
| 1098 | 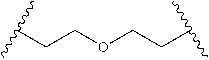 [SMILES: *CCOCC*] | — | 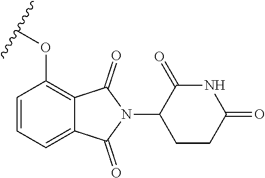 [SMILES: *Oc1cccc2c1C(=O)N(C1CCC(=O)NC1=O)C2=O] |
| 1099 | 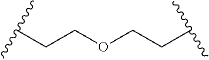 [SMILES: *CCOCC*] | — | 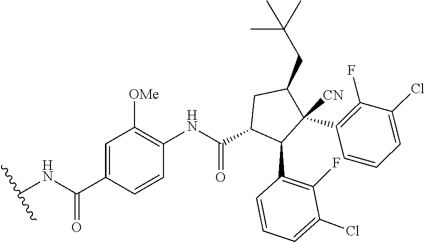 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1100 | 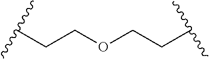 [SMILES: *CCOCC*] | — | 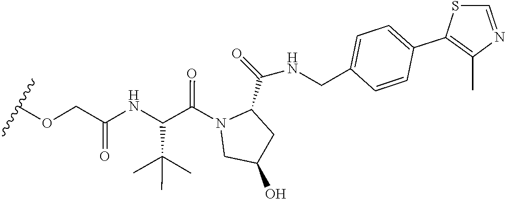 |
| 1101 | 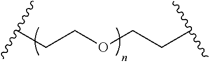 | 1 | 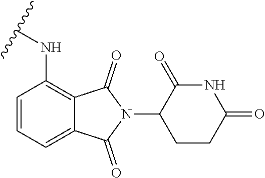 [SMILES: *Nc1cccc2c1C(=O)N(C1CCC(=O)NC1=O)C2=O] |
| 1102 | 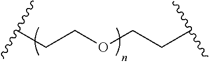 | 2 | 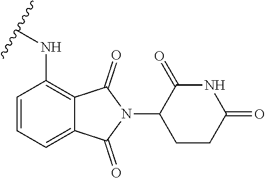 [SMILES: *Nc1cccc2c1C(=O)N(C1CCC(=O)NC1=O)C2=O] |
| 1103 | 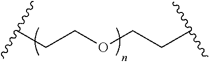 | 3 | 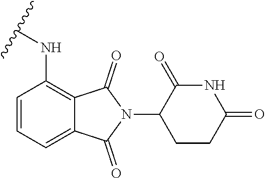 [SMILES: *Nc1cccc2c1C(=O)N(C1CCC(=O)NC1=O)C2=O] |
| 1104 | 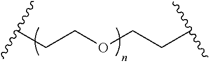 | 4 | 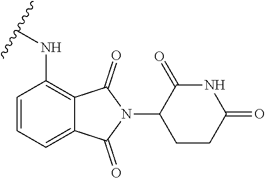 [SMILES: *Nc1cccc2c1C(=O)N(C1CCC(=O)NC1=O)C2=O] |
| 1105 | 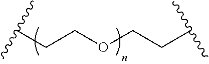 | 5 | 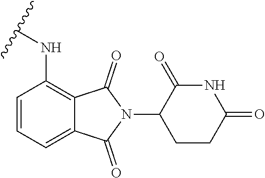 [SMILES: *Nc1cccc2c1C(=O)N(C1CCC(=O)NC1=O)C2=O] |
| 1106 | 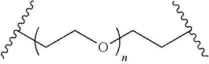 | 1 | 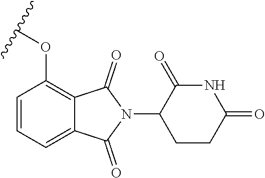 [SMILES: *Oc1cccc2c1C(=O)N(C1CCC(=O)NC1=O)C2=O] |
| 1107 | 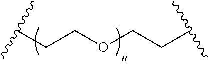 | 2 | 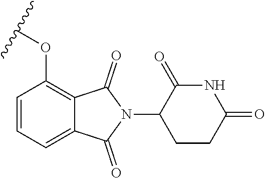 [SMILES: *Oc1cccc2c1C(=O)N(C1CCC(=O)NC1=O)C2=O] |
| 1108 | 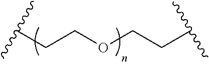 | 3 | 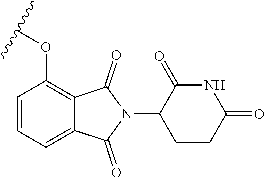 [SMILES: *Oc1cccc2c1C(=O)N(C1CCC(=O)NC1=O)C2=O] |
| 1109 | 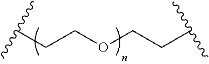 | 4 | 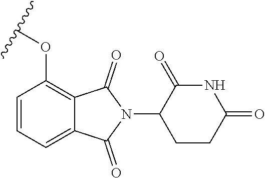 [SMILES: *Oc1cccc2c1C(=O)N(C1CCC(=O)NC1=O)C2=O] |
| 1110 | 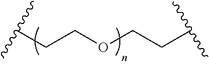 | 5 | 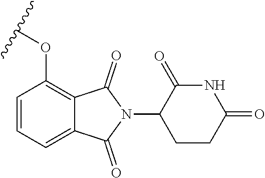 [SMILES: *Oc1cccc2c1C(=O)N(C1CCC(=O)NC1=O)C2=O] |
| 1111 | 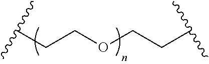 | 1 | 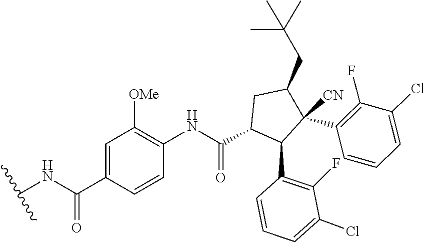 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1112 | 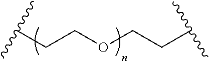 | 2 | 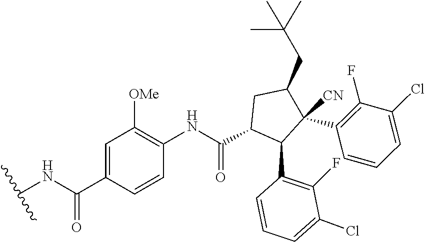 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1113 | 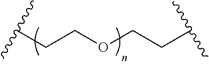 | 3 | 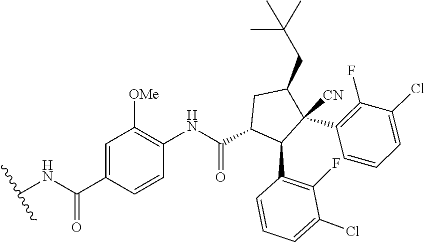 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1114 | 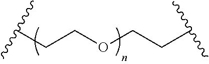 | 4 | 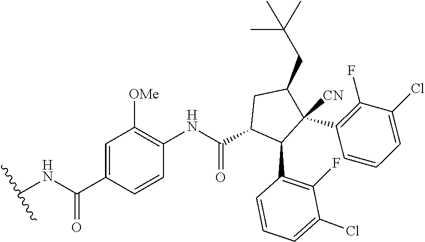 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1115 | 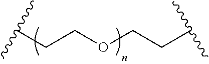 | 5 | 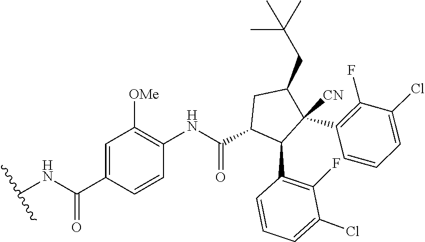 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1116 | 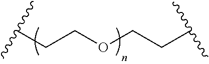 | 1 | 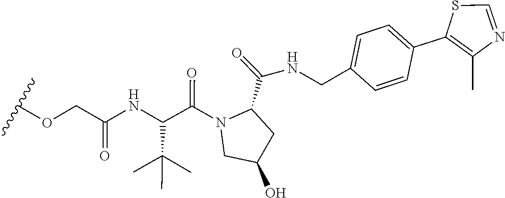 |
| 1117 | 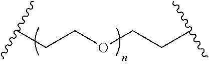 | 2 | 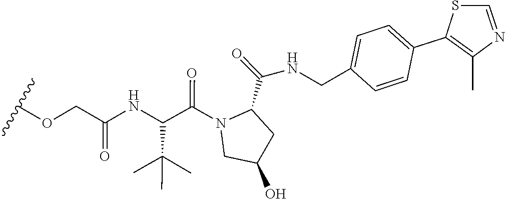 |
| 1118 | 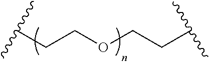 | 3 | 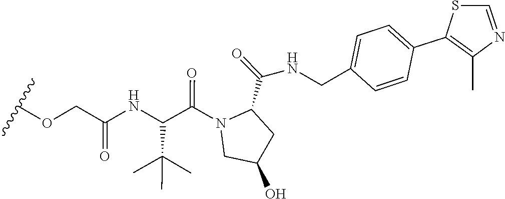 |
| 1119 | 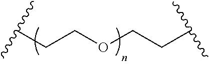 | 4 | 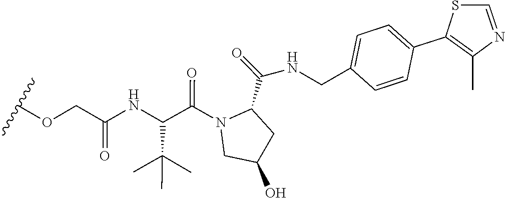 |
| 1120 | 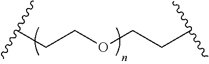 | 5 | 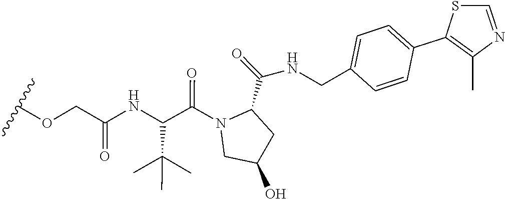 |
| 1121 | 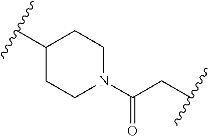 [SMILES: *CC(=O)N1CCC(*)CC1] | — | 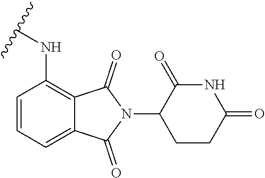 [SMILES: *Nc1cccc2c1C(=O)N(C1CCC(=O)NC1=O)C2=O] |
| 1122 | 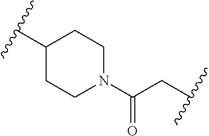 [SMILES: *CC(=O)N1CCC(*)CC1] | — | 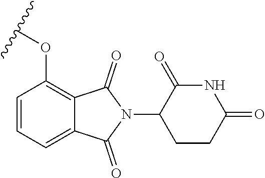 [SMILES: *Oc1cccc2c1C(=O)N(C1CCC(=O)NC1=O)C2=O] |
| 1123 | 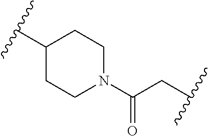 [SMILES: *CC(=O)N1CCC(*)CC1] | — | 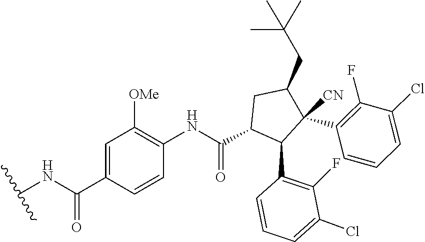 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1124 | 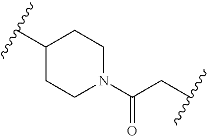 [SMILES: *CC(=O)N1CCC(*)CC1] | — | 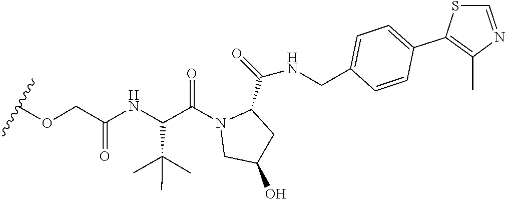 |
| 1125 | 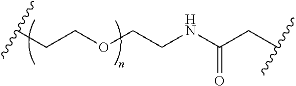 | 1 | 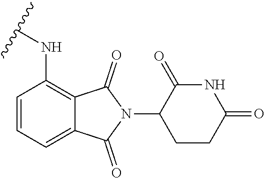 [SMILES: *Nc1cccc2c1C(=O)N(C1CCC(=O)NC1=O)C2=O] |
| 1126 | 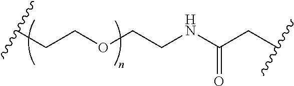 | 2 | 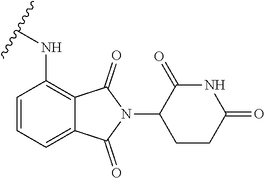 [SMILES: *Nc1cccc2c1C(=O)N(C1CCC(=O)NC1=O)C2=O] |
| 1127 | 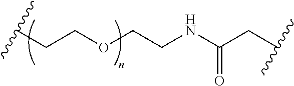 | 3 | 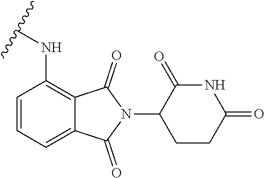 [SMILES: *Nc1cccc2c1C(=O)N(C1CCC(=O)NC1=O)C2=O] |
| 1128 | 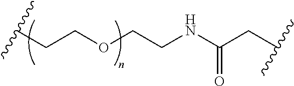 | 4 | 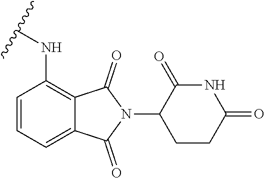 [SMILES: *Nc1cccc2c1C(=O)N(C1CCC(=O)NC1=O)C2=O] |
| 1129 | 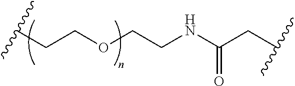 | 5 | 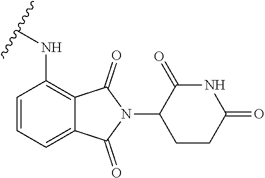 [SMILES: *Nc1cccc2c1C(=O)N(C1CCC(=O)NC1=O)C2=O] |
| 1130 | 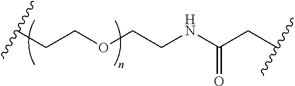 | 1 | 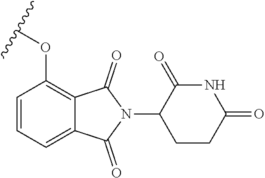 [SMILES: *Oc1cccc2c1C(=O)N(C1CCC(=O)NC1=O)C2=O] |
| 1131 | 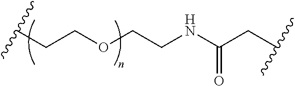 | 2 | 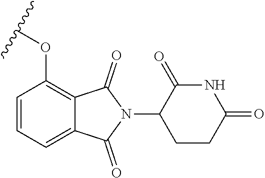 [SMILES: *Oc1cccc2c1C(=O)N(C1CCC(=O)NC1=O)C2=O] |
| 1132 | 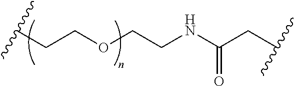 | 3 | 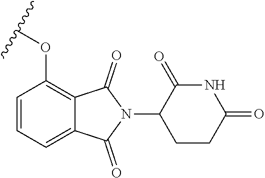 [SMILES: *Oc1cccc2c1C(=O)N(C1CCC(=O)NC1=O)C2=O] |
| 1133 | 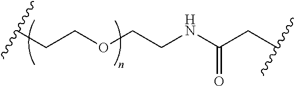 | 4 | 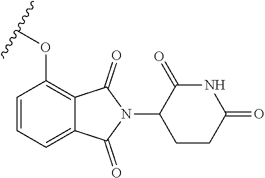 [SMILES: *Oc1cccc2c1C(=O)N(C1CCC(=O)NC1=O)C2=O] |
| 1134 | 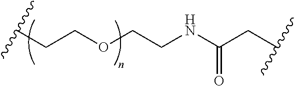 | 5 | 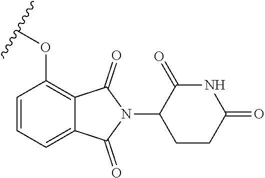 [SMILES: *Oc1cccc2c1C(=O)N(C1CCC(=O)NC1=O)C2=O] |
| 1135 | 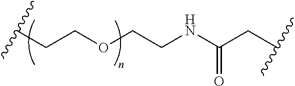 | 1 | 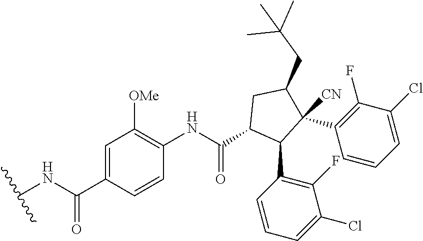 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1136 | 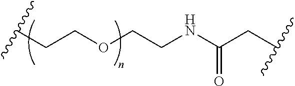 | 2 | 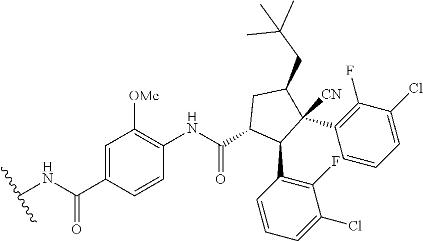 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1137 | 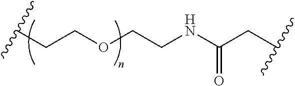 | 3 | 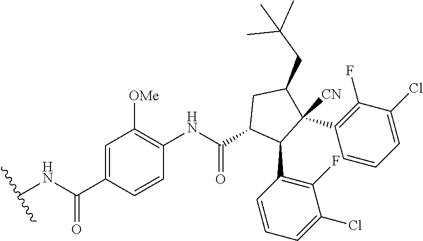 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1138 | 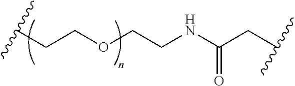 | 4 | 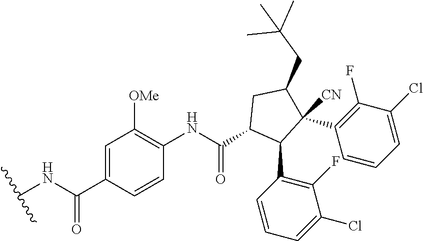 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1139 | 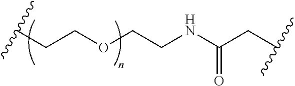 | 5 | 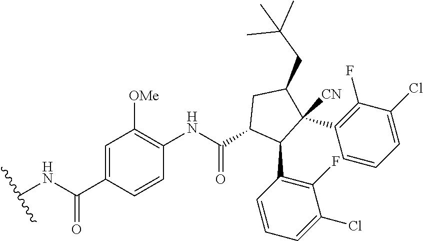 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1140 | 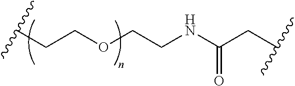 | 1 | 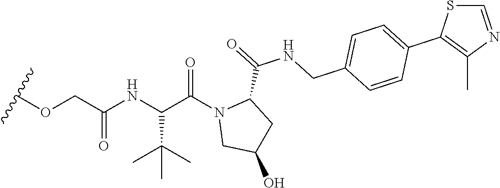 |
| 1141 | 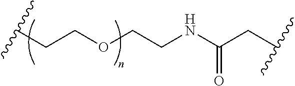 | 2 | 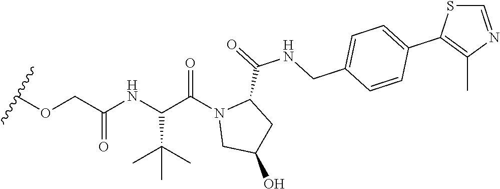 |
| 1142 | 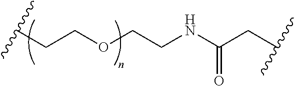 | 3 | 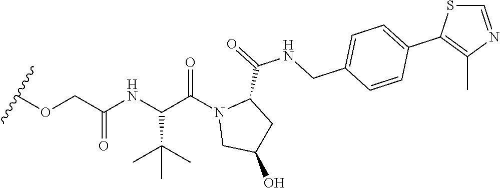 |
| 1143 | 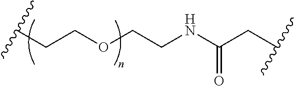 | 4 | 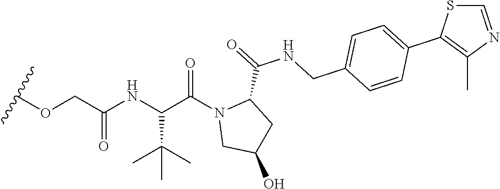 |
| 1144 | 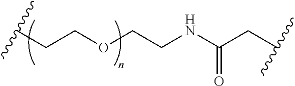 | 5 | 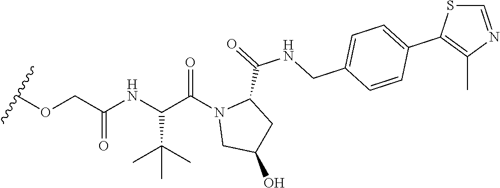 |
| 1145 | 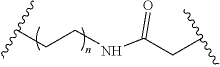 | 1 | 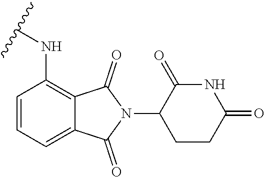 [SMILES: *Nc1cccc2c1C(=O)N(C1CCC(=O)NC1=O)C2=O] |
| 1146 | 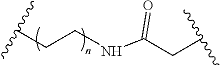 | 2 | 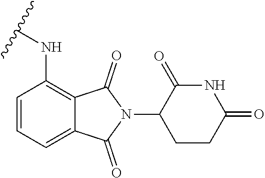 [SMILES: *Nc1cccc2c1C(=O)N(C1CCC(=O)NC1=O)C2=O] |
| 1147 | 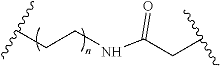 | 3 | 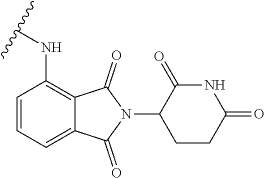 [SMILES: *Nc1cccc2c1C(=O)N(C1CCC(=O)NC1=O)C2=O] |
| 1148 | 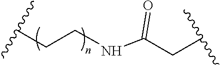 | 4 | 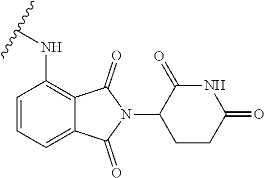 [SMILES: *Nc1cccc2c1C(=O)N(C1CCC(=O)NC1=O)C2=O] |
| 1149 | 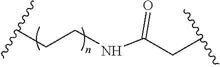 | 5 | 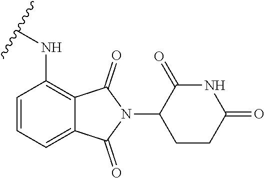 [SMILES: *Nc1cccc2c1C(=O)N(C1CCC(=O)NC1=O)C2=O] |
| 1150 | 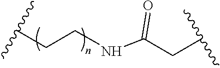 | 1 | 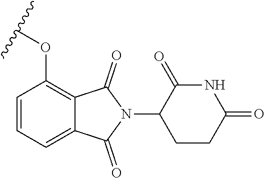 [SMILES: *Oc1cccc2c1C(=O)N(C1CCC(=O)NC1=O)C2=O] |
| 1151 | 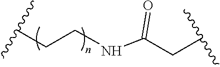 | 2 | 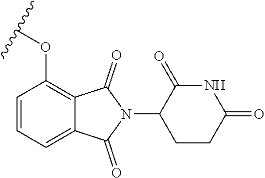 [SMILES: *Oc1cccc2c1C(=O)N(C1CCC(=O)NC1=O)C2=O] |
| 1152 | 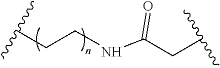 | 3 | 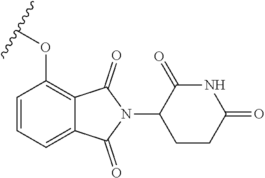 [SMILES: *Oc1cccc2c1C(=O)N(C1CCC(=O)NC1=O)C2=O] |
| 1153 | 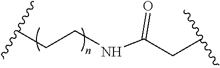 | 4 | 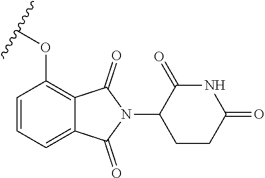 [SMILES: *Oc1cccc2c1C(=O)N(C1CCC(=O)NC1=O)C2=O] |
| 1154 | 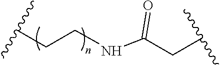 | 5 | 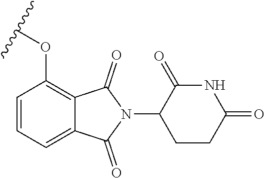 [SMILES: *Oc1cccc2c1C(=O)N(C1CCC(=O)NC1=O)C2=O] |
| 1155 | 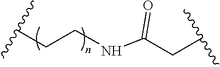 | 1 | 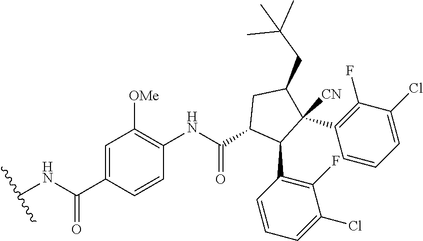 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1156 | 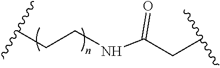 | 2 | 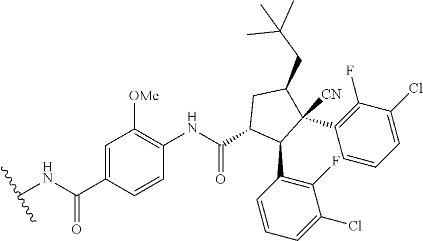 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1157 | 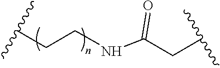 | 3 | 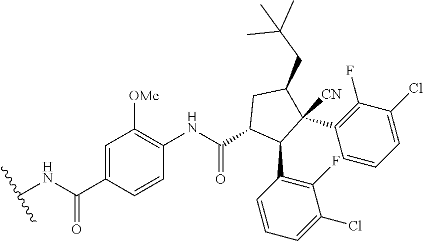 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1158 | 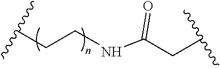 | 4 | 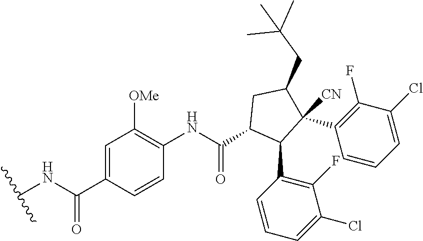 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1159 | 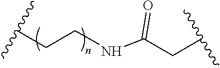 | 5 | 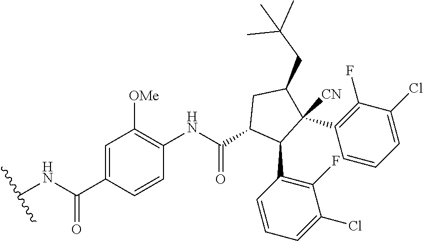 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1160 | 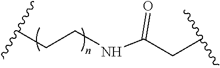 | 1 | 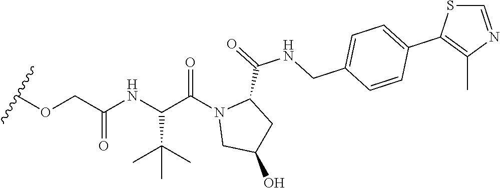 |
| 1161 | 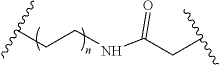 | 2 | 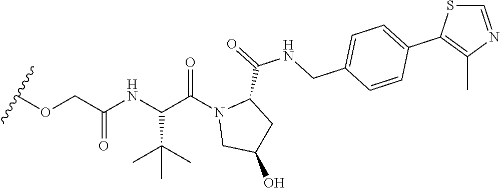 |
| 1162 | 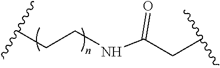 | 3 | 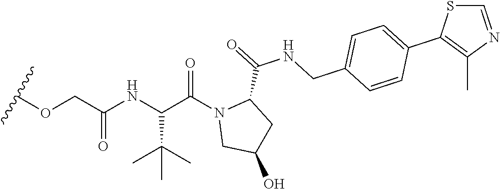 |
| 1163 | 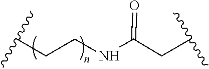 | 4 | 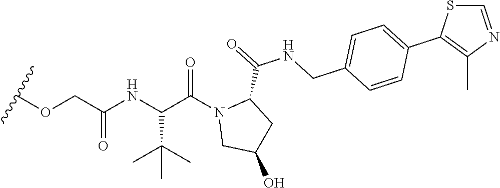 |
| 1164 | 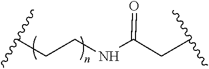 | 5 | 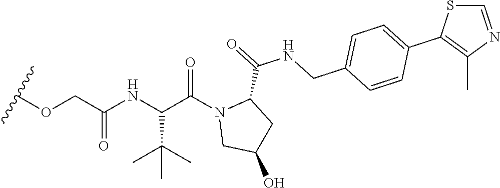 |
| 1165 | 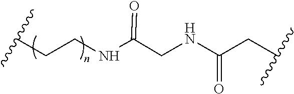 | 1 | 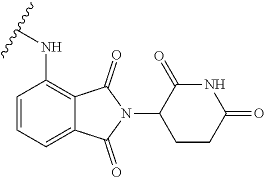 [SMILES: *Nc1cccc2c1C(=O)N(C1CCC(=O)NC1=O)C2=O] |
| 1166 | 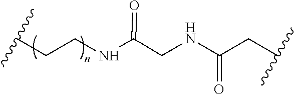 | 2 | 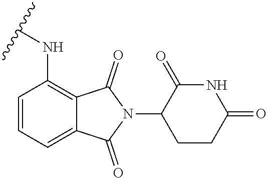 [SMILES: *Nc1cccc2c1C(=O)N(C1CCC(=O)NC1=O)C2=O] |
| 1167 | 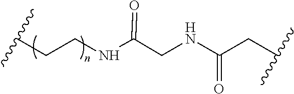 | 3 | 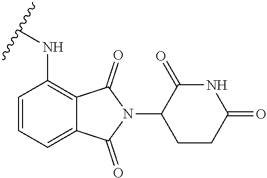 [SMILES: *Nc1cccc2c1C(=O)N(C1CCC(=O)NC1=O)C2=O] |
| 1168 | 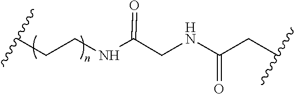 | 4 | 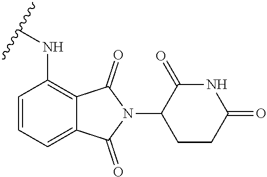 [SMILES: *Nc1cccc2c1C(=O)N(C1CCC(=O)NC1=O)C2=O] |
| 1169 | 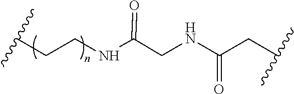 | 5 | 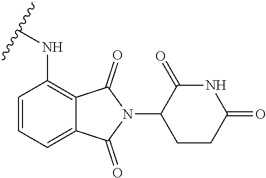 [SMILES: *Nc1cccc2c1C(=O)N(C1CCC(=O)NC1=O)C2=O] |
| 1170 | 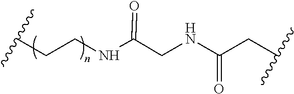 | 1 | 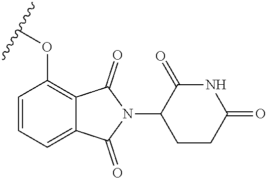 [SMILES: *Oc1cccc2c1C(=O)N(C1CCC(=O)NC1=O)C2=O] |
| 1171 | 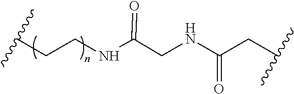 | 2 | 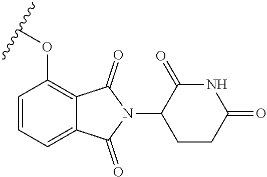 [SMILES: *Oc1cccc2c1C(=O)N(C1CCC(=O)NC1=O)C2=O] |
| 1172 | 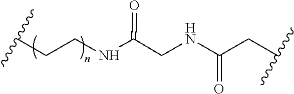 | 3 | 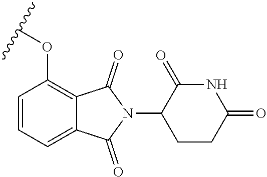 [SMILES: *Oc1cccc2c1C(=O)N(C1CCC(=O)NC1=O)C2=O] |
| 1173 | 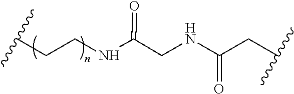 | 4 | 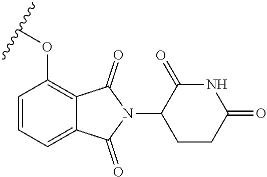 [SMILES: *Oc1cccc2c1C(=O)N(C1CCC(=O)NC1=O)C2=O] |
| 1174 | 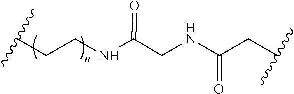 | 5 | 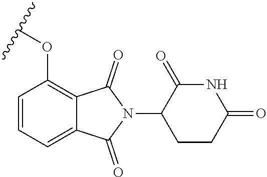 [SMILES: *Oc1cccc2c1C(=O)N(C1CCC(=O)NC1=O)C2=O] |
| 1175 | 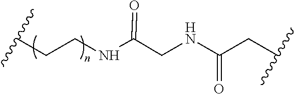 | 1 | 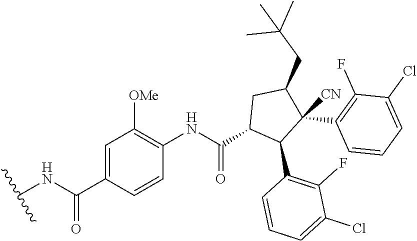 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1176 | 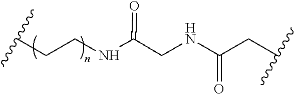 | 2 | 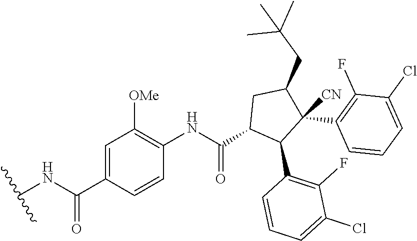 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1177 | 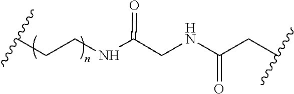 | 3 | 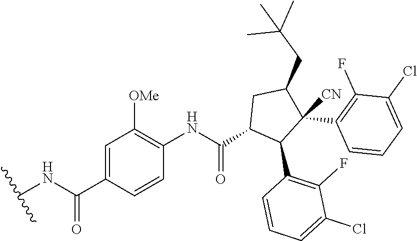 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1178 | 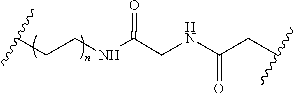 | 4 | 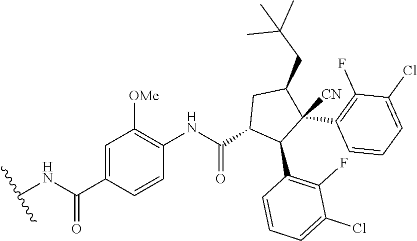 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1179 | 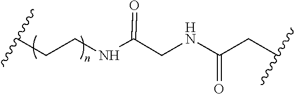 | 5 | 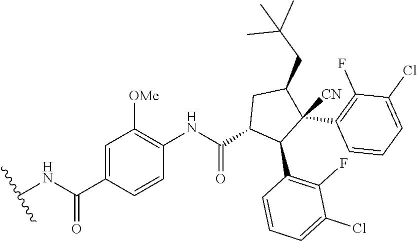 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1180 | 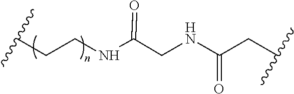 | 1 | 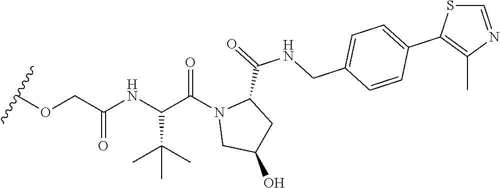 |
| 1181 | 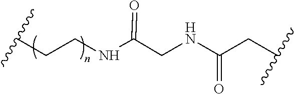 | 2 | 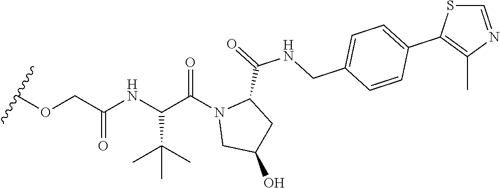 |
| 1182 | 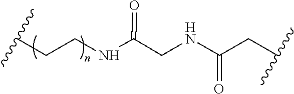 | 3 | 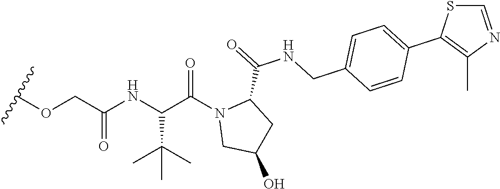 |
| 1183 | 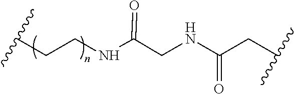 | 4 | 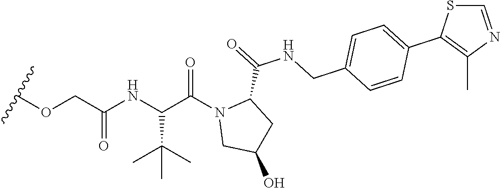 |
| 1184 | 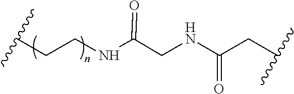 | 5 | 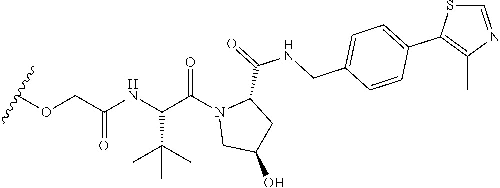 |

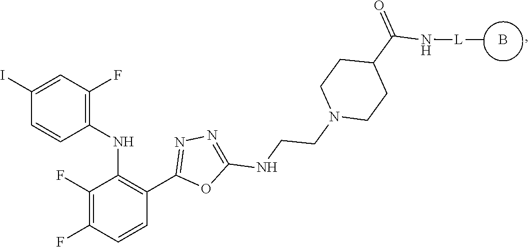
| Formula | | | |
|---|---|---|---|
| No. | L | n | B |
| 1185 | 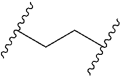 [SMILES: *CC*] | — | 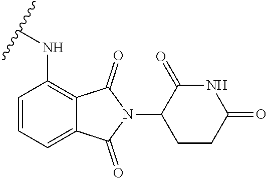 [SMILES: *Nc1cccc2c1C(=O)N(C1CCC(=O)NC1=O)C2=O] |
| 1186 | 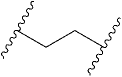 [SMILES: *CC*] | — | 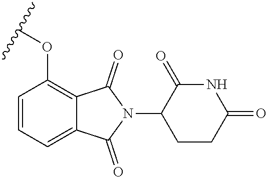 [SMILES: *Oc1cccc2c1C(=O)N(C1CCC(=O)NC1=O)C2=O] |
| 1187 | 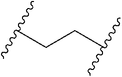 [SMILES: *CC*] | — | 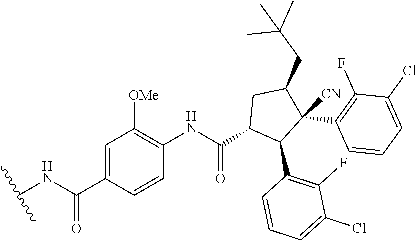 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1188 | 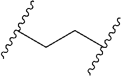 [SMILES: *CC*] | — | 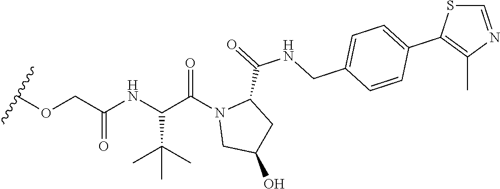 |
| 1189 | 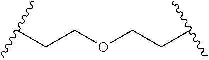 [SMILES: *CCOCC*] | — | 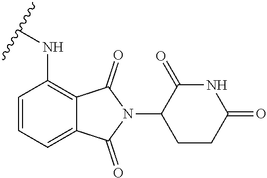 [SMILES: *Nc1cccc2c1C(=O)N(C1CCC(=O)NC1=O)C2=O] |
| 1190 | 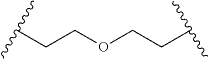 [SMILES: *CCOCC*] | — | 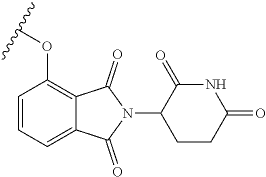 [SMILES: *Oc1cccc2c1C(=O)N(C1CCC(=O)NC1=O)C2=O] |
| 1191 | 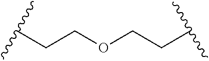 [SMILES: *CCOCC*] | — | 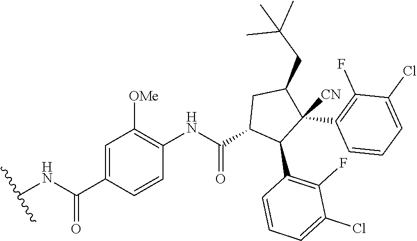 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1192 | 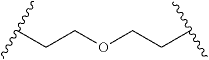 [SMILES: *CCOCC*] | — | 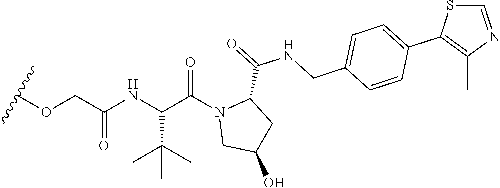 |
| 1193 | 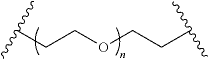 | 1 | 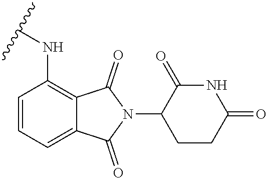 [SMILES: *Nc1cccc2c1C(=O)N(C1CCC(=O)NC1=O)C2=O] |
| 1194 | 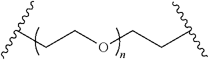 | 2 | 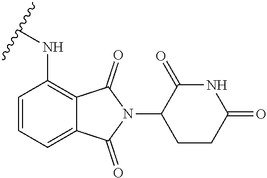 [SMILES: *Nc1cccc2c1C(=O)N(C1CCC(=O)NC1=O)C2=O] |
| 1195 | 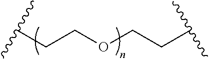 | 3 | 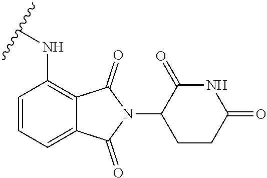 [SMILES: *Nc1cccc2c1C(=O)N(C1CCC(=O)NC1=O)C2=O] |
| 1196 | 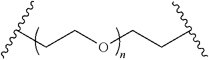 | 4 | 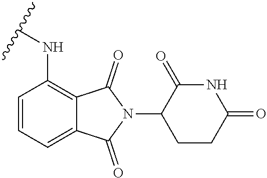 [SMILES: *Nc1cccc2c1C(=O)N(C1CCC(=O)NC1=O)C2=O] |
| 1197 | 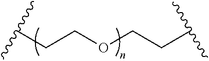 | 5 | 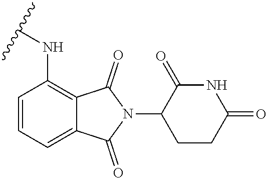 [SMILES: *Nc1cccc2c1C(=O)N(C1CCC(=O)NC1=O)C2=O] |
| 1198 | 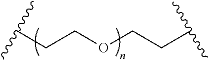 | 1 | 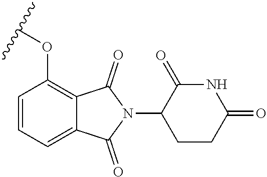 [SMILES: *Oc1cccc2c1C(=O)N(C1CCC(=O)NC1=O)C2=O] |
| 1199 | 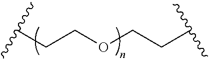 | 2 | 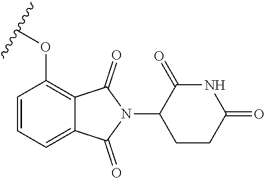 [SMILES: *Oc1cccc2c1C(=O)N(C1CCC(=O)NC1=O)C2=O] |
| 1200 | 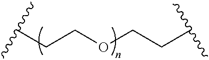 | 3 | 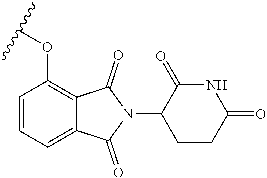 [SMILES: *Oc1cccc2c1C(=O)N(C1CCC(=O)NC1=O)C2=O] |
| 1201 | 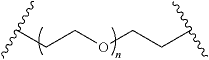 | 4 | 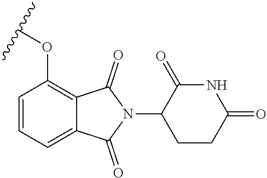 [SMILES: *Oc1cccc2c1C(=O)N(C1CCC(=O)NC1=O)C2=O] |
| 1202 | 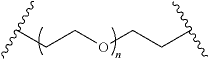 | 5 | 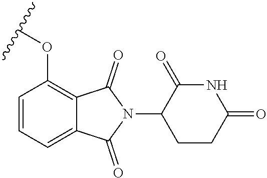 [SMILES: *Oc1cccc2c1C(=O)N(C1CCC(=O)NC1=O)C2=O] |
| 1203 | 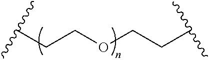 | 1 | 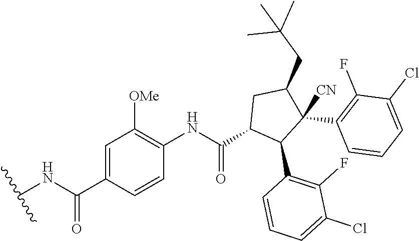 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1204 | 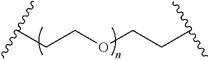 | 2 | 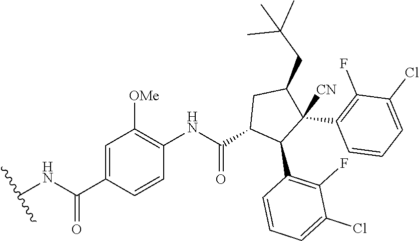 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1205 | 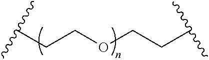 | 3 | 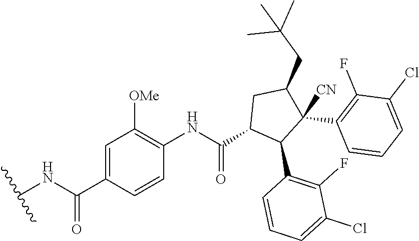 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1206 | 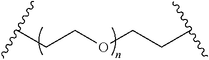 | 4 | 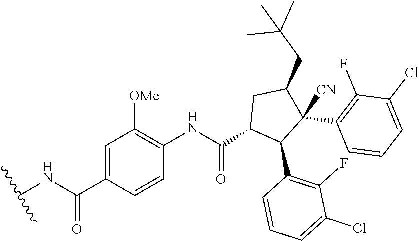 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1207 | 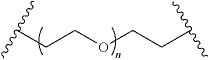 | 5 | 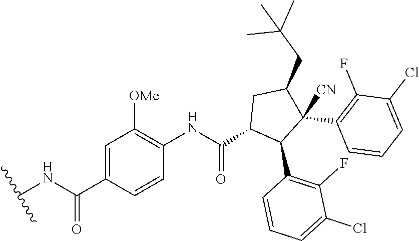 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1208 | 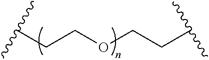 | 1 | 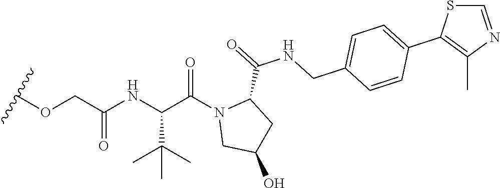 [SMILES: *OCC(=O)N[C@H](C(=O)N1C[C@H](O)C[C@H]1C(=O)NCc1ccc(-c2scnc2C)cc1)C(C)(C)C] |
| 1209 | 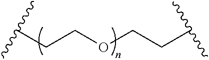 | 2 | 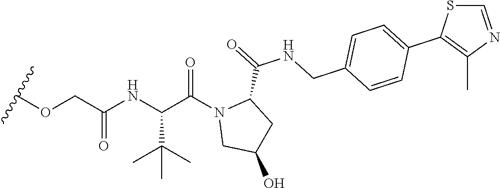 [SMILES: *OCC(=O)N[C@H](C(=O)N1C[C@H](O)C[C@H]1C(=O)NCc1ccc(-c2scnc2C)cc1)C(C)(C)C] |
| 1210 | 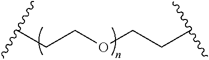 | 3 | 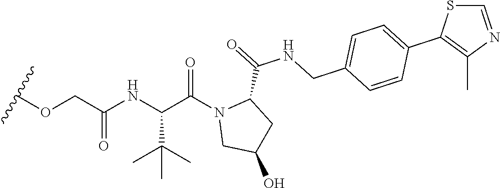 [SMILES: *OCC(=O)N[C@H](C(=O)N1C[C@H](O)C[C@H]1C(=O)NCc1ccc(-c2scnc2C)cc1)C(C)(C)C] |
| 1211 | 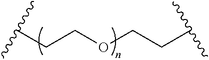 | 4 | 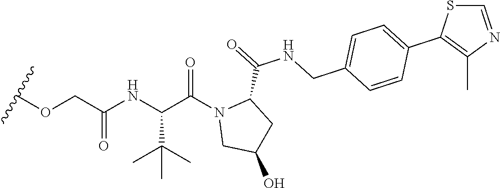 [SMILES: *OCC(=O)N[C@H](C(=O)N1C[C@H](O)C[C@H]1C(=O)NCc1ccc(-c2scnc2C)cc1)C(C)(C)C] |
| 1212 | 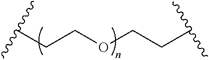 | 5 | 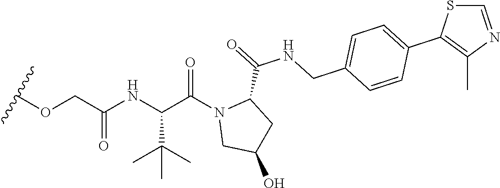 [SMILES: *OCC(=O)N[C@H](C(=O)N1C[C@H](O)C[C@H]1C(=O)NCc1ccc(-c2scnc2C)cc1)C(C)(C)C] |
| 1213 | 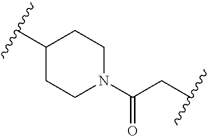 [SMILES: *CC(=O)N1CCC(*)CC1] | — | 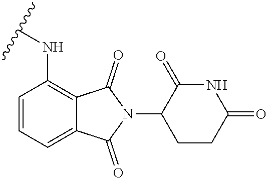 [SMILES: *Nc1cccc2c1C(=O)N(C1CCC(=O)NC1=O)C2=O] |
| 1214 | 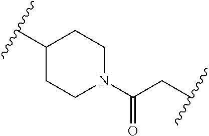 [SMILES: *CC(=O)N1CCC(*)CC1] | — | 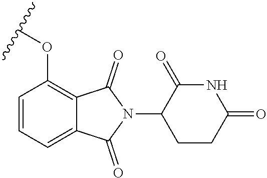 [SMILES: *Oc1cccc2c1C(=O)N(C1CCC(=O)NC1=O)C2=O] |
| 1215 | 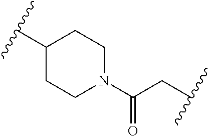 [SMILES: *CC(=O)N1CCC(*)CC1] | — | 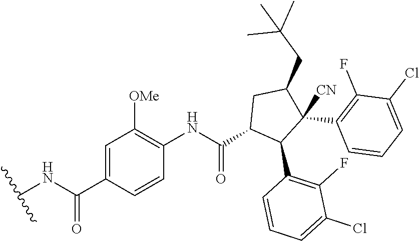 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1216 | 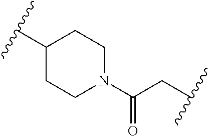 [SMILES: *CC(=O)N1CCC(*)CC1] | — | 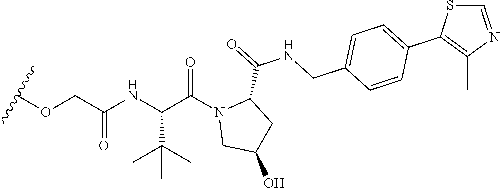 [SMILES: *OCC(=O)N[C@H](C(=O)N1C[C@H](O)C[C@H]1C(=O)NCc1ccc(-c2scnc2C)cc1)C(C)(C)C] |
| 1217 | 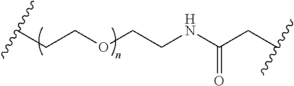 | 1 | 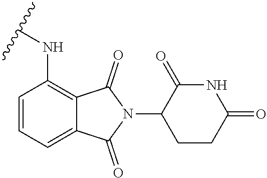 [SMILES: *Nc1cccc2c1C(=O)N(C1CCC(=O)NC1=O)C2=O] |
| 1218 | 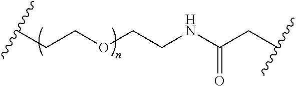 | 2 | 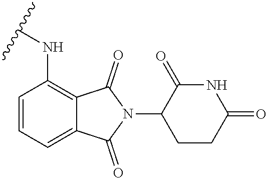 [SMILES: *Nc1cccc2c1C(=O)N(C1CCC(=O)NC1=O)C2=O] |
| 1219 | 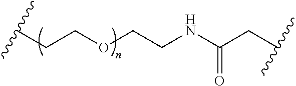 | 3 | 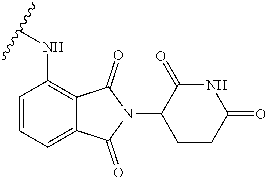 [SMILES: *Nc1cccc2c1C(=O)N(C1CCC(=O)NC1=O)C2=O] |
| 1220 | 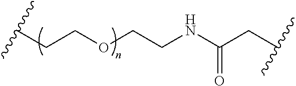 | 4 | 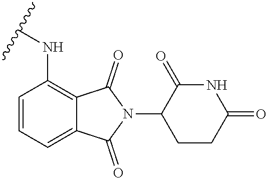 [SMILES: *Nc1cccc2c1C(=O)N(C1CCC(=O)NC1=O)C2=O] |
| 1221 | 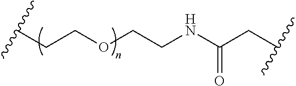 | 5 | 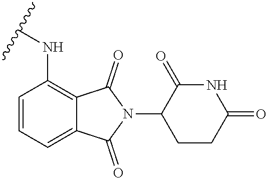 [SMILES: *Nc1cccc2c1C(=O)N(C1CCC(=O)NC1=O)C2=O] |
| 1222 | 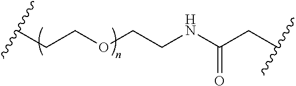 | 1 | 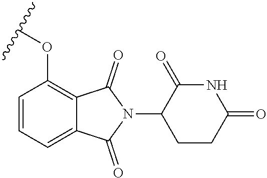 [SMILES: *Oc1cccc2c1C(=O)N(C1CCC(=O)NC1=O)C2=O] |
| 1223 | 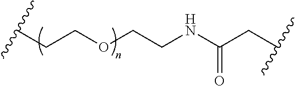 | 2 | 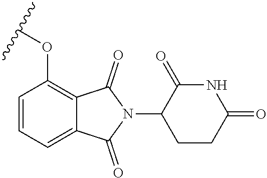 [SMILES: *Oc1cccc2c1C(=O)N(C1CCC(=O)NC1=O)C2=O] |
| 1224 | 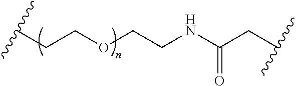 | 3 | 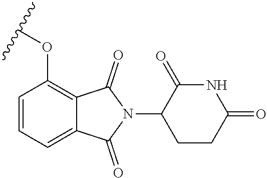 [SMILES: *Oc1cccc2c1C(=O)N(C1CCC(=O)NC1=O)C2=O] |
| 1225 | 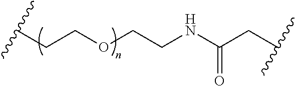 | 4 | 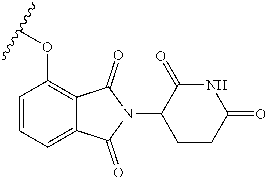 [SMILES: *Oc1cccc2c1C(=O)N(C1CCC(=O)NC1=O)C2=O] |
| 1226 | 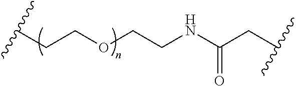 | 5 | 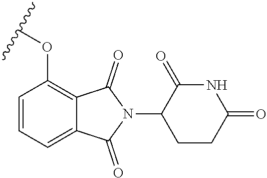 [SMILES: *Oc1cccc2c1C(=O)N(C1CCC(=O)NC1=O)C2=O] |
| 1227 | 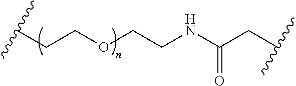 | 1 | 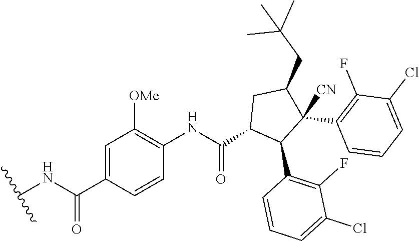 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1228 | 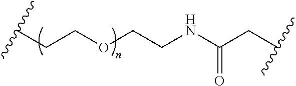 | 2 | 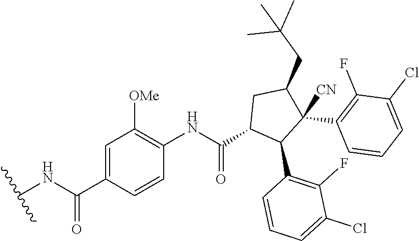 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1229 | 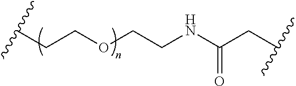 | 3 | 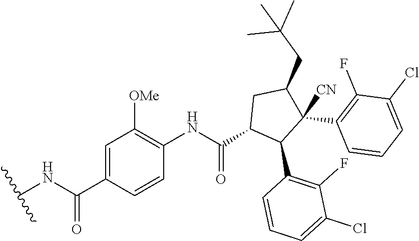 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1230 | 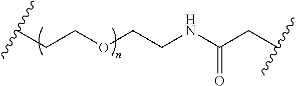 | 4 | 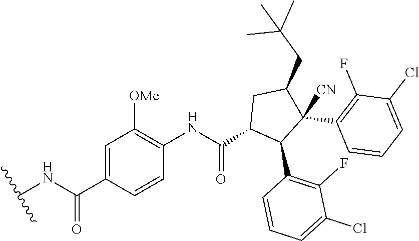 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1231 | 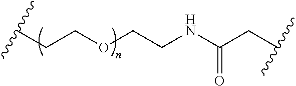 | 5 | 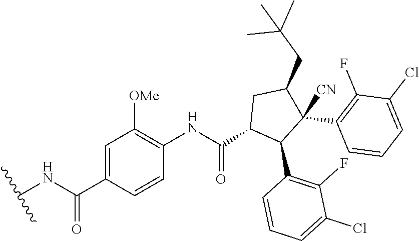 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1232 | 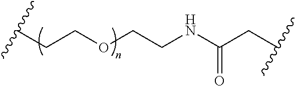 | 1 | 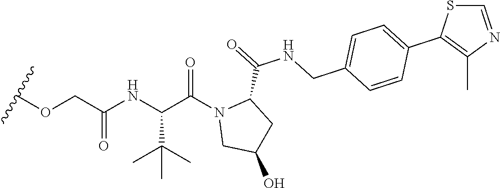 [SMILES: *OCC(=O)N[C@H](C(=O)N1C[C@H](O)C[C@H]1C(=O)NCc1ccc(-c2scnc2C)cc1)C(C)(C)C] |
| 1233 | 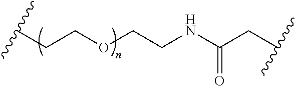 | 2 | 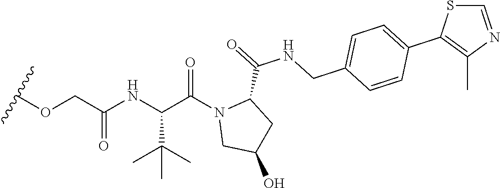 [SMILES: *OCC(=O)N[C@H](C(=O)N1C[C@H](O)C[C@H]1C(=O)NCc1ccc(-c2scnc2C)cc1)C(C)(C)C] |
| 1234 | 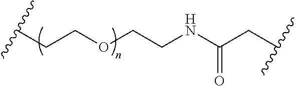 | 3 | 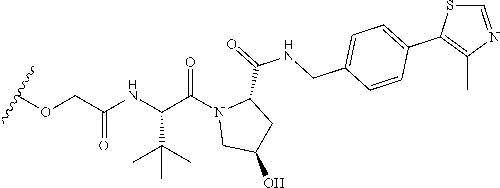 [SMILES: *OCC(=O)N[C@H](C(=O)N1C[C@H](O)C[C@H]1C(=O)NCc1ccc(-c2scnc2C)cc1)C(C)(C)C] |
| 1235 | 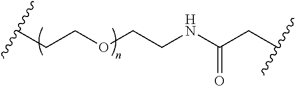 | 4 | 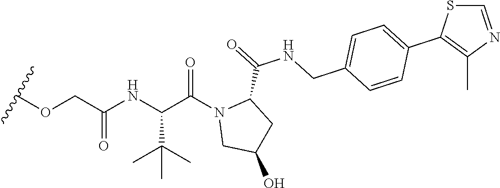 [SMILES: *OCC(=O)N[C@H](C(=O)N1C[C@H](O)C[C@H]1C(=O)NCc1ccc(-c2scnc2C)cc1)C(C)(C)C] |
| 1236 | 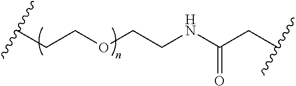 | 5 | 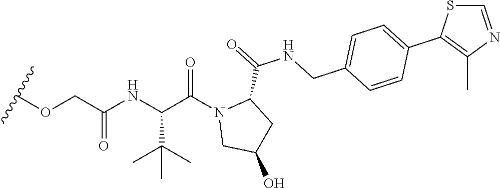 [SMILES: *OCC(=O)N[C@H](C(=O)N1C[C@H](O)C[C@H]1C(=O)NCc1ccc(-c2scnc2C)cc1)C(C)(C)C] |
| 1237 | 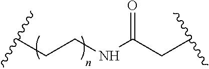 | 1 | 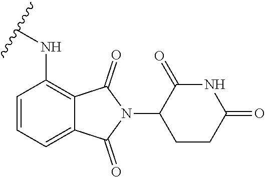 [SMILES: *Nc1cccc2c1C(=O)N(C1CCC(=O)NC1=O)C2=O] |
| 1238 | 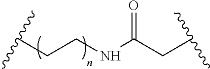 | 2 | 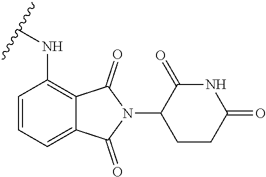 [SMILES: *Nc1cccc2c1C(=O)N(C1CCC(=O)NC1=O)C2=O] |
| 1239 | 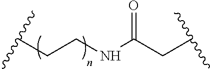 | 3 | 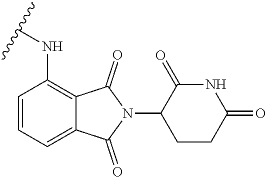 [SMILES: *Nc1cccc2c1C(=O)N(C1CCC(=O)NC1=O)C2=O] |
| 1240 | 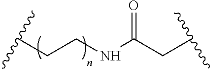 | 4 | 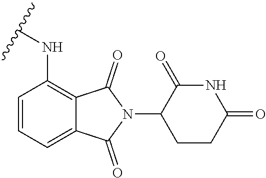 [SMILES: *Nc1cccc2c1C(=O)N(C1CCC(=O)NC1=O)C2=O] |
| 1241 | 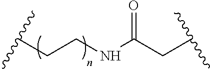 | 5 | 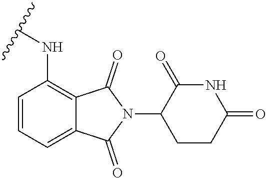 [SMILES: *Nc1cccc2c1C(=O)N(C1CCC(=O)NC1=O)C2=O] |
| 1242 | 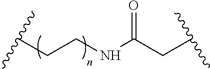 | 1 | 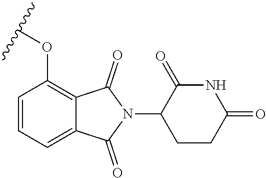 [SMILES: *Oc1cccc2c1C(=O)N(C1CCC(=O)NC1=O)C2=O] |
| 1243 | 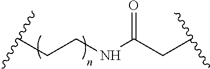 | 2 | 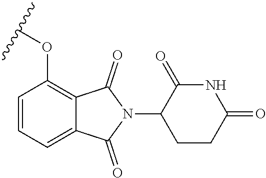 [SMILES: *Oc1cccc2c1C(=O)N(C1CCC(=O)NC1=O)C2=O] |
| 1244 | 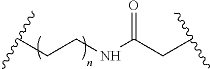 | 3 | 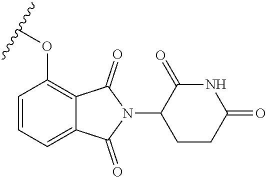 [SMILES: *Oc1cccc2c1C(=O)N(C1CCC(=O)NC1=O)C2=O] |
| 1245 | 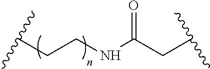 | 4 | 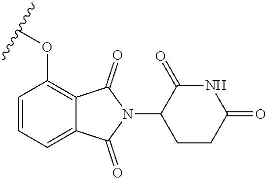 [SMILES: *Oc1cccc2c1C(=O)N(C1CCC(=O)NC1=O)C2=O] |
| 1246 | 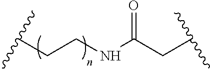 | 5 | 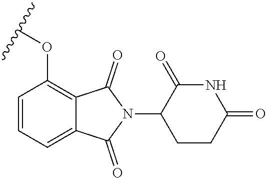 [SMILES: *Oc1cccc2c1C(=O)N(C1CCC(=O)NC1=O)C2=O] |
| 1247 | 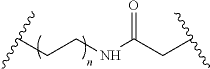 | 1 | 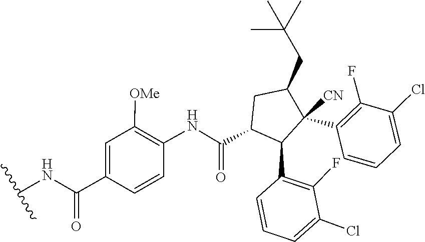 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1248 | 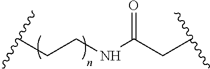 | 2 | 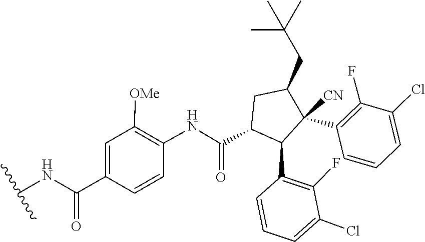 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1249 | 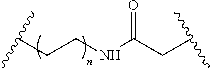 | 3 | 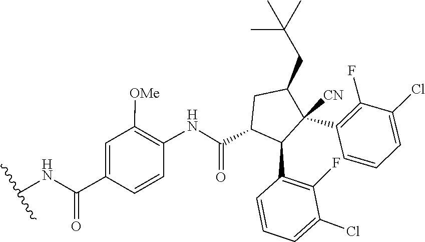 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1250 | 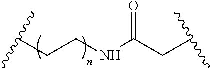 | 4 | 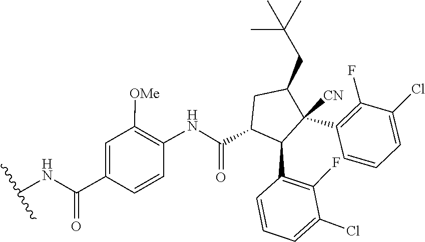 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1251 | 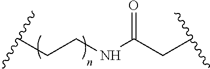 | 5 | 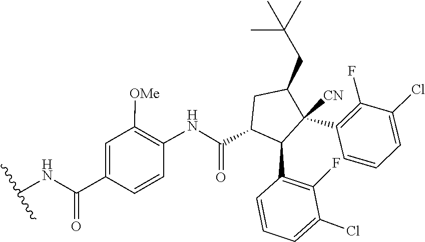 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1252 | 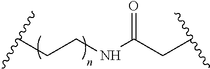 | 1 | 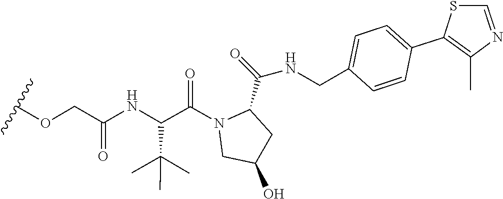 |
| 1253 | 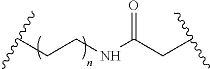 | 2 | 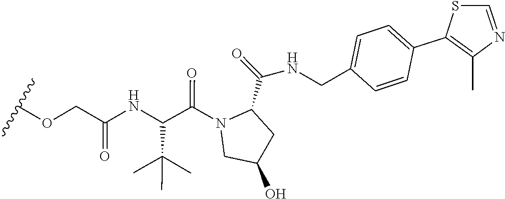 |
| 1254 | 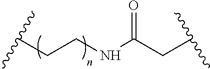 | 3 | 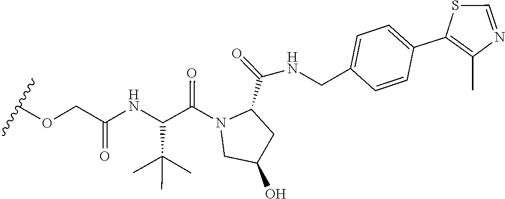 |
| 1255 | 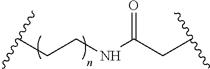 | 4 | 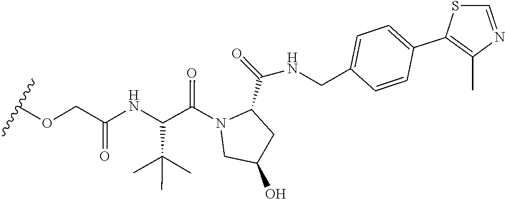 |
| 1256 | 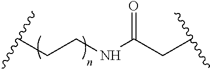 | 5 | 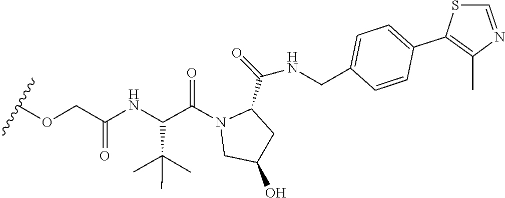 |
| 1257 | 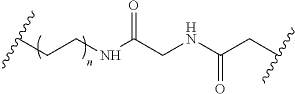 | 1 | 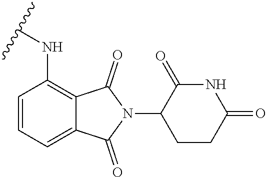 [SMILES: *Nc1cccc2c1C(=O)N(C1CCC(=O)NC1=O)C2=O] |
| 1258 | 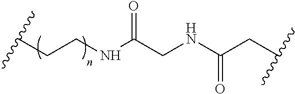 | 2 | 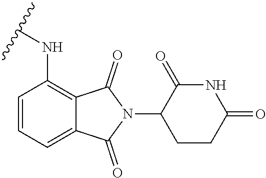 [SMILES: *Nc1cccc2c1C(=O)N(C1CCC(=O)NC1=O)C2=O] |
| 1259 | 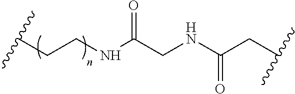 | 3 | 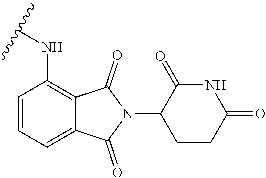 [SMILES: *Nc1cccc2c1C(=O)N(C1CCC(=O)NC1=O)C2=O] |
| 1260 | 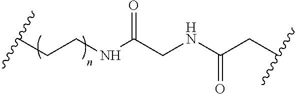 | 4 | 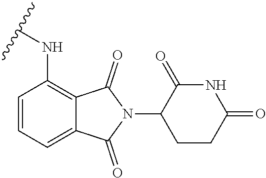 [SMILES: *Nc1cccc2c1C(=O)N(C1CCC(=O)NC1=O)C2=O] |
| 1261 | 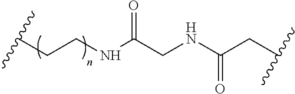 | 5 | 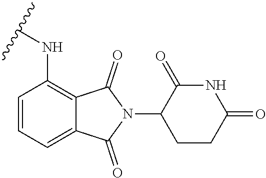 [SMILES: *Nc1cccc2c1C(=O)N(C1CCC(=O)NC1=O)C2=O] |
| 1262 | 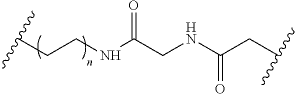 | 1 | 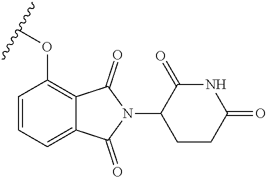 [SMILES: *Oc1cccc2c1C(=O)N(C1CCC(=O)NC1=O)C2=O] |
| 1263 | 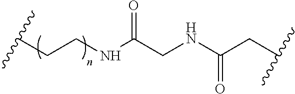 | 2 | 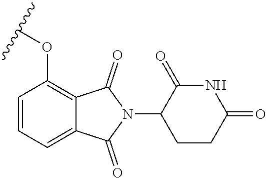 [SMILES: *Oc1cccc2c1C(=O)N(C1CCC(=O)NC1=O)C2=O] |
| 1264 | 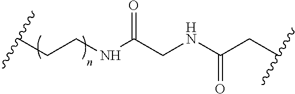 | 3 | 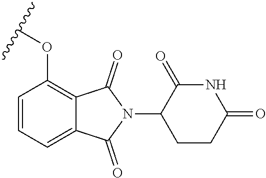 [SMILES: *Oc1cccc2c1C(=O)N(C1CCC(=O)NC1=O)C2=O] |
| 1265 | 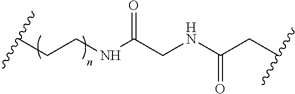 | 4 | 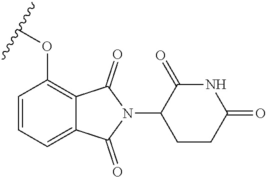 [SMILES: *Oc1cccc2c1C(=O)N(C1CCC(=O)NC1=O)C2=O] |
| 1266 | 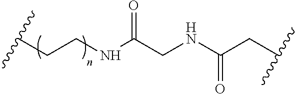 | 5 | 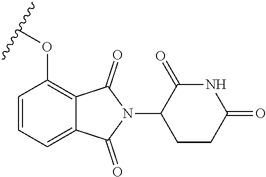 [SMILES: *Oc1cccc2c1C(=O)N(C1CCC(=O)NC1=O)C2=O] |
| 1267 | 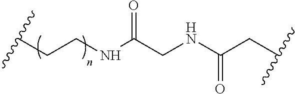 | 1 | 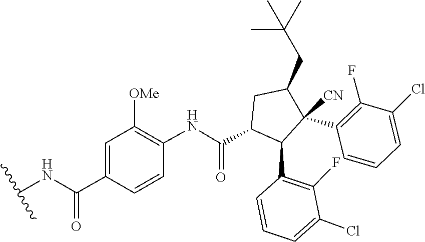 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1268 | 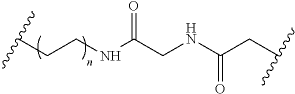 | 2 | 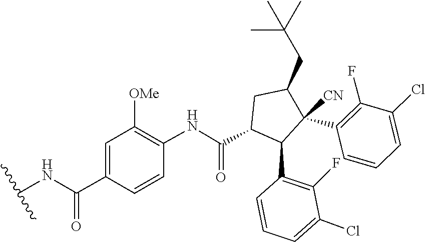 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1269 | 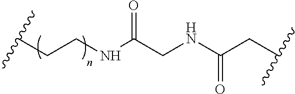 | 3 | 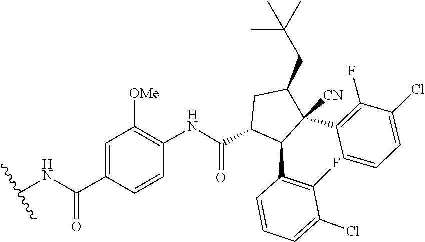 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1270 | 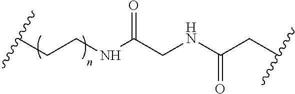 | 4 | 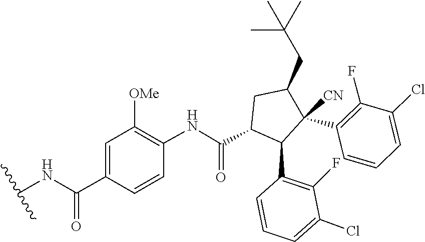 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1271 | 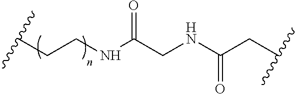 | 5 | 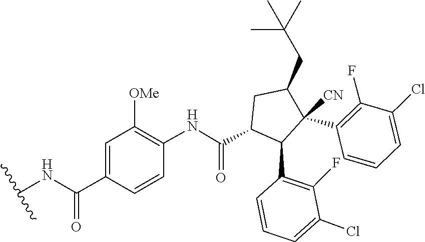 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1272 | 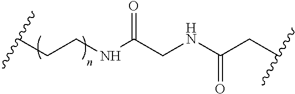 | 1 | 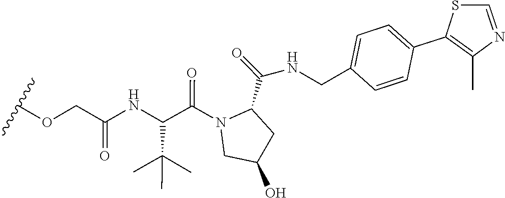 |
| 1273 | 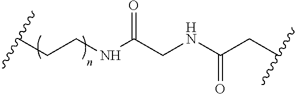 | 2 | 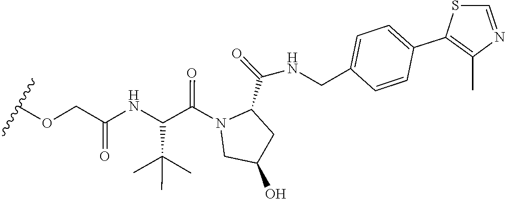 |
| 1274 | 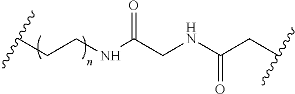 | 3 | 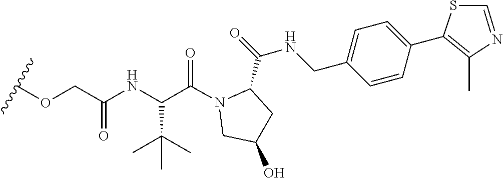 |
| 1275 | 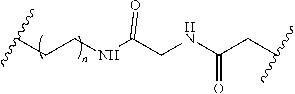 | 4 | 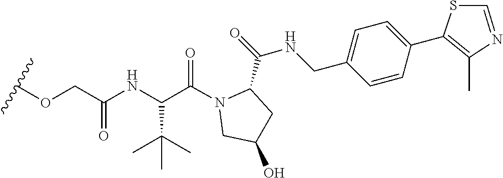 |
| 1276 | 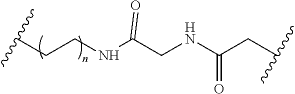 | 5 | 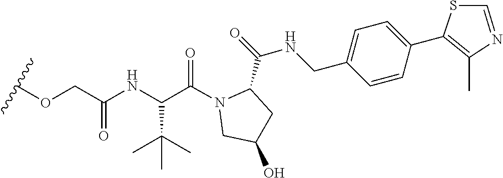 |

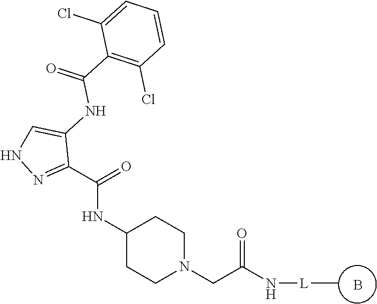
| Formula | | | |
|---|---|---|---|
| No. | L | n | B |
| 1277 | 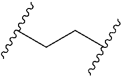 [SMILES: *CC*] | — | 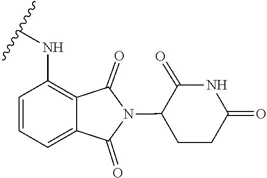 [SMILES: *Nc1cccc2c1C(=O)N(C1CCC(=O)NC1=O)C2=O] |
| 1278 | 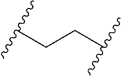 [SMILES: *CC*] | — | 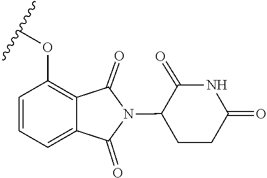 [SMILES: *Oc1cccc2c1C(=O)N(C1CCC(=O)NC1=O)C2=O] |
| 1279 | 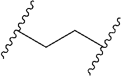 [SMILES: *CC*] | — | 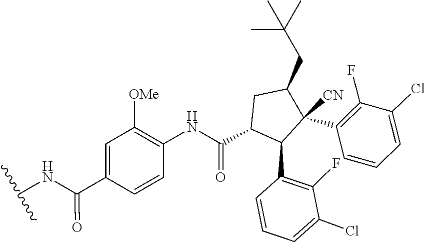 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1280 | 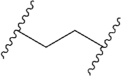 [SMILES: *CC*] | — | 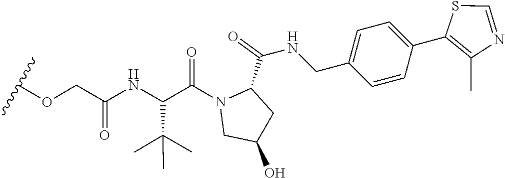 |
| 1281 | 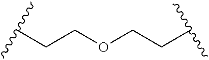 [SMILES: *CCOCC*] | — | 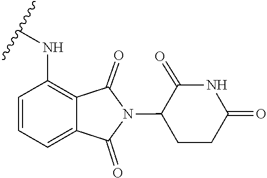 [SMILES: *Nc1cccc2c1C(=O)N(C1CCC(=O)NC1=O)C2=O] |
| 1282 | 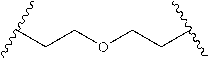 [SMILES: *CCOCC*] | — | 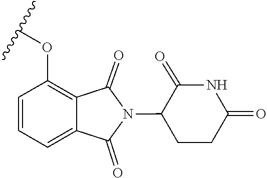 [SMILES: *Oc1cccc2c1C(=O)N(C1CCC(=O)NC1=O)C2=O] |
| 1283 | 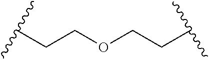 [SMILES: *CCOCC*] | — | 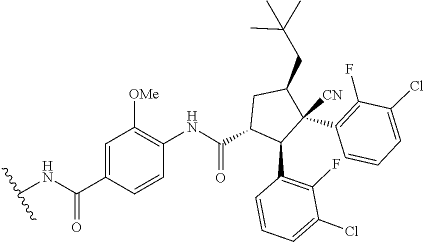 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1284 | 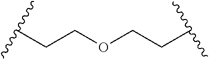 [SMILES: *CCOCC*] | — | 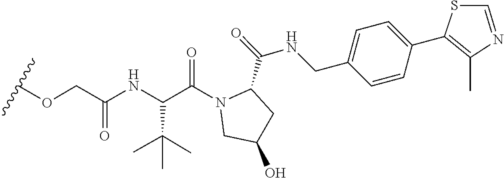 |
| 1285 | 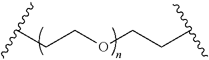 | 1 | 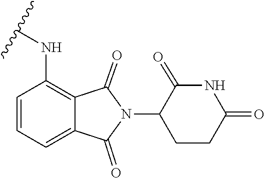 [SMILES: *Nc1cccc2c1C(=O)N(C1CCC(=O)NC1=O)C2=O] |
| 1286 | 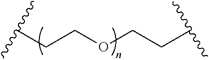 | 2 | 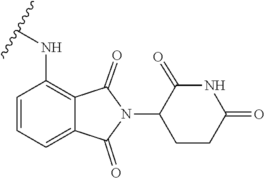 [SMILES: *Nc1cccc2c1C(=O)N(C1CCC(=O)NC1=O)C2=O] |
| 1287 | 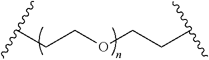 | 3 | 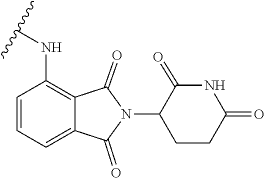 [SMILES: *Nc1cccc2c1C(=O)N(C1CCC(=O)NC1=O)C2=O] |
| 1288 | 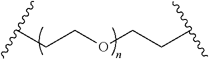 | 4 | 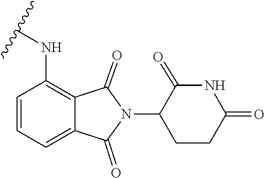 [SMILES: *Nc1cccc2c1C(=O)N(C1CCC(=O)NC1=O)C2=O] |
| 1289 | 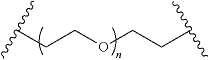 | 5 | 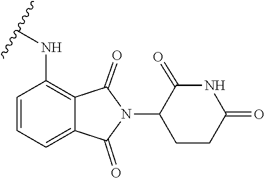 [SMILES: *Nc1cccc2c1C(=O)N(C1CCC(=O)NC1=O)C2=O] |
| 1290 | 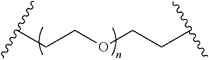 | 1 | 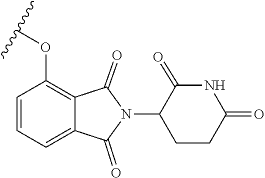 [SMILES: *Oc1cccc2c1C(=O)N(C1CCC(=O)NC1=O)C2=O] |
| 1291 | 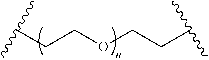 | 2 | 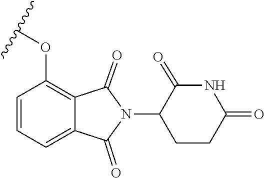 [SMILES: *Oc1cccc2c1C(=O)N(C1CCC(=O)NC1=O)C2=O] |
| 1292 | 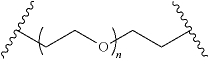 | 3 | 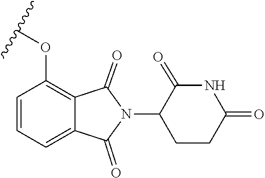 [SMILES: *Oc1cccc2c1C(=O)N(C1CCC(=O)NC1=O)C2=O] |
| 1293 | 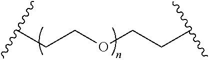 | 4 | 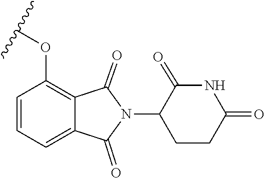 [SMILES: *Oc1cccc2c1C(=O)N(C1CCC(=O)NC1=O)C2=O] |
| 1294 | 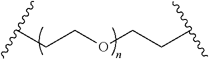 | 5 | 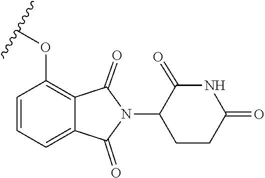 [SMILES: *Oc1cccc2c1C(=O)N(C1CCC(=O)NC1=O)C2=O] |
| 1295 | 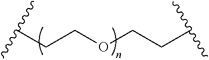 | 1 | 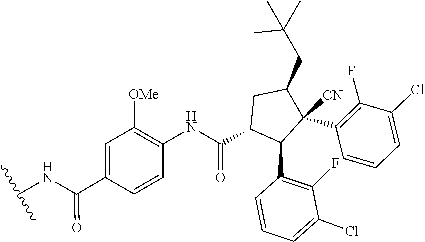 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1296 | 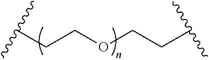 | 2 | 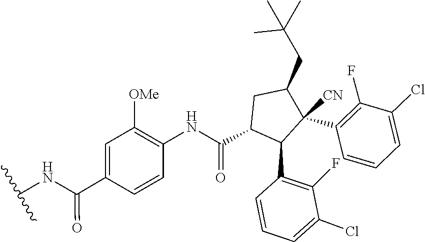 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1297 | 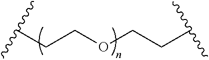 | 3 | 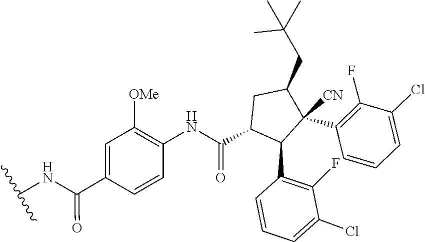 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1298 | 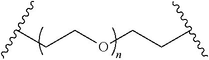 | 4 | 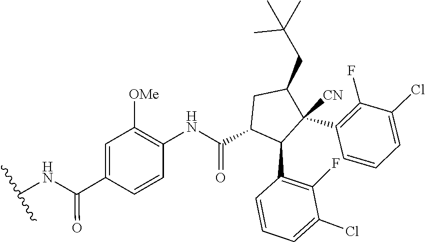 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1299 | 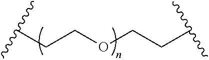 | 5 | 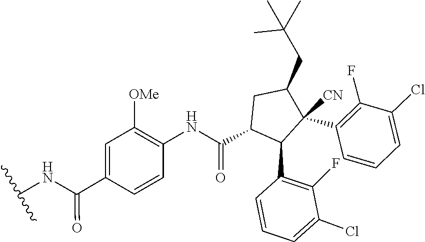 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1300 | 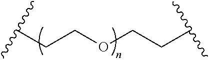 | 1 | 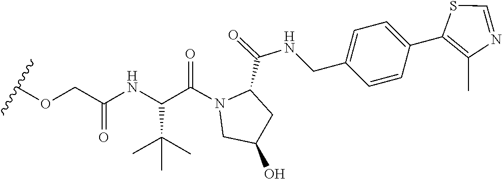 |
| 1301 | 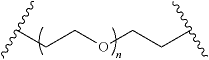 | 2 | 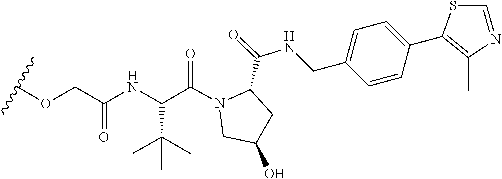 |
| 1302 | 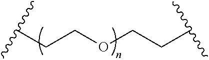 | 3 | 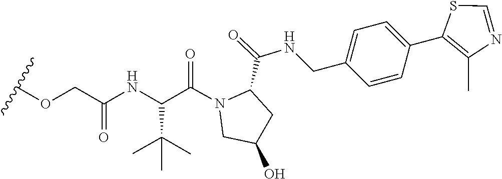 |
| 1303 | 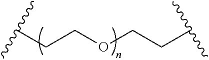 | 4 | 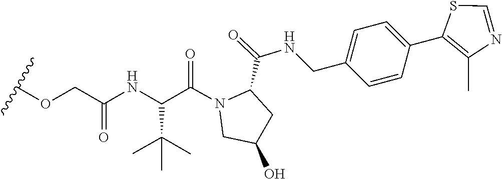 |
| 1304 | 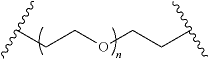 | 5 | 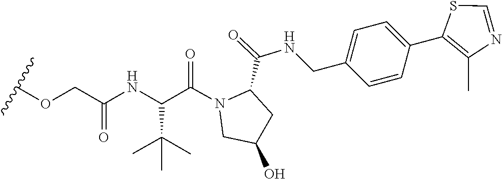 |
| 1305 | 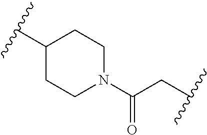 [SMILES: *CC(=O)N1CCC(*)CC1] | — | 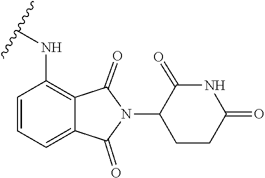 [SMILES: *Nc1cccc2c1C(=O)N(C1CCC(=O)NC1=O)C2=O] |
| 1306 | 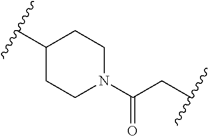 [SMILES: *CC(=O)N1CCC(*)CC1] | — | 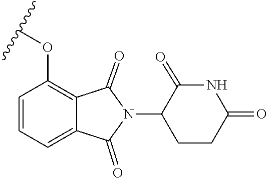 [SMILES: *Oc1cccc2c1C(=O)N(C1CCC(=O)NC1=O)C2=O] |
| 1307 | 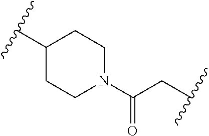 [SMILES: *CC(=O)N1CCC(*)CC1] | — | 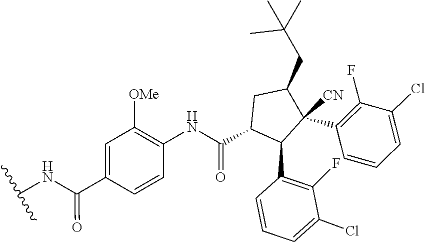 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1308 | 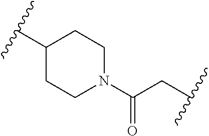 [SMILES: *CC(=O)N1CCC(*)CC1] | — | 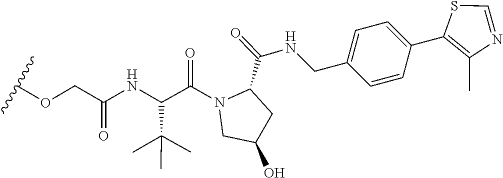 |
| 1309 | 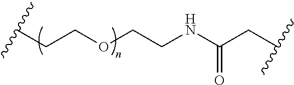 | 1 | 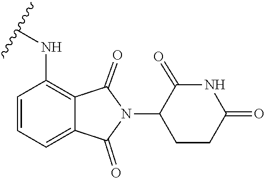 [SMILES: *Nc1cccc2c1C(=O)N(C1CCC(=O)NC1=O)C2=O] |
| 1310 | 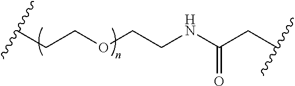 | 2 | 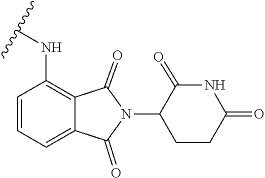 [SMILES: *Nc1cccc2c1C(=O)N(C1CCC(=O)NC1=O)C2=O] |
| 1311 | 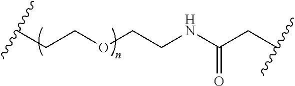 | 3 | 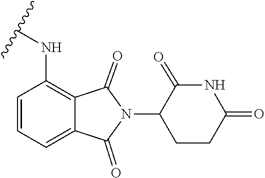 [SMILES: *Nc1cccc2c1C(=O)N(C1CCC(=O)NC1=O)C2=O] |
| 1312 | 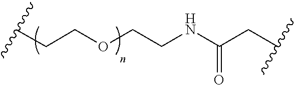 | 4 | 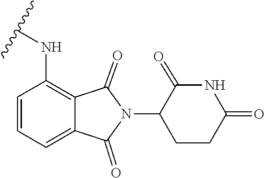 [SMILES: *Nc1cccc2c1C(=O)N(C1CCC(=O)NC1=O)C2=O] |
| 1313 | 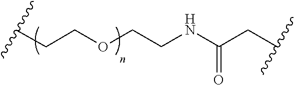 | 5 | 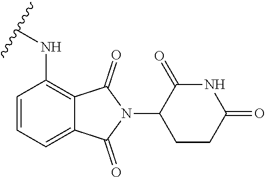 [SMILES: *Nc1cccc2c1C(=O)N(C1CCC(=O)NC1=O)C2=O] |
| 1314 | 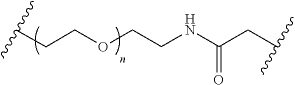 | 1 | 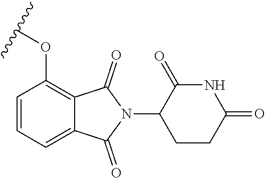 [SMILES: *Oc1cccc2c1C(=O)N(C1CCC(=O)NC1=O)C2=O] |
| 1315 | 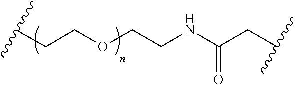 | 2 | 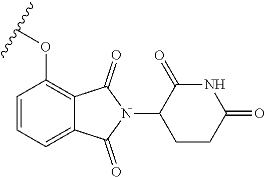 [SMILES: *Oc1cccc2c1C(=O)N(C1CCC(=O)NC1=O)C2=O] |
| 1316 | 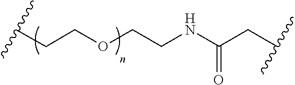 | 3 | 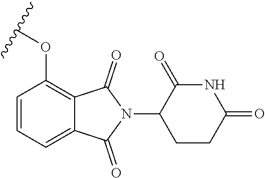 [SMILES: *Oc1cccc2c1C(=O)N(C1CCC(=O)NC1=O)C2=O] |
| 1317 | 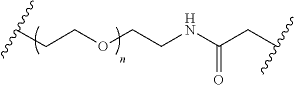 | 4 | 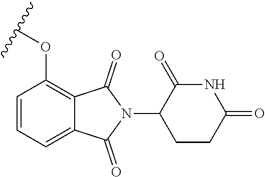 [SMILES: *Oc1cccc2c1C(=O)N(C1CCC(=O)NC1=O)C2=O] |
| 1318 | 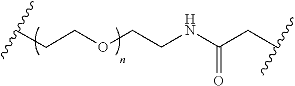 | 5 | 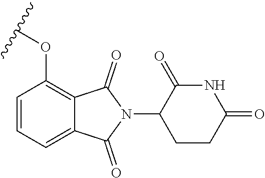 [SMILES: *Oc1cccc2c1C(=O)N(C1CCC(=O)NC1=O)C2=O] |
| 1319 | 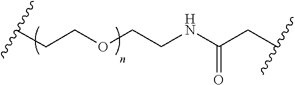 | 1 | 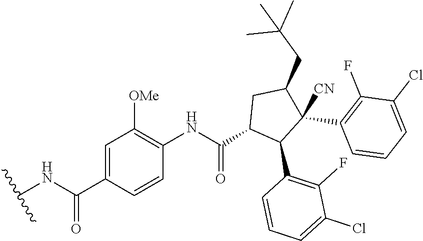 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1320 | 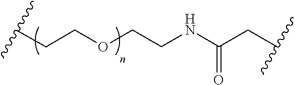 | 2 | 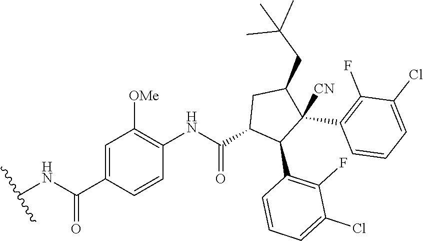 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1321 | 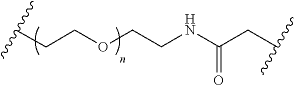 | 3 | 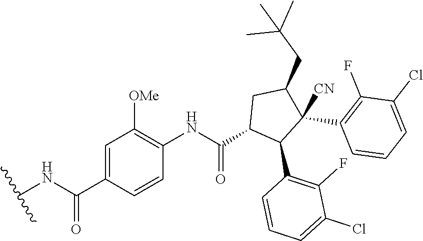 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1322 | 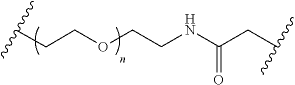 | 4 | 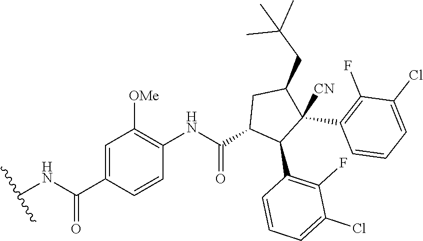 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1323 | 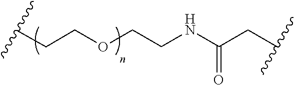 | 5 | 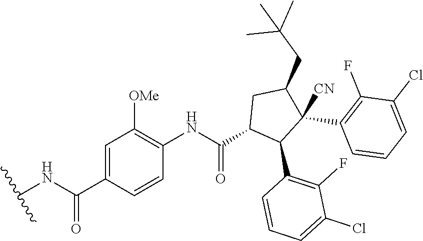 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1324 | 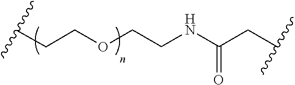 | 1 | 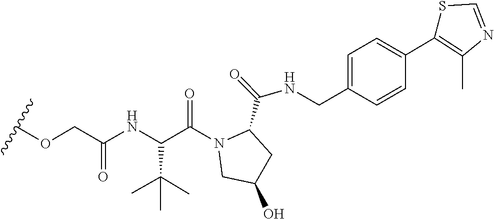 |
| 1325 | 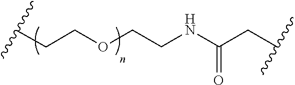 | 2 | 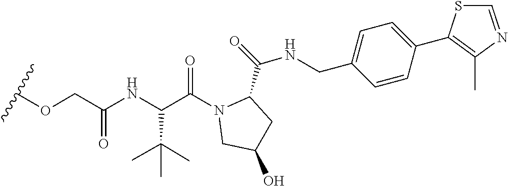 |
| 1326 | 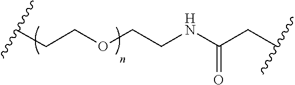 | 3 | 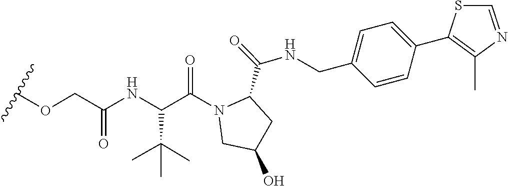 |
| 1327 | 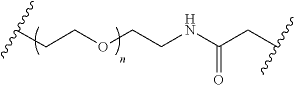 | 4 | 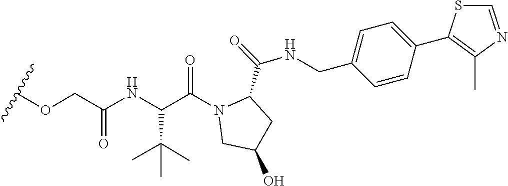 |
| 1328 | 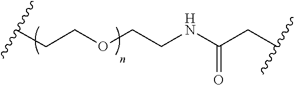 | 5 | 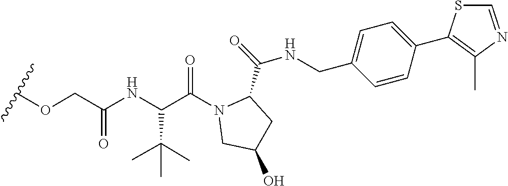 |
| 1329 | 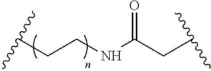 | 1 | 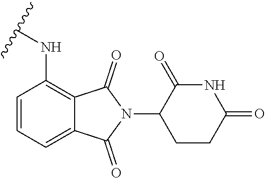 [SMILES: *Nc1cccc2c1C(=O)N(C1CCC(=O)NC1=O)C2=O] |
| 1330 | 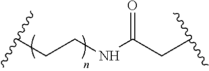 | 2 | 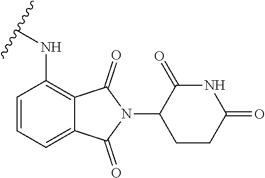 [SMILES: *Nc1cccc2c1C(=O)N(C1CCC(=O)NC1=O)C2=O] |
| 1331 | 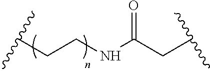 | 3 | 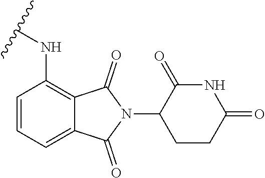 [SMILES: *Nc1cccc2c1C(=O)N(C1CCC(=O)NC1=O)C2=O] |
| 1332 | 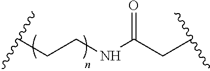 | 4 | 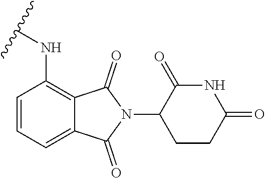 [SMILES: *Nc1cccc2c1C(=O)N(C1CCC(=O)NC1=O)C2=O] |
| 1333 | 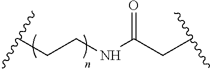 | 5 | 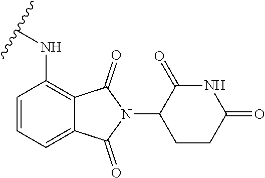 [SMILES: *Nc1cccc2c1C(=O)N(C1CCC(=O)NC1=O)C2=O] |
| 1334 | 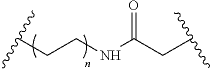 | 1 | 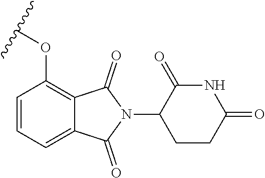 [SMILES: *Oc1cccc2c1C(=O)N(C1CCC(=O)NC1=O)C2=O] |
| 1335 | 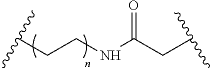 | 2 | 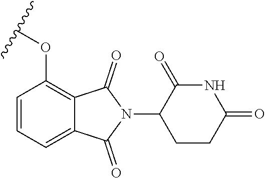 [SMILES: *Oc1cccc2c1C(=O)N(C1CCC(=O)NC1=O)C2=O] |
| 1336 | 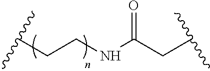 | 3 | 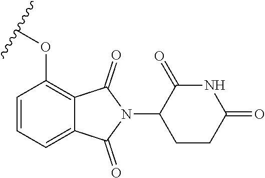 [SMILES: *Oc1cccc2c1C(=O)N(C1CCC(=O)NC1=O)C2=O] |
| 1337 | 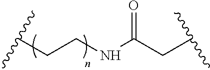 | 4 | 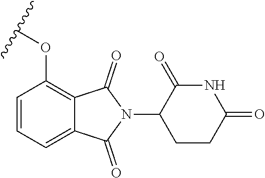 [SMILES: *Oc1cccc2c1C(=O)N(C1CCC(=O)NC1=O)C2=O] |
| 1338 | 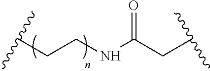 | 5 | 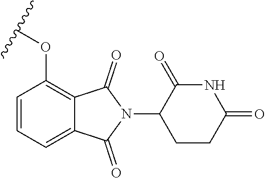 [SMILES: *Oc1cccc2c1C(=O)N(C1CCC(=O)NC1=O)C2=O] |
| 1339 | 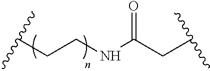 | 1 | 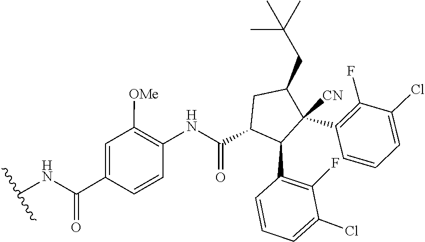 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1340 | 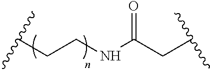 | 2 | 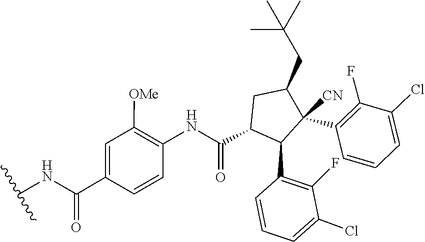 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1341 | 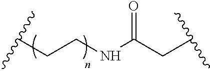 | 3 | 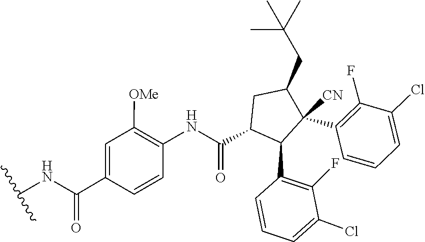 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1342 | 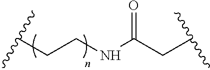 | 4 | 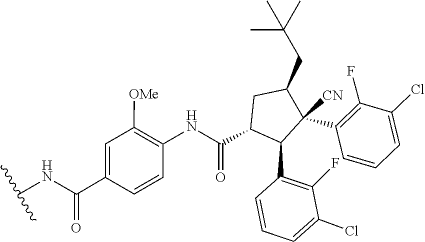 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1343 | 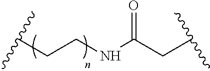 | 5 | 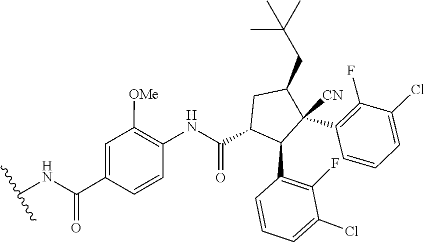 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1344 | 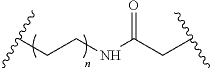 | 1 | 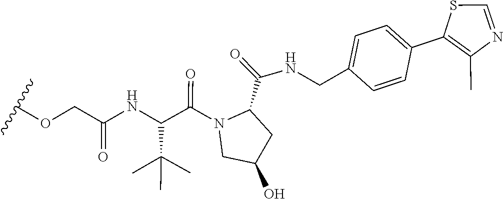 |
| 1345 | 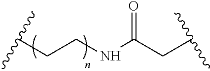 | 2 | 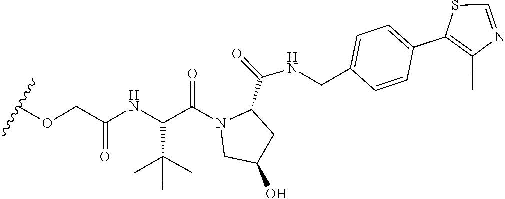 |
| 1346 | 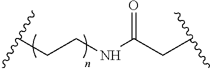 | 3 | 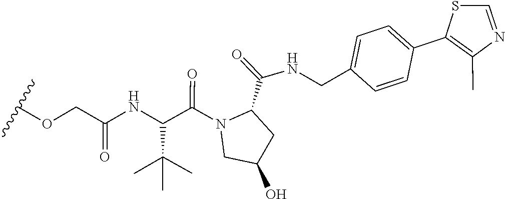 |
| 1347 | 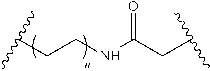 | 4 | 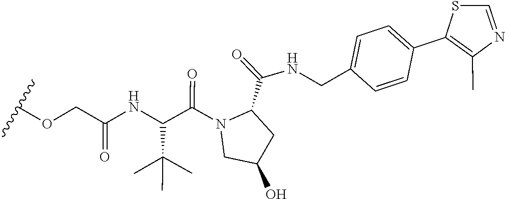 |
| 1348 | 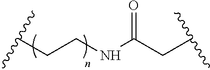 | 5 | 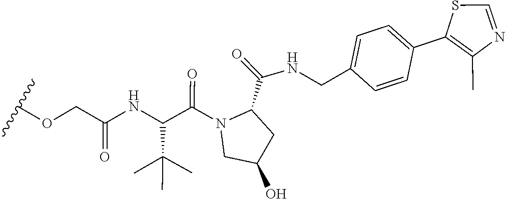 |
| 1349 | 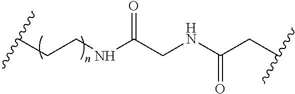 | 1 | 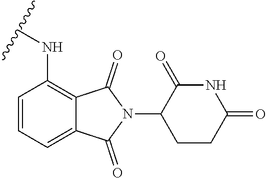 [SMILES: *Nc1cccc2c1C(=O)N(C1CCC(=O)NC1=O)C2=O] |
| 1350 | 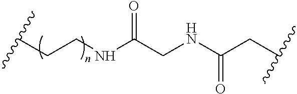 | 2 | 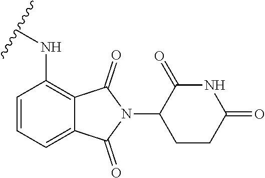 [SMILES: *Nc1cccc2c1C(=O)N(C1CCC(=O)NC1=O)C2=O] |
| 1351 | 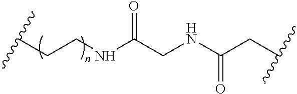 | 3 | 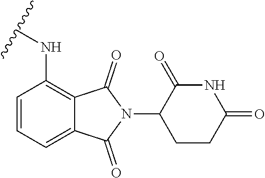 [SMILES: *Nc1cccc2c1C(=O)N(C1CCC(=O)NC1=O)C2=O] |
| 1352 | 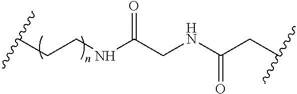 | 4 | 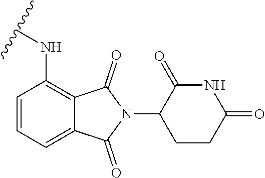 [SMILES: *Nc1cccc2c1C(=O)N(C1CCC(=O)NC1=O)C2=O] |
| 1353 | 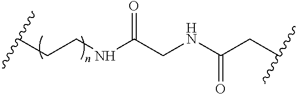 | 5 | 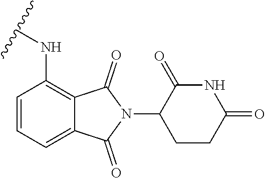 [SMILES: *Nc1cccc2c1C(=O)N(C1CCC(=O)NC1=O)C2=O] |
| 1354 | 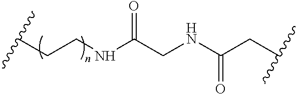 | 1 | 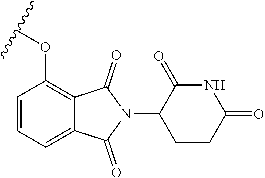 [SMILES: *Oc1cccc2c1C(=O)N(C1CCC(=O)NC1=O)C2=O] |
| 1355 | 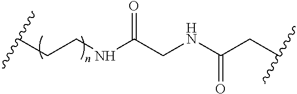 | 2 | 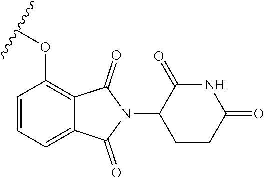 [SMILES: *Oc1cccc2c1C(=O)N(C1CCC(=O)NC1=O)C2=O] |
| 1356 | 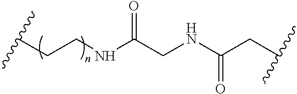 | 3 | 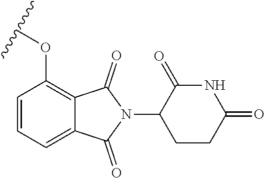 [SMILES: *Oc1cccc2c1C(=O)N(C1CCC(=O)NC1=O)C2=O] |
| 1357 | 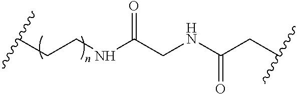 | 4 | 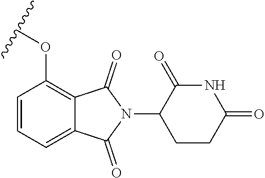 [SMILES: *Oc1cccc2c1C(=O)N(C1CCC(=O)NC1=O)C2=O] |
| 1358 | 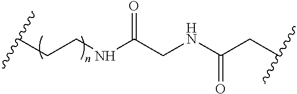 | 5 | 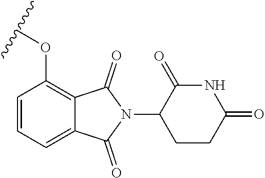 [SMILES: *Oc1cccc2c1C(=O)N(C1CCC(=O)NC1=O)C2=O] |
| 1359 | 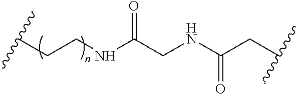 | 1 | 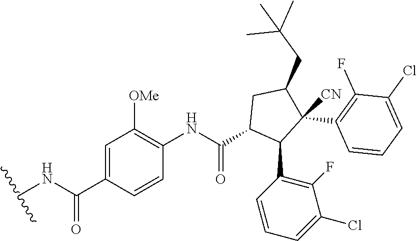 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1360 | 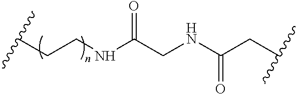 | 2 | 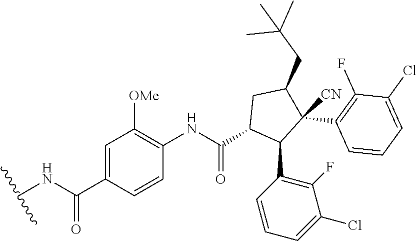 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1361 | 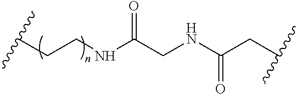 | 3 | 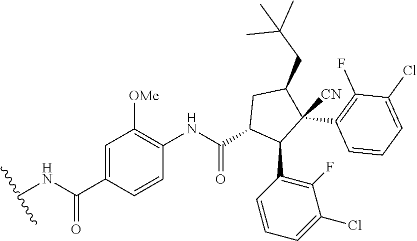 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1362 | 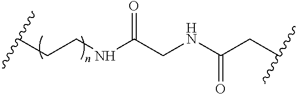 | 4 | 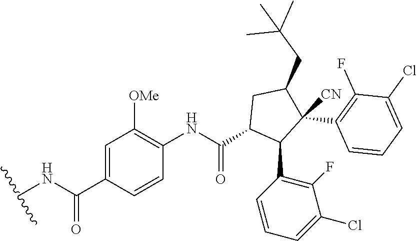 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1363 | 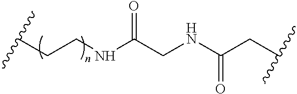 | 5 | 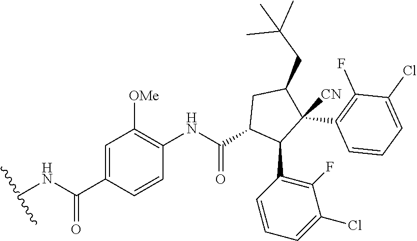 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1364 | 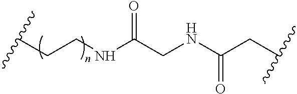 | 1 | 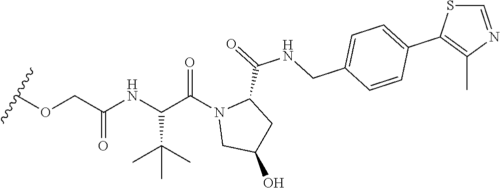 |
| 1365 | 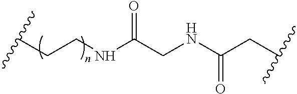 | 2 | 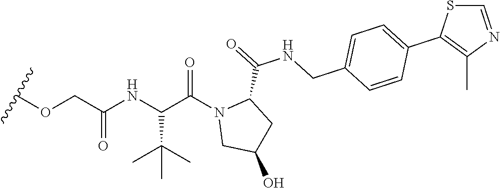 |
| 1366 | 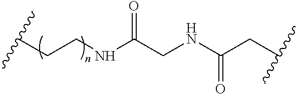 | 3 | 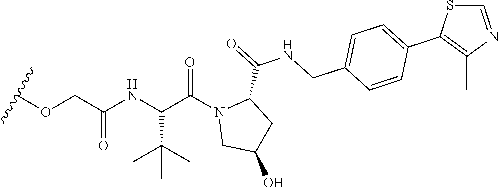 |
| 1367 | 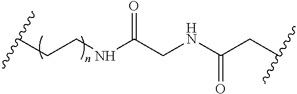 | 4 | 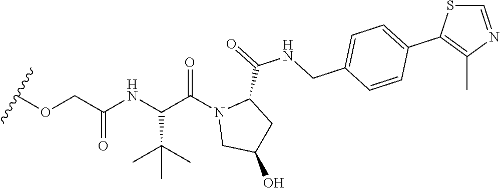 |
| 1368 | 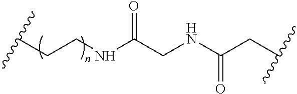 | 5 | 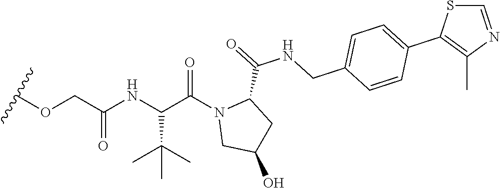 |

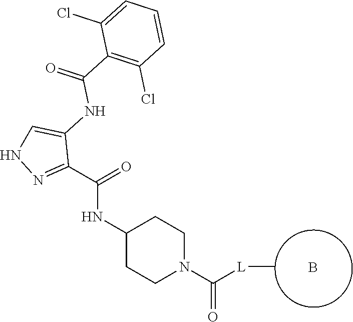
| Formula | | | |
|---|---|---|---|
| No. | L | n | B |
| 1369 | 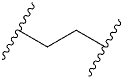 [SMILES: *CC*] | — | 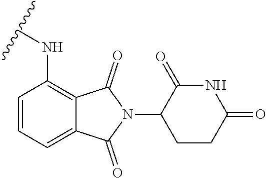 [SMILES: *Nc1cccc2c1C(=O)N(C1CCC(=O)NC1=O)C2=O] |
| 1370 | 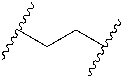 [SMILES: *CC*] | — | 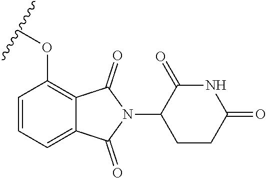 [SMILES: *Oc1cccc2c1C(=O)N(C1CCC(=O)NC1=O)C2=O] |
| 1371 | 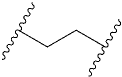 [SMILES: *CC*] | — | 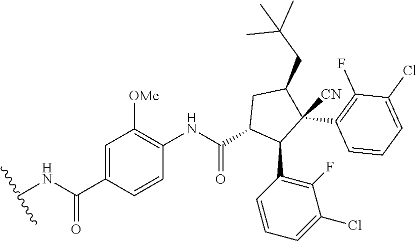 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1372 | 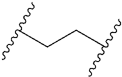 [SMILES: *CC*] | — | 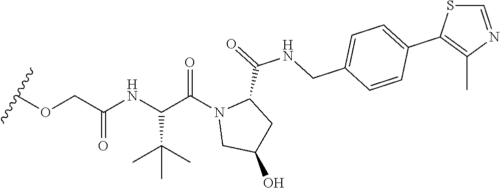 |
| 1373 | 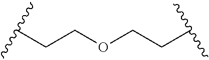 [SMILES: *CCOCC*] | — | 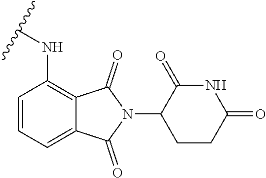 [SMILES: *Nc1cccc2c1C(=O)N(C1CCC(=O)NC1=O)C2=O] |
| 1374 | 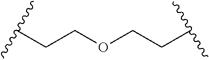 [SMILES: *CCOCC*] | — | 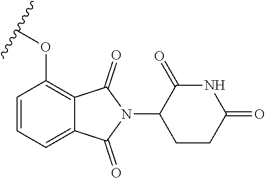 [SMILES: *Oc1cccc2c1C(=O)N(C1CCC(=O)NC1=O)C2=O] |
| 1375 | 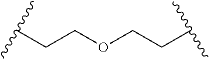 [SMILES: *CCOCC*] | — | 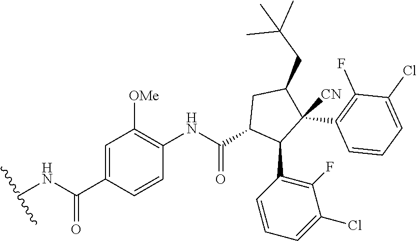 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1376 | 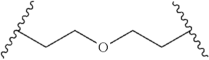 [SMILES: *CCOCC*] | — | 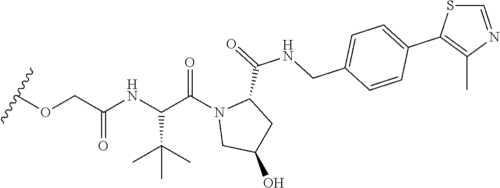 |
| 1377 | 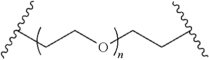 | 1 | 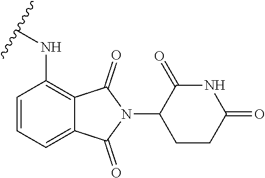 [SMILES: *Nc1cccc2c1C(=O)N(C1CCC(=O)NC1=O)C2=O] |
| 1378 | 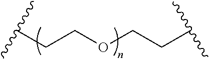 | 2 | 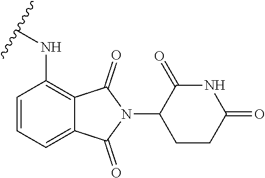 [SMILES: *Nc1cccc2c1C(=O)N(C1CCC(=O)NC1=O)C2=O] |
| 1379 | 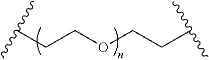 | 3 | 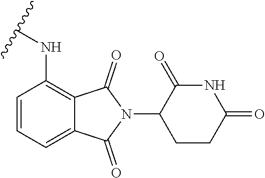 [SMILES: *Nc1cccc2c1C(=O)N(C1CCC(=O)NC1=O)C2=O] |
| 1380 | 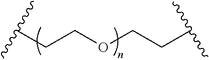 | 4 | 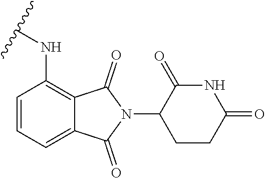 [SMILES: *Nc1cccc2c1C(=O)N(C1CCC(=O)NC1=O)C2=O] |
| 1381 | 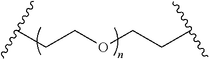 | 5 | 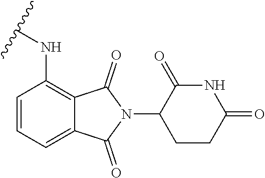 [SMILES: *Nc1cccc2c1C(=O)N(C1CCC(=O)NC1=O)C2=O] |
| 1382 | 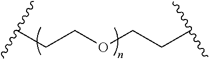 | 1 | 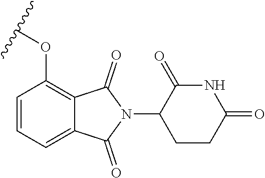 [SMILES: *Oc1cccc2c1C(=O)N(C1CCC(=O)NC1=O)C2=O] |
| 1383 | 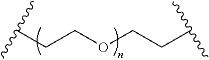 | 2 | 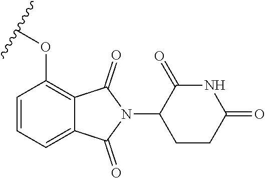 [SMILES: *Oc1cccc2c1C(=O)N(C1CCC(=O)NC1=O)C2=O] |
| 1384 | 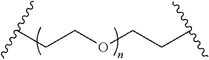 | 3 | 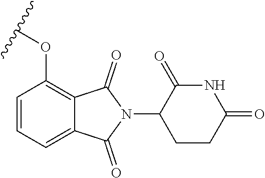 [SMILES: *Oc1cccc2c1C(=O)N(C1CCC(=O)NC1=O)C2=O] |
| 1385 | 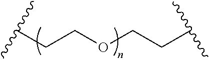 | 4 | 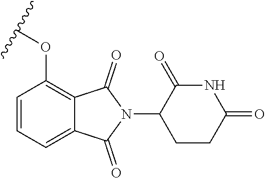 [SMILES: *Oc1cccc2c1C(=O)N(C1CCC(=O)NC1=O)C2=O] |
| 1386 | 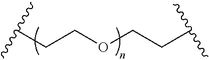 | 5 | 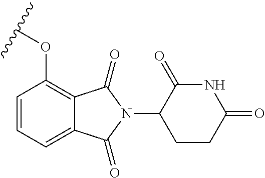 [SMILES: *Oc1cccc2c1C(=O)N(C1CCC(=O)NC1=O)C2=O] |
| 1387 | 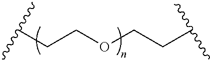 | 1 | 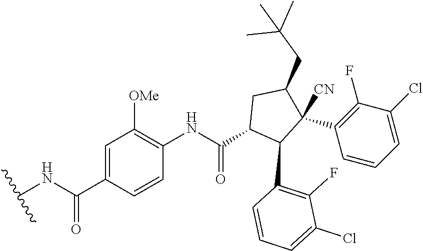 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1388 | 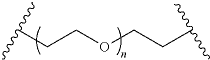 | 2 | 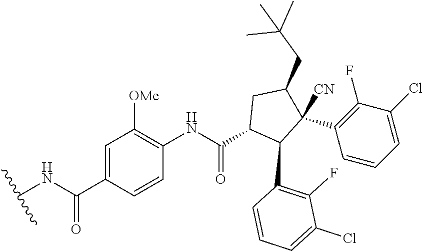 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1389 | 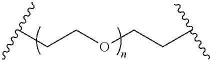 | 3 | 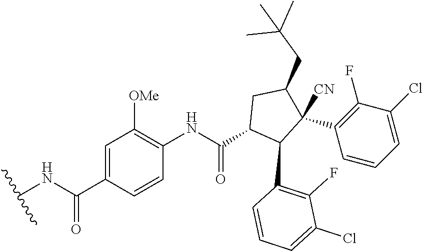 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1390 | 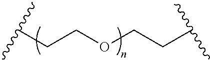 | 4 | 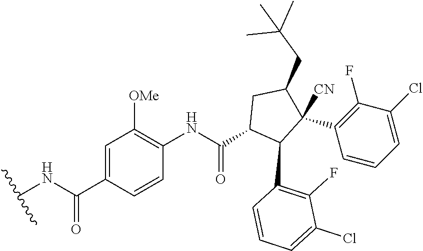 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1391 | 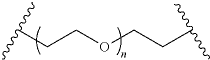 | 5 | 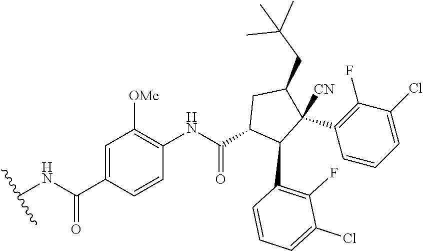 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1392 | 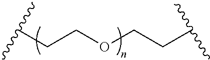 | 1 | 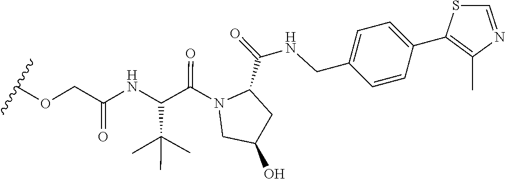 |
| 1393 | 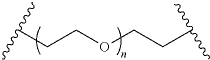 | 2 | 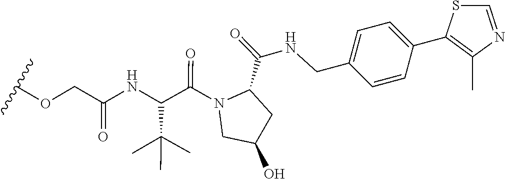 |
| 1394 | 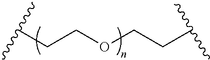 | 3 | 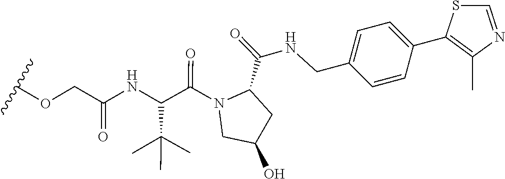 |
| 1395 | 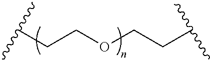 | 4 | 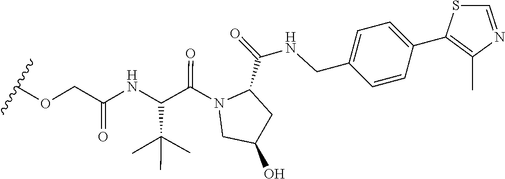 |
| 1396 | 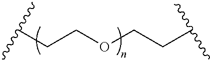 | 5 | 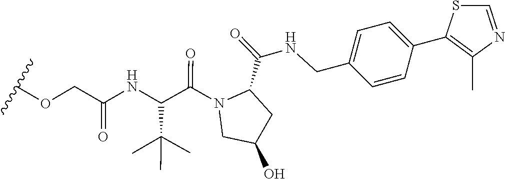 |
| 1397 | 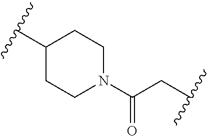 [SMILES: *CC(=O)N1CCC(*)CC1] | — | 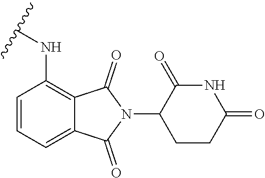 [SMILES: *Nc1cccc2c1C(=O)N(C1CCC(=O)NC1=O)C2=O] |
| 1398 | 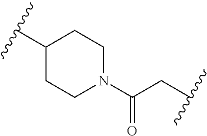 [SMILES: *CC(=O)N1CCC(*)CC1] | — | 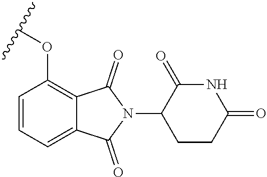 [SMILES: *Oc1cccc2c1C(=O)N(C1CCC(=O)NC1=O)C2=O] |
| 1399 | 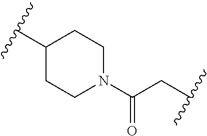 [SMILES: *CC(=O)N1CCC(*)CC1] | — | 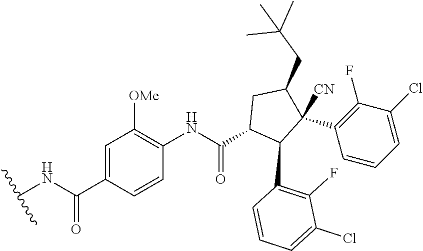 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1400 | 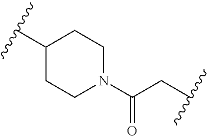 [SMILES: *CC(=O)N1CCC(*)CC1] | — | 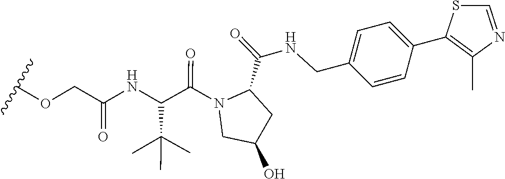 |
| 1401 | 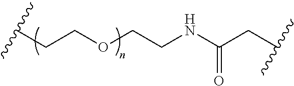 | 1 | 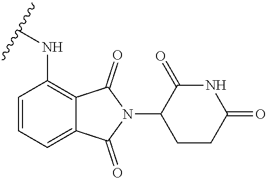 [SMILES: *Nc1cccc2c1C(=O)N(C1CCC(=O)NC1=O)C2=O] |
| 1402 | 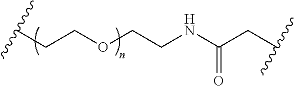 | 2 | 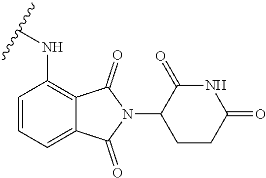 [SMILES: *Nc1cccc2c1C(=O)N(C1CCC(=O)NC1=O)C2=O] |
| 1403 | 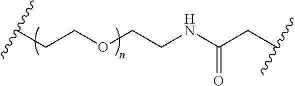 | 3 | 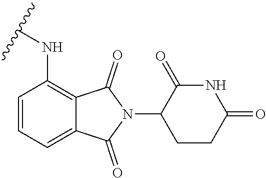 [SMILES: *Nc1cccc2c1C(=O)N(C1CCC(=O)NC1=O)C2=O] |
| 1404 | 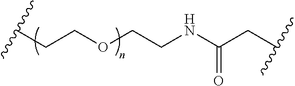 | 4 | 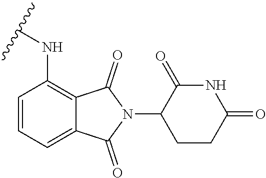 [SMILES: *Nc1cccc2c1C(=O)N(C1CCC(=O)NC1=O)C2=O] |
| 1405 | 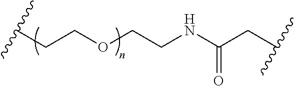 | 5 | 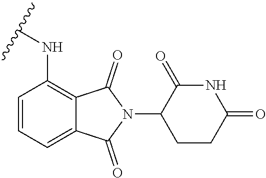 [SMILES: *Nc1cccc2c1C(=O)N(C1CCC(=O)NC1=O)C2=O] |
| 1406 | 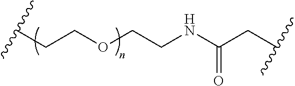 | 1 | 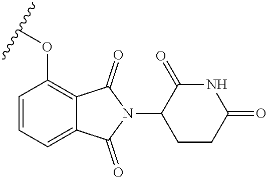 [SMILES: *Oc1cccc2c1C(=O)N(C1CCC(=O)NC1=O)C2=O] |
| 1407 | 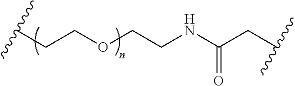 | 2 | 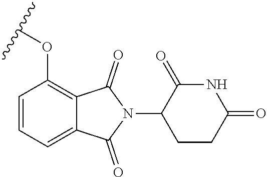 [SMILES: *Oc1cccc2c1C(=O)N(C1CCC(=O)NC1=O)C2=O] |
| 1408 | 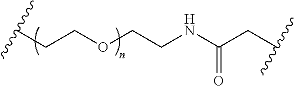 | 3 | 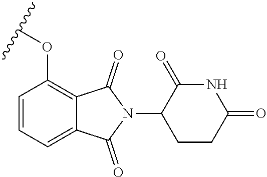 [SMILES: *Oc1cccc2c1C(=O)N(C1CCC(=O)NC1=O)C2=O] |
| 1409 | 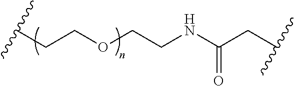 | 4 | 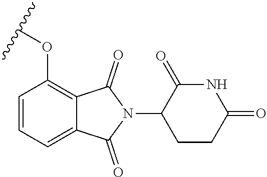 [SMILES: *Oc1cccc2c1C(=O)N(C1CCC(=O)NC1=O)C2=O] |
| 1410 | 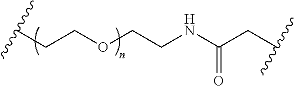 | 5 | 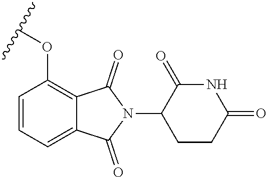 [SMILES: *Oc1cccc2c1C(=O)N(C1CCC(=O)NC1=O)C2=O] |
| 1411 | 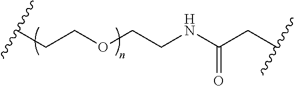 | 1 | 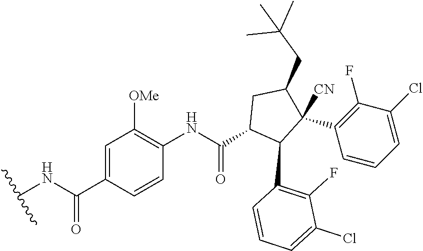 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1412 | 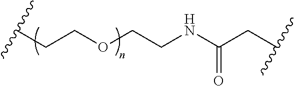 | 2 | 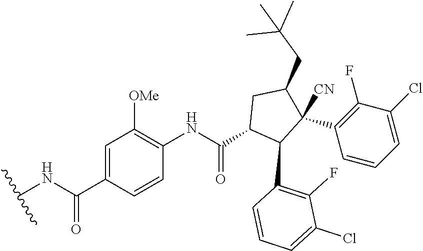 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1413 | 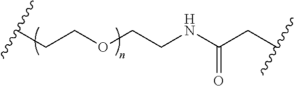 | 3 | 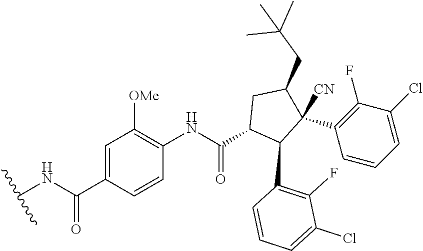 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1414 | 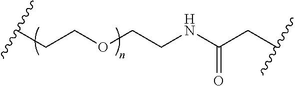 | 4 | 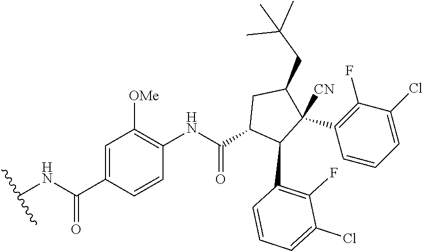 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1415 | 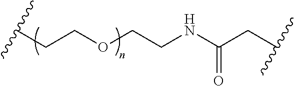 | 5 | 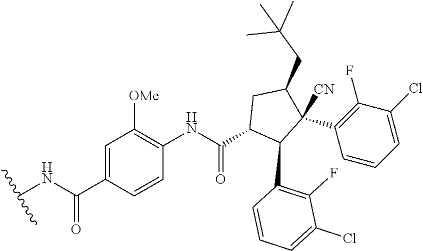 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1416 | 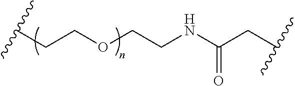 | 1 | 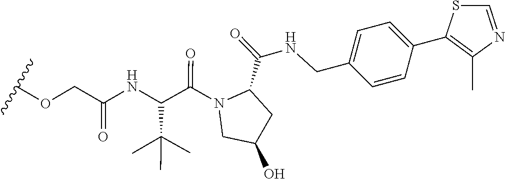 |
| 1417 | 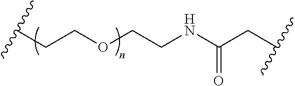 | 2 | 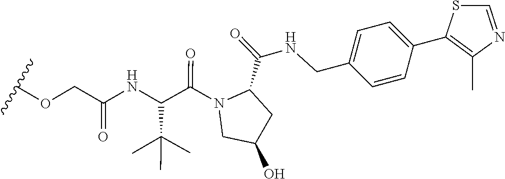 |
| 1418 | 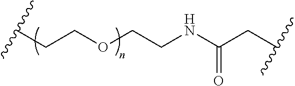 | 3 | 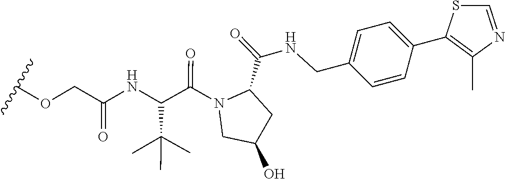 |
| 1419 | 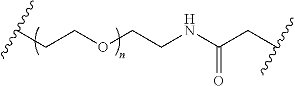 | 4 | 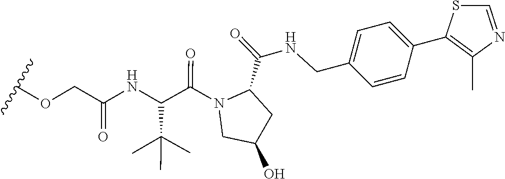 |
| 1420 | 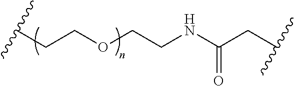 | 5 | 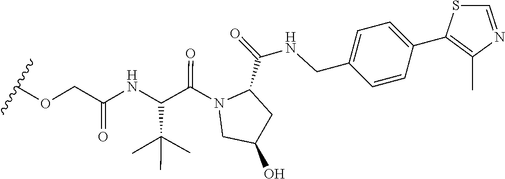 |
| 1421 | 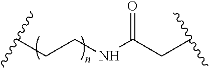 | 1 | 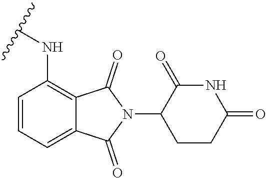 [SMILES: *Nc1cccc2c1C(=O)N(C1CCC(=O)NC1=O)C2=O] |
| 1422 | 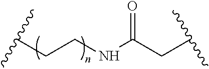 | 2 | 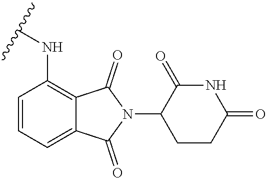 [SMILES: *Nc1cccc2c1C(=O)N(C1CCC(=O)NC1=O)C2=O] |
| 1423 | 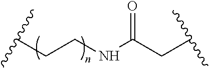 | 3 | 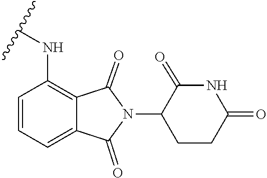 [SMILES: *Nc1cccc2c1C(=O)N(C1CCC(=O)NC1=O)C2=O] |
| 1424 | 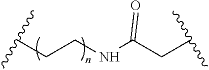 | 4 | 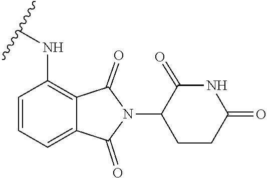 [SMILES: *Nc1cccc2c1C(=O)N(C1CCC(=O)NC1=O)C2=O] |
| 1425 | 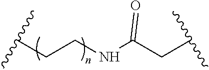 | 5 | 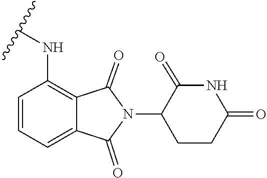 [SMILES: *Nc1cccc2c1C(=O)N(C1CCC(=O)NC1=O)C2=O] |
| 1426 | 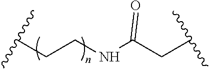 | 1 | 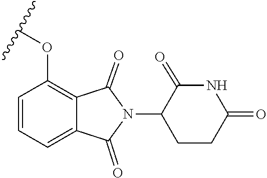 [SMILES: *Oc1cccc2c1C(=O)N(C1CCC(=O)NC1=O)C2=O] |
| 1427 | 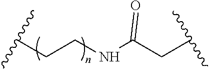 | 2 | 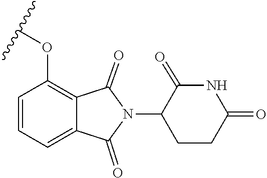 [SMILES: *Oc1cccc2c1C(=O)N(C1CCC(=O)NC1=O)C2=O] |
| 1428 | 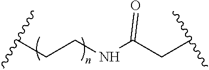 | 3 | 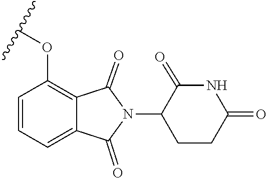 [SMILES: *Oc1cccc2c1C(=O)N(C1CCC(=O)NC1=O)C2=O] |
| 1429 | 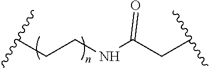 | 4 | 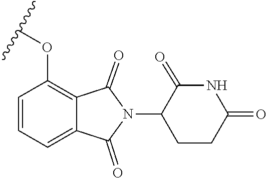 [SMILES: *Oc1cccc2c1C(=O)N(C1CCC(=O)NC1=O)C2=O] |
| 1430 | 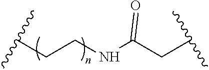 | 5 | 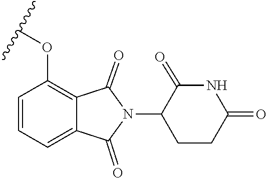 [SMILES: *Oc1cccc2c1C(=O)N(C1CCC(=O)NC1=O)C2=O] |
| 1431 | 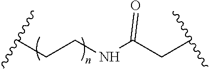 | 1 | 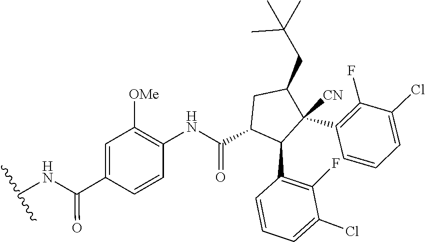 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1432 | 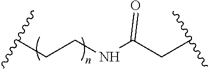 | 2 | 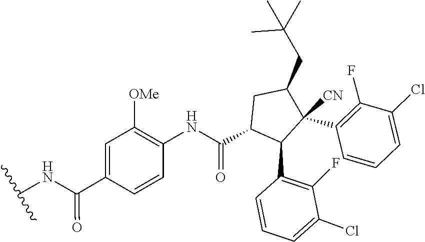 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1433 | 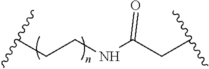 | 3 | 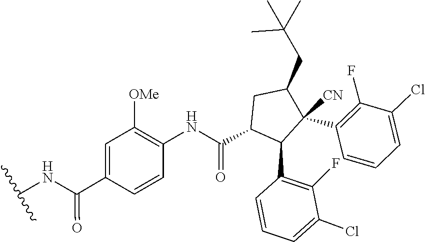 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1434 | 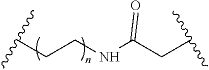 | 4 | 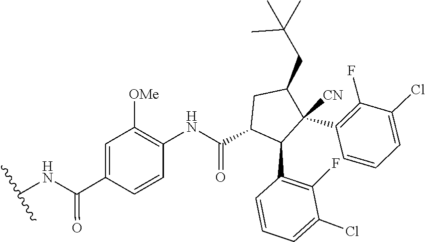 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1435 | 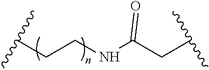 | 5 | 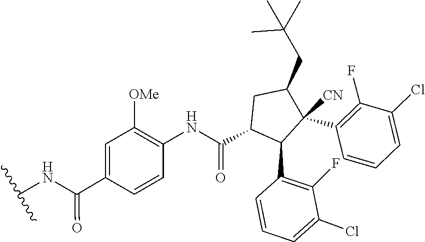 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1436 | 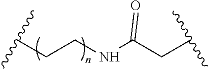 | 1 | 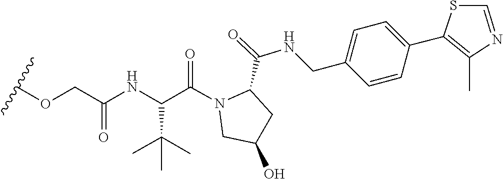 |
| 1437 | 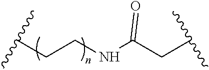 | 2 | 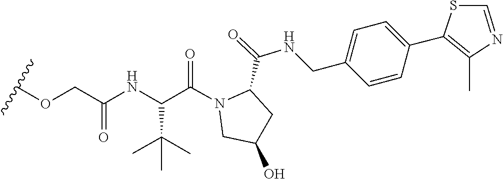 |
| 1438 | 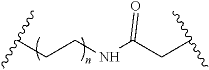 | 3 | 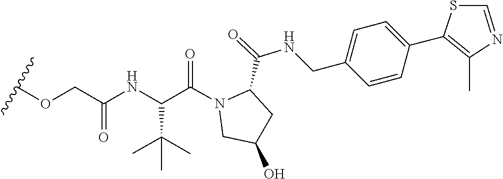 |
| 1439 | 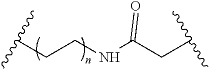 | 4 | 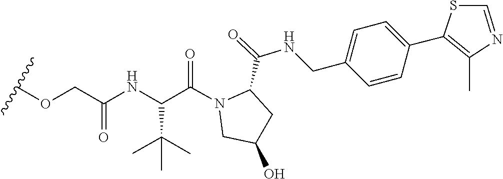 |
| 1440 | 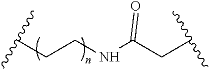 | 5 | 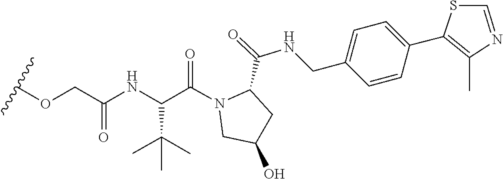 |
| 1441 | 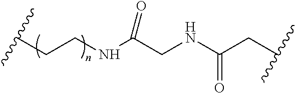 | 1 | 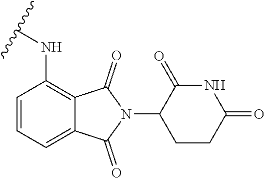 [SMILES: *Nc1cccc2c1C(=O)N(C1CCC(=O)NC1=O)C2=O] |
| 1442 | 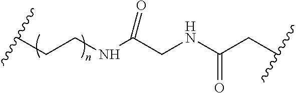 | 2 | 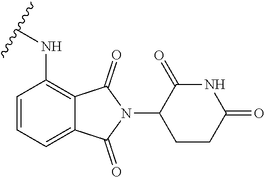 [SMILES: *Nc1cccc2c1C(=O)N(C1CCC(=O)NC1=O)C2=O] |
| 1443 | 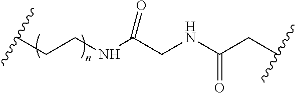 | 3 | 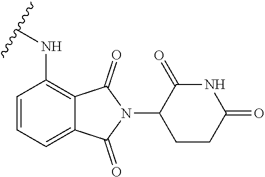 [SMILES: *Nc1cccc2c1C(=O)N(C1CCC(=O)NC1=O)C2=O] |
| 1444 | 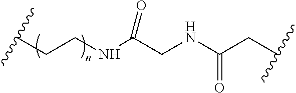 | 4 | 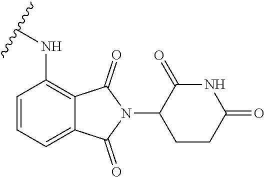 [SMILES: *Nc1cccc2c1C(=O)N(C1CCC(=O)NC1=O)C2=O] |
| 1445 | 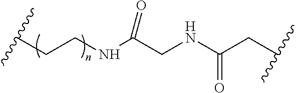 | 5 | 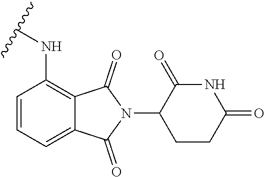 [SMILES: *Nc1cccc2c1C(=O)N(C1CCC(=O)NC1=O)C2=O] |
| 1446 | 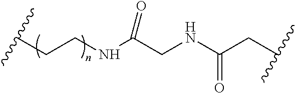 | 1 | 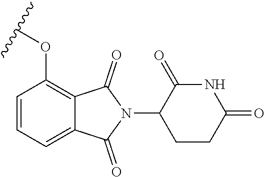 [SMILES: *Oc1cccc2c1C(=O)N(C1CCC(=O)NC1=O)C2=O] |
| 1447 | 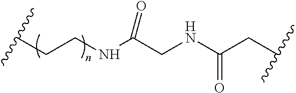 | 2 | 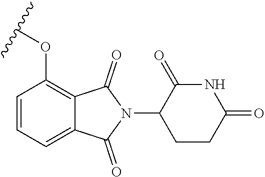 [SMILES: *Oc1cccc2c1C(=O)N(C1CCC(=O)NC1=O)C2=O] |
| 1448 | 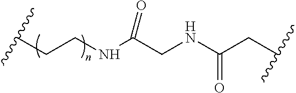 | 3 | 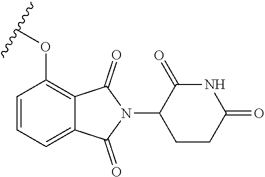 [SMILES: *Oc1cccc2c1C(=O)N(C1CCC(=O)NC1=O)C2=O] |
| 1449 | 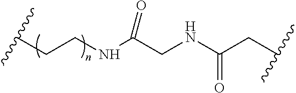 | 4 | 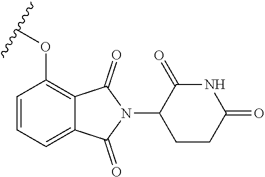 [SMILES: *Oc1cccc2c1C(=O)N(C1CCC(=O)NC1=O)C2=O] |
| 1450 | 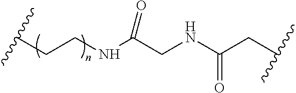 | 5 | 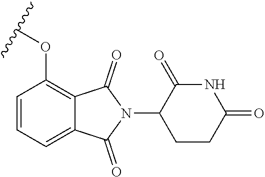 [SMILES: *Oc1cccc2c1C(=O)N(C1CCC(=O)NC1=O)C2=O] |
| 1451 | 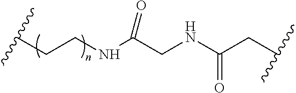 | 1 | 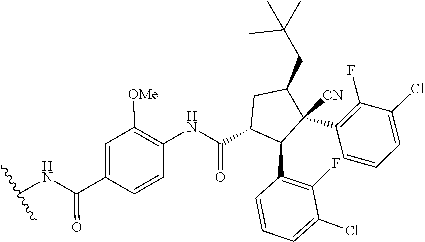 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1452 | 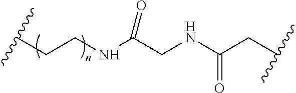 | 2 | 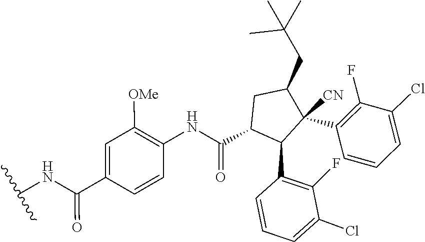 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1453 | 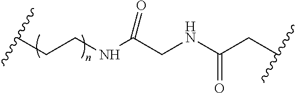 | 3 | 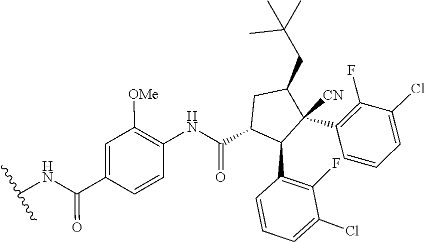 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1454 | 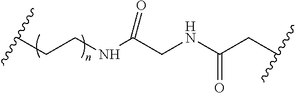 | 4 | 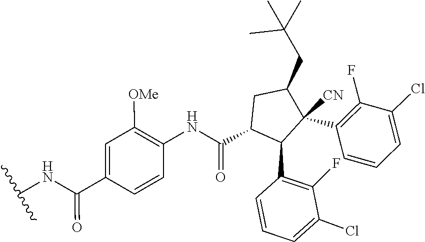 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1455 | 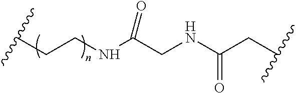 | 5 | 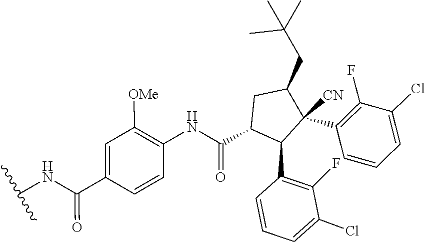 [SMILES: *NC(=O)c1ccc(NC(=O)[C@@H]2C[C@@H](CC(C)(C)C)[C@](C#N)(c3cccc(Cl)c3F)[C@H]2c2cccc(Cl)c2F)c(OC)c1] |
| 1456 | 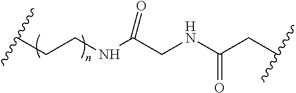 | 1 | 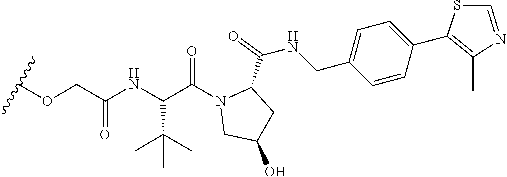 |
| 1457 | 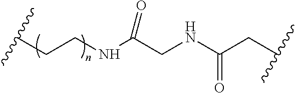 | 2 | 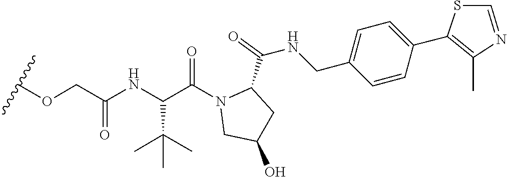 |
| 1458 | 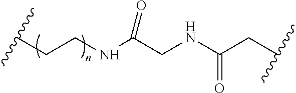 | 3 | 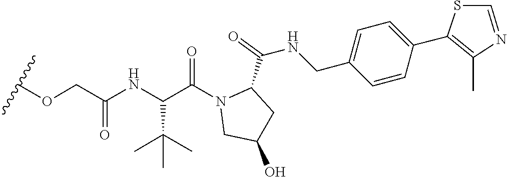 |
| 1459 | 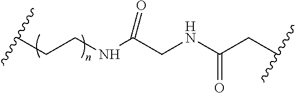 | 4 | 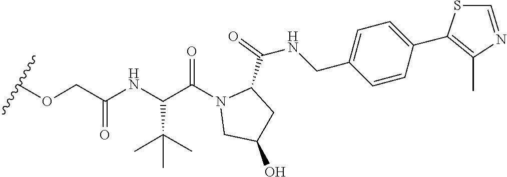 |
| 1460 | 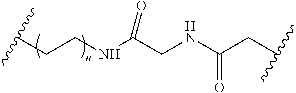 | 5 | 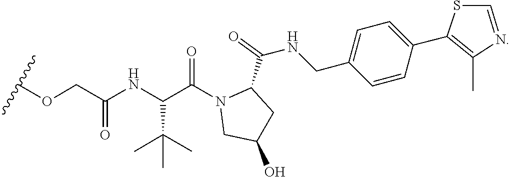 |

26. A pharmaceutical composition comprising one or more compounds of any one of claims 1-25 or a pharmaceutically acceptable salt thereof, and a physiologically compatible carrier medium.

27. A pharmaceutical composition for treating or preventing a disease or disorder alleviated by inhibiting and/or indirectly inhibiting Mcl-1 protein activity, the pharmaceutical composition comprising one or more compounds according to any one of claims 1-25, or a pharmaceutically acceptable salt thereof, and a physiologically compatible carrier medium.

28. The pharmaceutical composition of claim 27, wherein the disease or disorder is cancer.

29. The pharmaceutical composition of claim 28, wherein the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

30. The pharmaceutical composition of claim 28, wherein the cancer is a blood cancer.

31. The pharmaceutical composition of claim 30, wherein the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

32. The pharmaceutical composition of any one of claims 28-31, wherein the cancer is acute myeloid leukemia (AML).

33. A pharmaceutical composition for treating or preventing acute myeloid leukemia (AML), the pharmaceutical composition comprising one or more compounds according to any one of claims 1-25, or a pharmaceutically acceptable salt thereof, and a physiologically compatible carrier medium.

34. A method of treating or preventing a disease or disorder alleviated by inhibiting and/or indirectly inhibiting Mcl-1 protein activity in a patient in need of said treatment or prevention, the method comprising administering a therapeutically effective amount of one or more compounds of any one of claims 1-25, or a pharmaceutically acceptable salt thereof.

35. The method of claim 34, wherein the disease or disorder is cancer.

36. The method of claim 35, wherein the cancer is selected from acute myeloid leukemia (AML), pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

37. The method of claim 35, wherein the cancer is a blood cancer.

38. The method of claim 37, wherein the blood cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, and primary central nervous system lymphoma.

39. The method of any one of claims 35-38, wherein the cancer is acute myeloid leukemia (AML).

40. A method of treating or preventing acute myeloid leukemia (AML) in a patient in need of said treatment or prevention, the method comprising administering a therapeutically effective amount of one or more compounds of any one of claims 1-25, or a pharmaceutically acceptable salt thereof.
